(19) United States
(12) Patent Application Publication
(10) Pub. No.: US 2026/0108570 A1
(43) Pub. Date: Apr. 23, 2026

(54) BACTERIOPHAGE COMPOSITIONS AND METHODS FOR TREATMENT OF BACTERIAL INFECTIONS

(71) Applicant: THE UNIVERSITY OF CHICAGO, Chicago, IL (US)

(72) Inventors: Mark MIMEE, Chicago, IL (US); Ella ROTMAN, Chicago, IL (US)

(73) Assignee: THE UNIVERSITY OF CHICAGO, Chicago, IL (US)

(21) Appl. No.: 19/100,379

(22) PCT Filed: Aug. 1, 2023
(86) PCT No.: PCT/US2023/071414

§ 371 Date: Jan. 31, 2025

Related U.S. Application Data
(60) Provisional application No. 63/370,075, filed on Aug. 1, 2022.

(51) Int. Cl. A61K 35/76; A61P 43/00; C12N 7/02

(52) U.S. Cl. CPC A61K 35/76; A61P 43/00; C12N 7/02

(57) ABSTRACT

Disclosed are compositions, devices, kits, and methods for treatment of Enterobacteriaceae infection. Aspects of the present disclosure are directed to bacteriophage compositions comprising one or more isolated bacteriophage comprising a bacterio-phage sequence that is at least 99% identical to any one of SEQ ID NO:1-74. Further disclosed are devices and kits comprising such compositions and methods for use of such compositions in treatment and prevention of pathogenic Klebsiella infection.

### CROSS-REFERENCE TO RELATED APPLICATIONS

This application is a national phase application under 35 U.S.C. § 371 of International Application No. PCT/US2023/071414, filed Aug. 1, 2023, which claims priority to U.S. Provisional Application Ser. No. 63/370,075, filed Aug. 1, 2022, each of which are incorporated by reference herein in their entirety.

### SEQUENCE LISTING

The instant application contains a Sequence Listing which has been submitted in XML format and is hereby incorporated by reference in its entirety. Said XML copy, created on Sep. 12, 2023, is named “ARCDP0784 Sequence Listing (Sep. 12, 2023 Update).xml” and is 8,453,551 byes in size.

### BACKGROUND

### I. Technical Field

Aspects of this disclosure relate to at least the fields of microbiology and virology.

### II. Background

Multidrug-resistant (MDR) bacteria infect millions of people around the world, yearly. Many of these infections caused by these bacteria have become untreatable due to resistance to last resort antibiotics. Exacerbating this crisis, the pipeline for antibiotic development is slow and resistant strains rapidly develop in the wake of new drugs. The family Enterobacteriaceae provides an urgent threat of MDR infections, due in part to strains of Klebsiella, which can cause infection in the lungs, bloodstream, and urinary tract. Klebsiella species with the ability to produce extended-spectrum beta-lactamases (ESBL) are resistant to virtually all beta-lactam antibiotics, except carbapenems. Other frequent resistance targets include aminoglycosides, fluoroquinolones, tetracyclines, chloramphenicol, and trimethoprim/sulfamethoxazole.

Infection with carbapenem-resistant Enterobacteriaceae or carbapenemase-producing Enterobacteriaceae is emerging as an important challenge in health-care settings. One of many carbapenem-resistant Enterobacteriaceae is carbapenem-resistant Klebsiella pneumoniae. Over the past 10 years, a progressive increase in carbapenem-resistant Klebsiella pneumoniae has been seen worldwide. Carbapenem-resistant Klebsiella pneumoniae is resistant to almost all available antimicrobial agents, and infections with carbapenem-resistant Klebsiella pneumoniae have caused high rates of morbidity and mortality, in particular among persons with prolonged hospitalization and those critically ill and exposed to invasive devices (e.g., ventilators or central venous catheters).

Also complicating Klebsiella treatment has been the emergence of hypervirulent Klebsiella, a Klebsiella variant that is significantly more virulent than classical Klebsiella. While classical Klebsiella is an opportunistic pathogen responsible for nosocomial infections that usually affect immunocompromised patients, hypervirulent Klebsiella is clinically more concerning since it also causes disease in healthy individuals and can infect virtually every site of the body. Hypervirulent Klebsiella overproduce capsule components and siderophores for iron acquisition, among other factors, and such strains can acquire resistance plasmids and become multi-resistant to a variety of antibiotics, including carbapenems.

Carbapenem is often used as a drug of last resort when battling resistant bacterial strains, and mutant strains could result in infections for which healthcare professionals can do very little, if anything, to treat. Thus, new treatments are needed to contain the threat of MDR bacteria, including Klebsiella.

A promising response to MDR infections is bacteriophage (phage) therapy. Phages are environmentally ubiquitous, host-specific, and effective viruses that can infect MDR bacterial strains. Importantly, they have been shown to be safe and effective in animal and compassionate-use human trials. Because they use the replication machinery of their bacterial host, phage mutation rates are directly influenced by those of the host; as such, phages may rapidly adapt to target strains of bacteria.

There exists a need for new and improved methods and compositions for treatment of bacterial infections, including Klebsiella infections such as Klebsiella pneumoniae, Klebsiella quasipneumoniae, and Klebsiella variicola.

### SUMMARY

Aspects of the disclosure include bacteriophages, bacteriophage compositions, bacteriophage pharmaceutical formulations, kits, devices, medical devices, therapeutic devices, polynucleotides, methods for preparing a device, methods for treatment of a Klebsiella infection, and methods for preventing a Klebsiella infection.

In some aspects, there are compositions or pharmaceutical compositions comprising a collection of isolated bacteriophage comprising an Ackermannviridae bacteriophage, a Tevenvirinae bacteriophage, a Slopekvirus bacteriophage, a Siphoviridae bacteriophage, a Demerecviridae bacteriophage, or any combination thereof. In some aspects, the Ackermannviridae bacteriophage is an isolated bacteriophage comprising a bacteriophage sequence that is at least 99% identical to SEQ ID NO:1 or SEQ ID NO:37. In some aspects, the Tevenvirinae bacteriophage is an isolated bacteriophage comprising a bacteriophage sequence that is at least 99% identical to SEQ ID NO:2 or SEQ ID NO:40. In some aspects, the Slopekvirus bacteriophage is an isolated bacteriophage comprising a bacteriophage sequence that is at least 99% identical to SEQ ID NO:3 or SEQ ID NO:41. In some aspects, the Siphoviridae bacteriophage is an isolated bacteriophage comprising a bacteriophage sequence that is at least 99% identical to SEQ ID NO:4 or SEQ ID NO:52. In some aspects, the Demerecviridae bacteriophage is an isolated bacteriophage comprising a bacteriophage sequence that is at least 99% identical to SEQ ID NO:5 or SEQ ID NO:56. In some aspects, the Ackermannviridae bacteriophage is ΦER12; the Tevenvirinae bacteriophage is ΦER15; the Slopekvirus bacteriophage is ΦER16; the Siphoviridae bacteriophage is ΦER39; and the Demerecviridae bacteriophage is ΦER46.

In some aspects, there are compositions or pharmaceutical compositions comprising a collection of isolated bacteriophage comprising an Autographiviridae bacteriophage, a Tevenvirinae bacteriophage, one or more Slopekvirus bacteriophage, a Purpuraviridae bacteriophage, or any combination thereof. In some aspects, the Autographiviridae bacteriophage is an isolated bacteriophage comprising a bacteriophage sequence that is at least 99% identical to SEQ ID NO:74. In some aspects, the Tevenvirinae bacteriophage is an isolated bacteriophage comprising a bacteriophage sequence that is at least 99% identical to SEQ ID NO: 36. In some aspects, the one or more Slopekvirus bacteriophage comprise two Slopekvirus bacteriophage, and the Slopekvirus are isolated bacteriophage comprising a bacteriophage sequence that is at least 99% identical to SEQ ID NO:47 and SEQ ID NO:49. In some aspects, the Purpuraviridae bacteriophage is an isolated bacteriophage comprising a bacteriophage sequence that is at least 99% identical to SEQ ID NO:32. In some aspects, the Autographiviridae bacteriophage is ΦMM9; the Tevenvirinae bacteriophage is ΦER11; the one or more Slopekvirus bacteriophage comprise two Slopekvirus, and the two Slopekvirus are ER27 and ΦER36; and the Purpuraviridae bacteriophage is ΦER3.

In some aspects, there are compositions or pharmaceutical compositions comprising a collection of isolated bacteriophage comprising an Autographiviridae bacteriophage, a Tevenvirinae bacteriophage, one or more Slopekvirus bacteriophage, a Demerecviridae bacteriophage, a Purpuraviridae bacteriophage, or any combination thereof. In some aspects, the Autographiviridae bacteriophage is an isolated bacteriophage comprising a bacteriophage sequence that is at least 99% identical to SEQ ID NO:53. In some aspects, the Tevenvirinae bacteriophage is an isolated bacteriophage comprising a bacteriophage sequence that is at least 99% identical to SEQ ID NO:2 or SEQ ID NO:40. In some aspects, the Slopekvirus bacteriophage is an isolated bacteriophage comprising a bacteriophage sequence that is at least 99% identical to SEQ ID NO:47. In some aspects, the Demerecviridae bacteriophage is an isolated bacteriophage comprising a bacteriophage sequence that is at least 99% identical to SEQ ID NO:5 or SEQ ID NO:46. In some aspects, the Purpuraviridae bacteriophage is an isolated bacteriophage comprising a bacteriophage sequence that is at least 99% identical to SEQ ID NO:72. In some aspects, the Autographiviridae bacteriophage is ΦER43; the Tevenvirinae bacteriophage is ΦER15; the Slopekvirus bacteriophage is ΦER27; the Demerecviridae bacteriophage is ΦER46; and the Purpuraviridae bacteriophage is ΦMM7.

In some aspects, there are compositions or pharmaceutical compositions comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 3,6 37, 38, 39, 40 or more different isolated bacteriophage (or any range derivable therein) each comprising a bacteriophage sequence or an entire nucleic acid sequence that is at least, at most, equal to, or between any two of 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 99.0%, 99.1%, 99.2%, 99.3%, 99.4%, 99.5%, 99.6%, 99.7%, 99.8%, or 99.9% identical to (or any range derivable therein) any one of SEQ ID NO:1-74. In specific aspects, the SEQ ID NO(s) is/are any one or any combination of SEQ ID NO:1 OR SEQ ID NO:37, SEQ ID NO: 2 or SEQ ID NO:40, SEQ ID NO:3 or SEQ ID NO:41, SEQ ID NO:4 or SEQ ID NO:52, and SEQ ID NO:5 or SEQ ID NO:56. In specific aspects, the SEQ ID NO(s) is/are any one or any combination of SEQ ID NO:8 or SEQ ID NO:32, SEQ ID NO: 14 or SEQ ID NO:36, SEQ ID NO: 20 or SEQ ID NO:47, SEQ ID NO:22 or SEQ ID NO:49, and SEQ ID NO: 74. In specific aspects, the SEQ ID NO(s) is/are any one or any combination of SEQ ID NO:2 or SEQ ID NO: 40, SEQ ID NO:5 or SEQ ID NO:46, SEQ ID NO:20 or SEQ ID NO:47, SEQ ID NO:25 or SEQ ID NO:53, and SEQ ID NO:72.

Additionally or alternatively, in some aspects, there are compositions or pharmaceutical compositions comprising at least one isolated bacteriophage comprising a bacteriophage sequence or an entire nucleic acid sequence that has at least 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000 or more contiguous nucleotides from any one of SEQ ID NO:1-74. In specific aspects, the SEQ ID NO(s) is/are any one or any combination of SEQ ID NO:1 OR SEQ ID NO:37, SEQ ID NO:2 or SEQ ID NO:40, SEQ ID NO:3 or SEQ ID NO:41, SEQ ID NO:4 or SEQ ID NO:52, and SEQ ID NO:5 or SEQ ID NO:56. In specific aspects, the SEQ ID NO(s) is/are any one or any combination of SEQ ID NO:8 or SEQ ID NO:32, SEQ ID NO: 14 or SEQ ID NO:36, SEQ ID NO: 20 or SEQ ID NO:47, SEQ ID NO:22 or SEQ ID NO:49, and SEQ ID NO: 74. In specific aspects, the SEQ ID NO(s) is/are any one or any combination of SEQ ID NO:2 or SEQ ID NO: 40, SEQ ID NO:5 or SEQ ID NO:46, SEQ ID NO:20 or SEQ ID NO:47, SEQ ID NO:25 or SEQ ID NO:53, and SEQ ID NO: 72.

Additionally or alternatively, in some aspects, there are compositions or pharmaceutical compositions comprising at least one isolated bacteriophage comprising a bacteriophage sequence or an entire nucleic acid sequence that has, has at most, has at least, or has between any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000 nucleotide differences from any one of SEQ ID NO:1-74. In specific aspects, the SEQ ID NO(s) is/are any one or any combination of SEQ ID NO:1 OR SEQ ID NO: 37, SEQ ID NO:2 or SEQ ID NO:40, SEQ ID NO:3 or SEQ ID NO:41, SEQ ID NO:4 or SEQ ID NO:52, and SEQ ID NO:5 or SEQ ID NO:56. In specific aspects, the SEQ ID NO(s) is/are any one or any combination of SEQ ID NO:8 or SEQ ID NO:32, SEQ ID NO: 14 or SEQ ID NO: 36, SEQ ID NO:20 or SEQ ID NO:47, SEQ ID NO:22 or SEQ ID NO:49, and SEQ ID NO: 74. In specific aspects, the SEQ ID NO(s) is/are any one or any combination of SEQ ID NO: 2 or SEQ ID NO:40, SEQ ID NO:5 or SEQ ID NO:46, SEQ ID NO:20 or SEQ ID NO:47, SEQ ID NO:25 or SEQ ID NO:53, and SEQ ID NO: 72.

It is contemplated that in some aspects, a composition has a nucleic acid sequence that has a percent identity with respect to a SEQ ID NO and also at least a certain number of contiguous nucleotides of that same SEQ ID NO, such as at least 99.1% identity and at least 100,000 contiguous nucleotides from SEQ ID NO:1, for example. The term “bacteriophage sequence” refers to a sequence from bacteriophage and does not include a nucleotide sequence greater than 20 contiguous nucleotides that is not from bacteriophage. Such non-bacteriophage sequences would be heterologous, and in aspects of the disclosure, they could be used to purify, select, and/or provide additional functionality to the bacteriophage. The heterologous sequence encodes a peptide or polypeptide in some aspects.

In some aspects, there is no more than one isolated bacteriophage in the composition, wherein the bacteriophage sequence or the entire nucleic acid sequence comprises all or part of any of SEQ ID NO: 1-74. In specific aspects, the SEQ ID NO(s) is/are any one or any combination of SEQ ID NO: 1 OR SEQ ID NO:37, SEQ ID NO:2 or SEQ ID NO:40, SEQ ID NO: 3 or SEQ ID NO:41, SEQ ID NO:4 or SEQ ID NO:52, and SEQ ID NO:5 or SEQ ID NO: 56. In specific aspects, the SEQ ID NO(s) is/are any one or any combination of SEQ ID NO: 8 or SEQ ID NO:32, SEQ ID NO: 14 or SEQ ID NO:36, SEQ ID NO:20 or SEQ ID NO:47, SEQ ID NO:22 or SEQ ID NO:49, and SEQ ID NO: 74. In specific aspects, the SEQ ID NO(s) is/are any one or any combination of SEQ ID NO:2 or SEQ ID NO:40, SEQ ID NO:5 or SEQ ID NO: 46, SEQ ID NO:20 or SEQ ID NO:47, SEQ ID NO:25 or SEQ ID NO:53, and SEQ ID NO: 72. In additional aspects at least, equal to, or no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 3,6 37, 38, 39, 40 or more different isolated bacteriophage (or any range derivable therein) in a composition or used in a method described herein. In some aspects, a composition comprises or a methods comprises administering at least, equal to, or no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 3,6 37, 38, 39, 40 or more different isolated bacteriophage (or any range derivable therein), wherein the bacteriophage comprises all or part of any of SEQ ID NO:1-74. In specific aspects, the SEQ ID NO(s) is/are any one or any combination of SEQ ID NO:1 OR SEQ ID NO:37, SEQ ID NO:2 or SEQ ID NO:40, SEQ ID NO:3 or SEQ ID NO:41, SEQ ID NO:4 or SEQ ID NO: 52, and SEQ ID NO:5 or SEQ ID NO:56. In specific aspects, the SEQ ID NO(s) is/are any one or any combination of SEQ ID NO:8 or SEQ ID NO:32, SEQ ID NO: 14 or SEQ ID NO:36, SEQ ID NO:20 or SEQ ID NO:47, SEQ ID NO:22 or SEQ ID NO:49, and SEQ ID NO: 74. In specific aspects, the SEQ ID NO(s) is/are any one or any combination of SEQ ID NO:2 or SEQ ID NO: 40, SEQ ID NO:5 or SEQ ID NO:46, SEQ ID NO:20 or SEQ ID NO:47, SEQ ID NO:25 or SEQ ID NO:53, and SEQ ID NO: 72.

In a specific aspect, the composition or method comprises two different isolated bacteriophage. In a specific aspect, the composition or method comprises three different isolated bacteriophage. In a specific aspect, the composition or method comprises four different isolated bacteriophage. In a specific aspect, the composition or method comprises five different isolated bacteriophage. It is specifically contemplated that the different isolated bacteriophage may be similar, such as evolved from the same sequence, and/or have, have at least, have at most, or have between any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 600, 700, 800, 900, 1000, 2000, 3000, 4000, 5000, 6000, 7000, 8000, 9000, 10000, 20000, 30000, 40000, 50000 or more nucleotide differences between any two different isolated bacteriophage. Moreover, it is specifically contemplated that any composition may contain, or any method described herein may involve, 1, 2, 3, 4, 5, 6, 7, 8, 9, 10, or more bacteriophage that is/are not derived from any of SEQ ID NO: 1-74 or from a bacteriophage deposit corresponding to the bacteriophage with that SEQ ID NO.

Any composition may comprise about 10⁴, 10⁵, 10⁶, 10⁷, 10⁸, 10⁹, 10¹⁰, 10¹¹, 10¹², 10¹³, 10¹⁴, 10¹⁵ or more plaque forming units (PFUs) or viral particles. In some aspects, a composition includes or a method includes a step of administering about 10⁸ to about 10¹² plaque forming units (PFU) per milliliter (PFU/ml) of isolated bacteriophages.

In some aspects, a composition comprises 1, 2, 3, 4, or 5 different isolated bacteriophage, each comprising a bacteriophage sequence that is at least 99%, 99.1, 99.2%, 99.3%, 99.4%, 99.5%, 99.6%, 99.7%, 99.8%, or 99.9% or more identical to any one of SEQ ID NO: 1 or SEQ ID NO:37 (ΦER12), SEQ ID NO:2 or SEQ ID NO:40 (ΦER15), SEQ ID NO: 3 or SEQ ID NO:41 (ΦER16), SEQ ID NO:4 or SEQ ID NO:52 (ΦER39), or SEQ ID NO: 5 or SEQ ID NO:56 (ΦER46). In some aspects, a composition with at least two different isolated bacteriophage, wherein each bacteriophage comprises a bacteriophage sequence with no more than 20, 15, 10, 5 or fewer nucleotide changes in any one of SEQ ID NO: 1 or SEQ ID NO: 37, SEQ ID NO:2 or SEQ ID NO:40, SEQ ID NO:3 or SEQ ID NO:41, SEQ ID NO:4 or SEQ ID NO:52, or SEQ ID NO:5 or SEQ ID NO:56.

In some aspects, a composition comprises 1, 2, 3, 4, or 5 different isolated bacteriophage each comprising a bacteriophage sequence that is at least 99%, 99.1, 99.2%, 99.3%, 99.4%, 99.5%, 99.6%, 99.7%, 99.8%, or 99.9% or more identical to any one of SEQ ID NO: 8 or SEQ ID NO:32 (ΦER3), SEQ ID NO:14 or SEQ ID NO:36 (ΦER11), SEQ ID NO: 20 or SEQ ID NO:47 (ΦER27), SEQ ID NO:22 or SEQ ID NO:49 (ΦER36), and SEQ ID NO: 74 (ΦMM9). In some aspects, a composition with at least two different isolated bacteriophage, wherein each bacteriophage comprises a bacteriophage sequence with no more than 20, 15, 10, 5 or fewer nucleotide changes in any one of SEQ ID NO:8 or SEQ ID NO:32, SEQ ID NO: 14 or SEQ ID NO:36, SEQ ID NO:20 or SEQ ID NO:47, SEQ ID NO:22 or SEQ ID NO: 49, and SEQ ID NO:74.

In some aspects, a composition comprises 1, 2, 3, 4, or 5 different isolated bacteriophage each comprising a bacteriophage sequence that is at least 99%, 99.1, 99.2%, 99.3%, 99.4%, 99.5%, 99.6%, 99.7%, 99.8%, or 99.9% or more identical to any one of SEQ ID NO: 2 or SEQ ID NO:40 (ΦER15), SEQ ID NO:5 or SEQ ID NO:46 (ΦER46), SEQ ID NO: 20 or SEQ ID NO:47 (ΦER27), SEQ ID NO:25 or SEQ ID NO:53 (ΦER43), and SEQ ID NO: 72 (MM7). In some aspects, a composition with at least two different isolated bacteriophage, wherein each bacteriophage comprises a bacteriophage sequence with no more than 20, 15, 10, 5 or fewer nucleotide changes in any one of SEQ ID NO:2 or SEQ ID NO:40, SEQ ID NO:5 or SEQ ID NO:46, SEQ ID NO:20 or SEQ ID NO:47, SEQ ID NO:25 or SEQ ID NO: 53, and SEQ ID NO:72.

In some aspects, there is a composition comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or 74 different isolated bacteriophage, wherein the different isolated bacteriophage is Deposit Numbers ______ (sequence identical to SEQ ID NO: 1 and SEQ ID NO:37); Deposit Numbers ______ (sequence identical to SEQ ID NO:2 and SEQ ID NO:40); Deposit Numbers ______ (sequence identical to SEQ ID NO:3 and SEQ ID NO: 41), Deposit Numbers ______ (sequence identical to SEQ ID NO:4 and SEQ ID NO:52), Deposit Numbers ______ (sequence identical to SEQ ID NO:5 and SEQ ID NO:56), Deposit Numbers ______ (sequence identical to SEQ ID NO:6 and SEQ ID NO:30); Deposit Numbers ______(sequence identical to SEQ ID NO:7 and SEQ ID NO:31); Deposit Numbers ______(sequence identical to SEQ ID NO:8 or SEQ ID NO:32), Deposit Numbers ______ (sequence identical to SEQ ID NO:9 and SEQ ID NO:33), Deposit Numbers ______ (sequence identical to SEQ ID NO:10), Deposit Numbers ______ (sequence identical to SEQ ID NO:11); Deposit Numbers ______ (sequence identical to SEQ ID NO: 12 and SEQ ID NO:35); Deposit Numbers ______(sequence identical to SEQ ID NO: 13 and SEQ ID NO:34), Deposit Numbers ______(sequence identical to SEQ ID NO: 14 and SEQ ID NO:36), Deposit Numbers ______ (sequence identical to SEQ ID NO: 15 and SEQ ID NO:38), Deposit Numbers ______ (sequence identical to SEQ ID NO: 16 and SEQ ID NO:39); Deposit Numbers ______ (sequence identical to SEQ ID NO: 17 and SEQ ID NO:42); Deposit Numbers ______ (sequence identical to SEQ ID NO: 18 and SEQ ID NO:43), Deposit Numbers ______ (sequence identical to SEQ ID NO:19 and SEQ ID NO:45), Deposit Numbers ______ (sequence identical to SEQ ID NO:20 and SEQ ID NO: 47), Deposit Numbers ______ (sequence identical to SEQ ID NO:21 and SEQ ID NO: 48); Deposit Numbers ______ (sequence identical to SEQ ID NO:22 and SEQ ID NO:49); Deposit Numbers ______ (sequence identical to SEQ ID NO:23 and SEQ ID NO:50), Deposit Numbers ______ (sequence identical to SEQ ID NO:24 and SEQ ID NO:51), Deposit Numbers ______ (sequence identical to SEQ ID NO:25 and SEQ ID NO:53), Deposit Numbers ______(sequence identical to SEQ ID NO:26 and SEQ ID NO:54); Deposit Numbers ______(sequence identical to SEQ ID NO:27 and SEQ ID NO:55); Deposit Numbers ______(sequence identical to SEQ ID NO:28 and SEQ ID NO:59), Deposit Numbers ______ (sequence identical to SEQ ID NO:29 and SEQ ID NO:60). Deposit Numbers ______ (sequence identical to SEQ ID NO:61), Deposit Numbers ______ (sequence identical to SEQ ID NO:62), Deposit Numbers ______ (sequence identical to SEQ ID NO:63), Deposit Numbers ______ (sequence identical to SEQ ID NO:64), Deposit Numbers ______ (sequence identical to SEQ ID NO:65), Deposit Numbers ______ (sequence identical to SEQ ID NO:66), Deposit Numbers ______(sequence identical to SEQ ID NO:67), Deposit Numbers ______ (sequence identical to SEQ ID NO: 68), Deposit Numbers ______ (sequence identical to SEQ ID NO:69), Deposit Numbers ______(sequence identical to SEQ ID NO:70), Deposit Numbers ______ (sequence identical to SEQ ID NO:71), Deposit Numbers ______ (sequence identical to SEQ ID NO:73), Deposit Numbers ______ (sequence identical to SEQ ID NO:44), Deposit Numbers ______ (sequence identical to SEQ ID NO:46), Deposit Numbers ______ (sequence identical to SEQ ID NO:57), and Deposit Numbers ______ (sequence identical to SEQ ID NO:58).

In some aspects, the composition is in a pharmaceutical formulation or pharmacologically acceptable formulation comprising one or more additional ingredients and wherein the formulation is intended to be administered to an organism, such as a mammal or human. In some aspects, the composition further comprises one or more pharmaceutically acceptable carriers and/or excipients. In some aspects, the composition further comprises one or more antibiotic drugs. In some aspects, the composition is a solution or emulsion of solid. In some aspects, the composition is formulated for oral, gavage, intraperitoneal, intravenous, aerosol, or inhalant application.

Any of the bacteriophage compositions described herein can be implemented as a method or use. Methods and uses include but are not limited to treating a Klebsiella infection in a subject, preventing a Klebsiella infection in a subject, treating and/or preventing a Klebsiella infection in a subject, increasing the susceptibility of a drug-resistant Klebsiella strain in a subject to clearance by the subject's immune system, increasing the susceptibility of a pathogenic Klebsiella strain in a subject to clearance by the subject's immune system, increasing the susceptibility of a drug-resistant and/or pathogenic Klebsiella strain in a subject to clearance by the subject's immune system, increasing the susceptibility of a drug-resistant Klebsiella strain in a subject to conventional antibiotic treatment, increasing the susceptibility of a pathogenic Klebsiella strain in a subject to conventional antibiotic treatment, increasing the susceptibility of a drug-resistant and/or pathogenic Klebsiella strain in a subject to conventional antibiotic treatment, reducing the virulence of a drug-resistant Klebsiella strain in a subject, reducing the virulence of a pathogenic Klebsiella strain in a subject, reducing the virulence of a drug-resistant and/or pathogenic Klebsiella strain in a subject, reducing the level of a drug-resistant Klebsiella strain in a subject, reducing the level of a pathogenic Klebsiella strain in a subject, reducing the level of a drug-resistant and/or pathogenic Klebsiella strain in a subject, reducing the number of a Klebsiella bacteria in a subject, reducing the number of capsulated Klebsiella bacteria in a subject, improving symptoms in a subject related to infection from a Klebsiella bacteria, reducing the severity of symptoms in a subject related to infection from a Klebsiella bacteria, reducing the length of hospitalization or risk of hospitalization related to infection from a Klebsiella bacteria, treating a subject for a drug-resistant Klebsiella infection, treating a subject for an antibiotic-resistant Klebsiella infection; treating a subject for a hypervirulent Klebsiella infection; providing a device to a subject that reduces the incidence of, treats, or prevents an infection of Klebsiella infection; in some aspects, the Klebsiella bacteria is drug- or antibiotic-resistant and/or includes Klebsiella pneumoniae, Klebsiella quasipneumoniae, and/or Klebsiella variicola.

Methods and uses may include 1, 2, 3, 4, 5, or more steps such as detecting an infection in an individual, generating a bacteriophage composition, formulating a bacteriophage composition for therapeutic administration, administering or applying a bacteriophage composition to a subject one or more times, evaluating a subject for a Klebsiella infection, monitoring a subject for one or more symptoms of a Klebsiella infection, diagnosing a subject as having or at risk for a Klebsiella infection, administering to the subject one or more antibacterial therapies in addition to a bacteriophage composition, coating a device with a bacteriophage composition, and/or inserting or providing the subject a device coated or containing a bacteriophage composition. Any one or more of the preceding steps may be excluded from certain aspects.

A subject may be any organism that can be infected by Klebsiella or that can harbor, contain, or carry Klebsiella bacteria. In some aspects, the subject is a mammal, such as a human, horse, cow, pig, dog, cat, monkey, sheep, wolf, or deer. Any method or use described herein can be implemented on a subject that is human. In some aspects, the subject is a mammal; in some aspects, the subject is a human. In some aspects, the subject is at a higher risk for Klebsiella infection than the general population.

In some aspects there are methods for treating and/or preventing a Klebsiella infection in a subject comprising administering to the subject a bacteriophage composition as set forth herein, including in paragraphs [0010]-[0025]. In some aspects, the Klebsiella is from Klebsiella pneumoniae, Klebsiella quasipneumoniae, and/or Klebsiella variicola. In some aspects, the subject has symptoms of a Klebsiella infection or is at risk for a Klebsiella infection. In some aspects, the subject has been determined to be infected with Klebsiella. In some aspects, the source of the Klebsiella was from a beverage, comestible, another individual, or an environment. In some aspects, the environment is ground or surface water, water used to irrigate crops, a public water system, a hospital, a school, a nursing home, a petting zoo, a cruise ship, a train, or an airplane.

In some aspects, methods comprise administering a bacteriophage composition 1, 2, 3, 4, 5, 6, 7, 8, 9, 10, 11, 12, 13, 14, 15, 16, 17, 18, 19, or 20 times or more over the course of 5, 10, 15, 20, 25, 30, 35, 40, 45, 50, 55, 60 minutes and/or 1, 2, 3, 4, 5, 6, 7, 8, 9, 10, 11, 12, 13, 14, 15, 16, 17, 18, 19, 20, 21, 22, 23, 24 hours and/or 1, 2, 3, 4, 5, 6, 7 days and/or 1, 2, 3, 4, 5 weeks, and/or 1, 2, 3, 4, 5, 6, 7, 8, 9, 10, 11, 12 or more months (or any range derivable therein). In some aspects, a bacteriophage composition is administered multiple times and/or over multiple days.

In some aspects, methods or uses further comprise administering an antibacterial treatment, such as an antibiotic drug to the subject before, after, and/or during administration of the bacteriophage composition. In some aspects, the Klebsiella is drug-resistant or antibiotic-resistant, meaning the infection is resistant to at least one drug or antibiotic, but it may be resistant to more than one drug or antibiotic. In some aspects, the Klebsiella is hypervirulent, meaning the infection is more virulent than classical Klebsiella and is capable, in some aspects, of causing community-acquired infections, including in healthy individuals. In some aspects, the Klebsiella is drug-resistant and hypervirulent, meaning the infection is resistant to at least one drug or antibiotic, or more than one drug or antibiotic, and the infection is more virulent than classical Klebsiella and is capable, in some aspects, of causing community-acquired infections, including in healthy individuals.

In some aspects, the infection is a urinary tract, blood, gut, abdomen, stomach, lungs, skin, ear, eye, nose, oral, kidney, prostate, bladder, brain, vaginal tract, heart, liver, spleen, tendons, or wound infection, or a combination thereof. In some aspects, the subject has a urinary tract infection, neonatal meningitis, a blood-stream infection, pneumonia, sepsis, a surgical wound infection, a wound infection, a skin infection, an eye infection, an ear infection, an oral infection, a prostate infection, meningitis, a vaginal infection, or a combination thereof.

In some aspects, the subject is immunosuppressed. In some aspects, the subject has an immune cell defect, asplenia, impaired splenic function, nephrotic syndrome, or an autoimmune condition. In some aspects, the subject is administered the composition prior to a medical procedure or regimen. In some aspects, the subject will be subject to immunosuppressive conditions. In some aspects, the subject is receiving or will be receiving chemotherapy. In some aspects, the subject is receiving or will be receiving an immunosuppressant. In some aspects, the immunosuppressant is a glucocorticoid, a calcineurin inhibitor, an antimetabolite, or an antibody therapy. In some aspects, the medical procedure comprises insertion of a device in the subject.

In some aspects, there is a device, comprising, on, in, and/or around the device, compositions described in paragraphs [0010]-[0025]. In some aspects, additionally or alternatively, a subject receives or is provided a device that contains or is coated with a bacteriophage composition as described herein. In some aspects, the device is a catheter, drive line, syringe, tube, implant, defibrillator, artificial joint, pacemaker, screw, rod, disc, intrauterine device, pin, plate, stent, dental device, eye lens, shunt, valve, neurological or neurosurgical device, gastrointestinal device, genitourinary device, catheter cuff, vascular access device, or wound drain. In some aspects, the device is further defined as having a coating comprising compositions described in paragraphs [0010]-[0025].

In some aspects, there is a kit, comprising, housed in a suitable container, compositions described in paragraphs [0010]-[0025]. In some aspects, the kit further comprises a device. In some aspects, the device of the kit is further defined as having a coating comprising compositions described in paragraphs [0010]-[0025]. In some aspects, the compositions described in paragraphs [0010]-[0025] are separate from the device of the kit.

In some aspects, there are methods for producing a composition as discussed herein, including but not limited to compositions described in paragraphs [0010]-[0025]. In some aspects, such methods comprise infecting bacteria with an isolated bacteriophage comprising a bacteriophage sequence that is at least 99% identical to any one of SEQ ID NO: 1-74; culturing the bacteria under conditions to allow bacteriophage to replicate; and isolating bacteriophage from the bacteria culture. In some aspects, methods further comprise purifying the bacteriophage from the bacteria culture.

In some aspects, isolated bacteriophage are evolved to improve lytic capacity, reduce lysogenic capacity, reduce antibiotic resistance gene obtainment capacity, reduce bacterial virulence gene obtainment capacity, and/or increase the number of bacterial strains subject to lysis by the isolated bacteriophage lytic.

In some aspects, a composition is a lotion, cream, body butter, mask, scrub, wash, gel, serum, emulsion (e.g., oil-in-water, water-in-oil, silicone-in-water, water-in-silicone, water-in-oil-in-water, oil-in-water-in-oil, oil-in-water-in-silicone, etc.), solution (e.g., aqueous or hydro-alcoholic solutions), anhydrous bases (e.g., stick or a powder), ointment, milk, paste, aerosol, solid form, jelly, and/or powdered form (e.g., dried, lyophilized, particulate, etc). In some aspects, a composition is a solution, lotion, and/or cream. In some aspects, a composition is shelf-stable. In some embodiments, a composition is formulated for topical, oral, aural, nasal, and/or ophthalmic application. In some aspects, a composition is formulated for application more than once a day, once a day, twice a day, once a week, twice a week, once a month, or twice a month during use. In some aspects, a composition is housed in a delivery apparatus. In some aspects, a composition is comprised in a suitable container. In some aspects, a composition is comprised in a kit.

Throughout this application, the term “about” is used to indicate that a value includes the inherent variation of error for the measurement or quantitation method.

Recitation of ranges of values herein is merely intended to serve as a shorthand method of referring individually to each separate value falling within the range. Unless otherwise indicated herein, each individual value is incorporated into the specification as if it was individually recited herein.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

The term “effective,” as that term is used in the specification and/or claims, means adequate to accomplish a desired, expected, or intended result.

“Individual, “subject,” and “patient” are used interchangeably and can refer to either a human or non-human, such as primates, mammals, and vertebrates. In particular aspects, the subject is a human. The subject is of any age, gender, or race. The subject can be a patient, e.g., have or be suspected of having a disease (that may be referred to as a disorder or medical condition), such as one or more immune system-mediated disorders. The subject may be undergoing or have undergone treatment. The subject may be asymptomatic. The subject may be a healthy individual desirous of prevention of a disease or condition.

The terms “inhibiting” or “reducing” or “preventing” or “avoiding” or any variation of these terms, when used in the claims and/or the specification, includes any measurable decrease or complete inhibition to achieve a desired result.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one aspect of the disclosure may apply to any other aspect of the disclosure. Furthermore, any composition of the disclosure may be used in any method of the disclosure, and any method of the disclosure may be used to produce or to utilize any composition of the disclosure. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also aspects that may be implemented in the context of embodiments discussed elsewhere in a different Example or elsewhere in the application, such as in the Summary, Detailed Description, Claims, and Brief Description of the Drawings.

Other objects, features and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific aspects presented herein.

FIG. 1 shows electron microscopy of representative bacteriophage that infect Klebsiella, according to some aspects of the disclosure.

FIG. 2 shows the phylogenetic relationship of representative bacteriophage that infect Klebsiella, according to some aspects of the disclosure.

FIG. 3 shows the ability of various bacteriophage to infect clinical Klebsiella strains, according to some aspects of the disclosure.

FIG. 4 shows adsorption kinetics of representative bacteriophage disclosed herein.

FIGS. 5A-5C show pH tolerance of representative bacteriophage disclosed herein.

FIGS. 6A-6C show one-step growth of representative bacteriophage disclosed herein.

FIG. 7 shows virulence of a representative bacteriophage evolved over multiple passages.

FIG. 8 shows infection of a clinical Klebsiella strain (MH258) by a representative evolved bacteriophage.

FIG. 9 shows adsorption kinetics of a representative evolved bacteriophage.

FIG. 10 shows development of resistance over time by a clinical Klebsiella strain (MH258) to a representative bacteriophage disclosed herein.

FIGS. 11A-11C shows development of resistance over time by a clinical Klebsiella strain (MH258) to various combinations of representative bacteriophage disclosed herein.

FIG. 12 shows stochastic time to resistance by a clinical Klebsiella strain (MH258) to various combinations of representative bacteriophage disclosed herein.

FIG. 13 illustrates schematically a method of bacteriophage receptor identification, according to some aspects of the disclosure.

FIG. 14 shows bacterial factors determined to be required for infection of bacteria with representative bacteriophage disclosed herein.

FIG. 15 shows infection of Klebsiella MH258 by representative bacteriophage disclosed herein compared to infection by representative bacteriophage disclosed herein of an MH258 strain in which the wbaP gene is deleted (ΔwbaP) and infection by representative bacteriophage disclosed herein of an MH258 strain in which wbaP gene deletion is complemented (AwbaP pZE4D-wbaP).

FIG. 16 shows infection of Klebsiella MH258 by representative bacteriophage disclosed herein compared to infection by representative bacteriophage disclosed herein of an MH258 strain in which the wzc gene is deleted (Δwzc) and infection by representative bacteriophage disclosed herein of an MH258 strain in which wzc gene deletion is complemented (Δwzc pZE4D-wzc).

FIG. 17 shows infection of Klebsiella MH258 by representative bacteriophage disclosed herein compared to infection by representative bacteriophage disclosed herein of an MH258 strain in which the wbaN gene (also referred to herein as the walW gene) is deleted (ΔPSD) and infection by representative bacteriophage disclosed herein of an MH258 strain in which wbaN gene deletion is complemented (ΔPSD pZE4D-PSD).

FIG. 18 shows infection of Klebsiella MH258 by representative bacteriophage disclosed herein compared to infection by representative bacteriophage disclosed herein of an MH258 strain in which the csrD gene is deleted (ΔcsrD) and infection by representative bacteriophage disclosed herein of an MH258 strain in which csrD gene deletion is complemented (ΔcsrD pZE4D-csrD).

FIG. 19 shows infection of Klebsiella MH258 by representative bacteriophage disclosed herein compared to infection by representative bacteriophage disclosed herein of an MH258 strain in which the waaH gene is deleted (ΔwaaH) and infection by representative bacteriophage disclosed herein of an MH258 strain in which waaH gene deletion is complemented (ΔwaaH pZE4D-waaH).

FIG. 20 shows infection of Klebsiella MH258 by representative bacteriophage disclosed herein compared to infection by representative bacteriophage disclosed herein of an MH258 strain in which the ompC gene is deleted (ΔompC) and infection by representative bacteriophage disclosed herein of an MH258 strain in which ompC gene deletion is complemented (ΔompC pZE4D-ompC).

FIG. 21 shows infection of Klebsiella MH258 by representative bacteriophage disclosed herein compared to infection by representative bacteriophage disclosed herein of an MH258 strain in which the fhuA3 gene is deleted (ΔfhuA3) and infection by representative bacteriophage disclosed herein of an MH258 strain in which fhuA3 gene deletion is complemented (ΔfhuA3 pZE4D-fhuA3).

FIGS. 22A-22C show infection of Klebsiella MH258 by representative bacteriophage disclosed herein (FIG. 22A) and an area under curve analysis to illustrate lethality of representative bacteriophage infection of the Klebsiella MH258 (FIG. 22B) or of Klebsiella MH258 deletion mutants in which bacterial factors determined to be required for infection of bacteria with representative bacteriophage are deleted and then complemented (FIG. 22C).

FIGS. 23A-23B show phagocytosis by macrophages of Klebsiella MH258 deletion mutants in which bacterial genes wbaP, wbaN (PSD), wzc, or csrD are deleted or a Klebsiella MH258 strain having extra capsule (wzc-G565A; ER51) (FIG. 23A) and Klebsiella MH258 deletion mutants in which deleted bacterial genes wbaP, wbaN (PSD), wzc, or csrD are complemented (FIG. 23B).

FIG. 24 illustrates schematically a method for measuring phagocytosis by macrophages of bacteriophage-treated bacteria, according to some aspects of the disclosure.

FIG. 25 shows phagocytosis by macrophages of Klebsiella MH258 deletion mutants in which bacterial genes wbaP, wbaN (PSD), csrD, waaH, or fhuA3 are deleted or MH258 strains infected with representative bacteriophage.

FIG. 26 shows measurement of capsule amount of Klebsiella MH258 deletion mutants in which bacterial genes wbaP, wbaN (PSD), wzc, csrD, waaH, or fhuA3 are deleted or a Klebsiella MH258 strain having extra capsule (wzc-G565A; ER51).

FIG. 27 shows colonization of Klebsiella and the effect of bacteriophage treatment on Klebsiella colonization.

FIGS. 28A-28B show the effect of Klebsiella colonization and/or bacteriophage treatment on the mouse microbiome.

FIG. 29 shows that Klebsiella and bacteriophage co-exist in the mouse microbiome.

FIG. 30 shows that mice infected with a representative bacteriophage disclosed herein have Klebsiella populations without a capsule.

FIGS. 31A-31C. Genomic alignments of members of Klebsiella Phages. Whole genomes of phages within the same family or genus were aligned using progressive Mauve. The average length of phages genome within a genus or family is indicated to the right of the alignment. For reference, a similar previously published Klebsiella phage genome is included.

FIG. 32. Klebsiella Phages Exhibit Antimicrobial Activity against Clinically-Relevant Carbapanem-Resistant Klebsiella spp. Individual phages are columns and Klebsiella strains are rows in the host range matrix. Phylogenetic relatedness between phages genomes is determined by VICTOR (Meier-Kolthoff, J. P., and Göker, M. (2017)). Major phage families or genera (for Straboviridae) are denoted by color. Shading intensity indicates the degree of infectivity on spot assays according to an ordinal scale, wherein 0 (white) is fully resistant and 3 (dark shading) is full clearance. Shading values are the mean of three biological replicate spot assays. Representative electron microscopy images of bacteriophages from key families are depicted (scale bar=100 nm). The degree of predicted antimicrobial resistance (AMR) to beta lactam antibiotics is indicated, as are multi-locus sequencing type (MLST), capsule type, and O-antigen type. Kp=K. pneumoniae; Kq=K. quasipneumoniae; Kv=K. variicola.

FIGS. 33A-33L. Host Factors Necessary for Phage Infection. FIGS. 33A-33H. Kill curves for all phage-strain pairs summarized in FIG. 34C. The top plot depicts the kinetic of phage infection of a gene knockout and its respective gene complement is below (MOI=1). Lines show the mean of three independent biological replicates (n=3). FIG. 33I. Heatmap of top 18 INSeq hits ranked by log₂ fold change for ΦER15 and ΦER39 shows transposon-interrupted genes that were over-represented after 3 hours infection relative to uninfected culture. FIG. 33J. Over-expressing csrD makes cells more permissible to phages. A dilution of the five phages were spotted in duplicate on MH258 carrying an empty vector and MH258 constitutively expressing csrD. Large plaque sizes are seen with ΦER15, ΦER16e, ΦER39, and ΦER46e. FIG. 33K. RNA-seq analysis of ΔcsrD mutant as compared to wild-type. A volcano plot shows 73 down-regulated genes (red circles) and 13 up-regulated genes (green circles) in the ΔcsrD mutant (adjusted p-value<0.05 and log₂ fold change either >2 or <−2; DESeq; n=2). FIG. 33L. Gene set enrichment analysis of differential expressed genes described by the indicated SEED Subsystems. Red indicates high significance, with blue as less significant; the size of the circle indicates the number of genes in that set; while a negative value on the x-axis means that pathway is down-regulated in ΔcsrD and a positive value indicates that pathway is up-regulated in ΔcsrD.

FIGS. 34A-34C. Transposon Sequencing Identifies Cell Surface Structures, Outer Membrane Proteins, and a Carbon Storage Regulator as Essential Host Factors for Phage Infection. FIG. 34A. Schematic of gene products necessary for phage infection identified in loss-of-function transposon screen by INSeq and arbitrary-primed PCR. FIG. 34B. Representative spot assays depicting susceptibility of wild-type K. pneumoniae MH258, indicated gene knockouts, and complements to phages ΦER12, ΦER15, ΦER16e, ΦER39, and ΦER46e. Spot assays were conducted in biological triplicate. FIG. 34C. Resistance of wild-type K. pneumonia MH258, indicated gene knockouts, and complements to phages ΦER12, ΦER15, ΦER16e, ΦER39, and ΦER46e in 150 minute broth kill curve assays (MOI=1) (see FIGS. 33A-33H). The resistance metric (arbitrary units) represents the ratio between the area under the curve (AUC) of a strain-phage pair and uninfected culture divided by the ratio in AUC of a wild-type MH258-phage pair and uninfected culture. The mean resistance level for each phage-strain pair of three independent biological replicates is indicated (n=3). Shading intensity indicates a greater degree of resistance to a given phage.

FIGS. 35A-35D. Capsule production, membrane sensitivity, and macrophage engulfment in MH258 knockouts. FIG. 35A. Quantitative uronic acid assay of different mutants shows reduced capsule production in ΔwbaP, Δwzc, ΔwalW, and ΔwaaH mutants (*p<0.05, **p<0.01, ***p<0.001; Unpaired t test with Welch's correction of MH258 to mutant; n=3-5). FIG. 35B. SDS/EDTA plates destabilize the outer membrane in ΔtolB mutants. Representative picture of serial dilutions of each deletion strain and complement. In addition to ΔtolB, the ΔwalW and ΔcsrD (red rectangle) also show sensitivity to SDS/EDTA that is restore upon complementation. FIG. 35C. Macrophage engulfment assay with MH258, deletion mutants and complements. MH258 wzc_{G565A} overproduces capsule and is thus protected from phagocytosis. Values are normalized to the number of wild-type MH258 colonies engulfed by the macrophages during individual experiments. The assay was performed 8 times with each closed black dot representing a separate experiment (n=8). FIG. 35D. A heatmap showing sensitivity to colistin (CT), tigecycline (TGC), cefepime (FEP), amikacin (AK), piperacillin/tazobactam (TZP), meropenem (MEM), and imipenem (IPM). The values show the difference in diameter in mm between MH258, the deletion mutant or complement. Orange shading represents increased resistance/smaller zone of clearing, while dark blue represents increased susceptibility/larger zone of clearing in the mutant (n=4-5).

FIGS. 36A-36C. Phage-treated K. pneumoniae MH258 are more readily engulfed by macrophages and are sensitized to antibiotics. FIG. 36A. Schematic of macrophage phagocytosis assay. FIG. 36B. Phage-treated and deletion mutant bacteria cultures are enumerated inside murine macrophages using gentamicin protection assay. The values are normalized to the number of wild-type MH258 colonies engulfed by the macrophages during individual experiment. The assay was performed 3-5 times with each closed black dot representing a separate experiment. FIG. 36C. A heatmap showing sensitivity to colistin (CT), tigecycline (TGC), cefepime (FEP), amikacin (AK), piperacillin/tazobactam (TZP), meropenem (MEM), and imipenem (IPM). The values show the difference in diameter in mm between MH258 and the deletion or phage treatment. Orange shading represents increased resistance/smaller zone of clearing, while dark blue represents increased susceptibility/larger zone of clearing in the mutant (n=4-5 for deletion mutants; n=5-6 for phage-treated cultures).

FIGS. 37A-37C. Combinations of natural or evolved Klebsiella Phage Bank phages can effectively kill multi-drug resistant clinical isolates. FIG. 37A. Combining ΦER12 with phages that depend on a different host factor for infection increases the time resistant subpopulations emerge in kinetic experiments (MOI=10). The time to resistance was determined as the time at which monotonic growth resumed following phage treatment. Phages from various genera or family are colored according to the code in FIG. 32. FIG. 37B. Evolved ΦER16e produces plaques with a larger diameter than ancestral ΦER16. FIG. 37C. The efficiency of killing ΦER3 on various strains is altered depending on the bacterial host on which the lysate was amplified (MOI=1) (ΦER3_{MH258} was amplified on K. pneumoniae MH258, ΦER3_{UTI-7} was amplified on K. quasipneumoniae UTI-7, and ΦER3_{K6} on K. quasipneumoniae K6).

FIGS. 38A-38K. Combinations of natural or evolved Klebsiella phages can effectively kill multi-drug resistant clinical isolates. FIG. 38A. Schematic showing workflow for anti-Klebsiella cocktail design and optimization. For a specific Klebsiella strain, the best candidate phages from different categories in FIG. 32 are selected and can be further optimized for a given host through repeated passaging. FIGS. 38B-38G. Growth curves for K. pneumoniae strains MH258 (FIG. 38B, FIG. 38E), JHCK1 (FIG. 38D, FIG. 38G), and K. quasipneumoniae strain UTI-7 (FIG. 38C, FIG. 38F). The top row of plots (FIGS. 38B-38D) depict a 2.5 hour growth curve showing the antimicrobial activity of five individual phages selected for the cocktail (MOI=1). The bottom row of plots (FIGS. 38E-38G) depicts the resistance profiles after 16 hours of infection. Individual phage curves all show recovery while each cocktail (teal) shows little to no growth for all three strains (MOI=10). Line color represents the genus or family of the phages as depicted in FIG. 32 (n=3 independent biological replicates) (FIG. 38H, FIG. 38J) Growth curve of evolved phages ΦER16e (FIG. 38H) and ΦER46e (FIG. 38J) show improved killing of the evolved variant (darker, solid line) compared to the ancestral phage (lighter, dashed line) (MOI=1) (n=3 independent biological replicates). Evolved ΦER16e (FIG. 38I) and ΦER46e (FIG. 38K) show improved adsorption to bacterial cells compared to ancestral phages as measured by the rate of decrease of free phages in the supernatant normalized to the original titer (PFU/mL) over time. Evolved variants (darker, solid lines) have less free phages than the ancestral phages (light, dashed linse). Line indicates a non-linear fit to an exponential decay curve (R²>0.98 for ΦER16, ΦER16e, ΦER46e; R²=0.84 for ΦER46) (n=4 independent biological replicates).

FIGS. 39A-39F. Phage cocktail suppresses and alters Klebsiella population in the mouse gastrointestinal tract. FIG. 39A. K. pneumoniae MH258 titer in fecal samples (Days 3, 7, 8, 10) and cecal samples (Day 15) in gnotobiotic mice treated with PBS (black) or a phage cocktail comprised of ΦER12, ΦER15, ΦER16e, ΦER39, and ΦER46e (red) (˜10⁹ PFU each/mouse). Phages administered on Day 7 and Day 8 suppressed Kp burden in the gut (***p<0.001; Unpaired t test with Welch's correction; n=17 for PBS- and n=23 for cocktail-treated). Data is pooled from 5 separate experiments and error bars represent the standard deviation. See FIG. 40A for individual experiments. FIG. 39B. Phage titers for ΦER39 (purple), ΦER12 (yellow), ΦER15 and ΦER16e (blue), and ΦER46e (orange) in fecal samples over the course of the experiment, with cecal samples on Day 15. The open circle represents the limit of detection on Day 15. The bolded lines are the mean values of 4 separate experiments, with each individual experiment means shown in lighter colored lines (n=18). See FIG. 40C for individual experiments. FIG. 39C. Correlation of the total titer of phage on Day 8 and the fold decrease in K. pneumoniae levels (Spearman p=0.8436; p<0.0001) Threshold for effective Klebsiella killing occurs when at least 3×10⁸ PFU/g stool are present on the day following the first gavage. FIG. 39D. Representative relative 16S rRNA abundance from PBS- and cocktail-treated mice on Day 8. (Kp—Klebsiella pneumoniae; Ri—Roseburia inulinivorans; Am=Akkermansia muciniphila; Ba—Bifidobacterium adolescentis; Sv—Subdoligranulum variable; Ts—Turicibacter sanguinis; Lr—Limosilactobacillus reuteri; Rg—[Ruminoccocus] gnavus; Ac—Anaerostipes caccae; Ec=Escherichia coli; Af—Alistipes finegoldii; Pd—Parabacteroides distasonis; Bu—Bacteroides uniformis; Pv—Phocaeicola vulgatus; Bt—Bacteroides thetaiotaomicron.) FIG. 39E. Principle component analysis of the microbiome composition (16S rRNA relative abundance) of PBS—(black) and cocktail—(red) treated mice on Day 8 for all five experiments. FIG. 39F. The percent of non-capsulated Klebsiella colonies recovered from PBS—(black) and cocktail—(red) treated mice on Day 15 (***p<0.0001; Unpaired t test with Welch's correction; n=17 for PBS- and n=23 for cocktail-treated).

FIGS. 40A-40C. Phage cocktail suppresses and alters Klebsiella population in the mouse gastrointestinal tract. FIG. 40A. Data from individual trials of K. pneumoniae (Kp) MH258-targeting cocktail. K. pneumoniae MH258 titer in fecal samples (Days 3, 7, 8, 10) and cecal samples (Day 15) in gnotobiotic mice treated with PBS (black) or a phage cocktail comprised of ΦER12, ΦER15, ΦER16e, ΦER39, and ΦER46e (red) (˜10⁹ PFU each/mouse). Phages administered on Day 7 and Day 8 suppressed Kp burden in the gut. FIG. 40B. Aggregate data of K. pneumoniae MH258 fecal or cecal CFU counts partitioned by the recovered total phage titer on Day 8. Mice with fecal phage titers greater than 2×10⁸ PFU/g stool showed a more pronounced decline in fecal Klebsiella levels. FIG. 40C. Data from individual trials of K. pneumoniae (Kp) MH258-targeting cocktail. Phage titers for ΦER39 (purple), ΦER12 (yellow), ΦER15 and ΦER16e (blue), and ΦER46e (orange) in fecal samples over the course of the experiment, with cecal samples on Day 15. The bolded lines are the mean values of 4 separate experiments, with each individual experiment means shown in lighter colored lines.

### DETAILED DESCRIPTION

The present disclosure is based, at least in part, on the discovery and development of novel bacteriophages and bacteriophage combinations for treatment and prevention of Klebsiella infection, including multidrug-resistant (MDR) or hypervirulent Klebsiella infection. Accordingly, disclosed herein, in some aspects, are bacteriophage compositions comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or more isolated bacteriophage. In some aspects, disclosed are bacteriophage compositions comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or 74 isolated bacteriophage comprising a bacteriophage sequence corresponding to ΦER1, ΦER2, ΦER3, ΦER4, ΦER5, ΦER6, ΦER7/ΦER10, ΦER8, ΦER11, ΦER12, ΦER13, ΦER14, ΦER15, ΦER16, ΦER18, ΦER20, ΦER21, ΦER22, ΦER23, ΦER27, ΦER32, ΦER36, ΦER37, ΦER38, ΦER39, ΦER43, ΦER44, ΦER45, ΦER46, ΦER47, ΦER48, ΦER49, ΦER50, ΦER51, ΦER52, ΦER53, ΦER54, ΦER55, ΦMM1, ΦMM2, ΦMM3, ΦMM4, ΦMM5, ΦMM6, ΦMM7, ΦMM8, and/or ΦMM9. In some aspects, any one or more of the foregoing isolated bacteriophage may be excluded from the compositions and/or methods disclosed herein. Further disclosed are devices and kits comprising such compositions and methods for use of such compositions in treatment and/or prevention of pathogenic Klebsiella infection.

### I. Bacteriophage Compositions

Aspects of the disclosure are directed to compositions comprising one or more bacteriophage. As used herein, a “bacteriophage composition” describes any composition comprising one or more bacteriophage (also “phage” or “Φ”). A bacteriophage of the present disclosure may be a lysogenic phage. A bacteriophage of the present disclosure may be a lytic phage.

Methods of isolating and characterizing bacteriophage are known in the art and described in, e.g., Hyman P., “Phages for Phage Therapy: Isolation, Characterization, and Host Range Breadth,” Pharmaceuticals (Basel). 2019 March; 12(1): 35, incorporated herein by reference in its entirety. Isolation procedures and variations that are designed to isolate phages include, but are not limited to, e.g., enrichment from an environmental sample, plaque testing, culture lysis, and/or routine test dilution. Characterization procedures used to show that a phage may have utility in phage therapy include, but are not limited to, e.g., plating efficiency, phage morphology by microscopy, whole genome sequencing, one-step growth curves, and/or pulse-field gel electrophoresis. Any one or more of these procedures may be used to isolate and/or characterize a bacteriophage of the disclosure.

In some aspects, a bacteriophage of the disclosure is a phage of the order Caudovirales. In some aspects, a bacteriophage of the disclosure is a phage of the family Straboviridae, Ackermannviridae, Demerecviridae, Drexlerviridae, Vequintavirinae, Schitoviridae, Autographiviridae, Siphoviridae (proposed Purpuraviridae), Myoviridae, Podoviridae, Chaseviridae, Guelinviridae, Herelleviridae, Rountreeviridae, Salasmaviridae, and/or Zobellviridae. In some aspects, bacteriophages from any one or more of the foregoing bacteriophage families may be excluded from the compositions and/or methods disclosed herein.

In specific aspects, a bacteriophage of the disclosure is a phage of the family Straboviridae. In some aspects, the Straboviridae family phage is a phage of the genus Angelvirus, Biquartavirus, Bragavirus, Carettavirus, Chrysonvirus, Cinqassovirus, Emmerichvirinae, Gualtarvirus, Jiangsuvirus, Krischvirus, Marfavirus, Mylasvirus, Pseudotevenvirus, Schizotequatrovirus, Slopekvirus, Tevenvirinae, Tulanevirus, and/or Twarogvirinae. In specific aspects, a bacteriophage of the disclosure is a Ceceduovirus, Ishigurovirus, Dhakavirus, Gaprivervirus, Gelderlandvirus, Jiaodavirus, Kanagawavirus, Karamvirus, Moonvirus, Mosigvirus, Mosugukvirus, Roskildevirus, Tegunavirus, Tequatrovirus, Winklervirus, Acajnonavirus, Hadassahvirus, Lasallevirus, Lazarusvirus, and/or Zedzedvirus. In specific aspects, an Straboviridae bacteriophage of the disclosure is ΦER16, ER27, ΦER32, ΦER36, ΦER44, ΦER45, ΦER47, ΦER48, ΦER50, ΦER51, ΦER53, ΦER54. In specific aspects, an Straboviridae bacteriophage of the disclosure is ΦER8, ΦER11, ΦER13, ΦER15, or ΦER38. In specific aspects, a Straboviridae bacteriophage of the disclosure is ΦMM3 or ΦMM4. In some aspects, bacteriophage from any one or more of the foregoing bacteriophage genera, or species therein, may be excluded from the compositions and/or methods disclosed herein.

In specific aspects, a bacteriophage of the disclosure is a phage of the family Ackermannviridae. In some aspects, the Ackermannviridae family phage is a phage of the subfamily Aglimvirinae and/or Cvivirinae. In some aspects, the Ackermannviridae family phage is a phage of the genus Agtrevirus, Limestonevirus, Kuttervirus, Campanilevirus, Kujavirus, Miltonvirus, Nezavisimistyvirus, Taipeivirus, Tedavirus, and/or Vapseptimavirus. In specific aspects, a bacteriophage of the disclosure is a Ackermannviridae ViI virus (e.g., Kuttervirus ViI). In specific aspects, an Ackermannviridae bacteriophage of the disclosure is ΦER12, ΦER14, or ΦER20. In some aspects, any one or more of the foregoing bacteriophage may be excluded from the compositions and/or methods disclosed herein. In some aspects, bacteriophage from any one or more of the foregoing bacteriophage genera, or species therein, may be excluded from the compositions and/or methods disclosed herein.

In specific aspects, a bacteriophage of the disclosure is a phage of the family Demerecviridae. In some aspects, the Demerecviridae family phage is a phage of the subfamily Ermolyevavirinae, Markadamsvirinae, and/or Mccorquodalevirinae. In some aspects, the Demerecviridae family phage is a phage of the genus Cetovirus, Jesfedecavirus, Thalassavirus, Vipunavirus, Epseptimavirus, Haartmanvirus, Tequintavirus, Hongcheonvirus, Myunavirus, Novosibvirus, Pogseptimavirus, Shenzhenvirus, and/or Sugarlandvirus. In specific aspects, a bacteriophage of the disclosure is a phage of the genus Tequintavirus (i.e., a T5-like virus). In specific aspects, a Demerecviridae bacteriophage of the disclosure is ΦER46 or ΦER52. In some aspects, bacteriophage from any one or more of the foregoing bacteriophage genera, or species therein, may be excluded from the compositions and/or methods disclosed herein.

In specific aspects, a bacteriophage of the disclosure is a phage of the family Drexlerviridae. In some aspects, the Drexlerviridae family phage is a phage of the genus Braunvirinae, Eclunavirus, Hicfunavirus, Jhansiroadvirus, Kyungwonvirus, Nouzillyvirus, Rogunavirinae, Tempevirinae, Tunavirinae, Vilniusvirus, and/or Webervirus. In specific aspects, a bacteriophage of the disclosure is a Christensenvirus, Guelphvirus, Loudonvirus, Rtpvirus, Veterinaerplatzvirus, Eastlansingvirus, Lindendrivevirus, Rogunavirus, Wilsonroadvirus, Sauletekiovirus, Changchunvirus, Hanrivervirus, Henuseptimavirus, Tlsvirus, Warwickvirus, Badaguanvirus, Sertoctavirus, and/or Tunavirus. In specific aspects, a Drexlerviridae bacteriophage of the disclosure is PERI, ΦER2, ΦER7/10, ΦER21, ΦER22, ΦER23, or ΦER55. In some aspects, bacteriophage from any one or more of the foregoing bacteriophage genera, or species therein, may be excluded from the compositions and/or methods disclosed herein.

In specific aspects, a bacteriophage of the disclosure is a phage of the family Autographiviridae. In some aspects, the Autographiviridae family phage is a phage of the genus Aegirvirus, Anchaingvirus, Aqualcavirus, Ashivirus, Ayakvirus, Ayaqvirus, Banchanvirus, Beijerinckvirinae, Bifseptvirus, Bonnellvirus, Cheungvirus, Chosvirus, Colwellvirinae, Corkvirinae, Cuernavacavirus, Cyclitvirus, Ermolevavirus, Foturvirus, Foussvirus, Fussvirus, Gajwadongvirus, Gyeongsanvirus, Igirivirus, Jalkavirus, Jiaoyazivirus, Kafavirus, Kajamvirus, Kakivirus, Kalppathivirus, Kelmasvirus, Kembevirus, Krakvirus, Krylovirinae, Lauvirus, Limelightvirus, Lingvirus, Lirvirus, Lullwatervirus, Maculvirus, Melnykvirinae, Molineuxvirinae, Napahaivirus, Nohivirus, Oinezvirus, Okabevirinae, Paadamvirus, Pagavirus, Pairvirus, Pedosvirus, Pekhitvirus, Pelagivirus, Percyvirus, Piedvirus, Podivirus, Pollyceevirus, Poseidonvirus, Powvirus, Pradovirus, Qadamvirus, Scottvirus, Sednavirus, Serkorvirus, Sieqvirus, Slopekvirinae, Stompelvirus, Stopalavirus, Stopavirus, Studiervirinae, Stupnyavirus, Tangaroavirus, Tawavirus, Tiamatvirus, Tiilvirus, Tritonvirus, Voetvirus, Votkovvirus, Waewaevirus, and/or Wuhanvirus. In specific aspects, a bacteriophage of the disclosure is a Daemvirus, Friunavirus, Pettyvirus, Gutovirus, Kaohsiungvirus, Murciavirus, Trungvirus, Uliginvirus, Kantovirus, Kotilavirus, Phimunavirus, Stompvirus, Kirikabuvirus, Phikmvvirus, Stubburvirus, Tunggulvirus, Aerosvirus, Aghbyvirus, Ahphunavirus, Cronosvirus, Panjvirus, Pienvirus, Wanjuvirus, Acadevirus, Axomammavirus, Eracentumvirus, Tuodvirus, Vectrevirus, Zindervirus, Ampunavirus, Higashivirus, Mguuvirus, Risjevirus, Sukuvirus, Bucovirus, Drulisvirus, Koutsourovirus, Novosibovirus, Aarhusvirus, Apdecimavirus, Berlinvirus, Caroctavirus, Chatterjeevirus, Eapunavirus, Elunavirus, Foetvirus, Ghunavirus, Helsettvirus, Jarilovirus, Kayfunavirus, Minipunavirus, Ningirsuvirus, Pektosvirus, Phutvirus, Pifdecavirus, Pijolavirus, Przondovirus, Teetrevirus, Teseptimavirus, Troedvirus, Unyawovirus, and/or Warsawvirus. In specific aspects, an Autographiviridae bacteriophage of the disclosure is ΦER43 or ΦMM9. In some aspects, bacteriophage from any one or more of the foregoing bacteriophage genera, or species therein, may be excluded from the compositions and/or methods disclosed herein.

In specific aspects, a bacteriophage of the disclosure is a phage of the family Vequintavirinae. In some aspects, the Vequintavirinae family phage is a phage of the genus Avunavirus, Certrevirus, Henunavirus, Mydovirus, Seunavirus, and/or Vequintavirus. In specific aspects, a Vequintavirinae bacteriophage of the disclosure is ΦER37. In some aspects, bacteriophage from any one or more of the foregoing bacteriophage genera, or species therein, may be excluded from the compositions and/or methods disclosed herein.

In specific aspects, a bacteriophage of the disclosure is a phage of the family Schitoviridae. In some aspects, the Schitoviridae family phage is a phage of the genus Cbunavirus, Dendoorenvirus, Eceepunavirus, Efbeekayvirus, Electravirus, Enquatrovirinae, Erskinevirinae, Exceevirus, Fuhrmanvirinae, Humphriesvirinae, Littlefixvirus, Migulavirinae, Mukerjeevirus, Oliverunavirus, Pacinivirus, Pariacacavirus, Penintadodekavirus, Pokkenvirus, Pontosvirinae, Presleyvirus, Rhodovirinae, Riverridervirus, Rothmandenesvirinae, Shizishanvirus, Triduovirus, Vicoquintavirus, Waedenswilvirus, Zicotriavirus, and/or Zurivirus. In specific aspects, a Schitoviridae of the disclosure is a Enquatrovirus, Gamaleyavirus, Kaypoctavirus, Johnsonvirus, Yonginvirus, Matsuvirus, Stoningtonvirus, Huelvavirus, Ithacavirus, Pollockvirus, Pylasvirus, Litunavirus, Luzseptimavirus, Dorisvirus, Galateavirus, Nahantvirus, Dongdastvirus, Inbricusvirus, Jwalphavirus, Pourcelvirus, Aoqinvirus, Aorunvirus, Baltimorevirus, Plymouthvirus, Pomeroyivirus, Raunefjordenvirus, and/or Sanyabayvirus. In specific aspects, an Schitoviridae bacteriophage of the disclosure is ΦER49. In some aspects, bacteriophage from any one or more of the foregoing bacteriophage genera, or species therein, may be excluded from the compositions and/or methods disclosed herein.

In specific aspects, a bacteriophage of the disclosure is a phage of the family Siphoviridae. In some aspects, the Siphoviridae family phage is a phage of the proposed genus Purpuraviridae. In some aspects, the Siphoviridae family phage is a phage of the genus Arquatrovirinae, Bclasvirinae, Bronfenbrennervirinae, Chebruvirinae, Dclasvirinae, Deejayvirinae, Dolichocephalovirinae, Gochnauervirinae, Gutmannvirinae, Hendrixvirinae, Langleyhallvirinae, Mccleskeyvirinae, Mclasvirinae, Nymbaxtervirinae, Pclasvirinae, Queuovirinae, Skryabinvirinae, Trabyvirinae, and/or Tybeckvirinae. In specific aspects, a bacteriophage of the disclosure is a Arquatrovirus, Camvirus, Likavirus, Azeredovirinae, Dubowvirus, Phietavirus, Acadianvirus, Coopervirus, Pregunavirus, Pipefishvirus, Rosebushvirus, Biseptimavirus, Peevelvirus, Brujitavirus, Hawkeyvirus, Plotvirus, Kenoshvirus, Secretariavirus, Tanisvirus, Berteleyvirus, Colossusvirus, Poidextervirus, Spirovirus, Dragolirvirus, Harrisonvirus, Vegavirus, Wandersvirus, Guernseyvirinae, Cornelivirus, Jerseyvirus, Kagunavirus, Cauhtlivirus, Kwaitsingvirus, Nochtlivirus, Sainkungvirus, Shamshuipovirus, Wainchavirus, Wongtaivirus, Yautsimvirus, Carmenvirus, Pobcunavirus, Getalongvirus, Horusvirus, Phystavirus, Limdunavirus, Unaquatrovirus, Bongovirus, Reyvirus, Nclasvirinae, Bettesrvirus, Charlievirus, Redivirus, Baxtervirus, Nymphaduravirus, Bignuzvirus, Fisburnevirus, Phayoncevirus, Amoyvirus, Nipunavirus, Nonagvirus, Seuratvirus, Bambunaquatrovirus, Puchinovirus, Jelitavirus, Slepowrronvirus, Douglasvirus, Lenusvirus, Lideunavirus, and/or Maenadvirus. In specific aspects, a Siphoviridae bacteriophage of the disclosure is ΦER3, ΦER4, ΦER18, ΦER39, ΦMM1, ΦMM2, ΦMM5, ΦMM6, ΦMM7, or ΦMM8. In some aspects, bacteriophage from any one or more of the foregoing bacteriophage genera, or species therein, may be excluded from the compositions and/or methods disclosed herein.

In specific aspects, a bacteriophage of the disclosure is a phage of the family Myoviridae. In some aspects, the Myoviridae family phage is a phage of the subfamily Emmerichvirinae, Eucampyvirinae, Gorgonvirinae, Ounavirinae, Peduovirinae, Tevenvirinae (a.k.a. Tequatrovirinae), Twarogvirinae, and/or Vequintavirinae. In some aspects, the Myoviridae family phage is a phage of the genus Abouovirus, Acionnavirus, Agricanvirus, Ahtivirus, Alcyoneusvirus, Alexandravirus, Anamdongvirus, Anaposvirus, Aokuangvirus, Asteriusvirus, Atlauavirus, Aurunvirus, Ayohtrevirus, Baikalvirus, Bakolyvirus, Barbavirus, Bcepfunavirus, Bcepmuvirus, Becedseptimavirus, Bellamyvirus, Bendigovirus, Biquartavirus, Bixzunavirus, Borockvirus, Brigitvirus, Brizovirus, Brunovirus, Busanvirus, Carpasinavirus, Chakrabartyvirus, Charybdisvirus, Chiangmaivirus, (olneyvirus, Cymopoleiavirus, Derbicusvirus, Dibbivirus, Donellivirus, Elmenteitavirus, Elvirus, Emdodecavirus, Eneladusvirus, Eponavirus, Erskinevirus, Eurybiavirus, Ficleduovirus, Flaumdravirus, Fukuivirus, Gofduovirus, Goslarvirus, Haloferacalesvirus, Hapunavirus, Heilongjiangvirus, Iapetusvirus, Iodovirus, Ionavirus, Jedunavirus, Jilinvirus, Jimmervirus, Kanagawavirus, Kanaloavirus, Klausavirus, Kleczkowskavirus, Kungbxnavirus, Kylevirus, Lagaffevirus, Leucotheavirus, Libanvirus, Lietduovirus, Llyrvirus, Loughboroughvirus, Lubbockvirus, Machinavirus, Marfavirus, Marthavirus, Mazuvirus, Menderavirus, Metrivirus, Mieseafarmvirus, Mimasvirus, Moabitevirus, Moturavirus, Muldoonvirus, Mushuvirus, Muvirus, Myoalterovirus, Myohalovirus, Myosmarvirus, Naesvirus, Namakavirus, Nankokuvirus, Neptunevirus, Nereusvirus, Nerrivikvirus, Nodensvirus, Noxifervirus, Nylescharonvirus, Obolenskvirus, Otagovirus, Pakpunavirus, Palaemonvirus, Pbunavirus, Peatvirus, Pemunavirus, Petsuvirus, Phabquatrovirus, Phapecoctavirus, Phikzvirus, Pippivirus, Plaisancevirus, Plateaulakevirus, Polybotosvirus, Pontusvirus, Popoffvirus, Punavirus, Qingdaovirus, Rahariannevirus, Radnorvirus, Ripduovirus, Risingsunvirus, Ronodorvirus, Rosemountvirus, Saclayvirus, Saintgironsvirus, Salacisavirus, Salmondvirus, Sarumanvirus, Sasquatchvirus, Schmittlotzvirus, Seoulvirus, Shandongvirus, Sherbrookevirus, Shirahamavirus, Shalavirus, Svunavirus, Tabernariusvirus, Takahashivirus, Tamkungvirus, Taranisvirus, Tefnutvirus, Tegunavirus, Thaumasvirus, Thetisvirus, Thornevirus, Tijeunavirus, Toutatisvirus, Tulanevirus, Vellamovirus, Vhmlvirus, Vibakivirus, Wellingtonvirus, Wifcevirus, Winklervirus, Yokohamavirus, Yoloswagvirus, and/or Yongloolinvirus. In specific aspects, a bacteriophage of the disclosure is a Myoviridae μ-like virus. In specific aspects, a bacteriophage of the disclosure is a Myoviridae P1-like virus. In specific aspects, the Myoviridae family phage is a phage of the subfamily Tevenvirinae (a.k.a. Tequatrovirinae) (i.e., a T4-like virus). In specific aspects, a Myoviridae bacteriophage of the disclosure is ΦER12, ΦER14, ΦER20, or ΦER37. In some aspects, bacteriophage from any one or more of the foregoing bacteriophage genera, or species therein, may be excluded from the compositions and/or methods disclosed herein.

In specific aspects, a bacteriophage of the disclosure is a phage of the family Podoviridae. In some aspects, the Podoviridae family phage is a phage of the subfamily Beephvirinae, Eekayvirinae, and/or Sepvirinae. In some aspects, the Podoviridae family phage is a phage of the genus Flowerpowervirus, Immanueltrevirus, Manuelvirus, Akonivirus, Tinytimothyvirus, Diegovirus, Oslovirus, Traversvirus, Anjalivirus, Astrithrvirus, Badaztecvirus, Bjornvirus, Bruynoghevirus, Burrovirus, Chopinvirus, Cimandefvirus, Delislevirus, Dybvigvirus, Enhodamvirus, Enquatrovirus, Fipvunavirus, Firingavirus, Gervaisevirus, Giessenvirus, Hollowayvirus, Jasminevirus, Kafunavirus, Kelquatrovirus, Kochitakasuvirus, Kozyakovvirus, Krylovvirus, Kuravirus, Lahexavirus, Lastavirus, Lederbergvirus, Lessievirus, Lightbulbvirus, Myxoctovirus, Pagevirus, Parlovirus, Perisivirus, Privateervirus, Rauchvirus, Ryyoungvirus, Schmidvirus, Sendosyvirus, Skarprettervirus, Sortsnevirus, Uetakevirus, Vicosavirus, Wumpquatrovirus, Wumptrevirus, and/or Xuquatrovirus. In specific aspects, a bacteriophage of the disclosure is a Podoviridae T7-like virus. In specific aspects, a bacteriophage of the disclosure is a Podoviridae P22-like virus. In specific aspects, a Podoviridae bacteriophage of the disclosure is ΦER43 or ΦMM9. In some aspects, bacteriophage from any one or more of the foregoing bacteriophage genera, or species therein, may be excluded from the compositions and/or methods disclosed herein.

In specific aspects, a bacteriophage of the disclosure is a phage of the family Siphoviridae. In some aspects, the Siphoviridae family phage is a phage of the subfamily Arquatrovirinae, Azeredovirinae, Bclasvirinae, Bronfenbrennervirinae, Chebruvirinae, Dclasvirinae, Deejayvirinae, Dolichocephalovirinae, Gochnauervirinae, Guernseyvirinae, Gutmannvirinae, Hendrixvirinae, Langleyhallvirinae, Mccleskeyvirinae, Mclasvirinae, Nclasvirinae, Nymbaxtervirinae, Pclasvirinae, Queuovirinae, Skryabinvirinae, Trabyvirinae, and/or Tybeckvirinae. In some aspects, the Siphoviridae family phage is a phage of the genus Arquatrovirus, Camvirus, Likavirus, Dubowvirus, Phietavirus, Acadianvirus, Coopervirus, Pregunavirus, Pipefishvirus, Rosebushvirus, Biseptimavirus, Peevelvirus, Brujitavirus, Hawkeyvirus, Plotvirus, Kenoshvirus, Secretariavirus, Tanisvirus, Berteleyvirus, Colossusvirus, Poidextervirus, Spirovirus, Dragolirvirus, Harrisonvirus, Vegavirus, Wandersvirus, Cornelivirus, Jerseyvirus, Kagunavirus, Carmenvirus, Pobcunavirus, Brynievirus, Cauhtlivirus, Kwaitsingvirus, Nochtlivirus, Sainkungvirus, Shamshuipovirus, Wainchavirus, Wongtaivirus, Yautsimvirus, Getalongvirus, Horusvirus, Phystavirus, Limdunavirus, Unaquatrovirus, Bongovirus, Reyvirus, Bettesrvirus, Charlievirus, Redivirus, Baxtervirus, Nymphaduravirus, Bignuzvirus, Fisburnevirus, Phayoncevirus, Amoyvirus, Nipunavirus, Nonagvirus, Seuratvirus, Bambunaquatrovirus, Puchinovirus, Jelitavirus, Slepowrronvirus, Douglasvirus, Lenusvirus, Lideunavirus, Maenadvirus, Abbeymikolonvirus, Abidjanvirus, Agmunavirus, Aguilavirus, Ahduovirus, Alachuavirus, Alegriavirus, Amigovirus, Anatolevirus, Andrewvirus, Andromedavirus, Annadreamyvirus, Appavirus, Apricotvirus, Arawnvirus, Armstrongvirus, Ashduovirus, Attisvirus, Attoomivirus, Audreyjarvisvirus, Austintatiousvirus, Avanivirus, Bantamvirus, Barnyardvirus, Beceayunavirus, Beetrevirus, Behunavirus, Bernalvirus, Betterkatzvirus, Bievrevirus, Bingvirus, Bowservirus, Bridgettevirus, Britbratvirus, Bronvirus, Brussowvirus, Camtrevirus, Casadabanvirus, Cbastvirus, Cecivirus, Ceetrepovirus, Cequinquevirus, Chenonavirus, Cheoctovirus, Chertseyvirus, Chivirus, Chunghsingvirus, (impunavirus, (inunavirus, Coetzeevirus, Colunavirus, Coralvirus, Corndogvirus, Cornievirus, Coventryvirus, Cronusvirus, Cukevirus, Daredevilvirus, Decurrovirus, Delepquintavirus, Demosthenesvirus, Detrevirus, Deurplevirus, Dhillonvirus, Dinavirus, Dismasvirus, Doucettevirus, Edenvirus, Efquatrovirus, Eiauvirus, Eisenstarkvirus, Elerivirus, Emalynvirus, Eyrevirus, Fairfaxidumvirus, Farahnazvirus, Fattrevirus, Feofaniavirus, Fernvirus, Fibralongavirus, Fowlmouthvirus, Franklinbayvirus, Fremauxvirus, Fromanvirus, Gaiavirus, Galaxyvirus, Galunavirus, Gamtrevirus, Gesputvirus, Getseptimavirus, Ghobesvirus, Gilesvirus, Gillianvirus, Gilsonvirus, Glaedevirus, Godonkavirus, Goodmanvirus, Gordonvirus, Gordinkvirus, Gorganvirus, Gorjumvirus, Gustavvirus, Halcyonevirus, Hattifnattvirus, Hedwigvirus, Helsingorvirus, Hiyaavirus, Hnatkovirus, Holosalinivirus, Homburgvirus, Hubeivirus, laduovirus, Ikedavirus, Ilzatvirus, Incheonvirus, Indlulamithivirus, Inhavirus, Jacevirus, Jarrellvirus, Jenstvirus, Jouyvirus, Juiceboxvirus, Junavirus, Kairosalinivirus, Kamchatkavirus, Karimacvirus, Kelleziovirus, Kilunavirus, Klementvirus, Knuthellervirus, Kojivirus, Konstantinevirus, Korravirus, Kostyavirus, Krampusvirus, Kryptosalinivirus, Kuleanavirus, Labanvirus, Lacnuvirus, Lacusarxvirus, Lafunavirus, Lambdavirus, Lambovirus, Lanavirus, Larmunavirus, Laroyevirus, Latrobevirus, Leicestervirus, Lentavirus, Liebevirus, Liefievirus, Lillamyvirus, Lokivirus, Lomovskayavirus, Luckybarnesvirus, Luckytenvirus, Lughvirus, Lwoffvirus, Magadivirus, Majavirus, Manhattanvirus, Mapvirus, Mardecavirus, Marienburgvirus, Marvinvirus, Maxrubnervirus, Mementomorivirus, Metamorphoovirus, Minunavirus, Moineauvirus, Montyvirus, Mudcatvirus, Mufasoctovirus, Muminvirus, Murrayvirus, Nanhaivirus, Nazgulvirus, Neferthenavirus, Nesevirus, Nevevirus, Nickievirus, Nonanavirus, Nyceiraevirus, Oengusvirus, Omegavirus, Oneupvirus, Orchidvirus, Oshimavirus, Pahexavirus, Pamexvirus, Pankowvirus, Papyrusvirus, Patiencevirus, Pepyhexavirus, Phifelvirus, Picardvirus, Pikminvirus, Pleeduovirus, Priunavirus, Psavirus, Psimunavirus, Pleetrevirus, Poushouvirus, Predatorvirus, Pulverervirus, Questintvirus, Quhwahvirus, Radostvirus, Raleighvirus, Ravarandavirus, Ravinvirus, Rerduovirus, Rigallicvirus, Rimavirus, Rockefellervirus, Rockvillevirus, Rogerhendrixvirus, Ronaldovirus, Roufvirus, Rowavirus, Ruthyvirus, Samistivirus, Samunavirus, Samwavirus, Sandinevirus, Sanovirus, Sansavirus, Saphexavirus, Sashavirus, Sasvirus, Saundersvirus, Sawaravirus, Scapunavirus, Schnabeltiervirus, Schubertvirus, Seongbukvirus, Septimatrevirus, Seussvirus, Sextaecvirus, Skunavirus, Slashvirus, Sleepyheadvirus, Smoothievirus, Sonalivirus, Soupsvirus, Sourvirus, Sozzivirus, Sparkyvirus, Spbetavirus, Spizizenvirus, Squashvirus, Squirtyvirus, Stanholtvirus, Steinhofvirus, Sukhumvitvirus, Tandoganvirus, Tankvirus, Tantvirus, Terapinvirus, Teubervirus, Thetabobvirus, Tigunavirus, Timquatrovirus, Tinduovirus, Titanvirus, Tortellinivirus, Triavirus, Trigintaduovirus, Trinavirus, Trinevirus, Triplejayvirus, Unahavirus, Uwajimavirus, Vashvirus, Vedamuthuvirus, Vendettavirus, Vhulanivirus, Vidquintavirus, Vieuvirus, Vividuovirus, Vojvodinavirus, Waukeshavirus, Wbetavirus, Weaselvirus, Whackvirus, Whiteheadvirus, Wildcatvirus, Wilnyevirus, Wizardvirus, Woesvirus, Woodruffvirus, Xiamenvirus, Xipdecavirus, Yangvirus, Yonseivirus, Yuavirus, Yvonnevirus, and/or Zetavirus. In specific aspects, a bacteriophage of the disclosure is a Siphoviridae T1-like virus. In specific aspects, a bacteriophage of the disclosure is a Siphoviridae A-like virus. In specific aspects, a Siphoviridae bacteriophage of the disclosure is ΦER39. In some aspects, bacteriophage from any one or more of the foregoing bacteriophage genera, or species therein, may be excluded from the compositions and/or methods disclosed herein.

A bacteriophage of the present disclosure may be a phage capable of infecting one or more pathogenic bacteria. A bacteriophage of the present disclosure may co-exist with one or more pathogenic bacteria in the host's gut. A bacteriophage of the present disclosure may be a phage capable of infecting one or more bacteria of the family Enterobacteriaceae. In some aspects, a bacteriophage of the disclosure is a phage capable of infecting Klebsiella, such as Klebsiella pneumoniae (K. pneumoniae), Klebsiella quasipneumoniae (K. quasipneumoniae), and/or Klebsiella variicola (K. variicola). In some aspects, the Klebsiella is multidrug-resistant (MDR) Klebsiella. In some aspects, the Klebsiella is hypervirulent Klebsiella. In some aspects, any one or more of the foregoing Klebsiella strains may be excluded from the compositions and/or methods disclosed herein.

In some aspects, infection with a bacteriophage of the disclosure can result in capsule depolymerization of a capsule of the pathogenic bacteria and non-capsulated pathogenic bacteria. In addition to simply destroying the capsule, in some aspects, bacteriophage can select for bacteria that mutate capsule synthesis genes in order to evade bacteriophage predation and therefore lose their capsule. Since these mutations are passed on to daughter cells after cell division, the bacteriophage can select for a bacterial population with permanent and heritable capsule loss. Capsules of pathogenic bacteria can protect the bacteria from clearance by the host immune system as a result of the activity of antibodies, phagocytic cells, proteins of the complement cascade, and antimicrobial peptides. Capsules of pathogenic bacteria also cover bacterial lipopolysaccharides (LPS) that are the major outer surface membrane components present in almost all Gram-negative bacteria and that stimulate innate or natural immunity of the host.

Thus, in some aspects, capsule depolymerization upon infection with a bacteriophage of the disclosure can result in lipopolysaccharide exposure and can promote recognition of the pathogenic bacteria by the host immune system. For example, in some aspects, capsule depolymerization upon infection with a bacteriophage of the disclosure can result in lipopolysaccharide exposure and can promote macrophage engulfment of the pathogenic bacteria to clear the pathogenic bacteria from the host. In some aspects, capsule depolymerization upon infection with a bacteriophage of the disclosure can result in increased susceptibility of the pathogenic bacteria to infection by one or more additional bacteriophage.

A bacteriophage composition of the disclosure may comprise, for example, one or more bacteriophage capable of infecting a pathogenic bacteria. A bacteriophage composition of the disclosure may comprise, for example, one or more lytic bacteriophage capable of lysing a pathogenic bacteria. A bacteriophage composition of the present disclosure may comprise, consist essentially of, or consist of at least, at most, exactly, or between any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more different bacteriophages. In some aspects, a bacteriophage composition of the disclosure comprises one or more of ΦER3, ΦER11, ΦER12, ΦER15, ΦER16, ΦER27, ΦER36, ΦER39, ΦER43, ΦER46, ΦMM7, or ΦMM9, or any combination thereof. In some aspects, any one or more of the foregoing bacteriophage may be excluded from the compositions and/or methods disclosed herein.

In some aspects, a bacteriophage composition of the disclosure comprises, consists essentially of, or consists of ΦER3. In some aspects, a bacteriophage composition of the disclosure comprises, consists essentially of, or consists of ΦER11. In some aspects, a bacteriophage composition of the disclosure comprises, consists essentially of, or consists of ΦER12. In some aspects, a bacteriophage composition of the disclosure comprises, consists essentially of, or consists of ΦER15. In some aspects, a bacteriophage composition of the disclosure comprises, consists essentially of, or consists of ΦER16. In some aspects, a bacteriophage composition of the disclosure comprises, consists essentially of, or consists of ΦER27. In some aspects, a bacteriophage composition of the disclosure comprises, consists essentially of, or consists of ΦER36. In some aspects, a bacteriophage composition of the disclosure comprises, consists essentially of, or consists of ΦER39. In some aspects, a bacteriophage composition of the disclosure comprises, consists essentially of, or consists of ΦER43. In some aspects, a bacteriophage composition of the disclosure comprises, consists essentially of, or consists of ΦER46. In some aspects, a bacteriophage composition of the disclosure comprises, consists essentially of, or consists of ΦMM7. In some aspects, a bacteriophage composition of the disclosure comprises, consists essentially of, or consists of ΦMM9. In some aspects, a bacteriophage composition comprises, consists essentially of, or consists of two or more of ΦER3, ΦER11, ΦER12, ΦER15, ΦER16, ΦER27, ΦER36, ΦER39, ΦER43, ΦER46, ΦMM7, or ΦMM9. In some aspects, a bacteriophage composition comprises, consists essentially of, or consists of three or more of ΦER3, ΦER11, ΦER12, ΦER15, ΦER16, ΦER27, ΦER36, ΦER39, ΦER43, ΦER46, ΦMM7, or ΦMM9. In some aspects, a bacteriophage composition comprises, consists essentially of, or consists of four or more of ΦER3, ΦER11, ΦER12, ΦER15, ΦER16, ΦER27, ΦER36, ΦER39, ΦER43, ΦER46, ΦMM7, or ΦMM9.

In some aspects, a bacteriophage composition comprises, consists essentially of, or consists of ΦER12, ΦER15, ΦER16, ΦER39, ΦER46, or any combination thereof. In some aspects, a bacteriophage composition comprises, consists essentially of, or consists of ΦER3, ΦER11, ΦER27, ΦER36, ΦMM9, or any combination thereof. In some aspects, a bacteriophage composition comprises, consists essentially of, or consists of ΦER15, ΦER27, ΦER43, ΦER46, ΦMM7, or any combination thereof.

In some aspects, a bacteriophage composition comprises, consists essentially of, or consists of one of the bacteriophage combinations provided in Table 1.

TABLE 1
Bacteriophage Combinations

| | ΦER15 | ΦER16 | ΦER39 | ΦER46 |
|---|---|---|---|---|
| ΦER12 | + | | | |
| | + | + | | |
| | + | | + | |
| | + | | | + |
| | + | + | + | |
| | + | + | | + |
| | + | | + | + |
| | + | + | + | + |
| | | + | | |
| | | + | + | |
| | | + | | + |
| | | + | + | + |
| | | | + | |
| | | | + | + |
| | | | | + |

| | ΦER16 | ΦER39 | ΦER46 |
|---|---|---|---|
| ΦER15 | + | | |
| | | + | |
| | | | + |
| | + | + | |
| | + | | + |
| | | + | + |
| | + | + | + |

| | ΦER39 | ΦER46 |
|---|---|---|
| ΦER16 | + | |
| | | + |
| | + | + |

| | | ΦER46 | | |
|---|---|---|---|---|
| | ΦER39 | + | | |
| | ΦER11 | ΦER27 | ΦER36 | ΦMM9 |
| ΦER3 | + | | | |
| | + | + | | |
| | + | | + | |
| | + | | | + |
| | + | + | + | |
| | + | + | | + |
| | + | | + | + |
| | + | + | + | + |
| | | + | | |
| | | + | + | |
| | | + | | + |
| | | + | + | |
| | | | + | |
| | | | + | + |
| | | | | + |

| | ΦER27 | ΦER36 | ΦMM9 |
|---|---|---|---|
| ΦER11 | + | | |
| | | + | |
| | | | + |
| | + | + | |
| | + | | + |
| | | + | + |
| | + | + | + |

| | ΦER36 | ΦMM9 |
|---|---|---|
| ΦER27 | + | |
| | | + |
| | + | + |

| | | | ΦMM9 | |
|---|---|---|---|---|
| | ΦER36 | | + | |
| | ΦER27 | ΦER43 | ΦER46 | ΦMM7 |
| ΦER15 | + | | | |
| | + | + | | |
| | + | | + | |
| | + | | | + |
| | + | + | + | |
| | + | + | | + |
| | + | | + | + |
| | + | + | + | + |
| | | + | | |
| | | + | + | |
| | | + | | + |
| | | + | + | + |
| | | | + | |
| | | | + | + |
| | | | | + |

| | ΦER43 | ΦER46 | ΦMM9 |
|---|---|---|---|
| ΦER27 | + | | |
| | | + | |
| | | | + |
| | + | + | |
| | + | | + |
| | | + | + |
| | + | + | + |

| | ΦER46 | ΦMM9 |
|---|---|---|
| ΦER43 | + | |
| | | + |
| | + | + |

| | ΦMM9 |
|---|---|
| ΦER46 | + |

A bacteriophage composition comprising two or more different bacteriophages may comprise various amounts of each bacteriophage. For example, a composition may comprise substantially the same amount of each bacteriophage. Alternatively, a composition may comprise substantially different amounts of each bacteriophage.

A bacteriophage composition may comprise at least, at most, exactly, between any two of, or about 10³, 10⁴, 10⁵, 10⁶, 10⁷, 10⁸, 10⁹, 10¹⁰, 10¹¹, or 10¹² plaque forming units (PFU) of each of the one or more bacteriophage in the composition, or more. In some aspects, a bacteriophage composition comprises at least, at most, exactly, between any two of, or about 10³, 10⁴, 10⁵, 10⁶, 10⁷, 10⁸, 10⁹, 10¹⁰, 10¹¹, or 10¹² PFU of ΦER3. In some aspects, a bacteriophage composition comprises at least, at most, exactly, between any two of, or about 10³, 10⁴, 10⁵, 10⁶, 10⁷, 10⁸, 10⁹, 10¹⁰, 10¹¹, or 10¹² PFU of ΦER11. In some aspects, a bacteriophage composition comprises at least, at most, exactly, between any two of, or about 10³, 10⁴, 10⁵, 10⁶, 10⁷, 10⁸, 10⁹, 10¹⁰, 10¹¹, or 10¹² PFU of ΦER12. In some aspects, a bacteriophage composition comprises at least, at most, exactly, between any two of, or about 10³, 10⁴, 10⁵, 10⁶, 10⁷, 10⁸, 10⁹, 10¹⁰, 10¹¹, or 10¹² PFU of ΦER15. In some aspects, a bacteriophage composition comprises at least, at most, exactly, between any two of, or about 10³, 10⁴, 10⁵, 10⁶, 10⁷, 10⁸, 10⁹, 10¹⁰, 10¹¹, or 10¹² PFU of ΦER16. In some aspects, a bacteriophage composition comprises at least, at most, exactly, between any two of, or about 10³, 10⁴, 10⁵, 10⁶, 10⁷, 10⁸, 10⁹, 10¹⁰, 10¹¹, or 10¹² PFU of ΦER27. In some aspects, a bacteriophage composition comprises at least, at most, exactly, between any two of, or about 10³, 10⁴, 10⁵, 10⁶, 10⁷, 10⁸, 10⁹, 10¹⁰, 10¹¹, or 10¹² PFU of ΦER36. In some aspects, a bacteriophage composition comprises at least, at most, exactly, between any two of, or about 10³, 10⁴, 10⁵, 10⁶, 10⁷, 10⁸, 10⁹, 10¹⁰, 10¹¹, or 10¹² PFU of ΦER39. In some aspects, a bacteriophage composition comprises at least, at most, exactly, between any two of, or about 10³, 10⁴, 10⁵, 10⁶, 10⁷, 10⁸, 10⁹, 10¹⁰, 10¹¹, or 10¹² PFU of ΦER43. In some aspects, a bacteriophage composition comprises at least, at most, exactly, between any two of, or about 10³, 10⁴, 10⁵, 10⁶, 10⁷, 10⁸, 10⁹, 10¹⁰, 10¹¹, or 10¹² PFU of ΦER46. In some aspects, a bacteriophage composition comprises at least, at most, exactly, between any two of, or about 10³, 10⁴, 10⁵, 10⁶, 10⁷, 10⁸, 10⁹, 10¹⁰, 10¹¹, or 10¹² PFU of ΦMM7. In some aspects, a bacteriophage composition comprises at least, at most, exactly, between any two of, or about 10³, 10⁴, 10⁵, 10⁶, 10⁷, 10⁸, 10⁹, 10¹⁰, 10¹¹, or 10¹² PFU of ΦMM9.

In some aspects, a bacteriophage composition of the disclosure may comprise, in addition to one or more bacteriophages, one or more metals. The one or more metals may include, for example, calcium, magnesium, iron, sodium, and/or potassium. In some aspects, any one or more of the foregoing metals may be excluded from the compositions and/or methods disclosed herein.

Aspects of the present disclosure comprise, consist essentially of, or consist of bacteriophage compositions, kits, and devices comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or more bacteriophage, and methods for use. Aspects of the present disclosure comprise, consist essentially of, or consist of bacteriophage compositions, kits, and devices comprising ΦER3, ΦER11, ΦER12, ΦER15, ΦER16, ΦER27, ΦER36, ΦER39, ΦER43, ΦER46, ΦMM7, and/or ΦMM9, and methods for use. Bacteriophage genomes can be provided as bacteriophage sequences. The term “bacteriophage sequence” refers to a sequence from bacteriophage and does not include a nucleotide sequence greater than 20 contiguous nucleotides that is not from bacteriophage. Such non-bacteriophage sequences would be heterologous, and in aspects of the disclosure, they could be used to purify, select, and/or provide additional functionality to the bacteriophage. For example, in some aspects, heterologous sequences include, but are not limited to, capsule depolymerases that might expand or alter the host range of a bacteriophage, biofilm-degrading enzymes, antimicrobial proteins and/or peptides, or CRISPR-Cas systems and/or DNA-degrading enzymes. In some aspects, the heterologous sequence encodes a peptide or polypeptide.

In some aspects, a composition may comprise an isolated bacteriophage, wherein the bacteriophage were evolved to improve lytic capacity, reduce lysogenic capacity, reduce antibiotic resistance gene obtainment capacity, reduce bacterial virulence gene obtainment capacity, and/or increase the number of bacterial strains subject to lysis by the isolated bacteriophage lytic. Thus, variants or evolved bacteriophage sequences are also contemplated as aspects of the methods and compositions disclosed herein.

### A. Bacteriophage ER3

Aspects of the present disclosure comprise, consist essentially of, or consist of bacteriophage ER3 (also “ΦER3,” used synonymously herein), compositions, kits, and devices comprising ΦER3, and methods for use. A ΦER3 genome is described by GENBANK® Accession Number ______, which is noted as 48,293 bp in length, and the sequence of which is incorporated herein by reference as SEQ ID NO:8 or SEQ ID NO:32. Comparing SEQ ID NO:8 to SEQ ID NO:32, SEQ ID NO:32 is a reorganization of the phage genome encoded by SEQ ID NO: 8, including a different start site and excluding assembly artifacts.

In some aspects, a bacteriophage or composition thereof may comprise an isolated bacteriophage comprising ΦER3 (SEQ ID NO:8 or SEQ ID NO:32). In some aspects, a bacteriophage or composition thereof may comprise an isolated bacteriophage consisting essentially of ΦER3 (SEQ ID NO:8 or SEQ ID NO:32). In some aspects, a bacteriophage or composition thereof may comprise an isolated bacteriophage consisting of ΦER3 (SEQ ID NO: 8 or SEQ ID NO:32).

In some aspects, a bacteriophage may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more nucleotide substitutions to the genomic sequence of ΦER3 (SEQ ID NO:8 or SEQ ID NO:32). In some aspects, a bacteriophage may be at least, at most, exactly, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%, 99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83%, 99.84%, 99.85%, 99.86%, 99.87%, 99.88%, 99.89%, 99.9%, 99.91%, 99.92%, 99.93%, 99.94%, 99.95%, 99.96%, 99.97%, 99.98%, 99.99%, or 100% (or any derivable range therein) similar, identical, or homologous with the genomic sequence of ΦER3 (SEQ ID NO:8 or SEQ ID NO:32).

In some aspects, a bacteriophage or composition thereof may comprise an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequence of ΦER3 (SEQ ID NO:8 or SEQ ID NO:32).

Variants or evolved bacteriophage derived from ΦER3 having a genome comprising, consisting, or consisting essentially of SEQ ID NO:8 or SEQ ID NO:32 are also contemplated as aspects of the methods and compositions disclosed herein.

### B. Bacteriophage ER11

Aspects of the present disclosure comprise, consist essentially of, or consist of bacteriophage ER11 (also “ΦER11,” used synonymously herein), compositions, kits, and devices comprising ΦER11, and methods for use. A ΦER11 genome is described by GENBANK® Accession Number ______, which is noted as 164,356 bp in length, and the sequence of which is incorporated herein by reference as SEQ ID NO:14 or SEQ ID NO:36. Comparing SEQ ID NO: 14 to SEQ ID NO:36, SEQ ID NO:36 is a reorganization of the phage genome encoded by SEQ ID NO:14, including a different start site and excluding assembly artifacts.

In some aspects, a bacteriophage or composition thereof may comprise an isolated bacteriophage comprising ΦER11 (SEQ ID NO:14 or SEQ ID NO:36). In some aspects, a bacteriophage or composition thereof may comprise an isolated bacteriophage consisting essentially of ΦER11 (SEQ ID NO:14 or SEQ ID NO:36). In some aspects, a bacteriophage or composition thereof may comprise an isolated bacteriophage consisting of ΦER11 (SEQ ID NO: 14 or SEQ ID NO:36).

In some aspects, a bacteriophage may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more nucleotide substitutions to the genomic sequence of ΦER11 (SEQ ID NO:14 or SEQ ID NO:36). In some aspects, a bacteriophage may be at least, at most, exactly, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%, 99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83%, 99.84%, 99.85%, 99.86%, 99.87%, 99.88%, 99.89%, 99.9%, 99.91%, 99.92%, 99.93%, 99.94%, 99.95%, 99.96%, 99.97%, 99.98%, 99.99%, or 100% (or any derivable range therein) similar, identical, or homologous with the genomic sequence of ΦER11 (SEQ ID NO:14 or SEQ ID NO:36).

In some aspects, a bacteriophage or composition thereof may comprise an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequence of ΦER11 (SEQ ID NO:14 or SEQ ID NO:36).

Variants or evolved bacteriophage derived from ΦER11 having a genome comprising, consisting, or consisting essentially of SEQ ID NO:14 or SEQ ID NO:36 are also contemplated as aspects of the methods and compositions disclosed herein.

### C. Bacteriophage ER12

Aspects of the present disclosure comprise, consist essentially of, or consist of bacteriophage ER12 (also “ΦER12,” used synonymously herein), compositions, kits, and devices comprising ΦER12, and methods for use. A ΦER12 genome is described by GENBANK® Accession Number ______, which is noted as 157,838 bp in length, the sequence of which is incorporated herein by reference as SEQ ID NO:1, and GENBANK® Accession Number ______, which is noted as 157,764 bp in length, the sequence of which is incorporated herein by reference as SEQ ID NO:37. Comparing SEQ ID NO: 1 to SEQ ID NO:37, SEQ ID NO: 37 is a reorganization of the phage genome encoded by SEQ ID NO: 1, including a different start site and excluding assembly artifacts.

In some aspects, a bacteriophage or composition thereof may comprise an isolated bacteriophage comprising ΦER12 (SEQ ID NO:1 or SEQ ID NO:37). In some aspects, a bacteriophage or composition thereof may comprise an isolated bacteriophage consisting essentially of ΦER12 (SEQ ID NO:1 or SEQ ID NO:37). In some aspects, a bacteriophage or composition thereof may comprise an isolated bacteriophage consisting of ΦER12 (SEQ ID NO: 1 or SEQ ID NO:37).

In some aspects, a bacteriophage may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more nucleotide substitutions to the genomic sequence of ΦER12 (SEQ ID NO:1 or SEQ ID NO:37). In some aspects, a bacteriophage may be at least, at most, exactly, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%, 99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83%, 99.84%, 99.85%, 99.86%, 99.87%, 99.88%, 99.89%, 99.9%, 99.91%, 99.92%, 99.93%, 99.94%, 99.95%, 99.96%, 99.97%, 99.98%, 99.99%, or 100% (or any derivable range therein) similar, identical, or homologous with the genomic sequence of ΦER12 (SEQ ID NO:1 or SEQ ID NO:37).

In some aspects, a bacteriophage or composition thereof may comprise an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequence of ΦER12 (SEQ ID NO:1 or SEQ ID NO:37).

Variants or evolved bacteriophage derived from ΦER12 having a genome comprising, consisting, or consisting essentially of SEQ ID NO:1 or SEQ ID NO:37 are also contemplated as aspects of the methods and compositions disclosed herein.

### D. Bacteriophage ER15

Aspects of the present disclosure comprise, consist essentially of, or consist of bacteriophage ER15 (also “ΦER15,” used synonymously herein), compositions, kits, and devices comprising ΦER15, and methods for use. A ΦER15 genome is described by GENBANK® Accession Number ______, which is noted as 165,661 bp in length, the sequence of which is incorporated herein by reference as SEQ ID NO:2, and GENBANK® Accession Number ______, which is noted as 165,587 bp in length, the sequence of which is incorporated herein by reference as SEQ ID NO:40. Comparing SEQ ID NO:2 to SEQ ID NO:40, SEQ ID NO: 40 is a reorganization of the phage genome encoded by SEQ ID NO:2, including a different start site and excluding assembly artifacts.

In some aspects, a bacteriophage or composition thereof may comprise an isolated bacteriophage comprising ΦER15 (SEQ ID NO:2 or SEQ ID NO:40). In some aspects, a bacteriophage or composition thereof may comprise an isolated bacteriophage consisting essentially of ΦER15 (SEQ ID NO:2 or SEQ ID NO:40). In some aspects, a bacteriophage or composition thereof may comprise an isolated bacteriophage consisting of ΦER15 (SEQ ID NO: 2 or SEQ ID NO:40).

In some aspects, a bacteriophage may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more nucleotide substitutions to the genomic sequence of ΦER15 (SEQ ID NO:2 or SEQ ID NO:40). In some aspects, a bacteriophage may be at least, at most, exactly, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%, 99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83%, 99.84%, 99.85%, 99.86%, 99.87%, 99.88%, 99.89%, 99.9%, 99.91%, 99.92%, 99.93%, 99.94%, 99.95%, 99.96%, 99.97%, 99.98%, 99.99%, or 100% (or any derivable range therein) similar, identical, or homologous with the genomic sequence of ΦER15 (SEQ ID NO:2 or SEQ ID NO:40).

In some aspects, a bacteriophage or composition thereof may comprise an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequence of ER15 (SEQ ID NO:2 or SEQ ID NO:40).

Variants or evolved bacteriophage derived from ΦER15 having a genome comprising, consisting, or consisting essentially of SEQ ID NO:2 or SEQ ID NO:40 are also contemplated as aspects of the methods and compositions disclosed herein.

### E. Bacteriophage ER16

Aspects of the present disclosure comprise, consist essentially of, or consist of bacteriophage ER16 (also “ΦER16,” used synonymously herein), compositions, kits, and devices comprising ΦER16, and methods for use. A ΦER16 genome is described by GENBANK® Accession Number ______, which is noted as 175,688 bp in length, the sequence of which is incorporated herein by reference as SEQ ID NO:3, and GENBANK® Accession Number ______, which is noted as 175,617 bp in length, the sequence of which is incorporated herein by reference as SEQ ID NO:41. Comparing SEQ ID NO:3 to SEQ ID NO:41, SEQ ID NO: 41 is a reorganization of the phage genome encoded by SEQ ID NO:3, including a different start site and excluding assembly artifacts.

In some aspects, a bacteriophage or composition thereof may comprise an isolated bacteriophage comprising ΦER16 (SEQ ID NO:3 or SEQ ID NO:41). In some aspects, a bacteriophage or composition thereof may comprise an isolated bacteriophage consisting essentially of ΦER16 (SEQ ID NO:3 or SEQ ID NO:41). In some aspects, a bacteriophage or composition thereof may comprise an isolated bacteriophage consisting of ΦER16 (SEQ ID NO: 3 or SEQ ID NO:41).

In some aspects, a bacteriophage may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more nucleotide substitutions to the genomic sequence of ΦER16 (SEQ ID NO:3 or SEQ ID NO:41). In some aspects, a bacteriophage may be at least, at most, exactly, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%, 99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83%, 99.84%, 99.85%, 99.86%, 99.87%, 99.88%, 99.89%, 99.9%, 99.91%, 99.92%, 99.93%, 99.94%, 99.95%, 99.96%, 99.97%, 99.98%, 99.99%, or 100% (or any derivable range therein) similar, identical, or homologous with the genomic sequence of ΦER16 (SEQ ID NO:3 or SEQ ID NO:41).

In some aspects, a bacteriophage or composition thereof may comprise an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequence of ΦER16 (SEQ ID NO:3 or SEQ ID NO:41).

Variants or evolved bacteriophage derived from ΦER16 having a genome comprising, consisting, or consisting essentially of SEQ ID NO:3 or SEQ ID NO:41 are also contemplated as aspects of the methods and compositions disclosed herein.

### F. Bacteriophage ER27

Aspects of the present disclosure comprise, consist essentially of, or consist of bacteriophage ER27 (also “ΦER27,” used synonymously herein), compositions, kits, and devices comprising ΦER27, and methods for use. A ΦER27 genome is described by GENBANK® Accession Number ______, which is noted as 176,860 bp in length, and the sequence of which is incorporated herein by reference as SEQ ID NO:20 or SEQ ID NO:47. Comparing SEQ ID NO:20 to SEQ ID NO:47, SEQ ID NO:47 is a reorganization of the phage genome encoded by SEQ ID NO:20, including a different start site and excluding assembly artifacts.

In some aspects, a bacteriophage or composition thereof may comprise an isolated bacteriophage comprising ΦER27 (SEQ ID NO:20 or SEQ ID NO:47). In some aspects, a bacteriophage or composition thereof may comprise an isolated bacteriophage consisting essentially of ΦER27 (SEQ ID NO:20 or SEQ ID NO:47). In some aspects, a bacteriophage or composition thereof may comprise an isolated bacteriophage consisting of ΦER27 (SEQ ID NO: 20 or SEQ ID NO:47).

In some aspects, a bacteriophage may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant nucleotide substitutions to the genomic sequence of ΦER27 (SEQ ID NO:20 or SEQ ID NO: 47). In some aspects, a bacteriophage may be at least, at most, exactly, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%, 99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83%, 99.84%, 99.85%, 99.86%, 99.87%, 99.88%, 99.89%, 99.9%, 99.91%, 99.92%, 99.93%, 99.94%, 99.95%, 99.96%, 99.97%, 99.98%, 99.99%, or 100% (or any derivable range therein) similar, identical, or homologous with the genomic sequence of ΦER27 (SEQ ID NO:20 or SEQ ID NO:47).

In some aspects, a bacteriophage or composition thereof may comprise an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequence of ΦER27 (SEQ ID NO:20 or SEQ ID NO:47).

Variants or evolved bacteriophage derived from ΦER27 having a genome comprising, consisting, or consisting essentially of SEQ ID NO:20 or SEQ ID NO:47 are also contemplated as aspects of the methods and compositions disclosed herein.

### G. Bacteriophage ER36

Aspects of the present disclosure comprise, consist essentially of, or consist of bacteriophage ER36 (also “ΦER36,” used synonymously herein), compositions, kits, and devices comprising ΦER36, and methods for use. A ΦER36 genome is described by GENBANK® Accession Number ______, which is noted as 176,466 bp in length, and the sequence of which is incorporated herein by reference as SEQ ID NO:22 or SEQ ID NO:49. Comparing SEQ ID NO:22 to SEQ ID NO:49, SEQ ID NO:49 is a reorganization of the phage genome encoded by SEQ ID NO:22, including a different start site and excluding assembly artifacts.

In some aspects, a bacteriophage or composition thereof may comprise an isolated bacteriophage comprising ΦER36 (SEQ ID NO:22 or SEQ ID NO:49). In some aspects, a bacteriophage or composition thereof may comprise an isolated bacteriophage consisting essentially of ΦER36 (SEQ ID NO:22 or SEQ ID NO:49). In some aspects, a bacteriophage or composition thereof may comprise an isolated bacteriophage consisting of ΦER36 (SEQ ID NO: 22 or SEQ ID NO:49).

In some aspects, a bacteriophage may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant nucleotide substitutions to the genomic sequence of ER36 (SEQ ID NO:22 or SEQ ID NO: 49). In some aspects, a bacteriophage may be at least, at most, exactly, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%, 99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83%, 99.84%, 99.85%, 99.86%, 99.87%, 99.88%, 99.89%, 99.9%, 99.91%, 99.92%, 99.93%, 99.94%, 99.95%, 99.96%, 99.97%, 99.98%, 99.99%, or 100% (or any derivable range therein) similar, identical, or homologous with the genomic sequence of ΦER36 (SEQ ID NO:22 or SEQ ID NO:49).

In some aspects, a bacteriophage or composition thereof may comprise an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequence of ΦER36 (SEQ ID NO:22 or SEQ ID NO:49).

Variants or evolved bacteriophage derived from ΦER36 having a genome comprising, consisting, or consisting essentially of SEQ ID NO:22 or SEQ ID NO:49 are also contemplated as aspects of the methods and compositions disclosed herein.

### H. Bacteriophage ER39

Aspects of the present disclosure comprise, consist essentially of, or consist of bacteriophage ER39 (also “ΦER39,” used synonymously herein), compositions, kits, and devices comprising ΦER39, and methods for use. A ΦER39 genome is described by GENBANK® Accession Number ______, which is noted as 48, 196 bp in length, the sequence of which is incorporated herein by reference as SEQ ID NO:4, and GENBANK® Accession Number ______, which is noted as 48,149 bp in length, the sequence of which is incorporated herein by reference as SEQ ID NO:52. Comparing SEQ ID NO:4 to SEQ ID NO:39, SEQ ID NO: 39 is a reorganization of the phage genome encoded by SEQ ID NO:4, including a different start site and excluding assembly artifacts.

In some aspects, a bacteriophage or composition thereof may comprise an isolated bacteriophage comprising ΦER39 (SEQ ID NO:4 or SEQ ID NO:52). In some aspects, a bacteriophage or composition thereof may comprise an isolated bacteriophage consisting essentially of ΦER39 (SEQ ID NO:4 or SEQ ID NO:52). In some aspects, a bacteriophage or composition thereof may comprise an isolated bacteriophage consisting of ΦER39 (SEQ ID NO: 4 or SEQ ID NO:52).

In some aspects, a bacteriophage may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant nucleotide substitutions to the genomic sequence of ΦER39 (SEQ ID NO:4 or SEQ ID NO:52). In some aspects, a bacteriophage may be at least, at most, exactly, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%, 99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83%, 99.84%, 99.85%, 99.86%, 99.87%, 99.88%, 99.89%, 99.9%, 99.91%, 99.92%, 99.93%, 99.94%, 99.95%, 99.96%, 99.97%, 99.98%, 99.99%, or 100% (or any derivable range therein) similar, identical, or homologous with the genomic sequence of ΦER39 (SEQ ID NO:4 or SEQ ID NO:52).

In some aspects, a bacteriophage or composition thereof may comprise an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequence of ΦER39 (SEQ ID NO:4 or SEQ ID NO:52).

Variants or evolved bacteriophage derived from ΦER39 having a genome comprising, consisting, or consisting essentially of SEQ ID NO:4 or SEQ ID NO:52 are also contemplated as aspects of the methods and compositions disclosed herein.

### I. Bacteriophage ER43

Aspects of the present disclosure comprise, consist essentially of, or consist of bacteriophage ER43 (also “ΦER43,” used synonymously herein), compositions, kits, and devices comprising ΦER43, and methods for use. A ER43 genome is described by GENBANK® Accession Number ______, which is noted as 40,418 bp in length, and the sequence of which is incorporated herein by reference as SEQ ID NO:25 or SEQ ID NO:53. Comparing SEQ ID NO:25 to SEQ ID NO:53, SEQ ID NO:53 is a reorganization of the phage genome encoded by SEQ ID NO:25, including a different start site and excluding assembly artifacts.

In some aspects, a bacteriophage or composition thereof may comprise an isolated bacteriophage comprising ΦER43 (SEQ ID NO:25 or SEQ ID NO:53). In some aspects, a bacteriophage or composition thereof may comprise an isolated bacteriophage consisting essentially of ΦER43 (SEQ ID NO:25 or SEQ ID NO:53). In some aspects, a bacteriophage or composition thereof may comprise an isolated bacteriophage consisting of ΦER43 (SEQ ID NO: 25 or SEQ ID NO:53).

In some aspects, a bacteriophage may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant nucleotide substitutions to the genomic sequence of ΦER43 (SEQ ID NO:25 or SEQ ID NO: 53). In some aspects, a bacteriophage may be at least, at most, exactly, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%, 99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83%, 99.84%, 99.85%, 99.86%, 99.87%, 99.88%, 99.89%, 99.9%, 99.91%, 99.92%, 99.93%, 99.94%, 99.95%, 99.96%, 99.97%, 99.98%, 99.99%, or 100% (or any derivable range therein) similar, identical, or homologous with the genomic sequence of ΦER43 (SEQ ID NO:25 or SEQ ID NO:53).

In some aspects, a bacteriophage or composition thereof may comprise an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequence of ΦER43 (SEQ ID NO:25 or SEQ ID NO:53).

Variants or evolved bacteriophage derived from ΦER43 having a genome comprising, consisting, or consisting essentially of SEQ ID NO:25 or SEQ ID NO:53 are also contemplated as aspects of the methods and compositions disclosed herein.

### J. Bacteriophage ER46

Aspects of the present disclosure comprise, consist essentially of, or consist of bacteriophage ER46 (also “ΦER46,” used synonymously herein), compositions, kits, and devices comprising ΦER46, and methods for use. A ΦER46 genome is described by GENBANK® Accession Number ______, which is noted as 112,942 bp in length, the sequence of which is incorporated herein by reference as SEQ ID NO:5, and GENBANK® Accession Number ______, which is noted as 112,865 bp in length, the sequence of which is incorporated herein by reference as SEQ ID NO:56. Comparing SEQ ID NO:5 to SEQ ID NO:56, SEQ ID NO: 56 is a reorganization of the phage genome encoded by SEQ ID NO:5, including a different start site and excluding assembly artifacts.

In some aspects, a bacteriophage or composition thereof may comprise an isolated bacteriophage comprising ΦER46 (SEQ ID NO:5 or SEQ ID NO:56). In some aspects, a bacteriophage or composition thereof may comprise an isolated bacteriophage consisting essentially of ΦER46 (SEQ ID NO:5 or SEQ ID NO:56). In some aspects, a bacteriophage or composition thereof may comprise an isolated bacteriophage consisting of ΦER46 (SEQ ID NO: 5 or SEQ ID NO:56).

In some aspects, a bacteriophage may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant nucleotide substitutions to the genomic sequence of ΦER46 (SEQ ID NO:5 or SEQ ID NO:56). In some aspects, a bacteriophage may be at least, at most, exactly, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%, 99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83%, 99.84%, 99.85%, 99.86%, 99.87%, 99.88%, 99.89%, 99.9%, 99.91%, 99.92%, 99.93%, 99.94%, 99.95%, 99.96%, 99.97%, 99.98%, 99.99%, or 100% (or any derivable range therein) similar, identical, or homologous with the genomic sequence of ΦER46 (SEQ ID NO:5 or SEQ ID NO:56).

In some aspects, a bacteriophage or composition thereof may comprise an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequence of ΦER46 (SEQ ID NO:5 or SEQ ID NO:56).

Variants or evolved bacteriophage derived from ΦER46 having a genome comprising, consisting, or consisting essentially of SEQ ID NO:5 or SEQ ID NO:56 are also contemplated as aspects of the methods and compositions disclosed herein.

### K. Bacteriophage MM7

Aspects of the present disclosure comprise, consist essentially of, or consist of bacteriophage MM7 (also “ΦMM7,” used synonymously herein), compositions, kits, and devices comprising ΦMM7, and methods for use. A ΦMM7 genome is described by GENBANK® Accession Number ______, which is noted as 47,608 bp in length, and the sequence of which is incorporated herein by reference as SEQ ID NO:72.

In some aspects, a bacteriophage or composition thereof may comprise an isolated bacteriophage comprising ΦMM7 (SEQ ID NO:72). In some aspects, a bacteriophage or composition thereof may comprise an isolated bacteriophage consisting essentially of ΦMM7 (SEQ ID NO:72). In some aspects, a bacteriophage or composition thereof may comprise an isolated bacteriophage consisting of ΦMM7 (SEQ ID NO:72).

In some aspects, a bacteriophage may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant nucleotide substitutions to the genomic sequence of ΦMM7 (SEQ ID NO:72). In some aspects, a bacteriophage may be at least, at most, exactly, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%, 99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83%, 99.84%, 99.85%, 99.86%, 99.87%, 99.88%, 99.89%, 99.9%, 99.91%, 99.92%, 99.93%, 99.94%, 99.95%, 99.96%, 99.97%, 99.98%, 99.99%, or 100% (or any derivable range therein) similar, identical, or homologous with the genomic sequence of ΦMM7 (SEQ ID NO:72).

In some aspects, a bacteriophage or composition thereof may comprise an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequence of ΦMM7 (SEQ ID NO:72).

Variants or evolved bacteriophage derived from ΦMM7 having a genome comprising, consisting, or consisting essentially of SEQ ID NO:72 are also contemplated as aspects of the methods and compositions disclosed herein.

### L. Bacteriophage MM9

Aspects of the present disclosure comprise, consist essentially of, or consist of bacteriophage MM9 (also “ΦMM9,” used synonymously herein), compositions, kits, and devices comprising ΦMM9, and methods for use. A ΦMM9 genome is described by GENBANK® Accession Number ______, which is noted as 41,508 bp in length, and the sequence of which is incorporated herein by reference as SEQ ID NO:74.

In some aspects, a bacteriophage or composition thereof may comprise an isolated bacteriophage comprising ΦMM9 (SEQ ID NO:74). In some aspects, a bacteriophage or composition thereof may comprise an isolated bacteriophage consisting essentially of ΦMM9 (SEQ ID NO:74). In some aspects, a bacteriophage or composition thereof may comprise an isolated bacteriophage consisting of ΦMM9 (SEQ ID NO:74).

In some aspects, a bacteriophage may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant nucleotide substitutions to the genomic sequence of ΦMM9 (SEQ ID NO:74). In some aspects, a bacteriophage may be at least, at most, exactly, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%, 99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83%, 99.84%, 99.85%, 99.86%, 99.87%, 99.88%, 99.89%, 99.9%, 99.91%, 99.92%, 99.93%, 99.94%, 99.95%, 99.96%, 99.97%, 99.98%, 99.99%, or 100% (or any derivable range therein) similar, identical, or homologous with the genomic sequence of ΦMM9 (SEQ ID NO:74).

In some aspects, a bacteriophage or composition thereof may comprise an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequence of ΦMM9 (SEQ ID NO:74).

Variants or evolved bacteriophage derived from ΦMM9 having a genome comprising, consisting, or consisting essentially of SEQ ID NO:74 are also contemplated as aspects of the methods and compositions disclosed herein.

### M. Other Bacteriophages

A bacteriophage composition of the disclosure may comprise, in addition to one or more of ΦER3, ΦER11, ΦER12, ΦER15, ΦER16, ΦER27, ΦER36, ΦER39, ΦER43, ΦER46, ΦMM7, and ΦMM9, one or more additional bacteriophage (e.g., additional bacteriophage capable of infecting and/or lysing pathogenic Klebsiella, Staphylococcus, E. coli, Pseudomonas, etc.).

Aspects of the present disclosure can comprise additional bacteriophage ΦER1 (SEQ ID NO:6, SEQ ID NO:30), ΦER2 (SEQ ID NO:7, SEQ ID NO:31), ΦER4 (SEQ ID NO: 9, SEQ ID NO:33), ΦER5 (SEQ ID NO: 10), ΦER6 (SEQ ID NO:11), ΦER7/ΦER10 (SEQ ID NO: 12, SEQ ID NO:35), ΦER8 (SEQ ID NO: 13, SEQ ID NO:34), ΦER13 (SEQ ID NO: 15, SEQ ID NO:38), ΦER14 (SEQ ID NO:16, SEQ ID NO:39), ΦER18 (SEQ ID NO:17, SEQ ID NO: 42), ΦER20 (SEQ ID NO: 18, SEQ ID NO:43), ΦER21 (SEQ ID NO:44), ΦER22 (SEQ ID NO: 19, SEQ ID NO:45), ΦER23 (SEQ ID NO:46), ΦER32 (SEQ ID NO:21, SEQ ID NO: 48), ΦER37 (SEQ ID NO:23, SEQ ID NO:50), ΦER38 (SEQ ID NO:24, SEQ ID NO:51), ΦER44 (SEQ ID NO:26, SEQ ID NO:54), ΦER45 (SEQ ID NO:27, SEQ ID NO:55), ΦER47 (SEQ ID NO:57), ΦER48 (SEQ ID NO:58), ΦER49 (SEQ ID NO:28, SEQ ID NO:59), ΦER50 (SEQ ID NO:29, SEQ ID NO:60), ΦER51 (SEQ ID NO:61), ER52 (SEQ ID NO:62), ΦER53 (SEQ ID NO:63), ΦER54 (SEQ ID NO:64), ΦER55 (SEQ ID NO:65), ΦMM1 (SEQ ID NO: 66), ΦMM2 (SEQ ID NO:67), ΦMM3 (SEQ ID NO:68), ΦMM4 (SEQ ID NO:69), ΦMM5 (SEQ ID NO:70), ΦMM6 (SEQ ID NO:71), and ΦMM8 (SEQ ID NO:73), having genomes described by GENBANK® Accession Numbers respectively. Compositions, kits, and devices can comprise bacteriophage ΦER1 (SEQ ID NO:6, SEQ ID NO:30), ΦER2 (SEQ ID NO: 7, SEQ ID NO:31), ΦER4 (SEQ ID NO:9, SEQ ID NO:33), ΦER5 (SEQ ID NO:10), ΦER6 (SEQ ID NO:11), ΦER7/ΦER10 (SEQ ID NO:12, SEQ ID NO:35), ΦER8 (SEQ ID NO: 13, SEQ ID NO:34), ΦER13 (SEQ ID NO: 15, SEQ ID NO:38), ΦER14 (SEQ ID NO: 16, SEQ ID NO:39), ΦER18 (SEQ ID NO: 17, SEQ ID NO:42), ΦER20 (SEQ ID NO: 18, SEQ ID NO: 43), ΦER21 (SEQ ID NO:44), ΦER22 (SEQ ID NO: 19, SEQ ID NO:45), ΦER23 (SEQ ID NO: 46), ER32 (SEQ ID NO:21, SEQ ID NO:48), ΦER37 (SEQ ID NO:23, SEQ ID NO: 50), ΦER38 (SEQ ID NO:24, SEQ ID NO:51), ΦER44 (SEQ ID NO:26, SEQ ID NO:54), ΦER45 (SEQ ID NO:27, SEQ ID NO:55), ΦER47 (SEQ ID NO:57), ΦER48 (SEQ ID NO:58), ΦER49 (SEQ ID NO:28, SEQ ID NO:59), ΦER50 (SEQ ID NO:29, SEQ ID NO:60), ER51 (SEQ ID NO:61), ΦER52 (SEQ ID NO:62), ΦER53 (SEQ ID NO:63), ΦER54 (SEQ ID NO: 64), ER55 (SEQ ID NO:65), ΦMM1 (SEQ ID NO:66), ΦMM2 (SEQ ID NO:67), ΦMM3 (SEQ ID NO:68), ΦMM4 (SEQ ID NO:69), ΦMM5 (SEQ ID NO:70), ΦMM6 (SEQ ID NO: 71), and ΦMM8 (SEQ ID NO:73), having genomes described by GENBANK® Accession Numbers ______, respectively, and methods for use. In some aspects, any one or more of the foregoing bacteriophage may be excluded from the compositions and/or methods disclosed herein.

In some aspects, a bacteriophage or composition thereof may comprise an isolated bacteriophage comprising a genome provided any one of SEQ ID NOs: 6, 7, 9-13, 15-19, 21, 23, 24, 26-29, 30, 31, 33-35, 38, 39, 42-46, 48, 50, 51, 54, 55, 57-71, or 73. In some aspects, a bacteriophage or composition thereof may comprise an isolated bacteriophage consisting essentially of a genome provided any one of SEQ ID NOs: 6, 7, 9-13, 15-19, 21, 23, 24, 26-29, 30, 31, 33-35, 38, 39, 42-46, 48, 50, 51, 54, 55, 57-71, or 73. In some aspects, a bacteriophage or composition thereof may comprise an isolated bacteriophage consisting of a genome provided any one of SEQ ID NOs: 6, 7, 9-13, 15-19, 21, 23, 24, 26-29, 30, 31, 33-35, 38, 39, 42-46, 48, 50, 51, 54, 55, 57-71, or 73.

In some aspects, a bacteriophage may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant nucleotide substitutions to the genomic sequence of any one of SEQ ID NOs: 6, 7, 9-13, 15-19, 21, 23, 24, 26-29, 30, 31, 33-35, 38, 39, 42-46, 48, 50, 51, 54, 55, 57-71, or 73. In some aspects, a bacteriophage may be at least, at most, exactly, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%, 99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83%, 99.84%, 99.85%, 99.86%, 99.87%, 99.88%, 99.89%, 99.9%, 99.91%, 99.92%, 99.93%, 99.94%, 99.95%, 99.96%, 99.97%, 99.98%, 99.99%, or 100% (or any derivable range therein) similar, identical, or homologous with the genomic sequence of any one of SEQ ID NOs: 6, 7, 9-13, 15-19, 21, 23, 24, 26-29, 30, 31, 33-35, 38, 39, 42-46, 48, 50, 51, 54, 55, 57-71, or 73.

In some aspects, a bacteriophage or composition thereof may comprise an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequence of any one of SEQ ID NOs: 6, 7, 9-13, 15-19, 21, 23, 24, 26-29, 30, 31, 33-35, 38, 39, 42-46, 48, 50, 51, 54, 55, 57-71, or 73.

Variants or evolved bacteriophage derived from, e.g., ΦER1, ΦER2, ΦER4, ΦER5, ΦER6, ΦER7/ΦER10, ΦER8, ΦER13, ΦER14, ER18, ΦER20, ΦER21, ER22, ΦER23, ΦER32, ΦER37, ΦER38, ΦER44, ΦER45, ΦER47, ΦER48, ΦER49, ΦER50, ΦER51, ΦER52, ΦER53, ΦER54, ΦER55, ΦMM1, ΦMM2, ΦMM3, ΦMM4, ΦMM5, ΦMM6, and ΦMM8, having genomes comprising, consisting, or consisting essentially of any one or more of SEQ ID NOs: 6, 7, 9-13, 15-19, 21, 23, 24, 26-29, 30, 31, 33-35, 38, 39, 42-46, 48, 50, 51, 54, 55, 57-71, or 73, are also contemplated as aspects of the methods and compositions disclosed herein.

Aspects of methods and compositions the present disclosure can include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or 74 of the following bacteriophage having genomes comprising, consisting essentially of, or consisting of any of SEQ ID NOs: 1-74: ΦER1, ΦER2, ΦER3, ΦER4, ΦER5, ΦER6, ΦER7/ΦER10, ΦER8, ΦER11, ΦER12, ΦER13, ΦER14, ΦER15, ΦER16, ΦER18, ΦER20, ΦER21, ΦER22, ΦER23, ΦER27, ΦER32, ΦER36, ΦER37, ΦER38, ΦER39, ΦER43, ΦER44, ΦER45, ΦER46, ΦER47, ΦER48, ΦER49, ΦER50, ΦER51, ΦER52, ΦER53, ΦER54, ΦER55, ΦMM1, ΦMM2, ΦMM3, ΦMM4, ΦMM5, ΦMM6, ΦMM7, ΦMM8, and/or ΦMM9. It is contemplated that 1, 2, 3, 4, 5, 6, 7, 8, 9, 10, or more additional bacteriophage not included in list could be included in further aspects of additional compositions and methods.

Further bacteriophage contemplated herein include, but are not limited to, bacteriophages described in, e.g., Herridge W. P. et al., “Bacteriophages of Klebsiella spp., their diversity and potential therapeutic uses,” J. Med. Microbiol. 2020 February; 69 (2): 176-194, and bacteriophages EC1, CF2, ES12, ES21, ES26, HP3, and ES17 described in, e.g., Gibson S. B. et al. “Constructing and Characterizing Bacteriophage Libraries for Phage Therapy of Human Infections,” Front. Microbiol. 2019; 10:2537, each of which are incorporated herein by reference in their entirety.

### II. Treatment and Prevention of Bacterial Infection

Aspects of the present disclosure are directed to methods for treatment and prevention of a bacterial infection in an individual. In some aspects, disclosed are methods for treatment or prevention of a Klebsiella infection in an individual. In some aspects, disclosed are methods for treatment or prevention of a Klebsiella pneumoniae (K. pneumoniae) infection in an individual. In some aspects, disclosed are methods for treatment or prevention of a Klebsiella quasipneumoniae (K. quasipneumoniae) infection in an individual. In some aspects, disclosed are methods for treatment or prevention of a Klebsiella variicola (K. variicola) infection in an individual. In some aspects, disclosed are methods for treatment or prevention of a multidrug-resistant (MDR) Klebsiella infection in an individual. In some aspects, disclosed are methods for treatment or prevention of a hypervirulent Klebsiella infection in an individual. In particular aspects, the present disclosure provides methods for treatment or prevention of a Klebsiella infection in an individual comprising administering to the individual an effective amount of one or more bacteriophage, including bacteriophage disclosed herein.

Bacteriophage useful for such treatment methods include those described herein, e.g., ΦER1, ΦER2, ΦER3, ΦER4, ΦER5, ΦER6, ΦER7/ΦER10, ΦER8, ΦER11, ΦER12, ΦER13, ΦER14, ΦER15, ΦER16, ΦER18, ΦER20, ΦER21, ΦER22, ΦER23, ΦER27, ΦER32, ΦER36, ΦER37, ΦER38, ΦER39, ΦER43, ΦER44, ER45, ΦER46, ΦER47, ΦER48, ΦER49, ΦER50, ΦER51, ΦER52, ΦER53, ΦER54, ΦER55, ΦMM1, ΦMM2, ΦMM3, ΦMM4, ΦMM5, ΦMM6, ΦMM7, ΦMM8, and/or ΦMM9. In some aspects, any one or more of the foregoing bacteriophage can be excluded from a treatment method disclosed herein. In some aspects, ΦER3, ΦER11, ΦER12, ΦER15, ΦER16, ΦER27, ER36, ΦER39, ΦER43, ΦER46, ΦMM7, and/or ΦMM9 are useful for such treatment methods. In specific aspects, In some aspects, bacteriophage useful for such treatment methods include ΦER12, ΦER15, ΦER16, ΦER39, and ΦER46, or any combination thereof. Any one or more of the foregoing bacteriophage can be excluded from a treatment method disclosed herein. In some aspects, bacteriophage useful for such treatment methods include ΦER3, ΦER11, ΦER27, ΦER36, and ΦMM9, or any combination thereof. Any one or more of the foregoing bacteriophage can be excluded from a treatment method disclosed herein. In some aspects, bacteriophage useful for such treatment methods include ΦER15, ΦER27, ΦER43, ΦER46, and ΦMM7, or any combination thereof. Any one or more of the foregoing bacteriophage can be excluded from a treatment method disclosed herein.

In certain aspects, compositions and/or methods described herein are utilized to reduce Klebsiella carriage by an individual and/or in an environment (e.g., reducing the level of one or more drug-resistant, hypervirulent, and/or pathogenic Klebsiella strains in an individual and/or in an environment). In certain aspects, reduction of Klebsiella carriage is an infection preventative measure.

Accordingly, in some aspects, disclosed herein is a method for treatment or prevention of a Klebsiella infection in an individual comprising administering to the individual an effective amount of any one or more of ΦER1, ΦER2, ΦER3, ΦER4, ΦER5, ΦER6, ΦER7/ΦER10, ΦER8, ΦER11, ΦER12, ΦER13, ΦER14, ΦER15, ΦER16, ΦER18, ΦER20, ΦER21, ΦER22, ΦER23, ΦER27, ΦER32, ΦER36, ΦER37, ΦER38, ΦER39, ΦER43, ΦER44, ΦER45, ΦER46, ΦER47, ΦER48, ΦER49, ΦER50, ΦER51, ΦER52, ΦER53, ΦER54, ΦER55, ΦMM1, ΦMM2, ΦMM3, ΦMM4, ΦMM5, ΦMM6, ΦMM7, ΦMM8, and/or ΦMM9. In some aspects, disclosed herein is a method for treatment or prevention of a Klebsiella infection in an individual comprising administering to the individual an effective amount of ΦER3, ER11, ΦER12, ΦER15, ΦER16, ΦER27, ΦER36, ΦER39, ΦER43, ΦER46, ΦMM7, and/or ΦMM9, or one or more compositions thereof. Any one or more of the foregoing bacteriophage can be excluded from a method for treatment or prevention of a Klebsiella infection disclosed herein.

In specific aspects, a method for treatment or prevention of a Klebsiella infection in an individual comprises administering to the individual an effective amount of any one or more of ΦER3, ΦER11, ΦER12, ΦER15, ΦER16, ΦER27, ER36, ΦER39, ΦER43, ΦER46, ΦMM7, and/or ΦMM9, or one or more compositions thereof. In some aspects, the method comprises administering ΦER3 or a composition thereof. In some aspects, the method comprises administering ΦER11 or a composition thereof. In some aspects, the method comprises administering ΦER12 or a composition thereof. In some aspects, the method comprises administering ΦER15 or a composition thereof. In some aspects, the method comprises administering ΦER16 or a composition thereof. In some aspects, the method comprises administering ΦER27 or a composition thereof. In some aspects, the method comprises administering ΦER36 or a composition thereof. In some aspects, the method comprises administering ΦER39 or a composition thereof. In some aspects, the method comprises administering ΦER43 or a composition thereof. In some aspects, the method comprises administering ΦER46 or a composition thereof. In some aspects, the method comprises administering ΦER49 or a composition thereof. In some aspects, the method comprises administering ΦMM7 or a composition thereof. In some aspects, the method comprises administering ΦMM9 or a composition thereof.

In some aspects, the method comprises administering two or more of ΦER3, ΦER11, ΦER12, ΦER15, ΦER16, ΦER27, ΦER36, ΦER39, ΦER43, ΦER46, MM7, and/or ΦMM9, or one or more compositions thereof. In some aspects, the method comprises administering three or more of ΦER3, ΦER11, ΦER12, ΦER15, ΦER16, ΦER27, ΦER36, ΦER39, ΦER43, ΦER46, ΦMM7, and/or ΦMM9, or one or more compositions thereof. In some aspects, the method comprises administering four or more of ΦER3, ΦER11, ΦER12, ΦER15, ΦER16, ΦER27, ΦER36, ΦER39, ΦER43, ΦER46, ΦMM7, and/or ΦMM9, or one or more compositions thereof.

In some aspects, the method comprises administering two or more of ΦER12, ΦER15, ΦER16, ΦER39, ΦER46, or any combination thereof, or one or more compositions thereof. In some aspects, the method comprises administering three or more of ΦER12, ΦER15, ΦER16, ΦER39, ΦER46, or any combination thereof, or one or more compositions thereof. In some aspects, the method comprises administering four or more of ΦER12, ΦER15, ΦER16, ΦER39, ΦER46, or any combination thereof, or one or more compositions thereof.

In some aspects, the method comprises administering two or more of ΦER3, ΦER11, ΦER27, ΦER36, ΦMM9, or any combination thereof, or one or more compositions thereof. In some aspects, the method comprises administering three or more of ΦER3, ΦER11, ΦER27, ΦER36, ΦMM9, or any combination thereof, or one or more compositions thereof. In some aspects, the method comprises administering four or more of ΦER3, ΦER11, ΦER27, ΦER36, ΦMM9, or any combination thereof, or one or more compositions thereof.

In some aspects, the method comprises administering two or more of ΦER15, ΦER27, ΦER43, ΦER46, ΦMM7, or any combination thereof, or one or more compositions thereof. In some aspects, the method comprises administering three or more of ΦER15, ΦER27, ΦER43, ΦER46, ΦMM7, or any combination thereof, or one or more compositions thereof. In some aspects, the method comprises administering four or more of ΦER15, ΦER27, ΦER43, ΦER46, ΦMM7, or any combination thereof, or one or more compositions thereof.

In some aspects, the method comprises administering one of the bacteriophage combinations of Table 1, other bacteriophage combinations completed herein, or one or more compositions thereof.

In some aspects, multiple bacteriophage (e.g., two or more of ΦER3, ΦER11, ΦER12, ΦER15, ΦER16, ΦER27, ΦER36, ΦER39, ΦER43, ΦER46, ΦMM7, and/or ΦMM9) are administered to an individual in the same formulation. Alternatively, multiple bacteriophage may be administered to an individual in different formulations (e.g., 2, 3, 4, 5, or more formulations). Multiple bacteriophage may be administered to an individual at the same time or may be administered at different times.

Multiple bacteriophage may be administered to an individual substantially simultaneously, for example via a single composition. For example, any two or more of ΦER3, ΦER11, ΦER12, ΦER15, ΦER16, ΦER27, ΦER36, ΦER39, ΦER43, ΦER46, ΦMM7, and/or ΦMM9, may be administered to an individual having a pathogenic Klebsiella infection at the same time, as a single composition. Any one or more of the foregoing bacteriophage can be excluded from a single composition disclosed herein.

Multiple bacteriophage may be administered to an individual sequentially in any order. For example, an individual having a pathogenic Klebsiella infection may be administered any two or more of ΦER3, ΦER11, ΦER12, ΦER15, ΦER16, ΦER27, ΦER36, ΦER39, ΦER43, ΦER46, ΦMM7, and/or ΦMM9, in any order.

Example administration sequences for ΦER12, ΦER15, ΦER16, ΦER39, and/or ΦER46 include: ΦER12 and ΦER15; ΦER15 and ΦER12; ΦER12 and ΦER16; ΦER16 and ΦER12; ΦER12 and ΦER39; ΦER39 and ΦER12; ΦER12 and ΦER46; ΦER46 and ΦER12; ΦER12, ΦER15, and ΦER16; ΦER12, ΦER16, and ΦER15; ΦER15, ΦER12, and ΦER16; ΦER15, ΦER16, and ΦER12; ΦER16, ΦER12, and ΦER15; ΦER16, ΦER15, and ΦER12; ΦER12, ΦER15, and 39; ΦER12, ΦER39, and ΦER15; ΦER15, ΦER12, and ΦER39; ΦER15, ΦER39, and ΦER12; ΦER39, ΦER12, and ΦER15; ΦER39, ΦER15, and ΦER12; ΦER12, ΦER15, and 46; ΦER12, ΦER46, and ΦER15; ΦER15, ΦER12, and ΦER46; ΦER15, ΦER46, and ΦER12; ΦER46, ΦER12, and ΦER15; ΦER46, ΦER15, and ER12; ΦER12, ΦER16, and 39; ΦER12, ΦER39, and ΦER16; ΦER16, ΦER12, and ΦER39; ΦER16, ΦER39, and ΦER12; ΦER39, ΦER12, and ΦER16; ΦER39, ΦER16, and ΦER12; ΦER12, ΦER16, and 46; ΦER12, ΦER46, and ΦER16; ΦER16, ΦER12, and ΦER46; ΦER16, ΦER46, and ΦER12; ΦER46, ΦER12, and ΦER16; ΦER46, ΦER16, and ΦER12; ΦER12, ΦER36, and 46; ΦER12, ΦER46, and ΦER36; ΦER36, ΦER12, and ΦER46; ΦER36, ΦER46, and ΦER12; ΦER46, ΦER12, and ΦER36; ΦER46, ΦER36, and ΦER12; ΦER12, ΦER15, ΦER16, and ΦER39; ΦER12, ΦER15, ΦER39, and ΦER16; ΦER12, ΦER16, ΦER39, and ΦER15; ΦER12, ΦER16, ΦER15, and ΦER39; ΦER12, ΦER39, ΦER15, and ΦER16; ΦER12, ΦER39, ΦER16, and ΦER15; ΦER15, ΦER12, ΦER16, and ΦER39; ΦER15, ΦER12, ΦER39, and ΦER16; ΦER15, ΦER16, ΦER39, and ΦER12; ΦER15, ΦER16, ΦER12, and ΦER39; ΦER15, ΦER39, ΦER11, and ΦER16; ΦER15, ΦER39, ΦER16, and ΦER12; ΦER16, ΦER15, ΦER12, and ΦER39; ΦER16, ΦER15, ΦER39, and ΦER12; ΦER16, ΦER12, ΦER39, and ΦER15; ΦER16, ΦER12, ΦER15, and ΦER39; ΦER16, ΦER39, ΦER15, and ΦER12; ΦER16, ΦER39, ΦER12, and ΦER15; ΦER39, ΦER15, ΦER16, and ΦER12; ΦER39, ΦER15, ΦER12, and ΦER16; ΦER39, ΦER16, ΦER12, and ΦER15; ΦER39, ΦER16, ΦER15, and ΦER12; ΦER39, ΦER12, ΦER15, and ΦER16; ΦER39, ΦER12, ΦER16, and ΦER15; ΦER12, ΦER15, ΦER16, and ΦER46; ΦER12, ΦER15, ΦER46, and ΦER16; ΦER12, ΦER16, ΦER46, and ΦER15; ΦER12, ΦER16, ΦER15, and ΦER46; ΦER12, ΦER46, ΦER15, and ΦER16; ΦER12, ΦER46, ΦER16, and ΦER15; ΦER15, ΦER12, ΦER16, and ΦER46; ΦER15, ΦER12, ΦER46, and ΦER16; ΦER15, ΦER16, ΦER46, and ΦER12; ΦER15, ΦER16, ΦER12, and ΦER46; ΦER15, ΦER46, ΦER11, and ΦER16; ΦER15, ΦER46, ΦER16, and ΦER12; ΦER16, ΦER15, ΦER12, and ΦER46; ΦER16, ΦER15, ΦER46, and ΦER12; ΦER16, ΦER12, ΦER46, and ΦER15; ΦER16, ΦER12, ΦER15, and ΦER46; ΦER16, ΦER46, ΦER15, and ΦER12; ΦER16, ΦER46, ΦER12, and ΦER15; ΦER46, ΦER15, ΦER16, and ΦER12; ΦER46, ΦER15, ΦER12, and ΦER16; ΦER46, ΦER16, ΦER12, and ΦER15; ΦER46, ΦER16, ΦER15, and ΦER12; ΦER46, ΦER12, ΦER15, and ΦER16; ΦER46, ΦER12, ΦER16, and ΦER15; ΦER12, ΦER15, ΦER39, and ΦER46; ΦER12, ΦER15, ΦER46, and ΦER39; ΦER12, ΦER39, ΦER46, and ΦER15; ΦER12, ΦER39, ΦER15, and ΦER46; ΦER12, ΦER46, ΦER15, and ΦER39; ΦER12, ΦER46, ΦER39, and ΦER15; ΦER15, ΦER12, ΦER39, and ΦER46; ΦER15, ΦER12, ΦER46, and ΦER39; ΦER15, ΦER39, ΦER46, and ΦER12; ΦER15, ΦER39, ΦER12, and ΦER46; ΦER15, ΦER46, ΦER11, and ΦER39; ΦER15, ΦER46, ΦER39, and ΦER12; ΦER39, ΦER15, ΦER12, and ΦER46; ΦER39, ΦER15, ΦER46, and ΦER12; ΦER39, ΦER12, ΦER46, and ΦER15; ΦER39, ΦER12, ΦER15, and ΦER46; ΦER39, ΦER46, ΦER15, and ΦER12; ΦER39, ΦER46, ΦER12, and ΦER15; ΦER46, ΦER15, ΦER39, and ΦER12; ΦER46, ΦER15, ΦER12, and ΦER39; ΦER46, ΦER39, ΦER12, and ΦER15; ΦER46, ΦER39, ΦER15, and ΦER12; ΦER46, ΦER12, ΦER15, and ΦER39; ΦER46, ΦER12, ΦER39, and ΦER15; ΦER12, ΦER16, ΦER39, and ΦER46; ΦER12, ΦER16, ΦER46, and ΦER39; ΦER12, ΦER39, ΦER46, and ΦER16; ΦER12, ΦER39, ΦER16, and ΦER46; ΦER12, ΦER46, ΦER16, and ΦER39; ΦER12, ΦER46, ΦER39, and ΦER16; ΦER16, ΦER12, ΦER39, and ΦER46; ΦER16, ΦER12, ΦER46, and ΦER39; ΦER16, ΦER39, ΦER46, and ΦER12; ΦER16, ΦER39, ΦER12, and ΦER46; ΦER16, ΦER46, ΦER11, and ΦER39; ΦER16, ΦER46, ΦER39, and ΦER12; ΦER39, ΦER16, ΦER12, and ΦER46; ΦER39, ΦER16, ΦER46, and ΦER12; ΦER39, ΦER12, ΦER46, and ΦER16; ΦER39, ΦER12, ΦER16, and ΦER46; ΦER39, ΦER46, ΦER16, and ΦER12; ΦER39, ΦER46, ΦER12, and ΦER16; ΦER46, ΦER16, ΦER39, and ΦER12; ΦER46, ΦER16, ΦER12, and ΦER39; ΦER46, ΦER39, ΦER12, and ΦER16; ΦER46, ΦER39, ΦER16, and ΦER12; ΦER46, ΦER12, ΦER16, and ΦER39; ΦER46, ΦER12, ΦER39, and ΦER16; ΦER12, ΦER15, ΦER16, ΦER39, and ΦER46; ΦER12, ΦER16, ΦER39, ΦER46, and ΦER15; ΦER12, ΦER39, ΦER46, ΦER15, and ΦER16; ΦER12, ΦER46, ΦER15, ΦER16, and ΦER39; ΦER12, ΦER16, ΦER15, ΦER39, and ΦER46; ΦER12, ΦER15, ΦER39, ΦER46, and ΦER16; ΦER12, ΦER39, ΦER46, ΦER16, and ΦER15; ΦER12, ΦER46, ΦER16, ΦER15, and ΦER39; ΦER12, ΦER39, ΦER16, ΦER15, and ΦER46; ΦER12, ΦER16, ΦER15, ΦER46, and ΦER39; ΦER12, ΦER15, ΦER46, ΦER39, and ΦER16; ΦER12, ΦER46, ΦER39, ΦER16, and ΦER15; ΦER12, ΦER46, ΦER16, ΦER39, and ΦER15; ΦER12, ΦER16, ΦER39, ΦER15, and ΦER46; ΦER12, ΦER39, ΦER15, ΦER46, and ΦER16; ΦER12, ΦER15, ΦER46, ΦER16, and ΦER39; ΦER12, ΦER15, ΦER39, ΦER16, and ΦER46; ΦER12, ΦER39, ΦER16, ΦER46, and ΦER15; ΦER12, ΦER16, ΦER46, ΦER15, and ΦER39; ΦER12, ΦER46, ΦER15, ΦER39, and ΦER16; ΦER12, ΦER15, ΦER46, ΦER39, and ΦER16; ΦER12, ΦER46, ΦER39, ΦER16, and ΦER15; ΦER12, ΦER39, ΦER16, ΦER15, and ΦER46; ΦER12, ΦER16, ΦER15, ΦER46, and ΦER39; ΦER12, ΦER15, ΦER16, ΦER46, and ΦER39; ΦER12, ΦER16, ΦER46, ΦER39, and ΦER15; ΦER12, ΦER46, ΦER39, ΦER15, and ΦER16; ΦER12, ΦER39, ΦER15, ΦER16, and ΦER46; ΦER15, ΦER12, ΦER16, ΦER39, and ΦER46; ΦER15, ΦER16, ΦER39, ΦER46, and ΦER12; ΦER15, ΦER39, ΦER46, ΦER12, and ΦER16; ΦER15, ΦER46, ΦER12, ΦER16, and ΦER39; ΦER15, ΦER16, ΦER12, ΦER39, and ΦER46; ΦER15, ΦER12, ΦER39, ΦER46, and ΦER16; ΦER15, ΦER39, ΦER46, ΦER16, and ΦER12; ΦER15, ΦER46, ΦER16, ΦER12, and ΦER39; ΦER15, ΦER39, ΦER16, ΦER12, and ΦER46; ΦER15, ΦER16, ΦER12, ΦER46, and ΦER39; ΦER15, ΦER12, ΦER46, ΦER39, and ΦER16; ΦER15, ΦER46, ΦER39, ΦER16, and ΦER12; ΦER15, ΦER46, ΦER16, ΦER39, and ΦER12; ΦER15, ΦER16, ΦER39, ΦER12, and ΦER46; ΦER15, ΦER39, ΦER12, ΦER46, and ΦER16; ΦER15, ΦER12, ΦER46, ΦER16, and ΦER39; ΦER15, ΦER12, ΦER39, ΦER16, and ΦER46; ΦER15, ΦER39, ΦER16, ΦER46, and ΦER12; ΦER15, ΦER16, ΦER46, ΦER12, and ΦER39; ΦER15, ΦER46, ΦER12, ΦER39, and ΦER16; ΦER15, ΦER12, ΦER46, ΦER39, and ΦER16; ΦER15, ΦER46, ΦER39, ΦER16, and ΦER12; ΦER15, ΦER39, ΦER16, ΦER12, and ΦER46; ΦER15, ΦER16, ΦER12, ΦER46, and ΦER39; ΦER15, ΦER12, ΦER16, ΦER46, and ΦER39; ΦER15, ΦER16, ΦER46, ΦER39, and ΦER12; ΦER15, ΦER46, ΦER39, ΦER12, and ΦER16; ΦER15, ΦER39, ΦER12, ΦER16, and ΦER46; ΦER16, ΦER15, ΦER12, ΦER39, and ΦER46; ΦER16, ΦER12, ΦER39, ΦER46, and ΦER15; ΦER16, ΦER39, ΦER46, ΦER15, and ΦER12; ΦER16, ΦER46, ΦER15, ΦER12, and ΦER39; ΦER16, ΦER12, ΦER15, ΦER39, and ΦER46; ΦER16, ΦER15, ΦER39, ΦER46, and ΦER12; ΦER16, ΦER39, ΦER46, ΦER12, and ΦER15; ΦER16, ΦER46, ΦER12, ΦER15, and ΦER39; ΦER16, ΦER39, ΦER12, ΦER15, and ΦER46; ΦER16, ΦER12, ΦER15, ΦER46, and ΦER39; ΦER16, ΦER15, ΦER46, ΦER39, and ΦER12; ΦER16, ΦER46, ΦER39, ΦER12, and ΦER15; ΦER16, ΦER46, ΦER12, ΦER39, and ΦER15; ΦER16, ΦER12, ΦER39, ΦER15, and ΦER46; ΦER16, ΦER39, ΦER15, ΦER46, and ΦER12; ΦER16, ΦER15, ΦER46, ΦER12, and ΦER39; ΦER16, ΦER15, ΦER39, ΦER12, and ΦER46; ΦER16, ΦER39, ΦER12, ΦER46, and ΦER15; ΦER16, ΦER12, ΦER46, ΦER15, and ΦER39; ΦER16, ΦER46, ΦER15, ΦER39, and ΦER12; ΦER16, ΦER15, ΦER46, ΦER39, and ΦER12; ΦER16, ΦER46, ΦER39, ΦER12, and ΦER15; ΦER16, ΦER39, ΦER12, ΦER15, and ΦER46; ΦER16, ΦER12, ΦER15, ΦER46, and ΦER39; ΦER16, ΦER15, ΦER12, ΦER46, and ΦER39; ΦER16, ΦER12, ΦER46, ΦER39, and ΦER15; ΦER16, ΦER46, ΦER39, ΦER15, and ΦER12; ΦER16, ΦER39, ΦER15, ΦER12, and ΦER46; ΦER39, ΦER15, ΦER16, ΦER12, and ΦER46; ΦER39, ΦER16, ΦER12, ΦER46, and ΦER15; ΦER39, ΦER12, ΦER46, ΦER15, and ΦER16; ΦER39, ΦER46, ΦER15, ΦER16, and ΦER12; ΦER39, ΦER16, ΦER15, ΦER12, and ΦER46; ΦER39, ΦER15, ΦER12, ΦER46, and ΦER16; ΦER39, ΦER12, ΦER46, ΦER16, and ΦER15; ΦER39, ΦER46, ΦER16, ΦER15, and ΦER12; ΦER39, ΦER12, ΦER16, ΦER15, and ΦER46; ΦER39, ΦER16, ΦER15, ΦER46, and ΦER12; ΦER39, ΦER15, ΦER46, ΦER12, and ΦER16; ΦER39, ΦER46, ΦER12, ΦER16, and ΦER15; ΦER39, ΦER46, ΦER16, ER12, and ΦER15; ΦER39, ΦER16, ΦER12, ΦER15, and ΦER46; ΦER39, ΦER12, ΦER15, ΦER46, and ΦER16; ΦER39, ΦER15, ΦER46, ΦER16, and ΦER12; ΦER39, ΦER15, ΦER12, ΦER16, and ΦER46; ΦER39, ΦER12, ΦER16, ΦER46, and ΦER15; ΦER39, ΦER16, ΦER46, ΦER15, and ΦER12; ΦER39, ΦER46, ΦER15, ΦER12, and ΦER16; ΦER39, ΦER15, ΦER46, ΦER12, and ΦER16; ΦER39, ΦER46, ΦER12, ΦER16, and ΦER15; ΦER39, ΦER12, ΦER16, ΦER15, and ΦER46; ΦER39, ΦER16, ΦER15, ΦER46, and ΦER12; ΦER39, ΦER15, ΦER16, ΦER46, and ΦER12; ΦER39, ΦER16, ΦER46, ΦER12, and ΦER15; ΦER39, ΦER46, ΦER12, ΦER15, and ΦER16; ΦER39, ΦER12, ΦER15, ΦER16, and ΦER46; ΦER46, ΦER15, ΦER16, ΦER39, and ΦER12; ΦER46, ΦER16, ΦER39, ΦER12, and ΦER15; ΦER46, ΦER39, ΦER12, ΦER15, and ΦER16; ΦER46, ΦER12, ΦER15, ΦER16, and ΦER39; ΦER46, ΦER16, ΦER15, ΦER39, and ΦER12; ΦER46, ΦER15, ΦER39, ΦER12, and ΦER16; ΦER46, ΦER39, ΦER12, ΦER16, and ΦER15; ΦER46, ΦER12, ΦER16, ΦER15, and ΦER39; ΦER46, ΦER39, ΦER16, ΦER15, and ΦER12; ΦER46, ΦER16, ΦER15, ΦER12, and ΦER39; ΦER46, ΦER15, ΦER12, ΦER39, and ΦER16; ΦER46, ΦER12, ΦER39, ΦER16, and ΦER15; ΦER46, ΦER12, ΦER16, ΦER39, and ΦER15; ΦER46, ΦER16, ΦER39, ΦER15, and ΦER12; ΦER46, ΦER39, ΦER15, ΦER12, and ΦER16; ΦER46, ΦER15, ΦER12, ΦER16, and ΦER39; ΦER46, ΦER15, ΦER39, ΦER16, and ΦER12; ΦER46, ΦER39, ΦER16, ΦER12, and ΦER15; ΦER46, ΦER16, ΦER12, ΦER15, and ΦER39; ΦER46, ΦER12, ΦER15, ΦER39, and ΦER16; ΦER46, ΦER15, ΦER12, ΦER39, and ΦER16; ΦER46, ΦER12, ΦER39, ΦER16, and ΦER15; ΦER46, ΦER39, ΦER16, ΦER15, and ΦER12; ΦER46, ΦER16, ΦER15, ΦER12, and ΦER39; ΦER46, ΦER15, ΦER16, ΦER12, and ΦER39; ΦER46, ΦER16, ΦER12, ΦER39, and ΦER15; ΦER46, ΦER12, ΦER39, ΦER15, and ΦER16; ΦER46, ΦER39, ΦER15, ΦER16, and ΦER12. One or more of the preceding administration sequences may be excluded from aspects of the disclosure.

Example administration sequences for ΦER3, ΦER11, ΦER27, ΦER36, and/or ΦMM9 include: ΦER3 and ΦER11; PERIl and ΦER3; ΦER3 and ΦER27; ΦER27 and ΦER3; ΦER3 and ΦER36; ΦER36 and ΦER3; ΦER3 and ΦMM9; ΦMM9 and ΦER3; ΦER3, ΦER11, and ΦER27; ΦER3, ΦER27, and ΦER11; ΦER11, ΦER3, and ΦER27; ΦER11, ΦER27, and ΦER3; ΦER27, ΦER3, and ΦER11; ΦER27, ΦER11, and ΦER3; ΦER3, ΦER11, and 39; ΦER3, ΦER36, and ΦER11; ΦER11, ΦER3, and ΦER36; ΦER11, ΦER36, and ΦER3; ΦER36, ΦER3, and ΦER11; ΦER36, ΦER11, and ΦER3; ΦER3, ΦER11, and 46; ΦER3, ΦMM9, and ΦER11; ΦER11, ΦER3, and ΦMM9; ΦER11, ΦMM9, and ΦER3; ΦMM9, ΦER3, and ΦER11; ΦMM9, ΦER11, and ΦER3; ΦER3, ΦER27, and 39; ΦER3, ΦER36, and ΦER27; ΦER27, ΦER3, and ΦER36; ΦER27, ΦER36, and ΦER3; ΦER36, ΦER3, and ΦER27; ΦER36, ΦER27, and ΦER3; ΦER3, ΦER27, and 46; ΦER3, ΦMM9, and ΦER27; ΦER27, ΦER3, and ΦMM9; ΦER27, ΦMM9, and ΦER3; ΦMM9, ΦER3, and ΦER27; ΦMM9, ΦER27, and ΦER3; ΦER3, ΦER36, and 46; ΦER3, ΦMM9, and ΦER36; ΦER36, ΦER3, and ΦMM9; ΦER36, ΦMM9, and ΦER3; ΦMM9, ΦER3, and ΦER36; ΦMM9, ΦER36, and ΦER3; ΦER3, ΦER11, ΦER27, and ΦER36; ΦER3, ΦER11, ΦER36, and ΦER27; ΦER3, ΦER27, ΦER36, and ΦER11; ΦER3, ΦER27, ΦER11, and ΦER36; ΦER3, ΦER36, ΦER11, and ΦER27; ΦER3, ΦER36, ΦER27, and ΦER11; ΦER11, ΦER3, ΦER27, and ΦER36; ΦER11, ΦER3, ΦER36, and ΦER27; ΦER11, ΦER27, ΦER36, and ΦER3; ΦER11, ΦER27, ΦER3, and ΦER36; ΦER11, ΦER36, ΦER11, and ΦER27; ΦER11, ΦER36, ΦER27, and ΦER3; ΦER27, ΦER11, ΦER3, and ΦER36; ΦER27, ΦER11, ΦER36, and ΦER3; ΦER27, ΦER3, ΦER36, and ΦER11; ΦER27, ΦER3, ΦER11, and ΦER36; ΦER27, ΦER36, ΦER11, and ΦER3; ΦER27, ΦER36, ΦER3, and ΦER11; ΦER36, ΦER11, ΦER27, and ΦER3; ΦER36, ΦER11, ΦER3, and ΦER27; ΦER36, ΦER27, ΦER3, and ΦER11; ΦER36, ΦER27, ΦER11, and ΦER3; ΦER36, ΦER3, ΦER11, and ΦER27; ΦER36, ΦER3, ΦER27, and DER11; ΦER3, ΦER11, ΦER27, and ΦMM9; ΦER3, ΦER11, ΦMM9, and ΦER27; ΦER3, ΦER27, ΦMM9, and ΦER11; ΦER3, ΦER27, ΦER11, and ΦMM9; ΦER3, ΦMM9, ΦER11, and ΦER27; ΦER3, ΦMM9, ΦER27, and ΦER11; ΦER11, ΦER3, ΦER27, and ΦMM9; ΦER11, ΦER3, ΦMM9, and ΦER27; ΦER11, ΦER27, ΦMM9, and ΦER3; ΦER11, ΦER27, ΦER3, and ΦMM9; ΦER11, ΦMM9, ΦER11, and ΦER27; ΦER11, ΦMM9, ΦER27, and ΦER3; ΦER27, ΦER11, ΦER3, and ΦMM9; ΦER27, ΦER11, ΦMM9, and ΦER3; ΦER27, ΦER3, ΦMM9, and ΦER11; ΦER27, ΦER3, ΦER11, and ΦMM9; ΦER27, ΦMM9, ΦER11, and ΦER3; ΦER27, ΦMM9, ΦER3, and ΦER11; ΦMM9, ΦER11, ΦER27, and ΦER3; ΦMM9, ΦER11, ΦER3, and ΦER27; ΦMM9, ΦER27, ΦER3, and ΦER11; ΦMM9, ΦER27, ΦER11, and ΦER3; ΦMM9, ΦER3, ΦER11, and ΦER27; ΦMM9, ΦER3, ΦER27, and ΦER11; ΦER3, ΦER11, ΦER36, and ΦMM9; ΦER3, ΦER11, ΦMM9, and ΦER36; ΦER3, ΦER36, ΦMM9, and ΦER11; ΦER3, ΦER36, ΦER11, and ΦMM9; ΦER3, ΦMM9, ΦER11, and ΦER36; ΦER3, ΦMM9, ΦER36, and ΦER11; ΦER11, ΦER3, ΦER36, and ΦMM9; ΦER11, ΦER3, ΦMM9, and ΦER36; ΦER11, ΦER36, ΦMM9, and ΦER3; ΦER11, ΦER36, ΦER3, and ΦMM9; ΦER11, ΦMM9, ΦER11, and ΦER36; ΦER11, ΦMM9, ΦER36, and ΦER3; ΦER36, ΦER11, ΦER3, and ΦMM9; ΦER36, ΦER11, ΦMM9, and ΦER3; ΦER36, ΦER3, ΦMM9, and ΦER11; ΦER36, ΦER3, ΦER11, and ΦMM9; ΦER36, ΦMM9, ΦER11, and ΦER3; ΦER36, ΦMM9, ΦER3, and ΦER11; ΦMM9, ΦER11, ΦER36, and ΦER3; ΦMM9, ΦER11, ΦER3, and ΦER36; ΦMM9, ΦER36, ΦER3, and ΦER11; ΦMM9, ΦER36, ΦER11, and ΦER3; ΦMM9, ΦER3, ΦER11, and ΦER36; ΦMM9, ΦER3, ΦER36, and ΦER11; ΦER3, ΦER27, ΦER36, and ΦMM9; ΦER3, ΦER27, ΦMM9, and ΦER36; ΦER3, ΦER36, ΦMM9, and ΦER27; ΦER3, ΦER36, ΦER27, and ΦMM9; ΦER3, ΦMM9, ΦER27, and ΦER36; ΦER3, ΦMM9, ΦER36, and ΦER27; ΦER27, ΦER3, ΦER36, and ΦMM9; ΦER27, ΦER3, ΦMM9, and ΦER36; ΦER27, ΦER36, ΦMM9, and ΦER3; ΦER27, ΦER36, ΦER3, and ΦMM9; ΦER27, ΦMM9, ΦER11, and ΦER36; ΦER27, ΦMM9, ΦER36, and ΦER3; ΦER36, ΦER27, ΦER3, and ΦMM9; ΦER36, ΦER27, ΦMM9, and ΦER3; ΦER36, ΦER3, ΦMM9, and ΦER27; ΦER36, ΦER3, ΦER27, and ΦMM9; ΦER36, ΦMM9, ΦER27, and ΦER3; ΦER36, ΦMM9, ΦER3, and ΦER27; ΦMM9, ΦER27, ΦER36, and ΦER3; ΦMM9, ΦER27, ΦER3, and ΦER36; ΦMM9, ΦER36, ΦER3, and ΦER27; ΦMM9, ΦER36, ΦER27, and ΦER3; ΦMM9, ΦER3, ΦER27, and ΦER36; ΦMM9, ΦER3, ΦER36, and ΦER27; ΦER3, ΦER11, ΦER27, ΦER36, and ΦMM9; ΦER3, ΦER27, ΦER36, ΦMM9, and ΦER11; ΦER3, ΦER36, ΦMM9, ΦER11, and ΦER27; ΦER3, ΦMM9, ΦER11, ΦER27, and ΦER36; ΦER3, ΦER27, ΦER11, ER36, and ΦMM9; ΦER3, ΦER11, ER36, ΦMM9, and ΦER27; ΦER3, ΦER36, ΦMM9, ΦER27, and ΦER11; ΦER3, ΦMM9, ΦER27, ΦER11, and ΦER36; ΦER3, ΦER36, ΦER27, ΦER11, and ΦMM9; ΦER3, ΦER27, ΦER11, ΦMM9, and ΦER36; ΦER3, ΦER11, ΦMM9, ΦER36, and ΦER27; ΦER3, ΦMM9, ΦER36, ΦER27, and ΦER11; ΦER3, ΦMM9, ΦER27, ΦER36, and ΦER11; ΦER3, ΦER27, ΦER36, ΦER11, and ΦMM9; ΦER3, ΦER36, ΦER11, ΦMM9, and ΦER27; ΦER3, ΦER11, ΦMM9, ΦER27, and ΦER36; ΦER3, ΦER11, ΦER36, ΦER27, and ΦMM9; ΦER3, ΦER36, ΦER27, ΦMM9, and ΦER11; ΦER3, ΦER27, ΦMM9, ΦER11, and ΦER36; ΦER3, ΦMM9, ΦER11, ΦER36, and ΦER27; ΦER3, ΦER11, ΦMM9, ΦER36, and ΦER27; ΦER3, ΦMM9, ΦER36, ΦER27, and ΦER11; ΦER3, ΦER36, ΦER27, ΦER11, and ΦMM9; ΦER3, ΦER27, ΦER11, ΦMM9, and ΦER36; ΦER3, ΦER11, ΦER27, ΦMM9, and ΦER36; ΦER3, ΦER27, ΦMM9, ΦER36, and ΦER11; ΦER3, ΦMM9, ΦER36, ΦER11, and ΦER27; ΦER3, ΦER36, ΦER11, ΦER27, and ΦMM9; ΦER11, ΦER3, ΦER27, ΦER36, and ΦMM9; ΦER11, ΦER27, ΦER36, ΦMM9, and ΦER3; ΦER11, ΦER36, ΦMM9, ΦER3, and ΦER27; ΦER11, ΦMM9, ΦER3, ΦER27, and ΦER36; ΦER11, ΦER27, ΦER3, ΦER36, and ΦMM9; ΦER11, ΦER3, ΦER36, ΦMM9, and ΦER27; ΦER11, ΦER36, ΦMM9, ΦER27, and ΦER3; ΦER11, ΦMM9, ΦER27, ΦER3, and ΦER36; ΦER11, ΦER36, ΦER27, ΦER3, and ΦMM9; ΦER11, ΦER27, ΦER3, ΦMM9, and ΦER36; ΦER11, ΦER3, ΦMM9, ΦER36, and ΦER27; ΦER11, ΦMM9, ΦER36, ΦER27, and ΦER3; ΦER11, ΦMM9, ΦER27, ΦER36, and ΦER3; ΦER11, ΦER27, ΦER36, ΦER3, and ΦMM9; ΦER11, ΦER36, ΦER3, ΦMM9, and ΦER27; ΦER11, ΦER3, ΦMM9, ΦER27, and ΦER36; ΦER11, ΦER3, ΦER36, ΦER27, and ΦMM9; ΦER11, ΦER36, ΦER27, ΦMM9, and ΦER3; ΦER11, ΦER27, ΦMM9, ΦER3, and ΦER36; ΦER11, ΦMM9, ΦER3, ΦER36, and ΦER27; ΦER11, ΦER3, ΦMM9, ΦER36, and ΦER27; ΦER11, ΦMM9, ΦER36, ΦER27, and ΦER3; ΦER11, ΦER36, ΦER27, ΦER3, and ΦMM9; ΦER11, ΦER27, ΦER3, ΦMM9, and ΦER36; ΦER11, ΦER3, ΦER27, ΦMM9, and ΦER36; ΦER11, ΦER27, ΦMM9, ΦER36, and ΦER3; ΦER11, ΦMM9, ΦER36, ΦER3, and ΦER27; ΦER11, ΦER36, ΦER3, ΦER27, and ΦMM9; ΦER27, ΦER11, ΦER3, ΦER36, and ΦMM9; ΦER27, ΦER3, ΦER36, ΦMM9, and ΦER11; ΦER27, ΦER36, ΦMM9, ΦER11, and ΦER3; ΦER27, ΦMM9, ΦER11, ΦER3, and ΦER36; ΦER27, ΦER3, ΦER11, ΦER36, and ΦMM9; ΦER27, ΦER11, ΦER36, ΦMM9, and ΦER3; ΦER27, ΦER36, ΦMM9, ΦER3, and ΦER11; ΦER27, ΦMM9, ΦER3, ΦER11, and ΦER36; ΦER27, ΦER36, ΦER3, ΦER11, and ΦMM9; ΦER27, ΦER3, ΦER11, ΦMM9, and ΦER36; ΦER27, ΦER11, ΦMM9, ΦER36, and ΦER3; ΦER27, ΦMM9, ΦER36, ΦER3, and ΦER11; ΦER27, ΦMM9, ΦER3, ΦER36, and ΦER11; ΦER27, ΦER3, ΦER36, ΦER11, and ΦMM9; ΦER27, ΦER36, ΦER11, ΦMM9, and ΦER3; ΦER27, ΦER11, ΦMM9, ΦER3, and ΦER36; ΦER27, ΦER11, ΦER36, ΦER3, and ΦMM9; ΦER27, ΦER36, ΦER3, ΦMM9, and ΦER11; ΦER27, ΦER3, ΦMM9, ΦER11, and ΦER36; ΦER27, ΦMM9, ΦER11, ΦER36, and ΦER3; ΦER27, ΦER11, ΦMM9, ΦER36, and ΦER3; ΦER27, ΦMM9, ΦER36, ΦER3, and ΦER11; ΦER27, ΦER36, ΦER3, ΦER11, and ΦMM9; ΦER27, ΦER3, ΦER11, ΦMM9, and ΦER36; ΦER27, ΦER11, ΦER3, ΦMM9, and ΦER36; ΦER27, ΦER3, ΦMM9, ΦER36, and ΦER11; ΦER27, ΦMM9, ΦER36, ΦER11, and ΦER3; ΦER27, ΦER36, ΦER11, ΦER3, and ΦMM9; ΦER36, ΦER11, ΦER27, ΦER3, and ΦMM9; ΦER36, ΦER27, ΦER3, ΦMM9, and ΦER11; ΦER36, ΦER3, ΦMM9, ΦER11, and ΦER27; ΦER36, ΦMM9, ΦER11, ΦER27, and ΦER3; ΦER36, ΦER27, ΦER11, ΦER3, and ΦMM9; ΦER36, ΦER11, ΦER3, ΦMM9, and ΦER27; ΦER36, ΦER3, ΦMM9, ΦER27, and ΦER11; ΦER36, ΦMM9, ΦER27, ΦER11, and ΦER3; ΦER36, ΦER3, ΦER27, ΦER11, and ΦMM9; ΦER36, ΦER27, ΦER11, ΦMM9, and ΦER3; ΦER36, ΦER11, ΦMM9, ΦER3, and ΦER27; ΦER36, ΦMM9, ΦER3, ΦER27, and ΦER11; ΦER36, ΦMM9, ΦER27, ΦER3, and ΦER11; ΦER36, ΦER27, ΦER3, ΦER11, and ΦMM9; ΦER36, ΦER3, ΦER11, ΦMM9, and ΦER27; ΦER36, ΦER11, ΦMM9, ΦER27, and ΦER3; ΦER36, ΦER11, ΦER3, ΦER27, and ΦMM9; ΦER36, ΦER3, ΦER27, ΦMM9, and ΦER11; ΦER36, ΦER27, ΦMM9, ΦER11, and ΦER3; ΦER36, ΦMM9, ΦER11, ΦER3, and ΦER27; ΦER36, ΦER11, ΦMM9, ΦER3, and ΦER27; ΦER36, ΦMM9, ΦER3, ΦER27, and ΦER11; ΦER36, ΦER3, ΦER27, ΦER11, and ΦMM9; ΦER36, ΦER27, ΦER11, ΦMM9, and ΦER3; ΦER36, ΦER11, ΦER27, ΦMM9, and ΦER3; ΦER36, ΦER27, ΦMM9, ΦER3, and ΦER11; ΦER36, ΦMM9, ΦER3, ΦER11, and ΦER27; ΦER36, ΦER3, ΦER11, ΦER27, and ΦMM9; ΦMM9, ΦER11, ΦER27, ΦER36, and ΦER3; ΦMM9, ΦER27, ΦER36, ΦER3, and ΦER11; ΦMM9, ΦER36, ΦER3, ΦER11, and ΦER27; ΦMM9, ΦER3, ΦER11, ΦER27, and ΦER36; ΦMM9, ΦER27, ΦER11, ΦER36, and ΦER3; ΦMM9, ΦER11, ΦER36, ΦER3, and ΦER27; ΦMM9, ΦER36, ΦER3, ΦER27, and ΦER11; ΦMM9, ΦER3, ΦER27, ΦER11, and ΦER36; ΦMM9, ΦER36, ΦER27, ΦER11, and ΦER3; ΦMM9, ΦER27, ΦER11, ΦER3, and ΦER36; ΦMM9, ΦER11, ΦER3, ΦER36, and ΦER27; ΦMM9, ΦER3, ΦER36, ΦER27, and ΦER11; ΦMM9, ΦER3, ΦER27, ΦER36, and ΦER11; ΦMM9, ΦER27, ΦER36, ΦER11, and ΦER3; ΦMM9, ΦER36, ΦER11, ΦER3, and ΦER27; ΦMM9, ΦER11, ΦER3, ΦER27, and ΦER36; ΦMM9, ΦER11, ΦER36, ΦER27, and ΦER3; ΦMM9, ΦER36, ΦER27, ΦER3, and ΦER11; ΦMM9, ΦER27, ΦER3, ΦER11, and ΦER36; ΦMM9, ΦER3, ΦER11, ΦER36, and ΦER27; ΦMM9, ΦER11, ΦER3, ΦER36, and ΦER27; ΦMM9, ΦER3, ΦER36, ΦER27, and ΦER11; ΦMM9, ΦER36, ΦER27, ΦER11, and ΦER3; ΦMM9, ΦER27, ΦER11, ΦER3, and ΦER36; ΦMM9, ΦER11, ΦER27, ΦER3, and ΦER36; ΦMM9, ΦER27, ΦER3, ΦER36, and ΦER11; ΦMM9, ΦER3, ΦER36, ΦER11, and ΦER27; ΦMM9, ΦER36, ΦER11, ΦER27, and ΦER3. One or more of the preceding administration sequences may be excluded from aspects of the disclosure.

Example administration sequences for ΦER15, ΦER43, ΦER27, ΦER46, and/or ΦMM7 include: ΦER15 and ΦER43; ΦER43 and ΦER15; ΦER15 and ΦER27; ΦER27 and ΦER15; ΦER15 and ΦER46; ΦER46 and ΦER15; ΦER15 and ΦMM7; ΦMM7 and ΦER15; ΦER15, ΦER43, and ΦER27; ΦER15, ΦER27, and ΦER43; ΦER43, ΦER15, and ΦER27; ΦER43, ΦER27, and ΦER15; ΦER27, ΦER15, and ΦER43; ΦER27, ΦER43, and ΦER15; ΦER15, ΦER43, and 39; ΦER15, ΦER46, and ΦER43; ΦER43, ΦER15, and ΦER46; ΦER43, ΦER46, and ΦER15; ΦER46, ΦER15, and ΦER43; ΦER46, ΦER43, and ΦER15; ΦER15, ΦER43, and 46; ΦER15, ΦMM7, and ΦER43; ΦER43, ΦER15, and ΦMM7; ΦER43, ΦMM7, and ΦER15; ΦMM7, ΦER15, and ΦER43; ΦMM7, ΦER43, and ΦER15; ΦER15, ΦER27, and 39; ΦER15, ΦER46, and ΦER27; ΦER27, ΦER15, and ΦER46; ΦER27, ΦER46, and ΦER15; ΦER46, ΦER15, and ΦER27; ΦER46, ΦER27, and ΦER15; ΦER15, ΦER27, and 46; ΦER15, ΦMM7, and ΦER27; ΦER27, ΦER15, and ΦMM7; ΦER27, ΦMM7, and ΦER15; ΦMM7, ΦER15, and ΦER27; ΦMM7, ΦER27, and ΦER15; ΦER15, ΦER46, and 46; ΦER15, ΦMM7, and ΦER46; ΦER46, ΦER15, and ΦMM7; ΦER46, ΦMM7, and ΦER15; ΦMM7, ΦER15, and ΦER46; ΦMM7, ΦER46, and ΦER15; ΦER15, ΦER43, ΦER27, and ΦER46; ΦER15, ΦER43, ΦER46, and ΦER27; ΦER15, ΦER27, ΦER46, and ΦER43; ΦER15, ΦER27, ΦER43, and ΦER46; ΦER15, ΦER46, ΦER43, and ΦER27; ΦER15, ΦER46, ΦER27, and ΦER43; ΦER43, ΦER15, ΦER27, and ΦER46; ΦER43, ΦER15, ΦER46, and ΦER27; ΦER43, ΦER27, ΦER46, and ΦER15; ΦER43, ΦER27, ΦER15, and ΦER46; ΦER43, ΦER46, ΦER43, and ΦER27; ΦER43, ΦER46, ΦER27, and ΦER15; ΦER27, ΦER43, ΦER15, and ΦER46; ΦER27, ΦER43, ΦER46, and ΦER15; ΦER27, ΦER15, ΦER46, and ΦER43; ΦER27, ΦER15, ΦER43, and ΦER46; ΦER27, ΦER46, ΦER43, and ΦER15; ΦER27, ΦER46, ΦER15, and ΦER43; ΦER46, ΦER43, ΦER27, and ΦER15; ΦER46, ΦER43, ΦER15, and ΦER27; ΦER46, ΦER27, ΦER15, and ΦER43; ΦER46, ΦER27, ΦER43, and ΦER15; ΦER46, ΦER15, ΦER43, and ΦER27; ΦER46, ΦER15, ΦER27, and ΦER43; ΦER15, ΦER43, ΦER27, and ΦMM7; ΦER15, ΦER43, ΦMM7, and ΦER27; ΦER15, ΦER27, ΦMM7, and ΦER43; ΦER15, ΦER27, ΦER43, and ΦMM7; ΦER15, ΦMM7, ΦER43, and ΦER27; ΦER15, ΦMM7, ΦER27, and ΦER43; ΦER43, ΦER15, ΦER27, and ΦMM7; ΦER43, ΦER15, ΦMM7, and ΦER27; ΦER43, ΦER27, ΦMM7, and ΦER15; ΦER43, ΦER27, ΦER15, and ΦMM7; ΦER43, ΦMM7, ΦER43, and ΦER27; ΦER43, ΦMM7, ΦER27, and ΦER15; ΦER27, ΦER43, ΦER15, and ΦMM7; ΦER27, ΦER43, ΦMM7, and ΦER15; ΦER27, ΦER15, ΦMM7, and ΦER43; ΦER27, ΦER15, ΦER43, and ΦMM7; ΦER27, ΦMM7, ΦER43, and ΦER15; ΦER27, ΦMM7, ΦER15, and ΦER43; ΦMM7, ΦER43, ΦER27, and ΦER15; ΦMM7, ER43, ΦER15, and ΦER27; ΦMM7, ΦER27, ΦER15, and ΦER43; ΦMM7, ΦER27, ΦER43, and ΦER15; ΦMM7, ΦER15, ΦER43, and ΦER27; ΦMM7, ΦER15, ΦER27, and ΦER43; ΦER15, ΦER43, ΦER46, and ΦMM7; ΦER15, ΦER43, ΦMM7, and ΦER46; ΦER15, ΦER46, ΦMM7, and ΦER43; ΦER15, ΦER46, ΦER43, and ΦMM7; ΦER15, ΦMM7, ΦER43, and ΦER46; ΦER15, ΦMM7, ΦER46, and ΦER43; ΦER43, ΦER15, ΦER46, and ΦMM7; ΦER43, ΦER15, ΦMM7, and ΦER46; ΦER43, ΦER46, ΦMM7, and ΦER15; ΦER43, ΦER46, ΦER15, and ΦMM7; ΦER43, ΦMM7, ΦER43, and ΦER46; ΦER43, ΦMM7, ΦER46, and ΦER15; ΦER46, ΦER43, ΦER15, and ΦMM7; ΦER46, ΦER43, ΦMM7, and ΦER15; ΦER46, ΦER15, ΦMM7, and ΦER43; ΦER46, ΦER15, ΦER43, and ΦMM7; ΦER46, ΦMM7, ΦER43, and ΦER15; ΦER46, ΦMM7, ΦER15, and ΦER43; ΦMM7, ΦER43, ΦER46, and ΦER15; ΦMM7, ΦER43, ΦER15, and ΦER46; ΦMM7, ΦER46, ΦER15, and ΦER43; ΦMM7, ΦER46, ΦER43, and ΦER15; ΦMM7, ΦER15, ΦER43, and ΦER46; ΦMM7, ΦER15, ΦER46, and ΦER43; ΦER15, ΦER27, ΦER46, and ΦMM7; ΦER15, ΦER27, ΦMM7, and ΦER46; ΦER15, ΦER46, ΦMM7, and ΦER27; ΦER15, ΦER46, ΦER27, and ΦMM7; ΦER15, ΦMM7, ΦER27, and ΦER46; ΦER15, ΦMM7, ΦER46, and ΦER27; ΦER27, ΦER15, ΦER46, and ΦMM7; ΦER27, ΦER15, ΦMM7, and ΦER46; ΦER27, ΦER46, ΦMM7, and ΦER15; ΦER27, ΦER46, ΦER15, and ΦMM7; ΦER27, ΦMM7, ΦER43, and ΦER46; ΦER27, ΦMM7, ΦER46, and ΦER15; ΦER46, ΦER27, ΦER15, and ΦMM7; ΦER46, ΦER27, ΦMM7, and ΦER15; ΦER46, ΦER15, ΦMM7, and ΦER27; ΦER46, ΦER15, ΦER27, and ΦMM7; ΦER46, ΦMM7, ΦER27, and ΦER15; ΦER46, ΦMM7, ΦER15, and ΦER27; ΦMM7, ΦER27, ΦER46, and ΦER15; ΦMM7, ΦER27, ΦER15, and ΦER46; ΦMM7, ΦER46, ΦER15, and ΦER27; ΦMM7, ΦER46, ΦER27, and ΦER15; ΦMM7, ΦER15, ΦER27, and ΦER46; ΦMM7, ΦER15, ΦER46, and ΦER27; ΦER15, ΦER43, ΦER27, ΦER46, and ΦMM7; ΦER15, ΦER27, ΦER46, ΦMM7, and ΦER43; ΦER15, ΦER46, ΦMM7, ΦER43, and ΦER27; ΦER15, ΦMM7, ΦER43, ΦER27, and ΦER46; ΦER15, ΦER27, ΦER43, ΦER46, and ΦMM7; ΦER15, ΦER43, ΦER46, ΦMM7, and ΦER27; ΦER15, ΦER46, ΦMM7, ΦER27, and ΦER43; ΦER15, ΦMM7, ΦER27, ΦER43, and ΦER46; ΦER15, ΦER46, ΦER27, ΦER43, and ΦMM7; ΦER15, ΦER27, ΦER43, ΦMM7, and ΦER46; ΦER15, ΦER43, ΦMM7, ΦER46, and ΦER27; ΦER15, ΦMM7, ΦER46, ΦER27, and ΦER43; ΦER15, ΦMM7, ΦER27, ΦER46, and ΦER43; ΦER15, ΦER27, ΦER46, ΦER43, and ΦMM7; ΦER15, ΦER46, ΦER43, ΦMM7, and ΦER27; ΦER15, ΦER43, ΦMM7, ΦER27, and ΦER46; ΦER15, ΦER43, ΦER46, ΦER27, and ΦMM7; ΦER15, ΦER46, ΦER27, ΦMM7, and ΦER43; ΦER15, ΦER27, ΦMM7, ΦER43, and ΦER46; ΦER15, ΦMM7, ΦER43, ΦER46, and ΦER27; ΦER15, ΦER43, ΦMM7, ΦER46, and ΦER27; ΦER15, ΦMM7, ΦER46, ΦER27, and ΦER43; ΦER15, ΦER46, ΦER27, ΦER43, and ΦMM7; ΦER15, ΦER27, ΦER43, ΦMM7, and ΦER46; ΦER15, ΦER43, ΦER27, ΦMM7, and ΦER46; ΦER15, ΦER27, ΦMM7, ΦER46, and ΦER43; ΦER15, ΦMM7, ΦER46, ΦER43, and ΦER27; ΦER15, ΦER46, ΦER43, ΦER27, and ΦMM7; ΦER43, ΦER15, ΦER27, ΦER46, and ΦMM7; ΦER43, ΦER27, ΦER46, ΦMM7, and ΦER15; ΦER43, ΦER46, ΦMM7, ΦER15, and ΦER27; ΦER43, ΦMM7, ΦER15, ΦER27, and ΦER46; ΦER43, ΦER27, ΦER15, ΦER46, and ΦMM7; ΦER43, ΦER15, ΦER46, ΦMM7, and ΦER27; ΦER43, ΦER46, ΦMM7, ΦER27, and ΦER15; ΦER43, ΦMM7, ΦER27, ΦER15, and ΦER46; ΦER43, ΦER46, ΦER27, ΦER15, and ΦMM7; ΦER43, ΦER27, ΦER15, ΦMM7, and ΦER46; ΦER43, ΦER15, ΦMM7, ΦER46, and ΦER27; ΦER43, ΦMM7, ΦER46, ΦER27, and ΦER15; ΦER43, ΦMM7, ΦER27, ΦER46, and ΦER15; ΦER43, ΦER27, ΦER46, ΦER15, and ΦMM7; ΦER43, ΦER46, ΦER15, ΦMM7, and ΦER27; ΦER43, ΦER15, ΦMM7, ΦER27, and ΦER46; ΦER43, ΦER15, ΦER46, ΦER27, and ΦMM7; ΦER43, ΦER46, ΦER27, ΦMM7, and ΦER15; ΦER43, ΦER27, ΦMM7, ΦER15, and ΦER46; ΦER43, ΦMM7, ΦER15, ΦER46, and ΦER27; ΦER43, ΦER15, ΦMM7, ΦER46, and ΦER27; ΦER43, ΦMM7, ΦER46, ΦER27, and ΦER15; ΦER43, ΦER46, ΦER27, PER 15, and ΦMM7; ΦER43, ΦER27, ΦER15, ΦMM7, and ΦER46; ΦER43, ΦER15, ΦER27, ΦMM7, and ΦER46; ΦER43, ΦER27, ΦMM7, ΦER46, and ΦER15; ΦER43, ΦMM7, ΦER46, ΦER15, and ΦER27; ΦER43, ΦER46, ΦER15, ΦER27, and ΦMM7; ΦER27, ΦER43, ΦER15, ΦER46, and ΦMM7; ΦER27, ΦER15, ΦER46, ΦMM7, and ΦER43; ΦER27, ΦER46, ΦMM7, ΦER43, and ΦER15; ΦER27, ΦMM7, ΦER43, ΦER15, and ΦER46; ΦER27, ΦER15, ΦER43, ΦER46, and ΦMM7; ΦER27, ΦER43, ΦER46, ΦMM7, and ΦER15; ΦER27, ΦER46, ΦMM7, ΦER15, and ΦER43; ΦER27, ΦMM7, ΦER15, ΦER43, and ΦER46; ΦER27, ΦER46, ΦER15, ΦER43, and ΦMM7; ΦER27, ΦER15, ΦER43, ΦMM7, and ΦER46; ΦER27, ΦER43, ΦMM7, ΦER46, and ΦER15; ΦER27, ΦMM7, ΦER46, ΦER15, and ΦER43; ΦER27, ΦMM7, ΦER15, ΦER46, and ΦER43; ΦER27, ΦER15, ΦER46, ΦER43, and ΦMM7; ΦER27, ΦER46, ΦER43, ΦMM7, and ΦER15; ΦER27, ΦER43, ΦMM7, ΦER15, and ΦER46; ΦER27, ΦER43, ΦER46, ΦER15, and ΦMM7; ΦER27, ΦER46, ΦER15, ΦMM7, and ΦER43; ΦER27, ΦER15, ΦMM7, ΦER43, and ΦER46; ΦER27, ΦMM7, ΦER43, ΦER46, and ΦER15; ΦER27, ΦER43, ΦMM7, ΦER46, and ΦER15; ΦER27, ΦMM7, ΦER46, ΦER15, and ΦER43; ΦER27, ΦER46, ΦER15, ΦER43, and ΦMM7; ΦER27, ΦER15, ΦER43, ΦMM7, and ΦER46; ΦER27, ΦER43, ΦER15, ΦMM7, and ΦER46; ΦER27, ΦER15, ΦMM7, ΦER46, and ΦER43; ΦER27, ΦMM7, ΦER46, ΦER43, and ΦER15; ΦER27, ΦER46, ΦER43, ΦER15, and ΦMM7; ΦER46, ΦER43, ΦER27, ΦER15, and ΦMM7; ΦER46, ΦER27, ΦER15, ΦMM7, and ΦER43; ΦER46, ΦER15, ΦMM7, ΦER43, and ΦER27; ΦER46, ΦMM7, ΦER43, ΦER27, and ΦER15; ΦER46, ΦER27, ΦER43, ΦER15, and ΦMM7; ΦER46, ΦER43, ΦER15, ΦMM7, and ΦER27; ΦER46, ΦER15, ΦMM7, ΦER27, and ΦER43; ΦER46, ΦMM7, ΦER27, ΦER43, and ΦER15; ΦER46, ΦER15, ΦER27, ΦER43, and ΦMM7; ΦER46, ΦER27, ΦER43, ΦMM7, and ΦER15; ΦER46, ΦER43, ΦMM7, ΦER15, and ΦER27; ΦER46, ΦMM7, ΦER15, ΦER27, and ΦER43; ΦER46, ΦMM7, ΦER27, PER 15, and ΦER43; ΦER46, ΦER27, ΦER15, ΦER43, and ΦMM7; ΦER46, ΦER15, ΦER43, ΦMM7, and ΦER27; ΦER46, ΦER43, ΦMM7, ΦER27, and ΦER15; ΦER46, ΦER43, ΦER15, ΦER27, and ΦMM7; ΦER46, ΦER15, ΦER27, ΦMM7, and ΦER43; ΦER46, ΦER27, ΦMM7, ΦER43, and ΦER15; ΦER46, ΦMM7, ΦER43, ΦER15, and ΦER27; ΦER46, ΦER43, ΦMM7, ΦER15, and ΦER27; ΦER46, ΦMM7, ΦER15, ΦER27, and ΦER43; ΦER46, ΦER15, ΦER27, ΦER43, and ΦMM7; ΦER46, ΦER27, ΦER43, ΦMM7, and ΦER15; ΦER46, ΦER43, ΦER27, ΦMM7, and ΦER15; ΦER46, ΦER27, ΦMM7, ΦER15, and ΦER43; ΦER46, ΦMM7, ΦER15, ΦER43, and ΦER27; ΦER46, ΦER15, ΦER43, ΦER27, and ΦMM7; ΦMM7, ΦER43, ΦER27, ΦER46, and ΦER15; ΦMM7, ΦER27, ΦER46, ΦER15, and ΦER43; ΦMM7, ΦER46, ΦER15, ΦER43, and ΦER27; ΦMM7, ΦER15, ΦER43, ΦER27, and ΦER46; ΦMM7, ΦER27, ΦER43, ΦER46, and ΦER15; ΦMM7, ΦER43, ΦER46, ΦER15, and ΦER27; ΦMM7, ΦER46, ΦER15, ΦER27, and ΦER43; ΦMM7, ΦER15, ΦER27, ΦER43, and ΦER46; ΦMM7, ΦER46, ΦER27, ΦER43, and ΦER15; ΦMM7, ΦER27, ΦER43, ΦER15, and ΦER46; ΦMM7, ΦER43, ΦER15, ΦER46, and ΦER27; ΦMM7, ΦER15, ΦER46, ΦER27, and ΦER43; ΦMM7, ΦER15, ΦER27, ΦER46, and ΦER43; ΦMM7, ΦER27, ΦER46, ΦER43, and ΦER15; ΦMM7, ΦER46, ΦER43, ΦER15, and ΦER27; ΦMM7, ΦER43, ΦER15, ΦER27, and ΦER46; ΦMM7, ΦER43, ΦER46, ΦER27, and ΦER15; ΦMM7, ΦER46, ΦER27, ΦER15, and ΦER43; ΦMM7, ΦER27, ΦER15, ΦER43, and ΦER46; ΦMM7, ΦER15, ΦER43, ΦER46, and ΦER27; ΦMM7, ER43, ΦER15, ΦER46, and ΦER27; ΦMM7, ΦER15, ΦER46, ΦER27, and ΦER43; ΦMM7, ΦER46, ΦER27, ΦER43, and ΦER15; ΦMM7, ΦER27, ΦER43, ΦER15, and ΦER46; ΦMM7, ER43, ΦER27, ΦER15, and ΦER46; ΦMM7, ΦER27, ΦER15, ΦER46, and ΦER43; ΦMM7, ΦER15, ΦER46, ΦER43, and ΦER27; ΦMM7, ΦER46, ΦER43, ΦER27, and ΦER15. One or more of the preceding administration sequences may be excluded from aspects of the disclosure.

Additionally, an individual having a pathogenic Klebsiella infection may be administered any two or more ΦER1, ΦER2, ΦER3, ΦER4, ΦER5, ΦER6, ΦER7/ER10, ΦER8, ΦER11, ΦER12, ΦER13, ΦER14, ΦER15, ΦER16, ΦER18, ΦER20, ΦER21, ΦER22, ΦER23, ΦER27, ΦER32, ΦER36, ΦER37, ΦER38, ΦER39, ΦER43, ΦER44, ΦER45, ΦER46, ΦER47, ΦER48, ΦER49, ΦER50, ΦER51, ΦER52, ΦER53, ΦER54, ΦER55, ΦMM1, ΦMM2, ΦMM3, ΦMM4, ΦMM5, ΦMM6, ΦMM7, ΦMM8, and/or ΦMM9. In some aspects, an individual having a pathogenic Klebsiella infection may be administered ΦER3, ΦER11, ΦER12, ΦER15, ΦER16, ΦER27, ΦER36, ΦER39, ΦER43, ΦER46, ΦMM7, and/or ΦMM9, or one or more compositions thereof, in any order. In some aspects, an individual having a pathogenic Klebsiella infection may be administered ΦER12, ΦER15, ΦER16, ΦER39, ΦER46, or any combination thereof, in any order. In some aspects, an individual having a pathogenic Klebsiella infection may be administered ΦER3, ΦER11, ΦER27, ΦER36, ΦMM9, or any combination thereof, in any order. In some aspects, an individual having a pathogenic Klebsiella infection may be administered ΦER15, ΦER27, ΦER43, ΦER46, ΦMM7, or any combination thereof, in any order.

Bacteriophages of the disclosure may be administered to an individual once, or may be administered multiple times (e.g., 2, 3, 4, 5, 6, 7, 8, 9, 10, 11, 12, 13, 14, 15, or more times). Bacteriophages may be administered via an appropriate treatment regimen for an appropriate length of time, e.g., for effective treatment or prevention of a pathogenic Klebsiella infection. For example, a bacteriophage composition may be administered to an individual 1, 2, 3, or 4 times per day (or more); 1, 2, 3, 4, 5, 6, or 7 times per week (or more); or 1, 2, 3, 4, 5, 6, 7, 8, 9, 10, 11, 12, 13, 14, 15, 16, 17, 18, 19, or 20 times per month (or more). A bacteriophage composition may be administered for at least, at most, exactly, or between any two of 1, 2, 3, 4, 5, 6, 7, 8, 9, 10, 11, 12, 13, or 14 days (or more); 1, 2, 3, 4, 5, 6, 7, 8, 9, 10, 11, 12, 13, 14, 15, 16, 17, 18, 19, 20, or 21 weeks (or more); or 1, 2, 3, 4, 5, 6, 7, 8, 9, 10, 11, or 12 months (or more). In some aspects, a bacteriophage is administered twice per week for between 6 and 12 weeks.

An individual receiving a treatment of the disclosure may have or be at risk of having a pathogenic infection. An individual receiving a treatment of the disclosure may have or be at risk of having an infection in, for example, one or more of a urinary tract, blood, gut, abdomen, stomach, lungs, skin, wound (e.g., burns, scratches, surgical wounds, etc.), eyes, ears, mouth, nose, kidneys, prostate, bladder, brain, vaginal tract, heart, liver, and spleen. In some aspects, an individual has or is at risk of having a lung infection. In some aspects, an individual has or is at risk of having a bloodstream infection. In some aspects, an individual has or is at risk of having a urinary tract infection. In some aspects, an individual has or is at risk of having an infection of the urinary tract, blood, gut, abdomen, stomach, lungs, skin, wound (e.g., burns, scratches, surgical wounds, etc.), eyes, ears, mouth, nose, kidneys, prostate, bladder, brain, vaginal tract, heart, liver, and/or spleen that is associated with a catheter, drive line, syringe, tube, implant, defibrillator, artificial joint, pacemaker, screw, rod, disc, intrauterine device, pin, plate, stent, dental device, eye lens, shunt, valve, neurological or neurosurgical device, gastrointestinal device, genitourinary device, catheter cuff, vascular access device, and wound drain. In some aspects, the infection of the urinary tract, blood, gut, abdomen, stomach, lungs, skin, wound (e.g., burns, scratches, surgical wounds, etc.), eyes, ears, mouth, nose, kidneys, prostate, bladder, brain, vaginal tract, heart, liver, and/or spleen is caused by a pathogen (e.g., Klebsiella). An individual may have or be at risk of having one or more of a urinary tract infection, a blood-stream infection, pneumonia, sepsis, a surgical wound infection, a skin infection, a respiratory tract infection, a prostate infection, meningitis, pyogenic liver abscess, intra-abdominal infection, and a vaginal infection.

An individual may have one or more symptoms of a pathogenic infection (e.g., a Klebsiella infection), for example, fever, chills, coughing, shortness of breath, chest pain, yellow or bloody mucus, redness, swelling, pain, fatigue, wounds, ulcers, headache, diarrhea, stomach cramping, nausea, and/or vomiting. In some aspects, an individual does not have any symptoms of a pathogenic infection (e.g., a Klebsiella infection). Accordingly, any one or more bacteriophage of the disclosure (e.g., ΦER1, ΦER2, ΦER3, ΦER4, ΦER5, ΦER6, ΦER7/ΦER10, ΦER8, ΦER11, ΦER12, ΦER13, ΦER14, ΦER15, ΦER16, ΦER18, ΦER20, ΦER21, ΦER22, ΦER23, ΦER27, ΦER32, ΦER36, ΦER37, ΦER38, ΦER39, ΦER43, ΦER44, ΦER45, ΦER46, ΦER47, ΦER48, ΦER49, ΦER50, ΦER51, ΦER52, ΦER53, ΦER54, ΦER55, ΦMM1, ΦMM2, ΦMM3, ΦMM4, ΦMM5, ΦMM6, ΦMM7, ΦMM8, and/or ΦMM9) may be administered to treat or prevent one or more symptoms of a pathogenic infection (e.g., a Klebsiella infection). In some aspects, ΦER3, ΦER11, ΦER12, ΦER15, ΦER16, ΦER27, ΦER36, ΦER39, ΦER43, ΦER46, ΦMM7, and/or ΦMM9) may be administered to treat or prevent one or more symptoms of a pathogenic infection (e.g., a Klebsiella infection). In some aspects, a ΦER12, ER15, ΦER16, ΦER39, ΦER46, or any combination thereof, may be administered to treat or prevent one or more symptoms of a pathogenic infection (e.g., a Klebsiella infection). In some aspects, ΦER3, ΦER11, ΦER27, ΦER36, ΦMM9, or any combination thereof, may be administered to treat or prevent one or more symptoms of a pathogenic infection (e.g., a Klebsiella infection). In some aspects ΦER15, ΦER27, ΦER43, ΦER46, ΦMM7, or any combination thereof, may be administered to treat or prevent one or more symptoms of a pathogenic infection (e.g., a Klebsiella infection). Any one or more of the preceding bacteriophage(s) may be excluded from administration to treat or prevent one or more symptoms of a pathogenic infection (e.g., a Klebsiella infection).

In some aspects, an individual receiving a treatment of the disclosure has a Klebsiella infection. In some aspects, an individual has been diagnosed with a Klebsiella infection. In some aspects, an individual has one or more symptoms of a Klebsiella infection (including, e.g., diarrhea, stomach cramping, nausea, and/or vomiting). In some aspects, the individual does not have any symptoms of a Klebsiella infection. In some aspects, the Klebsiella is multidrug-resistant (i.e., is a multidrug-resistant a Klebsiella). In some aspects, the Klebsiella is hypervirulent Klebsiella. In some aspects, the Klebsiella is Klebsiella pneumoniae (K. pneumoniae). In some aspects, the Klebsiella is Klebsiella quasipneumoniae (K. quasipneumoniae). In some aspects, the Klebsiella is Klebsiella variicola (K. variicola). An individual may have received a Klebsiella infection from various sources. For example, in some aspects, the Klebsiella was from a beverage, comestible (e.g., undercooked meat, unpasteurized milk, apple juice or cider; or soft cheese made from raw milk), another individual, or an environment (e.g., ground or surface water, water used to irrigate crops, a public water system, a hospital, a school, a nursing home, a petting zoo, a cruise ship, a train, or an airplane).

An individual may have one or more risk factors for contracting a pathogenic infection (e.g., a Klebsiella infection), for example, age, antibiotic use, corticosteroid use, hospitalization, ventilation, use of an i.v. or urinary catheter, surgery, wounds, diabetes, alcohol use, COPD, chronic liver disease, lung disease, kidney failure, dialysis, solid-organ transplantation, cancer, and/or chemotherapy. In some aspects, an individual does not have any risk factors for contracting a pathogenic infection (e.g., a Klebsiella infection). Accordingly, in certain aspects, bacteriophages of the disclosure may be administered to an individual before, during, and/or after the individual develops one or more risk factors for contracting a pathogenic infection (e.g., a Klebsiella infection). For example, any one or more bacteriophage of the disclosure (e.g., ΦER1, ΦER2, ΦER3, ΦER4, ΦER5, ΦER6, ΦER7/ΦER10, ΦER8, ΦER11, ΦER12, ΦER13, ΦER14, ΦER15, ΦER16, ΦER18, ΦER20, ΦER21, ΦER22, ΦER23, ΦER27, ΦER32, ΦER36, ΦER37, ΦER38, ΦER39, ΦER43, ΦER44, ΦER45, ΦER46, ΦER47, ΦER48, ΦER49, ΦER50, ΦER51, ΦER52, ΦER53, ΦER54, ΦER55, ΦMM1, ΦMM2, ΦMM3, ΦMM4, ΦMM5, ΦMM6, ΦMM7, ΦMM8, and/or ΦMM9. In some aspects, ΦER3, ΦER11, ΦER12, ΦER15, ΦER16, ΦER27, ΦER36, ΦER39, ΦER43, ΦER46, ΦMM7, and/or ΦMM9) may be administered to an individual before, during, and/or after insertion of an i.v. or urinary catheter, thereby treating or preventing a pathogenic infection (e.g., a Klebsiella infection).

An individual receiving a treatment of the disclosure may be an immunosuppressed individual. An immunosuppressed individual may be an individual having, for example, an immune cell defect, asplenia, impaired splenic function, nephrotic syndrome, or an autoimmune condition. An immunosuppressed individual may be an individual subject to immunosuppressive conditions, for example chemotherapeutic agents or immunosuppressant agents (e.g., a glucocorticoid, a calcineurin inhibitor, an antimetabolite, a medication to reduce stomach acid such as a proton pump inhibitor, or an antibody therapy). Accordingly, in certain aspects, bacteriophages of the disclosure may be administered to an individual before, during, and/or after subjecting the individual to immunosuppressive conditions. For example, any one or more bacteriophage of the disclosure (e.g., ΦER1, ER2, ΦER3, ΦER4, ΦER5, ΦER6, ΦER7/ΦER10, ΦER8, ΦER11, ΦER12, ΦER13, ΦER14, ΦER15, ΦER16, ΦER18, ΦER20, ΦER21, ΦER22, ΦER23, ΦER27, ΦER32, ΦER36, ΦER37, ΦER38, ΦER39, ΦER43, ΦER44, ER45, ΦER46, ΦER47, ΦER48, ΦER49, ΦER50, ΦER51, ΦER52, ΦER53, ΦER54, ΦER55, ΦMM1, ΦMM2, ΦMM3, ΦMM4, ΦMM5, ΦMM6, ΦMM7, ΦMM8, and/or ΦMM9. In some aspects, ΦER3, ΦER11, ΦER12, ΦER15, ΦER16, ΦER27, ΦER36, ΦER39, ΦER43, ΦER46, ΦMM7, and/or ΦMM9) may be administered to an individual having cancer during treatment with chemotherapy, thereby treating or preventing a pathogenic Klebsiella infection.

The bacteriophage of the disclosure may be administered with one or more other treatments for Klebsiella infection, for example, cephalosporins (e.g., cefotaxime, ceftriaxone), quinolones, carbapenems (e.g., imipenem, cilastatin), minoglycosides (e.g., gentamicin, amikacin), ceftazidime/avibactam, ceftolozane/tazobactam, meropenem/vaborbactam, aztreonam, ampicillin/sulbactam, piperacillin/tazobactam, ticarcillin/clavulanate, ceftazidime, cefepime, levofloxacin, norfloxacin, moxifloxacin, meropenem, ertapenem, or a combination thereof. The bacteriophage and treatment(s) for Klebsiella infection may be administered in any order (e.g., simultaneously or sequentially). The bacteriophage and treatment(s) for Klebsiella infection may be comprised in the same or different compositions.

### III. Pharmaceutical Compositions

In certain aspects, the compositions or agents for use in the disclosed methods include 1, 2, 3, 4, 5, 6, 7, 8, 9, 10, or more bacteriophage(s). In certain aspects, the compositions or agents for use in the disclosed methods include ΦER1, ΦER2, ΦER3, ΦER4, ΦER5, ΦER6, ΦER7/ΦER10, ΦER8, ΦER11, ΦER12, ΦER13, ΦER14, ΦER15, ΦER16, ΦER18, ΦER20, ΦER21, ΦER22, ΦER23, ΦER27, ΦER32, ΦER36, ΦER37, ER38, ΦER39, ΦER43, ΦER44, ER45, ΦER46, ΦER47, ΦER48, ER49, ΦER50, ΦER51, ΦER52, ΦER53, ΦER54, ΦER55, ΦMM1, ΦMM2, ΦMM3, ΦMM4, ΦMM5, ΦMM6, ΦMM7, ΦMM8, and/or ΦMM9. Any one or more of the foregoing bacteriophage may be excluded from a composition or as an agent for use in the methods disclosed herein.

In some aspects, ΦER3, ΦER11, ΦER12, ΦER15, ΦER16, ΦER27, ΦER36, ΦER39, ΦER43, ΦER46, ΦMM7, and/or ΦMM9, are suitably contained in a pharmaceutically acceptable carrier. In some aspects, ΦER12, ΦER15, ΦER16, ΦER39, ΦER46, or any combination thereof, are suitably contained in a pharmaceutically acceptable carrier. In some aspects, ΦER3, ΦER11, ΦER27, ER36, ΦMM9, or any combination thereof, are suitably contained in a pharmaceutically acceptable carrier. In some aspects, ΦER15, ER27, ΦER43, ΦER46, ΦMM7, or any combination thereof, are suitably contained in a pharmaceutically acceptable carrier.

In some aspects, the carrier is non-toxic, biocompatible, and is selected so as not to detrimentally affect the biological activity of the agent. The agents in some aspects of the disclosure may be formulated into preparations for local delivery (i.e., to a specific location of the body) or systemic delivery, in solid, semi-solid, gel, liquid, or gaseous forms such as tablets, capsules, powders, granules, ointments, solutions, depositories, inhalants, and injections allowing for oral, parenteral, or surgical administration. Certain aspects of the disclosure also contemplate local administration of the compositions by coating medical devices and the like.

Suitable carriers for parenteral delivery via injectable, infusion, or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose, any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability, or pharmacokinetics of the therapeutic agent(s). Such a delivery vehicle may include, by way of non-limiting examples, microparticles, microspheres, nanospheres, or nanoparticles composed of proteins, liposomes, carbohydrates, synthetic organic compounds, inorganic compounds, polymeric or copolymeric hydrogels, and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient, and the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof, and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil, and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components of the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate, and the like. The compositions take the form of solutions, suspensions, tablets, pills, capsules, sustained release formulations, or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers, or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability, and toxicity of the particular therapeutic substance.

It is contemplated that other agents may be used in combination with certain aspects of the present disclosure to improve the therapeutic efficacy of treatment. These additional agents include agents having antibacterial properties (e.g., antibiotics). For example, one or more therapeutic phage of the disclosure may be used in combination with one or more antibiotics. Various antibiotics are recognized in the art and contemplated herein including, for example, cephalosporins ceftriaxone), carbapenems (e.g., (e.g., cefotaxime, imipenem/cilastatin, meropenem, ertapenem), aminoglycosides (e.g., gentamicin, amikacin), beta-lactams (e.g., ceftolozane/tazobactam, ceftazidime/avibactam, vaborbactam), quinolones (e.g., ciprofloxacin, levofloxacin, norfloxacin, moxifloxacin), ampicillin, avibactam, aztreonam, rifampin, sulbactam, piperacillin, tazobactam, ticarcillin, clavulanate, ceftazidime, cefepime, colistin, tigecycline, trimethoprim, sulfamethoxazole, metronidazole, or combinations of any two or more of the foregoing.

### IV. Devices

Also contemplated herein, in some aspects, are devices (e.g., medical devices) comprising one or more bacteriophages or bacteriophage compositions of the present disclosure. A device of the disclosure may comprise 1, 2, 3, 4, 5, 6, 7, 8, 9, 10 or more bacteriophage(s). A device of the disclosure may comprise one or more of ΦER1, ΦER2, ΦER3, ER4, ΦER5, ΦER6, ΦER7/ΦER10, ΦER8, ΦER11, ΦER12, ΦER13, ΦER14, ΦER15, ΦER16, ΦER18, ΦER20, ΦER21, ΦER22, ΦER23, ΦER27, ΦER32, ER36, ΦER37, ΦER38, ΦER39, ΦER43, ΦER44, ΦER45, ΦER46, ΦER47, ΦER48, ΦER49, ΦER50, ΦER51, ΦER52, ΦER53, ΦER54, ΦER55, ΦMM1, ΦMM2, ΦMM3, ΦMM4, ΦMM5, ΦMM6, ΦMM7, ΦMM8, and/or ΦMM9. Any one or more of the foregoing bacteriophage(s) may be excluded from a device of the disclosure. In some aspects, a device of the disclosure may comprise ΦER3, ΦER11, ΦER12, ΦER15, ΦER16, ΦER27, ΦER36, ΦER39, ΦER43, ΦER46, ΦMM7, and/or ΦMM9, or one or more compositions thereof. In some aspects, the device comprises ΦER3 or a composition thereof. In some aspects, the device comprises ΦER11 or a composition thereof. In some aspects, the device comprises ΦER12 or a composition thereof. In some aspects, the device comprises ΦER15 or a composition thereof. In some aspects, the device comprises ΦER16 or a composition thereof. In some aspects, the device comprises ΦER27 or a composition thereof. In some aspects, the device comprises ΦER36 or a composition thereof. In some aspects, the device comprises ΦER39 or a composition thereof. In some aspects, the device comprises ΦER43 or a composition thereof. In some aspects, the device comprises ΦER46 or a composition thereof. In some aspects, the device comprises ΦMM7 or a composition thereof. In some aspects, the device comprises ΦMM9 or a composition thereof. In some aspects, the device comprises two or more of ΦER1, ΦER2, ΦER3, ΦER4, ΦER5, ΦER6, ΦER7/ΦER10, ΦER8, ΦER11, ΦER12, ΦER13, ΦER14, ΦER15, ΦER16, ΦER18, ΦER20, ΦER21, ΦER22, ΦER23, ΦER27, ΦER32, ΦER36, ΦER37, ΦER38, ΦER39, ΦER43, ΦER44, ΦER45, ΦER46, ΦER47, ΦER48, ΦER49, ΦER50, ΦER51, ΦER52, ΦER53, ΦER54, ΦER55, ΦMM1, ΦMM2, ΦMM3, ΦMM4, ΦMM5, ΦMM6, ΦMM7, ΦMM8, and/or ΦMM9. In some aspects, ΦER3, ΦER11, ΦER12, ΦER15, ΦER16, ΦER27, ΦER36, ΦER39, ΦER43, ΦER46, ΦMM7, and/or ΦMM9, or one or more compositions thereof. In some aspects, the device comprises three or more of ΦER1, ΦER2, ΦER3, ΦER4, ΦER5, ER6, ΦER7/ΦER10, ΦER8, ΦER11, ΦER12, ER13, ΦER14, ΦER15, ΦER16, ΦER18, ΦER20, ΦER21, ΦER22, ΦER23, ΦER27, ER32, ΦER36, ΦER37, ΦER38, ΦER39, ΦER43, ΦER44, ΦER45, ΦER46, ΦER47, ER48, ΦER49, ΦER50, ΦER51, ΦER52, ΦER53, ΦER54, ΦER55, ΦMM1, ΦMM2, ΦMM3, ΦMM4, ΦMM5, ΦMM6, ΦMM7, ΦMM8, and/or ΦMM9. In some aspects, ΦER3, ER11, ΦER12, ΦER15, ΦER16, ΦER27, ΦER36, ΦER39, ΦER43, ΦER46, ΦMM7, and/or ΦMM9, or one or more compositions thereof. In some aspects, the device comprises four or more of ΦER1, ΦER2, ΦER3, ΦER4, ΦER5, ΦER6, ΦER7/ΦER10, ΦER8, ΦER11, ΦER12, ΦER13, ΦER14, ΦER15, ΦER16, ΦER18, ΦER20, ΦER21, ΦER22, ΦER23, ΦER27, ΦER32, ΦER36, ΦER37, ΦER38, ΦER39, ΦER43, ΦER44, ΦER45, ΦER46, ΦER47, ΦER48, ΦER49, ΦER50, ΦER51, ΦER52, ΦER53, ΦER54, ΦER55, ΦMM1, ΦMM2, ΦMM3, ΦMM4, ΦMM5, ΦMM6, ΦMM7, ΦMM8, and/or ΦMM9. In some aspects, ΦER3, ΦER11, ΦER12, ΦER15, ΦER16, ΦER27, ΦER36, ΦER39, ΦER43, ΦER46, ΦMM7, and/or ΦMM9, or one or more compositions thereof.

In some aspects, the device comprises ΦER12, ΦER15, ΦER16, ΦER39, ΦER46, or any combination thereof. In some aspects, the device comprises ΦER3, ΦER11, ΦER27, ΦER36, ΦMM9, or any combination thereof. In some aspects, the device comprises ΦER15, ΦER27, ΦER43, ΦER46, ΦMM7, or any combination thereof.

In some aspects, the device comprises one of the bacteriophage combinations of Table 1, other bacteriophage combinations completed herein, or one or more compositions thereof.

A device is described herein as “comprising” a bacteriophage or bacteriophage composition where the device has in, on, or around it, or is attached to, the bacteriophage or bacteriophage composition. In some aspects, a device of the disclosure is a medical device. In such cases, it may be desirable for a medical device to comprise bacteriophages capable of treating or preventing a pathogenic infection, such as a Klebsiella infection. Various medical devices are recognized in the art and contemplated herein. Examples of devices contemplated herein include, but are not limited to, a catheter, drive line, syringe, tube, implant, defibrillator, artificial joint, pacemaker, screw, rod, disc, intrauterine device, pin, plate, stent, dental device, eye lens, shunt, valve, neurological or neurosurgical device, gastrointestinal device, genitourinary device, catheter cuff, vascular access device, and wound drain. In some aspects, the device is a stent. In some aspects, the device is a catheter. In some aspects, the device is an implant.

Aspects of the disclosure comprise methods for preparing a device comprising subjecting a device to a bacteriophage or composition thereof of the disclosure (e.g., a bacteriophage composition comprising ΦER1, ΦER2, ΦER3, ΦER4, ΦER5, ΦER6, ΦER7/ΦER10, ΦER8, ΦER11, ΦER12, ΦER13, ΦER14, ΦER15, ΦER16, ΦER18, ΦER20, ΦER21, ΦER22, ΦER23, ΦER27, ΦER32, ΦER36, ΦER37, ΦER38, ΦER39, ΦER43, ΦER44, ΦER45, ΦER46, ΦER47, ΦER48, ΦER49, ΦER50, ΦER51, ΦER52, ΦER53, ΦER54, ΦER55, ΦMM1, ΦMM2, ΦMM3, ΦMM4, ΦMM5, ΦMM6, ΦMM7, ΦMM8, and/or ΦMM9). In some aspects, methods for preparing a device comprise subjecting a device to a bacteriophage or composition thereof of the disclosure (e.g., a bacteriophage composition comprising ΦER3, ΦER11, ΦER12, ΦER15, ΦER16, ΦER27, ΦER36, ΦER39, ΦER43, ΦER46, ΦMM7, and/or ΦMM9). Disclosed are methods comprising placing a bacteriophage or composition thereof on a device, placing a bacteriophage or composition thereof around a device, placing a bacteriophage or composition thereof in a device, and coating a device with a bacteriophage or composition thereof (e.g., coating one or more surfaces of a device). Aspects further comprise delivering the device to an individual following such preparation.

### V. Kits

Any of the compositions described herein may be comprised in a kit. In a non-limiting example, phage, reagents to produce or isolate phage, vectors, and reagents to produce vectors, and/or components thereof may be comprised in a kit. In certain aspects, phage may be comprised in a kit. Such a kit may or may not have one or more reagents for manipulation of phage. Such reagents include small molecules, proteins, nucleic acids, antibodies, buffers, primers, nucleotides, salts, and/or a combination thereof, for example. Nucleotides that encode components of phage may be included in the kit, including reagents to generate same.

In particular aspects, the kit comprises one or more bacteriophages, and/or pharmaceutical compositions comprising the same, alone or in any together with a pharmaceutically acceptable carrier or excipient. In some cases, the kit, in addition to the one or more bacteriophages, also includes an additional therapy, such as a treatment for Klebsiella infection, for example, cephalosporins (e.g., cefotaxime, ceftriaxone), quinolones, carbapenems (e.g., imipenem, cilastatin), minoglycosides (e.g., gentamicin, amikacin), ceftazidime/avibactam, ceftolozane/tazobactam, meropenem/vaborbactam, aztreonam, ampicillin/sulbactam, piperacillin/tazobactam, ticarcillin/clavulanate, ceftazidime, cefepime, levofloxacin, norfloxacin, moxifloxacin, meropenem, ertapenem, or a combination thereof. The kit(s) may be tailored to a particular disease, disorder, or condition for an individual and comprise respective additional therapies for the individual.

A kit may comprise, comprise at least, or comprise at most 1, 2, 3, 4, 5, 6, 7, 8, 9, or 10 different bacteriophages, or more. In some aspects, a kit of the disclosure comprises at least, as most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or all of ΦER1, ΦER2, ΦER3, ΦER4, ΦER5, ΦER6, ΦER7/ΦER10, ΦER8, ΦER11, ΦER12, ΦER13, ΦER14, ΦER15, ΦER16, ΦER18, ΦER20, ΦER21, ΦER22, ΦER23, ΦER27, ΦER32, ΦER36, ΦER37, ΦER38, ΦER39, ΦER43, ΦER44, ΦER45, ΦER46, ΦER47, ΦER48, ΦER49, ΦER50, ΦER51, ΦER52, ΦER53, ΦER54, ΦER55, ΦMM1, ΦMM2, ΦMM3, ΦMM4, ΦMM5, ΦMM6, ΦMM7, ΦMM8, and/or ΦMM9. In some aspects, ΦER3, ΦER11, ΦER12, ΦER15, ΦER16, ΦER27, ΦER36, ΦER39, ΦER43, ΦER46, MM7, and/or ΦMM9. In some aspects, a kit of the disclosure comprises at least, at most, or exactly 1, 2, 3, 4, or more of ΦER3, ΦER11, ER12, ΦER15, ΦER16, ΦER27, ΦER36, ΦER39, ΦER43, ΦER46, ΦMM7, and ΦMM9. One or more phage(s) may be specifically excluded from certain aspects. In some aspects, a kit of the disclosure comprises ΦER3 or a composition thereof. In some aspects, a kit of the disclosure comprises ΦER11 or a composition thereof. In some aspects, a kit of the disclosure comprises ΦER12 or a composition thereof. In some aspects, a kit of the disclosure comprises ΦER15 or a composition thereof. In some aspects, a kit of the disclosure comprises ΦER16 or a composition thereof. In some aspects, a kit of the disclosure comprises ΦER27 or a composition thereof. In some aspects, a kit of the disclosure comprises ΦER36 or a composition thereof. In some aspects, a kit of the disclosure comprises ΦER39 or a composition thereof. In some aspects, a kit of the disclosure comprises ΦER43 or a composition thereof. In some aspects, a kit of the disclosure comprises ΦER46 or a composition thereof. In some aspects, a kit of the disclosure comprises ΦMM7 or a composition thereof. In some aspects, a kit of the disclosure comprises ΦMM9 or a composition thereof. In some aspects, a kit of the disclosure comprises two or more of ΦER3, ΦER11, ΦER12, ΦER15, ΦER16, ΦER27, ΦER36, ΦER39, ΦER43, ΦER46, ΦMM7, and ΦMM9, or one or more compositions thereof. In some aspects, a kit of the disclosure comprises three or more of ΦER3, ΦER11, ΦER12, ΦER15, ΦER16, ΦER27, ΦER36, ΦER39, ΦER43, ΦER46, ΦMM7, and ΦMM9, or one or more compositions thereof. In some aspects, a kit of the disclosure comprises four or more of ΦER3, ΦER11, ΦER12, ΦER15, ΦER16, ΦER27, ΦER36, ΦER39, ΦER43, ΦER46, ΦMM7, and ΦMM9, or one or more compositions thereof.

In some aspects, kit of the disclosure comprises ΦER12, ΦER15, ΦER16, ΦER39, ΦER46, or any combination thereof. In some aspects, kit of the disclosure comprises ΦER3, ΦER11, ΦER27, ΦER36, ΦMM9, or any combination thereof. In some aspects, kit of the disclosure comprises ΦER15, ΦER27, ΦER43, ΦER46, ΦMM7, or any combination thereof.

In some aspects, a kit of the disclosures comprises one of the bacteriophage combinations of Table 1, other bacteriophage combinations completed herein, or one or more compositions thereof.

The kit can further comprise a package insert comprising instructions to treat or delay progression of disease, for example, Klebsiella infection, in an individual or to enhance treatment of for a Klebsiella infection. Any of the one or more bacteriophages, and/or pharmaceutical compositions comprising the same, alone or in any together with a pharmaceutically acceptable carrier or excipient, as described herein, may be included in the article of manufacture or kits. Kits may comprise components, which may be individually packaged or placed in a container. Suitable containers include, for example, bottles, vials, bags, tubes, syringes, and other suitable container means. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a treatment for Klebsiella infection, for example, cephalosporins (e.g., cefotaxime, ceftriaxone), quinolones, carbapenems (e.g., imipenem, cilastatin), minoglycosides (e.g., gentamicin, amikacin), ceftazidime/avibactam, ceftolozane/tazobactam, meropenem/vaborbactam, aztreonam, ampicillin/sulbactam, piperacillin/tazobactam, ticarcillin/clavulanate, ceftazidime, cefepime, levofloxacin, norfloxacin, moxifloxacin, meropenem, ertapenem, or a combination thereof). Suitable containers for the one or more agent include, for example, bottles, vials, bags and syringes.

A kit of the disclosure may further comprise a device. A kit may comprise a device having one or more bacteriophage or compositions thereof of the disclosure on, in, and/or around the device. A kit may comprise a device separate from one or more bacteriophage or compositions thereof.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

### EXAMPLES

The following examples are included to demonstrate certain aspects of the disclosure. It should be appreciated by those of skill in the art that the techniques disclosed in the examples which follow represent techniques discovered by the inventor to function well in the practice of the disclosure, and thus can be considered to constitute certain modes for its practice. However, those of skill in the art should, in light of the present disclosure, appreciate that many changes can be made in the specific aspects which are disclosed and still obtain a like or similar result without departing from the spirit and scope of the disclosure.

### Example 1—Identification of Bacteriophage of Interest

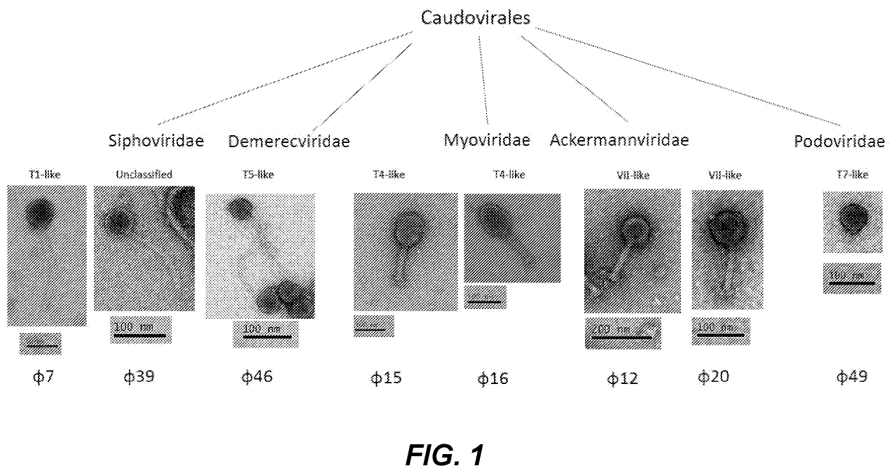

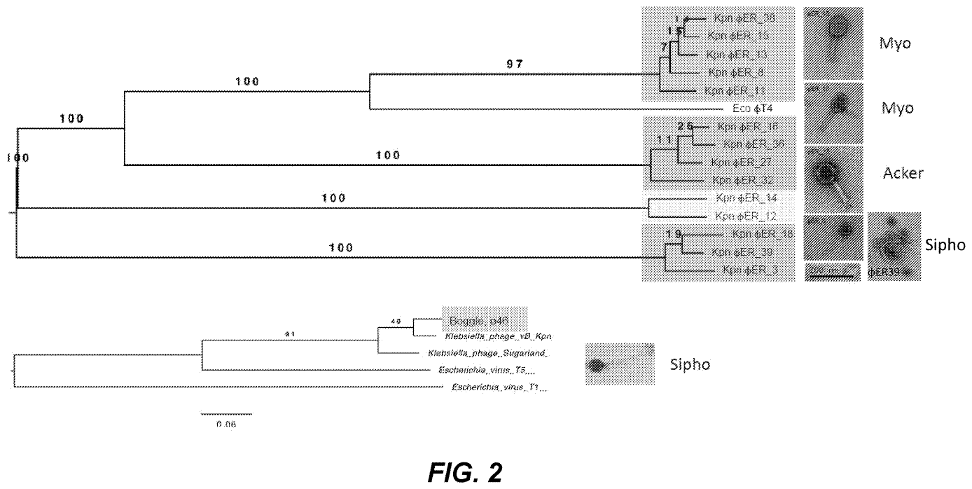

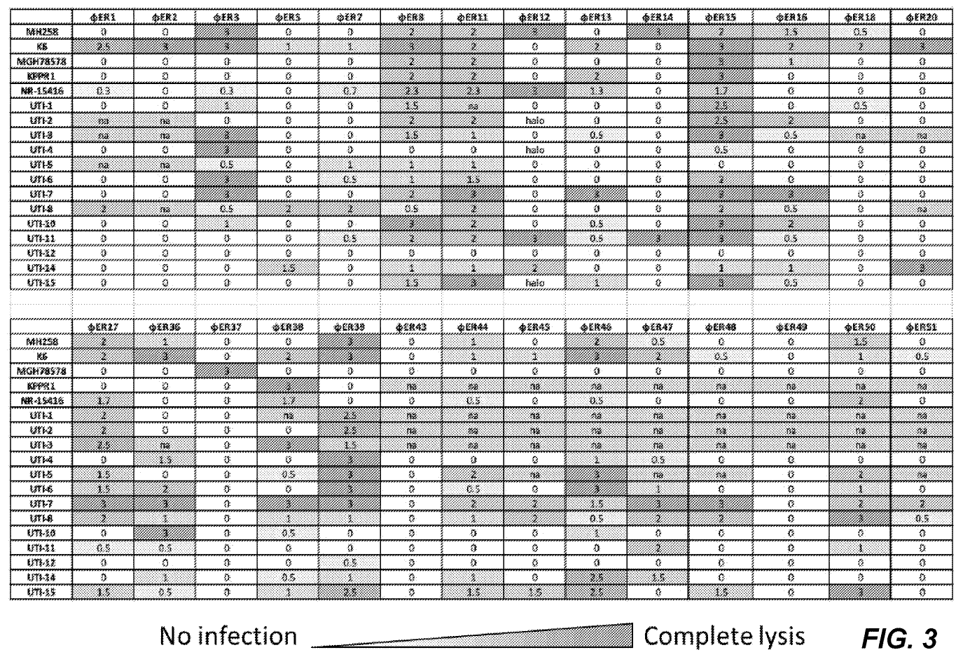

A collection of bacteriophages that can infect clinical Klebsiella isolates were isolated from sewage. FIG. 1 shows electron microscopy of representative bacteriophage that infect Klebsiella, according to some aspects of the disclosure. FIG. 2 shows the phylogenetic relationship of representative bacteriophage that infect Klebsiella, according to some aspects of the disclosure. FIG. 3 shows the ability of various bacteriophage to infect clinical Klebsiella strains, according to some aspects of the disclosure.

Through selection on a variety of different Klebsiella hosts, including carbapenem-resistant strains of K. pneumoniae (MH258, BAA-2472, MGH78578, JHCK1, and BAA-2342) and extended-spectrum beta-lactamase (ESBL) positive strains of K. quasipneumoniae (K6), phages representing the major families of tailed phages were isolated. Two of these host bacteria (MH258 and BAA-2342) belong to sequence type 258 (ST258) strains of K. pneumoniae that account for the majority of carbapenem-resistant infections in the United States (Wyres, K. L., Lam, M. M. C., and Holt, K. E. (2020)). With future considerations for cocktails (where resistance could result in acapsular variants), a subset of the phages was isolated on non-capsulated variants of MH258, JHCK1, and K6. The isolated phages reported herein are all obligately lytic and lack any deleterious genes that may contraindicate them for therapy (e.g., antibiotic resistance, virulence, or toxin genes). Genomic content and synteny was largely shared between phages in the same genus, with the highest degree of diversity in annotated receptor binding proteins, hypothetical proteins, or homing endonucleases.

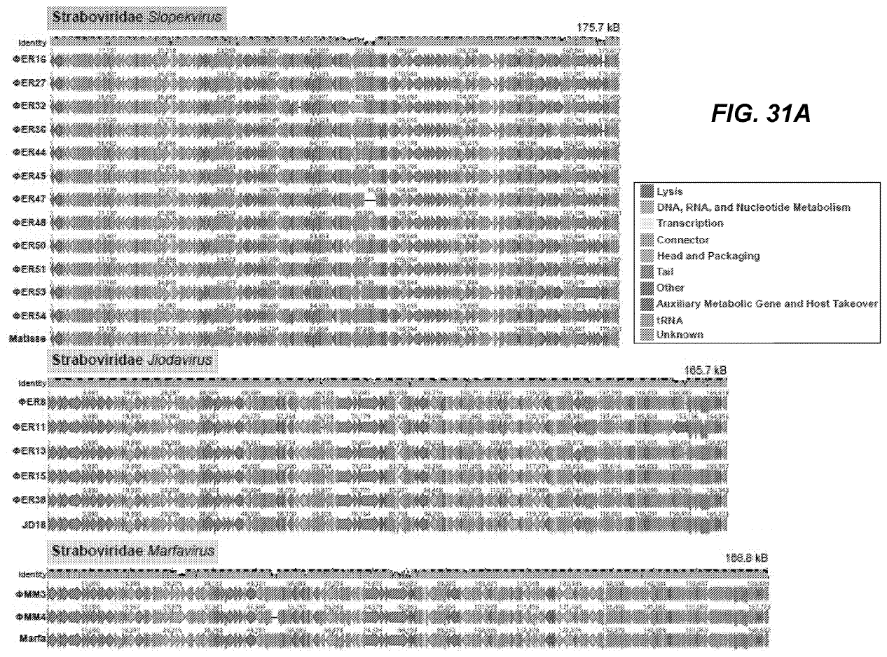

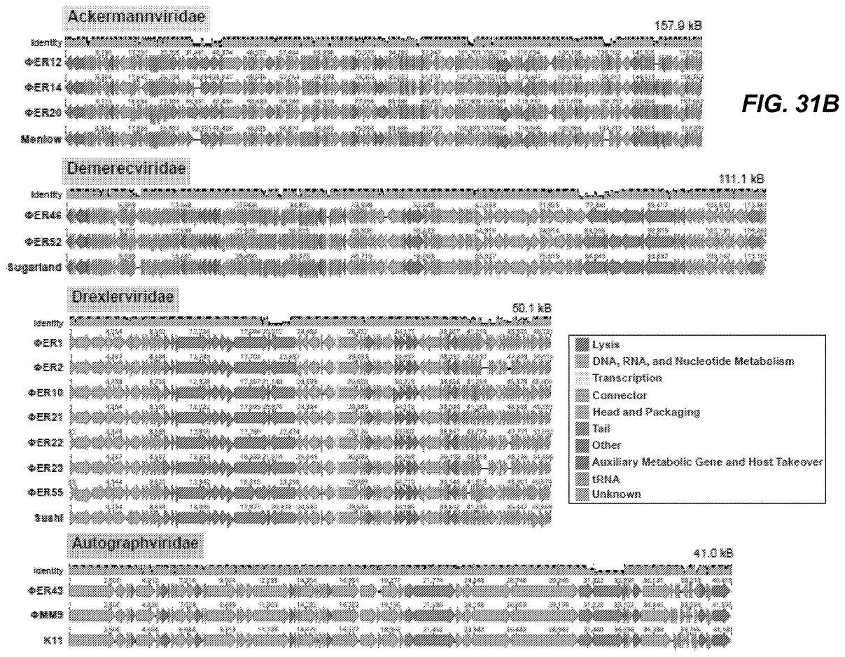

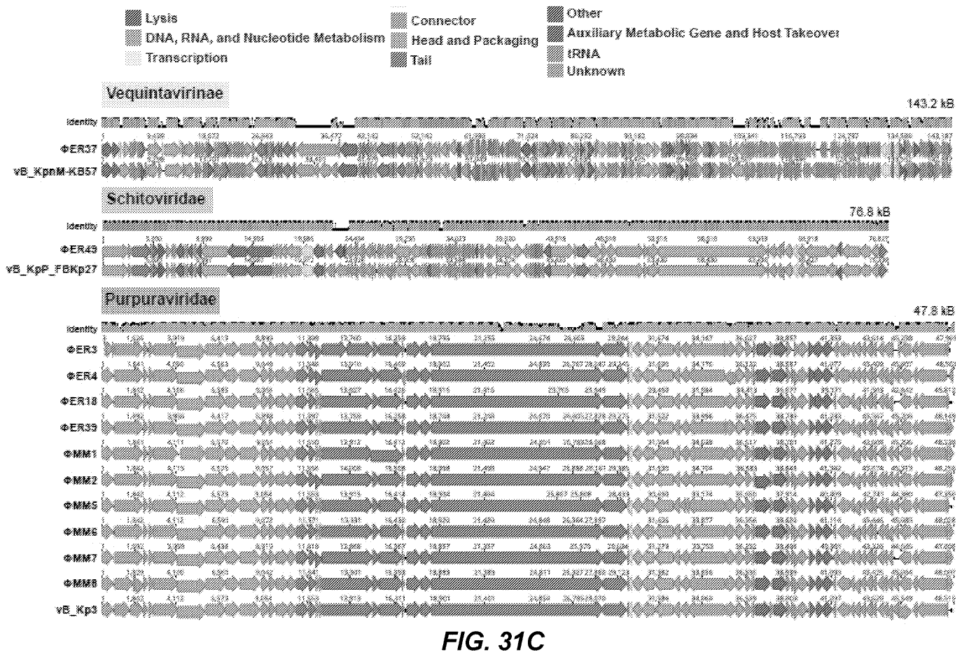

Four distinct families of T4-like phages including 19 T4-like Straboviridae were identified (FIG. 31A). These included 12 Slopekviruses and one Pseudotevenvirus (in red), 2 Marfaviruses (in teal), and 5 Jiaodaviruses (in blue), which belong to the Tevenvirinae subfamily and are more similar to bacteriophage T4. The phages produced tiny pinprick plaques on a capsulated host and larger plaques on a non-capsulated host. The Tevenvirinae contain multiple tRNA genes, while the Slopekviruses only have one tRNA gene. Bacteriophage T4 contains 8 tRNA genes, while the reported Tevenvirinae contain more, up to 15. The presence of the tRNAs may facilitate translation of phage proteins after destruction of the host machinery and allow for codon preference of the phage genes compared to the host. These bacteriophages recognize lipopolysaccharide (LPS) of host bacteria as the receptor for entry, and bacterial resistance to the bacteriophage can be mediated by mutations to genes involved in LPS synthesis.

Also identified were Ackermannviridae (FIG. 31B), once classified as Myoviridae, which are large phages (160 kb) with a large head and medium tail. The tail fibers are present as rosettes at the tip of the tail. The phages make medium sized plaques surrounded by a halo. These bacteriophages use a capsule for bacterial strain recognition and entry, and bacterial resistance to the bacteriophage is by capsule loss, with non-capsulated bacterial being less virulent. The tail fibers of the bacteriophage recognize the host bacteria capsule, and over 100 different Klebsiella capsule types have been identified. These phages do not plaque on or infect strains lacking a capsule. The Ackermannviridae also contain 5-6 tRNA genes.

One other phage that formerly would have been classified as Myoviridae was also identified: a vRV-like Vequintavirinae subfamily, Mydovirus genus (143 kb) (FIG. 31C).

Also identified were two T7-like Autographviridae (FIG. 31B), which are small (40 kb) Podoviruses, with a small head and a very short tail. The plaques are medium sized with a very large halo that increases over time as a depolymerase diffuses outwards. The T7-like phages have their own DNA and RNA polymerases. Two T7-like phages were isolated, and another Podovirus, ΦER49, related to the bacteriophage N4, which at 77 kb, also formed a small head and no tail, but was taxonomically distinct (Schitoviridae family) was also isolated (FIG. 31C).

Also identified were two T5-like Demerecviridae, which are formerly Siphoviridae with a medium head (110 kb) and a long tail (FIG. 31B). T5-like phages contain proteins A1 and A2, which facilitate transfer and degradation of the host DNA after the first portion of the DNA is inject in a two-step process. When assembling the genome of Demerecviridae, an increase in read depth for the initial 9.2 kb (8% of the genome) was observed. The two Demerecviridae also contain a large (>10 kb) tail fiber gene, similar to phage vB_Kpn_B01. Like bacteriophages T4 and T5, phages ΦER46 and ΦER52 also contain multiple tRNA genes, as many as 21 (including two tRNA-Met genes).

Also isolated were seven T1-like Drexlerviridae, also formerly Siphoviridae, which have a small head (50 kb) and a very long contractile tail (FIG. 31B). Ten of these phages were isolated in the Webervirus genus. Phages ΦER10 and ΦER55 were isolated on non-capsulated strains. In general, the T1-like phages tended to plaque more broadly on non-capsulated Klebsiella strains.

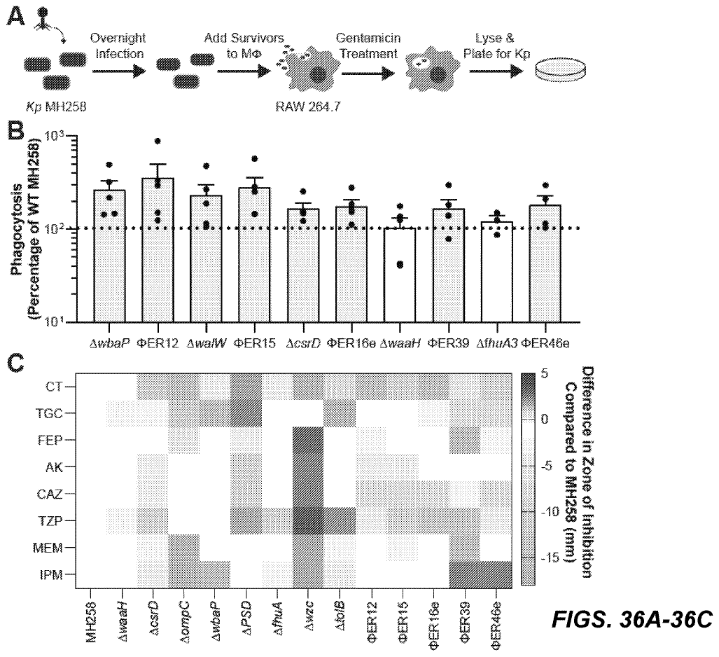

The remaining 10 phages cluster together in an unclassified Siphoviridae family, a proposed name for which is Purpuraviridae (FIG. 31C). These phages are small (48 kb) with a long tail, medium-sized plaques, and moderate virulence in liquid assays (FIG. 36B) and have yet to be thoroughly studied or employed in therapeutic settings. These bacteriophages recognize protein receptors of host bacteria as the receptor for entry, and bacterial resistance to the bacteriophage is generally by mutation to the receptor.

### Example 2—Characterization of Bacteriophage Host Range

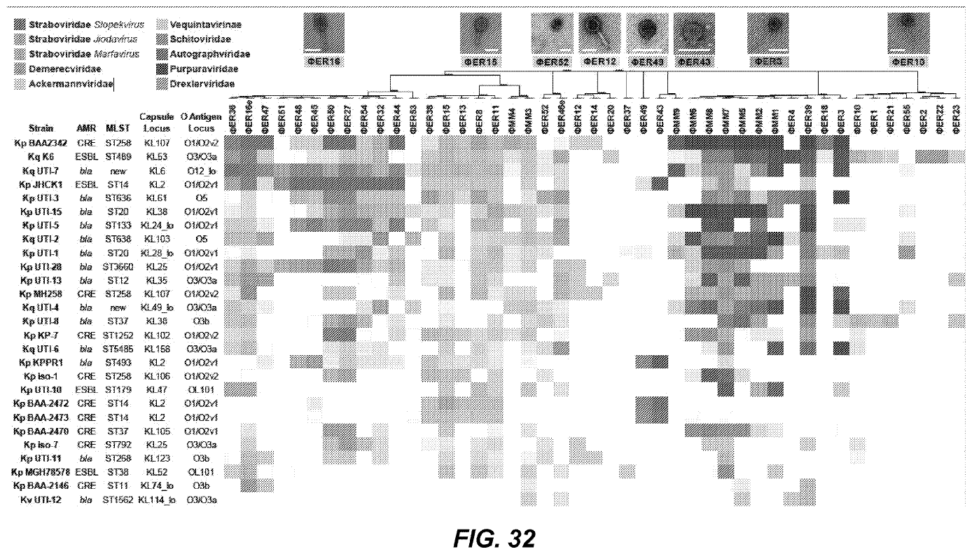

The host-range of the isolated Klebsiella phages was assessed on 27 different Klebsiella strains that span 23 sequence types (STs) (including clinically-relevant ST14, ST37, and ST25829), 21 capsule types, and 8 O-antigen types (FIG. 32). Additionally, the strain set includes carbapenem-resistant and ESBL positive strains as well as 14 new clinical isolates from recurrent urinary tract infections. Although strains exhibited varying levels of susceptibility to the phages, each strain was susceptible to multiple bacteriophages.

Bacteria can avoid phage infection through a variety of means, including preventing phage adsorption by lacking the proper receptor, degrading the injected phage DNA, and various abortive infection strategies (Huiting, E., and Bondy-Denomy, J. (2023); Labrie, S. J., Samson, J. E., and Moineau, S. (2010)). A phage with a broad host range will likely infect using a conserved receptor on the bacterial surface and contain anti-defense systems, such as DNA modifications, lack of restriction sites, or anti-CRISPR proteins (Li, Y., and Bondy-Denomy, J. (2021)). Phages with narrow host range tend to be the T1-like Drexlerviridae (green), the Ackermannviridae (yellow), the Podoviruses (brown and grey), and also the one vR5-like virus (lime green). Also isolated were numerous T1-like viruses on non-capsulated Klebsiella strains (FIG. 32), which are likely prevented from entry by the capsule. On the other hand, the Ackermannviridae require specific capsule types for entry and are thus excluded by most capsule types.

Phages with broad host ranges include the T4-like Straboviridae phages (shown in red, blue, and teal), the class of Siphoviridae shown in Purple (proposed Purpuraviridae), and the T5-like viruses shown in orange. T4 is known to modify its DNA by incorporating hydroxymethyl-cytosine (HMC) instead of cytosine and glycosylating that residue to prevent recognition by the host defense systems. Indeed, both the blue Tevenvirinae and red Slopekviruses contain genes for a putative dCMP hydroxymethylase and the blue Tevenvirinae also contain a beta-glucosyl-HMC-alpha-glucosyl-transferase. Additionally, the red Slopekviruses also lack KpnI restriction sites, presumably to prevent degradation by that Klebsiella pneumoniae restriction enzyme. The Purpuraviridae are currently understudied and do not possess any hallmark proteins that suggest resistance to anti-phage systems. However, their genomes are devoid of KpnI restriction sites, which would allow for evasion of restriction-modification systems.

### Example 3—Characterization of Bacteriophage of Interest

The collection of bacteriophages that can infect clinical Klebsiella isolates were assessed for their ability to infect clinical Klebsiella strains (FIG. 3; darker shading is indicative of a greater ability of the bacteriophage to infect the clinical Klebsiella strains; scale of 0=no infection to 3=complete lysis; “na” indicates no data).

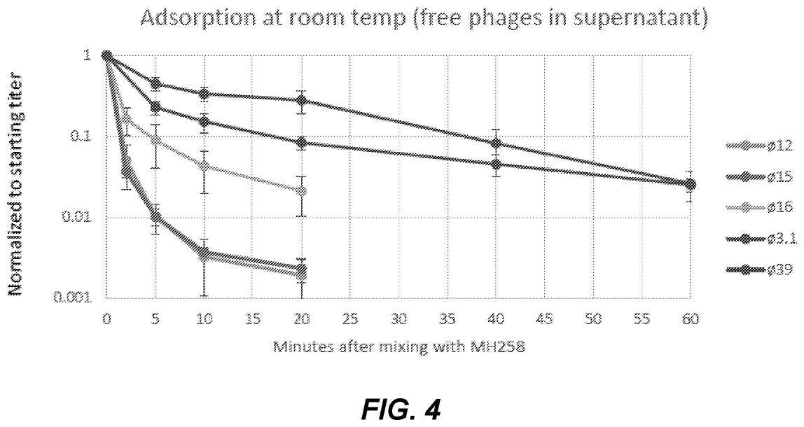

FIG. 4 shows phage adsorption kinetics for various bacteriophage of interest. ΦER12 and ΦER15 displayed rapid binding to bacterial cell surface. ΦER16, ΦER39, and ΦER3 also showed binding to the bacterial cell surface.

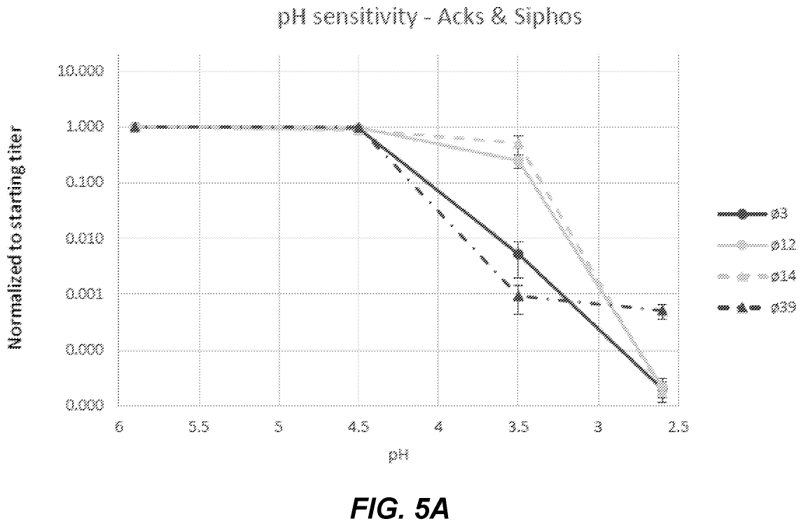

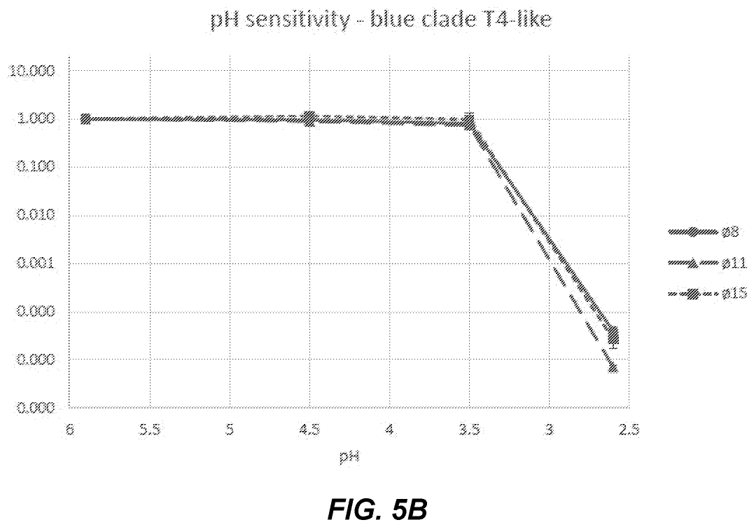

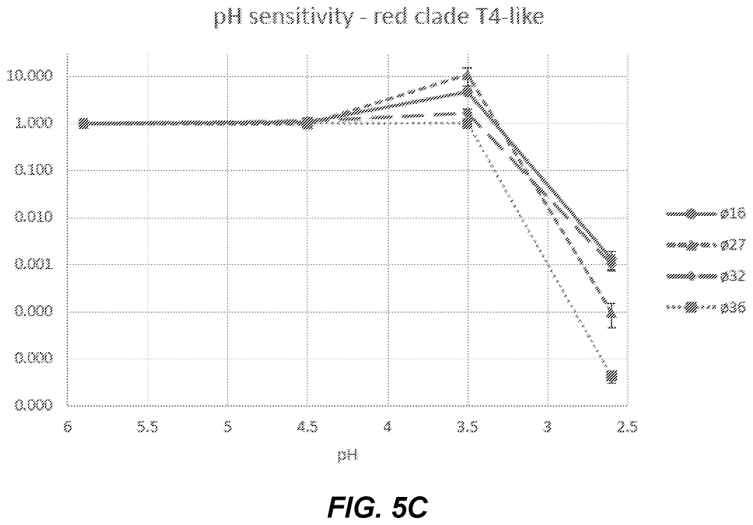

FIGS. 5A-5C show pH tolerance of representative bacteriophage disclosed herein. All bacteriophages were inactivated by pH 2.6. T4-like bacteriophage (e.g., φ8, ΦER11, ΦER15, ΦER16, ΦER27, φ32, and ΦER36) were not sensitive to pH 3.5 (FIGS. 5B, 5C), while Ackermannviridae family bacteriophage (e.g., ΦER12, φ14) were mildy sensitive to pH 3.5 (FIG. 5A), and Siphoviridae family bacteriophage (e.g., ΦER3, ΦER39) were very susceptible to pH 3.5 (FIG. 5A).

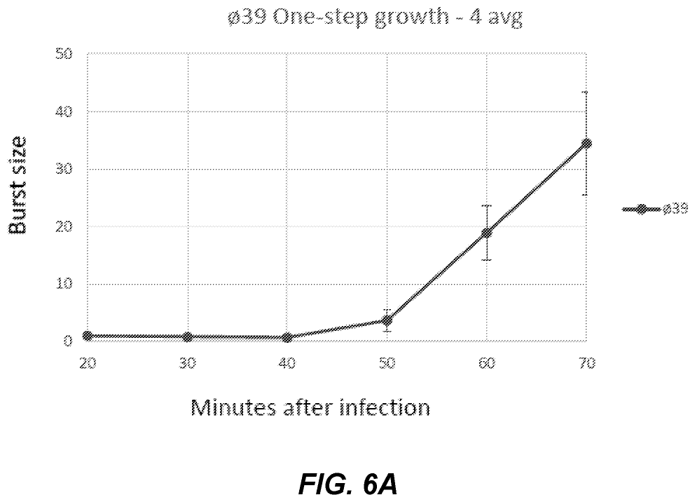

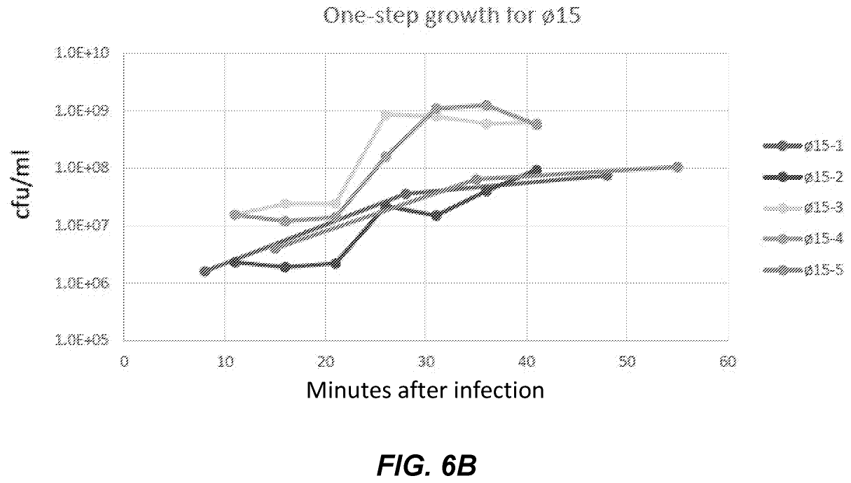

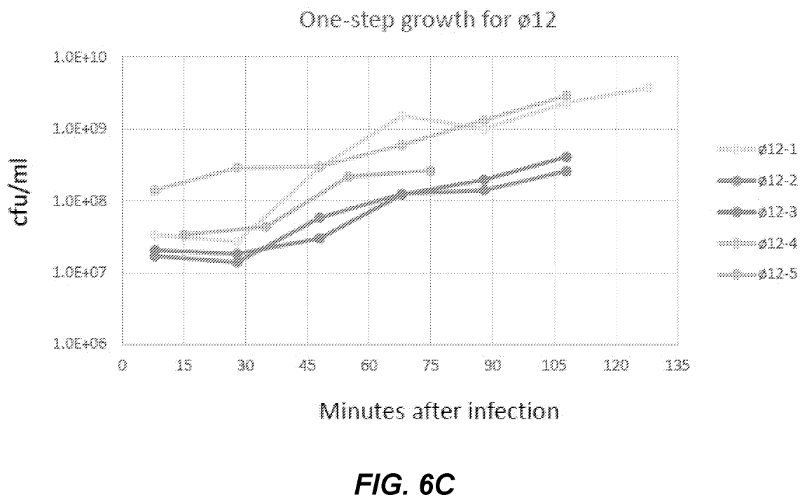

FIGS. 6A-6C show one-step growth of representative bacteriophage disclosed herein. Siphoviridae family bacteriophage (e.g., ΦER39) had a 40 min latent period and a 30-40× burst (FIG. 6A). T4-like bacteriophage (e.g., ΦER15) had a 20 min latent period and a 10-80× burst (FIG. 6B). Ackermannviridae family bacteriophage (e.g., ΦER12) had a 30 min latent period and a 10-80× burst (FIG. 6C).

### Example 4—Identification of Bacterial Host Factors Required for Phage Infection

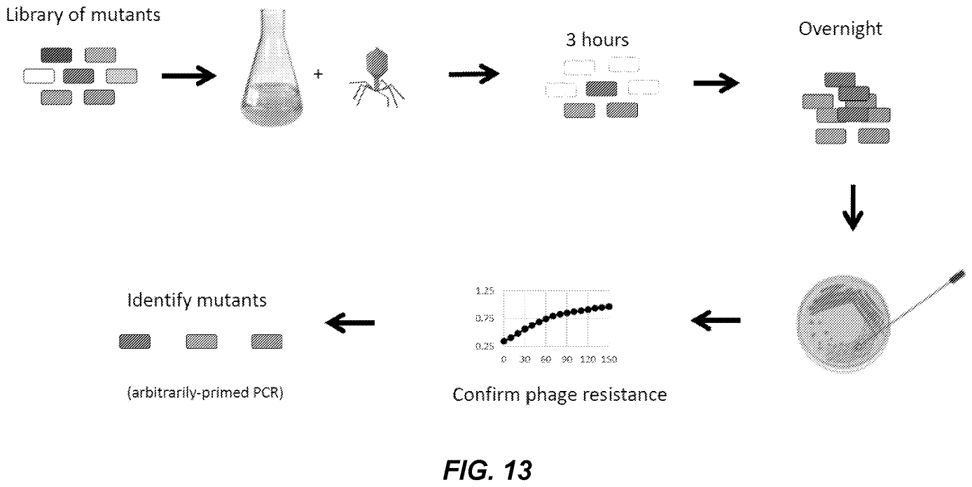

Bacterial host factors required for bacteriophage infection were identified using a method illustrated by FIG. 13. To determine how the different phages enter the cells, we sought to identify the cell receptors and other host factors by using a transposon library of Klebsiella pneumoniae MH258, a carbapenem-resistant ST258 strain (Jung, H.-J., Littmann, E. R., Seok, R., Leiner, I. M., Taur, Y., Peled, J., van den Brink, M., Ling, L., Chen, L., Kreiswirth, B. N., et al. (2019)). We first used insertion sequencing (INSeq) (Goodman, A. L., McNulty, N. P., Zhao, Y., Leip, D., Mitra, R. D., Lozupone, C. a, Knight, R., and Gordon, J. I. (2009)) on ΦER15 and ΦER39 infected cultures to quantify the differential abundance of transposon mutants in uninfected and infected populations. Additionally, we identified transposon mutants that survived infection of ΦER12, ΦER15, ΦER16, ΦER39, or ΦER46 by the location of the transposon insertions with arbitrarily-primed nested PCR.

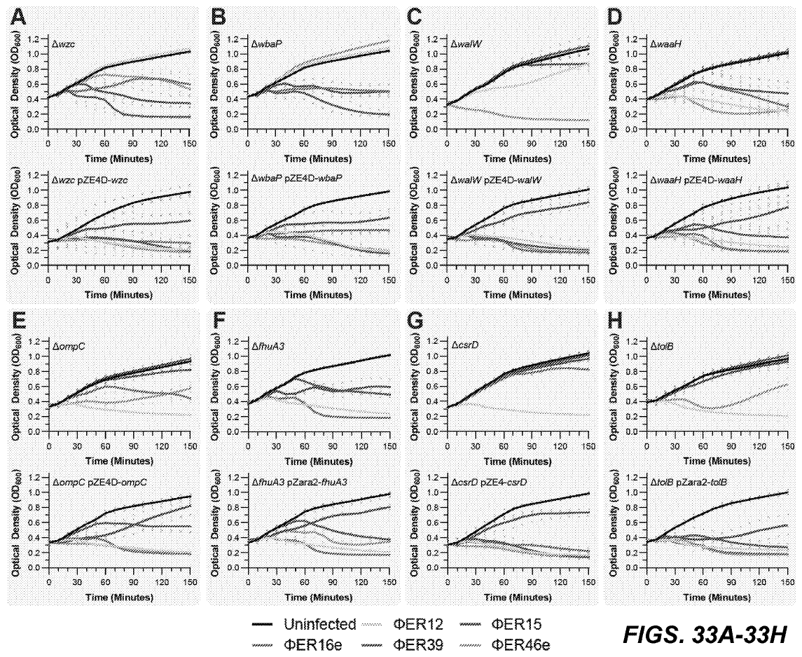

We first independently infected cultures with ΦER15 and ΦER39 for 3 hours. After extracting DNA from uninfected and infected cultures and processing for INSeq (Goodman, A. L., McNulty, N. P., Zhao, Y., Leip, D., Mitra, R. D., Lozupone, C. a, Knight, R., and Gordon, J. I. (2009)), we compared the pool of mutants before and after infection for an enrichment of interrupted genes in the presence of phage (FIG. 33I). The top ΦER15-enriched genes included walW (wabN; a polysaccharide deacetylase in the LPS pathway), galU (catalyzes the synthesis of UDP-D-glucose, a precursor in capsule and LPS biosynthesis), rfaZ (LPS biosynthesis) and tolB, a member of the envelope-spanning Tol-Pal system for cell division and outer membrane integrity. The top hits for ΦER39 included tolB, rfaZ, pstB (part of a phosphate-sensing system in the membrane), and csrD (sugar metabolism regulation). The INSeq method provides useful information at the population level and highlights processes in cell metabolism that may not just be as simple as a surface receptor Mutalik, V. K., Adler, B. A., Rishi, H. S., Piya, D., Zhong, C., Koskella, B., Kutter, E. M., Calendar, R., Novichkov, P. S., Price, M. N., et al. (2020)).

To complement the INSeq process, the transposon library of MH258 (a clinical isolate of multi-drug resistant ST258 Klebsiella pneumoniae) was infected with ΦER12, ΦER15, ΦER16, φ16e, ΦER39, ΦER46, or φ46e for 3 hours to lyse bacterial host cells, followed by culture of the surviving bacterial host cells overnight, plating of the cultured surviving bacterial host cells, confirmation of bacteriophage resistance of the surviving bacterial host cells, and identification of surviving bacterial host cell strains using arbitrarily-primed PCR for transposon location. ΦER16e and ΦER46e are evolved variants of ΦER16 and ΦER46, respectively, discussed in greater detail with respect to FIG. 36A.

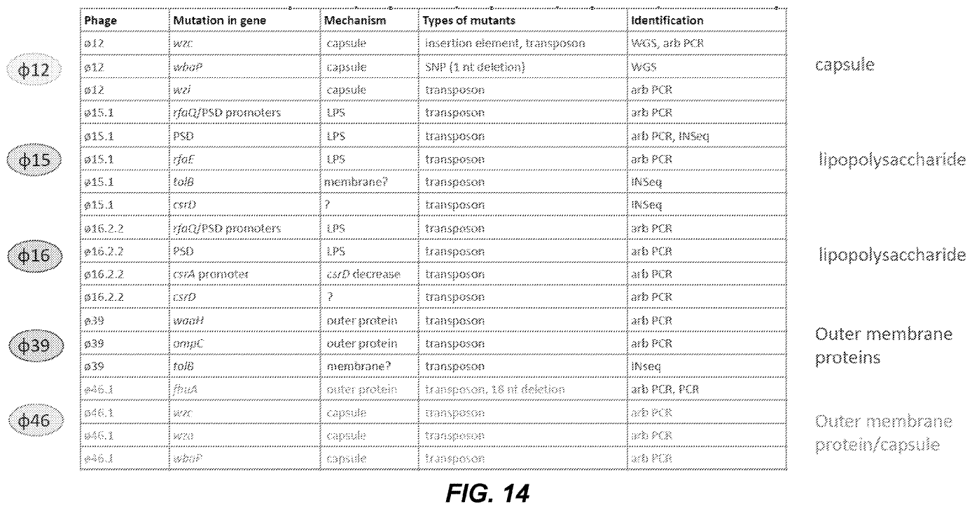

After confirming resistance to the infecting phage, the location of the transposon insertions were identified (Table 2). As shown in FIG. 14 and FIG. 34, several Klebsiella factors were determined to be required for infection of Klebsiella with representative bacteriophage (e.g., ΦER12, ΦER15, ΦER16, φ16e, ΦER39, ΦER46, or φ46e).

TABLE 2

| Phage | Gene hit | Gene function | Capsulated | Times |
|---|---|---|---|---|
| ER12 | wzc | capsule transport | no | 2 |
| ER12 | wzi | capsule transport | no | 1 |
| ER15 | glycosyl | LPS biosynthesis | no | 1 |
| | transferase | | | |
| ER15 | rfaQ/walW | LPS biosynthesis | no | 2 |
| | promoter | | | |
| ER15 | walW | LPS biosynthesis | no | 1 |
| ER15 | rfaE/hldE | LPS biosynthesis, | partial | 1 |
| | | inner core | | |
| ER15 | rfaZ | LPS biosynthesis | no | 1 |
| ER16 | csrA promoter | Sugar metabolism | yes | 2 |
| ER16 | walW | LPS biosynthesis | no | 2 |
| ER16 | csrD | Sugar metabolism | yes | 2 |
| ER16 | rfaQ/walW | LPS biosynthesis | no | 2 |
| | promoter | | | |
| ER39 | waaH | LPS decoration | yes | 4 |
| ER39 | ompC | Outer membrane | yes | 2 |
| | | protein | | |
| ER46 | fhuA | Iron Transport | yes | 3 |
| | | protein | | |
| ER46 | wbaP | capsule biosynthesis | no | 1 |
| ER46 | wza | capsule transport | no | 1 |
| ER46 | wzc | capsule transport | no | 1 |
| Independent infections: | | | | |
| ER12 | 2 | | | |
| ER15 | 3 | | | |
| ER16 | 4 | | | |
| ER39 | 5 | | | |
| ER46 | 3 | | | |

For ΦER12, survivors of infection were always non-capsulated, and all non-capsulated MH258 strains were resistant to ΦER12. Three selected non-capsulated survivors from ΦER12 infection mapped to the capsule locus (wzi and wzc). For the T4-like Straboviridae ΦER15 and ΦER16, transposon hits to the LPS locus were expected, which is a known receptor for bacteriophage T4. Six hits were sequenced for ΦER15 and 8 hits for ΦER16, and the majority were indeed disruptions to LPS biosynthesis (glycosyltransferases, rfaZ KdoIII attachment to inner core, rfaE/hldE inner core building block, a divergent promoter to rfaQ addition of side branch to inner core and the walW polysaccharide deacetylase, and the walW gene itself; Table 2). In agreement with the INSeq data, survivors of ΦER16 were disrupted in csrD and in the promoter region of csrA (since csrA is known to be essential in E. coli grown in LB (Timmermans, J., and Van Melderen, L. (2009)). As a Siphovirus, suppressors of ΦER39 were expected to be a disruption to an outer membrane protein receptor. The top survivor hits were to ompC, an outer membrane protein, and to waaH, an LPS decoration protein. WaaH is responsible for the addition of a heptose III residue to the inner core of LPS, but it may also be an outer membrane-associated protein (Klein, G., Müller-Loennies, S., Lindner, B., Kobylak, N., Brade, H., and Raina, S. (2013)). OmpC is a canonical outer membrane receptor and is likely to be host receptor where the phage genome is injected. For the T5-like ΦER46e, hits were expected to be in fhuA (also known historically as tonA in E. coli for its resistance to bacteriophage T1, as well as being the receptor for T5). Indeed, most of the survivors of ΦER46e infection were transposon insertions in the fhuA3 gene of MH258. Since some of the surviving colonies were non-capsulated, three candidates were sequenced, and hits to the capsule biosynthesis pathway (wza, wzc, and wbaP) were found. Thus, ΦER46 requires both the host receptor protein FhuA3 and the capsule.

In summary, ΦER12, representing the Ackermannviridae, requires the capsule, andmutations in wzc, wbaP, and wzi capsule genes impaired infection by ΦER12 (FIG. 34A). ΦER15 and ΦER16, representing the T4-like Straboviridae, use the LPS, as mutations in rfaQ)/PSD promoters, wbaN (PSD), rfaE, and tolB LPS and membrane genes as well as csrD regulator genes impaired infection by ΦER15, and mutations in rfaQ/PSD promoters and wbaN (PSD) LPS genes as well as csrA and csrD regulator genes impaired infection by ΦER16 (FIG. 34A). ΦER39 (Siphoviridae) and ΦER46 (T5-like Demerecviridae) use outer membrane proteins such as OmpC and FhuA, respectively (FIG. 34A), as mutations in waaH, ompC, and tolB outer membrane protein genes impaired infection by ΦER39, and mutations in fhuA, wzc, wza, and wbaP outer membrane protein and capsule genes impaired infection by ΦER46. It was also found that the csrD carbohydrate regulator and the Tol-Pal membrane stability system influence the ability of the Straboviridae, Siphoviridae, and Demerecviridae to infect (FIG. 34A)

Clean deletions of the genes determined to be required for infection of Klebsiella with representative bacteriophage was performed in Klebsiella MH258, and the mutant Klebsiella MH258 strains were infected with various bacteriophage. To confirm the top hits from the INSeq and phage survivor screen, we selected the following eight genes to delete and complement: wbaP, wzc, walW, waaH, ompC, fhuA3, csrD, and tolB. Two of the gene deletions were constructed by homologous recombination with a plasmid integration intermediate (van Aartsen, J. J., and Rajakumar, K. (2011)), while the other six were constructed using the Lambda-Red method of deletion-replacement (Huang, T.-W., Lam, I., Chang, H.-Y., Tsai, S.-F., Palsson, B. O., and Charusanti, P. (2014); Sharan, S. K., Thomason, L. C., Kuznetsov, S. G., and Court, D. L. (2009)). All deleted genes contained the start codon and final six amino acids (based on those constructed in the Keio collection of E. coli mutants) to avoid potential downstream effects (Baba, T., Ara, T., Hasegawa, M., Takai, Y., Okumura, Y., Baba, M., Datsenko, K. A., Tomita, M., Wanner, B. L., and Mori, H. (2006)).

It was then determined whether the constructed mutant was resistant to the panel of five phages (ΦER12, ΦER15, ΦER16e, ΦER39, and ΦER46e) both by spot test with a high titer stock on a lawn of bacteria (FIG. 34B) and by measuring the area under the curve normalized to uninfected culture of kill curve assays in liquid broth (FIG. 34C and FIGS. 33A-H). For each mutant, each gene was complemented by providing a copy in trans with a constitutively expressed promoter. All the phenotypes described below in the deletion mutants were able to be mostly, if not fully, rescued by the presence of the plasmid.

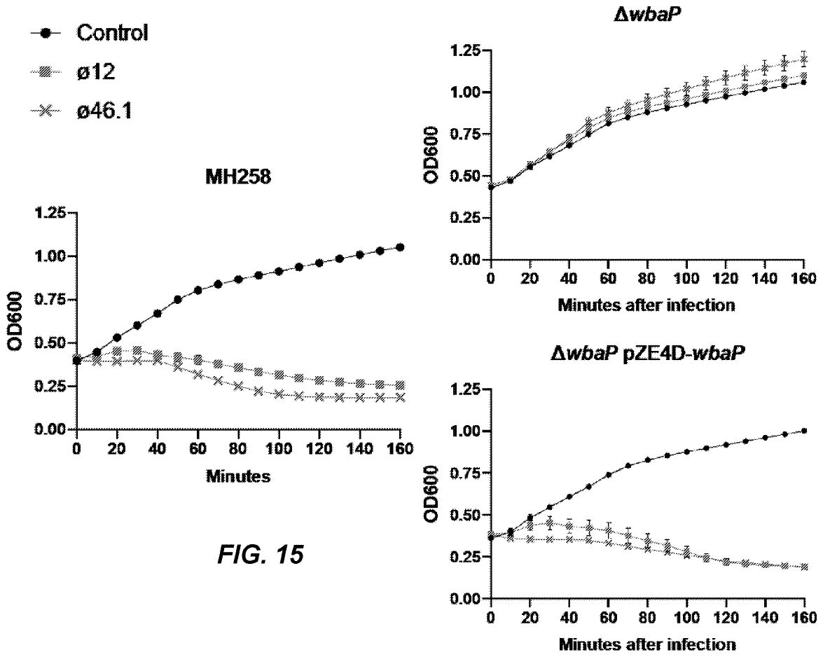

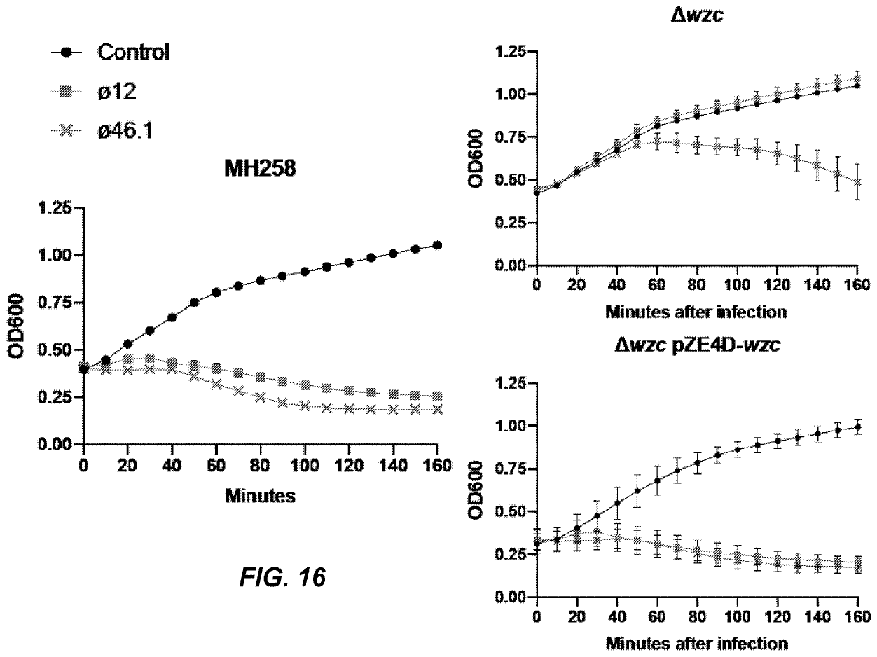

Capsule Pathway Deletions. wbaP was deleted in Klebsiella MH258, and the wbaP deletion Klebsiella MH258 mutant (ΔwbaP) was treated with ΦER12 or ΦER46 (FIG. 15; FIG. 33B; FIG. 34). Deletion of wbaP in Klebsiella MH258 reduced infection and lysis by ΦER12 and ΦER46, whereas complementing the wbaP gene deletion (ΔwbaP pZE4D-wbaP) rescued the ability of ΦER12 and ΦER46 to infect and lyse the wbaP deletion Klebsiella MH258 mutant. wzc was deleted in Klebsiella MH258, and the wzc deletion Klebsiella MH258 mutant (Δwzc) was treated with ΦER12 or ΦER46 (FIG. 16; FIG. 33A; FIG. 34). Deletion of wzc in Klebsiella MH258 reduced infection and lysis by ΦER12 and to a lesser extent ΦER46, whereas complementing the wzc gene deletion (Δwzc pZE4D-wzc) rescued the ability of ΦER12 and ΦER46 to infect and lyse the wzc deletion Klebsiella MH258 mutant.

Both wbaP and wzc are part of the capsule biosynthesis operon, with WbaP (or WcaJ) performing the first step in assembly of the sugar oligomers and Wzc being a tyrosine protein kinase which helps regulate the transport of the capsule to the surface (Patro, L. P. P., and Rathinavelan, T. (2019)). ΔwbaP is completely unable to synthesize the capsule, while the Δwzc mutant still makes some capsule, but not to the wild-type levels, and most of the synthesized capsule is not on the cell surface. Both capsule mutants were fully resistant to ΦER12, showing the strong requirement of the Ackermannviridae for the Klebsiella capsule. Both ΔwbaP and Δwzc capsule mutants also showed resistance to ΦER46e in liquid culture. However, this T5-like phage can fully infect on a lawn of Δwzc and partially infect a lawn of ΔwbaP, indicating that ΦER46e likely utilizes the capsule as a co-receptor to stabilize phage-bacterial interaction. In liquid culture, the Δwzc mutant was partially resistant to ΦER16e, perhaps indicating that the capsule facilitates entry for T4-like Slopekviruses.

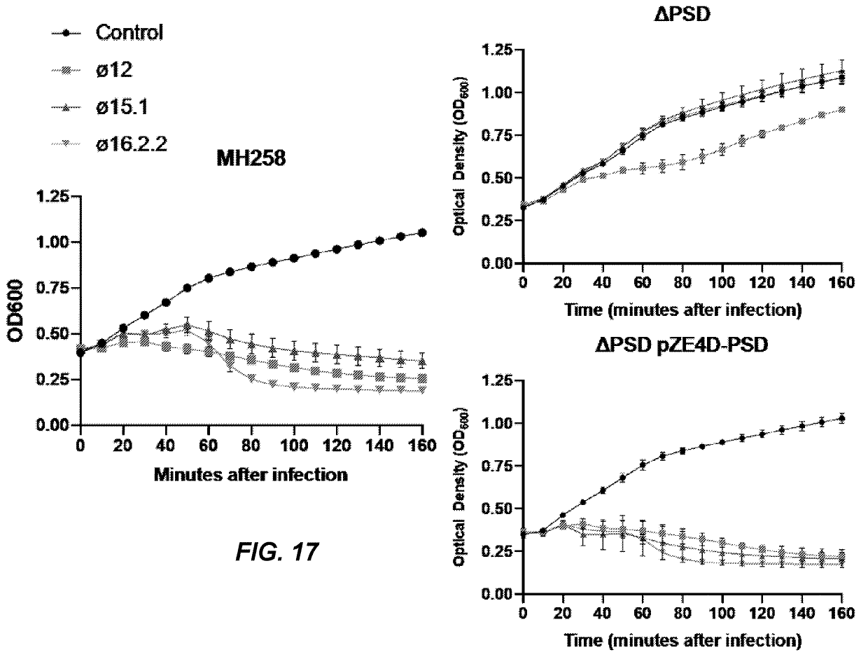

LPS Pathway Deletions. wbaN (also referred to herein as walW) was deleted in Klebsiella MH258, and the wbaN deletion Klebsiella MH258 mutant (ΔPSD) was treated with ΦER12, ΦER15, or ΦER16 (FIG. 17). Deletion of wbaN in Klebsiella MH258 reduced infection and lysis by ΦER15 or ΦER16 and to a lesser extent ΦER12, whereas complementing the wbaN gene deletion (ΔPSD pZE4D-PSD) rescued the ability of ΦER12, ER15, and ΦER16 to infect and lyse the wbaN deletion Klebsiella MH258 mutant.

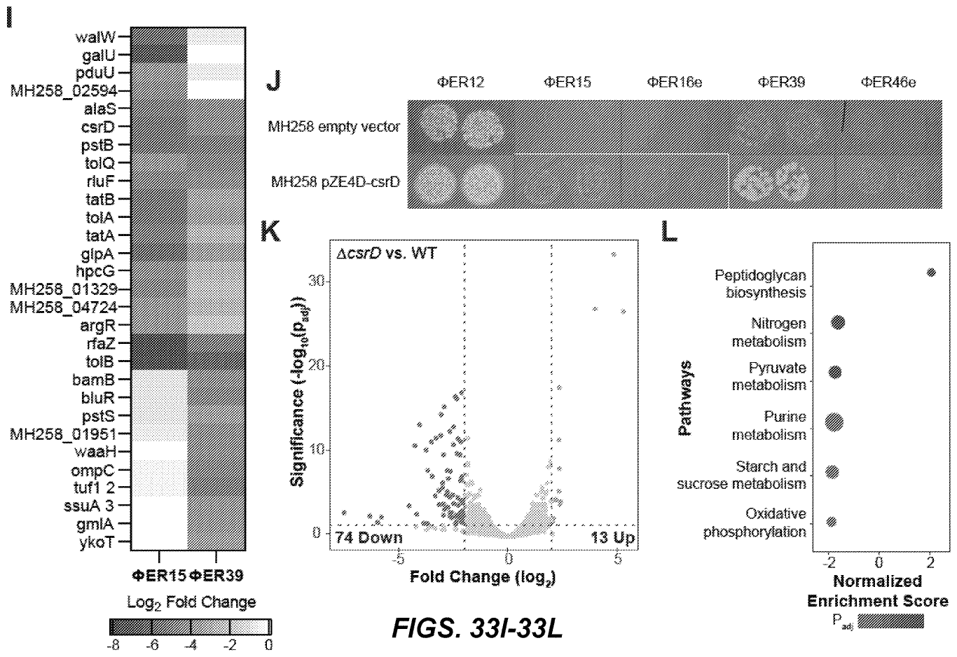

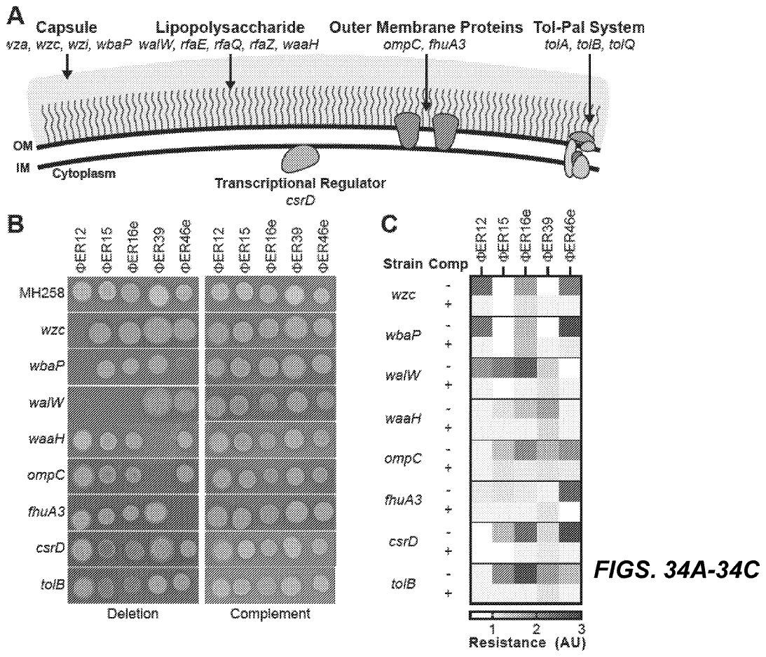

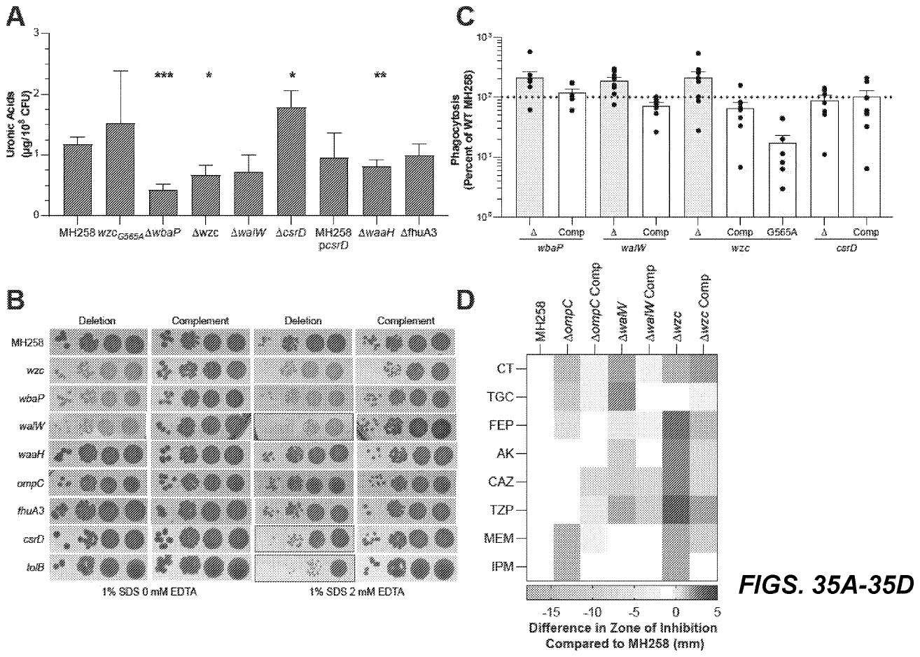

The walW gene is part of the LPS pathway, an uncharacterized polysaccharide deacetylase in the LPS biosynthesis operon. This gene was chosen because it appeared both in the INSeq and survivor screens as required for both groups of T4-like phages represented by ΦER15 and ΦER16e. The ΔwalW mutant exhibited a non-capsulated appearance on plates and was fully resistant to phages ΦER15 and ΦER16. Capsule production was partially eliminated in the ΔwalW (FIG. 35A), which is corroborated by the fact that ΦER12 can partially infect in this background (FIG. 33C).

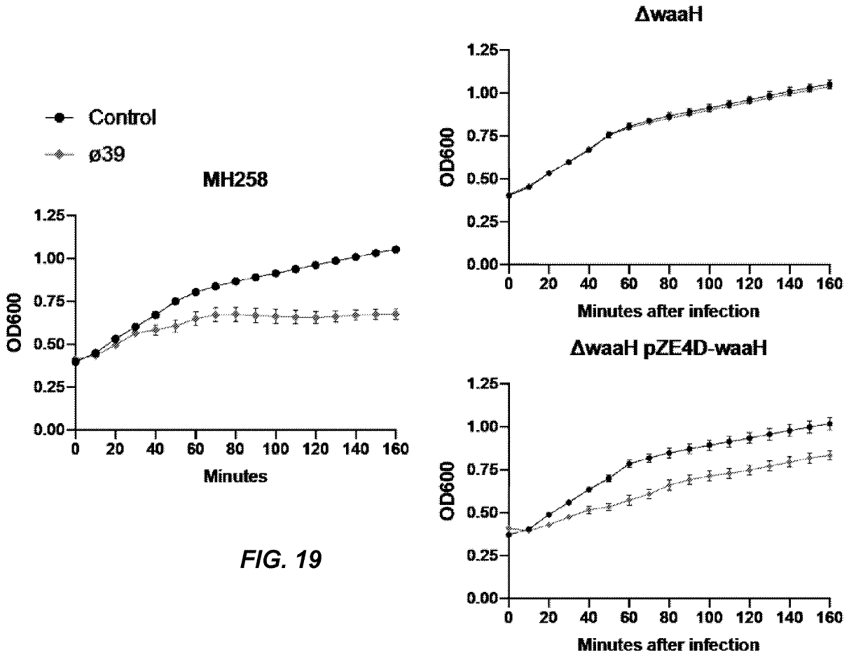

waaH was deleted in Klebsiella MH258, and the waaH deletion Klebsiella MH258 mutant (ΔwaaH) was treated with ΦER39 (FIG. 19; FIG. 33D; FIG. 34). The waaH gene is responsible for adding a HepIII residue to the HepII of the inner core of the LPS (Klein, G., Müller-Loennies, S., Lindner, B., Kobylak, N., Brade, H., and Raina, S. (2013)). Deletion of waaH in Klebsiella MH258 reduced infection and lysis by ΦER39, whereas complementing the waaH gene deletion (ΔwaaH pZE4D-waaH) rescued the ability of ΦER39 to infect and lyse the waaH deletion Klebsiella MH258 mutant. A transposon disrupting this gene was found multiple times in the survivors of ΦER39 infection. It is interesting to note that no other major LPS biosynthesis genes were isolated as survivors of ΦER39 infection and that the waaH was found in 5 independent infections. However, the rfaZ gene, also affecting the inner LPS core, and ykoT, a glycosyltransferase predicted to be involved in LPS biosynthesis, were increased in the INSeq data after ΦER39 infection (FIG. 33I). The ΔwaaH mostly prevented infection by ΦER39, with faint plaquing that can be seen in FIG. 34B, and moderate resistance in liquid culture. In contrast, deletion of the ompC gene resulted in full resistance to ΦER39, implying that OmpC is the outer membrane porin receptor for ΦER39, while the modification to LPS is also required for optimum infection, but not strictly necessary.

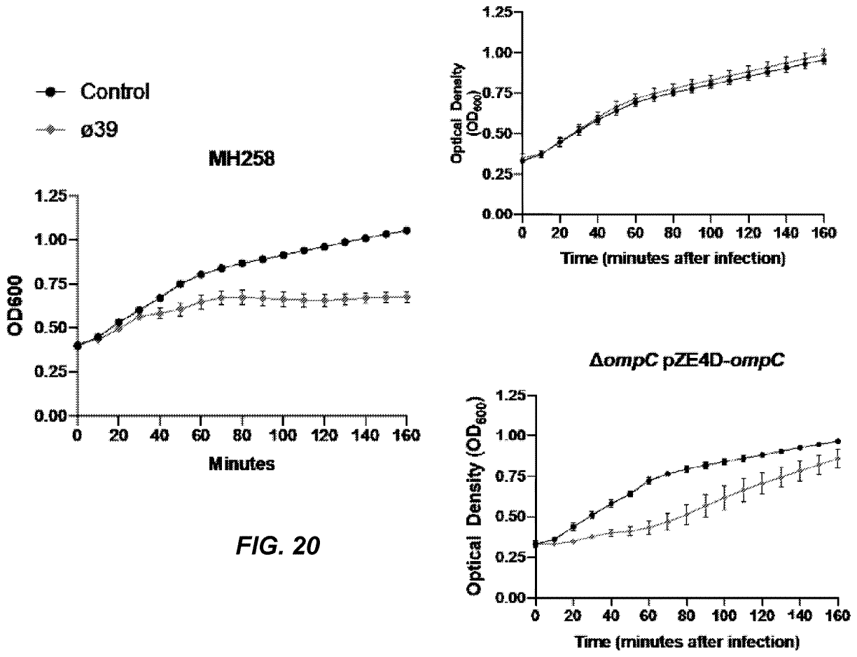

Outer Membrane Protein Deletions. ompC was deleted in Klebsiella MH258, and the ompC deletion Klebsiella MH258 mutant (ΔompC) was treated with ΦER39 (FIG. 20; FIG. 33E; FIG. 34). OmpC (OmpK36), along with OmpF (OmpK35), is one of two major outer membrane porins responsible for the diffusion of small molecules into the cell (Tsai, Y.-K., Fung, C.-P., Lin, J.-C., Chen, J.-H., Chang, F.-Y., Chen, T.-L., and Siu, L. K. (2011)). Bacteriophage T4, in addition to requiring LPS for infection, also requires OmpC to facilitate entry into the cell (Yu, F., and Mizushima, S. (1982)). Deletion of ompC in Klebsiella MH258 reduced infection and lysis by ΦER39, whereas complementing the ompC gene deletion (ΔompC pZE4D-ompC) rescued the ability of ΦER39 to infect and lyse the ompC deletion Klebsiella MH258 mutant. Unlike with T4, the ΔompC mutant only showed a slight resistance to infection with phages ΦER15 and ΦER16e, perhaps indicating a lesser role for OmpC in the two T4-like phages. The ΔompC mutant also showed slight resistance to ΦER46e, perhaps by affecting small molecules or cation transport near the cell surface. The membrane integrity of ΔompC was tested by plating the mutants on plates containing SDS and EDTA (FIG. 35B), however there did not seem to be a membrane defect.

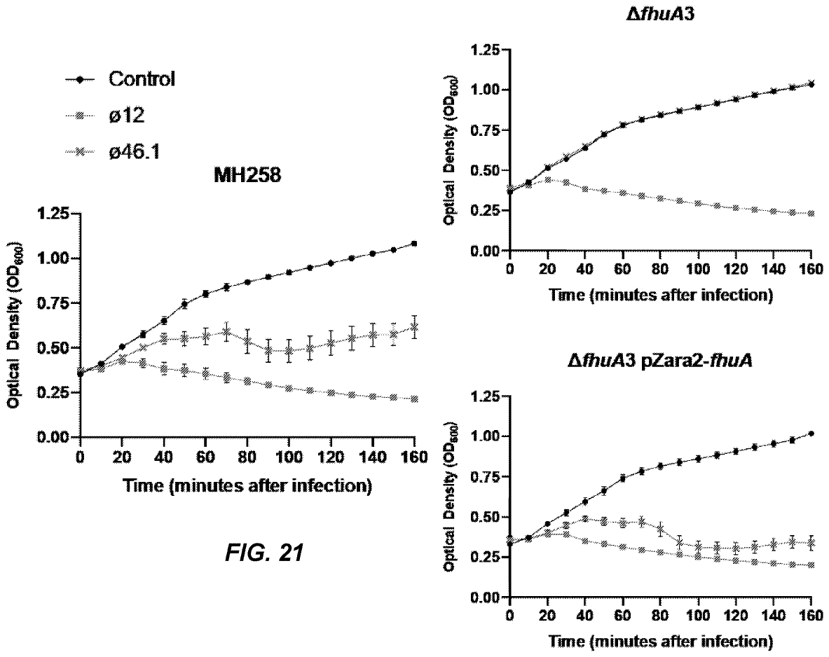

fhuA3 was deleted in Klebsiella MH258, and the fhuA3 deletion Klebsiella MH258 mutant (ΔfhuA3) was treated with ΦER12 or ΦER46 (FIG. 21; FIG. 33F; FIG. 34). The fhuA gene is responsible for iron transport into the cell, however it is also known as the receptor for both bacteriophages T1 and T5. Deletion of fhuA3 in Klebsiella MH258 reduced infection and lysis by ΦER46, whereas complementing the fhuA3 gene deletion (ΔfhuA3 pZE4D-fhuA3) rescued the ability of ΦER46 to infect and lyse the fhuA3 deletion Klebsiella MH258 mutant. In the ΔfhuA3 strain, only ΦER46e was unable to infect in this background, indicating that the FhuA3 is the cell receptor for ΦER46. Additionally, sequencing the fhuA3 gene of a spontaneous survivor of ΦER46e infection revealed a six amino acid deletion.

Disruption to Membrane Stability: A gene in the Tol-Pal system was also deleted, since both ΦER15 and ΦER39 contained tolB in the top over-represented genes for phage resistance (FIG. 33I). The Tol-Pal system comprises proteins TolA, TolQ, and TolR in the inner membrane and TolB and Pal in the outer membrane. Disruptions to the Tol-Pal system result in a pleiotropic effect that disrupts the outer membrane integrity, susceptibility to certain antibiotics like vancomyin and beta-lactams, and changes in cell division and cell morphology (Clavel, T., Lazzaroni, J. C., Vianney, A., and Portalier, R. (1996); Hirakawa, H., Suzue, K., and Tomita, H. (2022)). Additionally, there is evidence that loss of the Tol-Pal system plays a role in reducing virulence (Hirakawa, H., Suzue, K., and Tomita, H. (2022)). ΔtolB was constructed, and it was confirmed that the cells have altered cell membrane integrity by plating colonies on LB plates supplemented with 1% SDS and 2 mM EDTA. The ΔtolB mutant formed smaller colonies in the presence of these membrane-destabilizing agents and complementation with tolB restored colony size (FIG. 35B). The ΔtolB showed striking resistance to phages ΦER15, ΦER16, ΦER39, and ΦER46 in liquid culture, indicating that membrane stability could play a role in allowing these phages to enter the cell (FIG. 33H).

Carbon Storage Regulator Deletion: One of the unexpected hits in both INSeq (ΦER15 and ΦER39) and in survivors of infection (ΦER16) was to the carbon storage regulator pathway, mediated by CsrABCD. CsrD negatively regulates the small RNAs CsrB and CsrC that sequester CsrA, which in turn acts as a regulator for a variety of different sugar pathways (Esquerré, T., Bouvier, M., Turlan, C., Carpousis, A. J., Girbal, L., and Cocaign-Bousquet, M. (2016)). The survivors of ΦER16 had multiple hits to both the csrA promoter (likely reducing its expression) and to the csrD coding region. Since sugars are the main component of both the capsule and the LPS, we hypothesized that altered capsule or LPS abundance was the means to provide resistance.

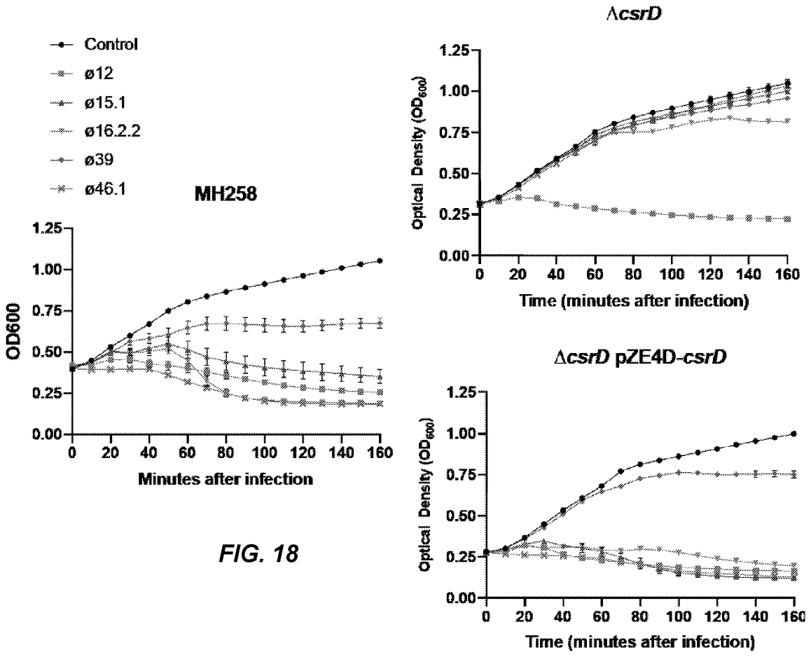

csrD was deleted in Klebsiella MH258, and the csrD deletion Klebsiella MH258 mutant (ΔcsrD) was treated with ΦER12, ΦER15, ΦER16, ΦER39, or ΦER46 (FIG. 18; FIG. 33G; FIG. 34). Deletion of csrD in Klebsiella MH258 reduced infection and lysis by ΦER15, ΦER16, ER39, and ΦER46, whereas complementing the csrD gene deletion (ΔcsrD pZE4D-csrD) rescued the ability of ΦER15, ΦER16, ΦER39, and ΦER46 to infect and lyse the csrD deletion Klebsiella MH258 mutant. The ΔcsrD mutant of MH258 exhibited increased capsule levels (FIG. 35A), confirming previous reports (Mike, L. A., Stark, A. J., Forsyth, V. S., Vornhagen, J., Smith, S. N., Bachman, M. A., and Mobley, H. L. T. (2021)). The ΔcsrD showed resistance to ΦER15, ΦER16e, ΦER39, and ΦER46e in liquid culture. Conversely, cells were more permissive to phages ΦER15, ΦER16, ΦER39, and ΦER46 when over-expressing the csrD on a plasmid (FIG. 33J). Although the ΔcsrD strain showed resistance in liquid assays, phages were still able to plaque in high titer spots, indicating that CsrD likely modulates cellular physiology to permit phage infection rather than acting as a strict requirement for infection.

To further investigate the effect of ΔcsrD on cell metabolism, RNA-seq was performed on MH258 and ΔcsrD. In the mutant strain, 13 genes were upregulated and 74 downregulated (log₂ Fold Change>2, p_{adj}<0.05) (FIG. 33K). Gene set enrichment analysis revealed decreased expression of genes involved in purine metabolism, starch and sucrose metabolism, and oxidative phosphorylate pathways in the deletion mutant (FIG. 33L), suggesting that the loss of the carbon storage regulator affects phage infection by modulating the metabolic state of the cell to one less conducive for phage infection. Altered sugar metabolism in ΔcsrD may modulate accessibility of cell surface receptors by increasing substrate availability for capsule biosynthesis and thickening the capsule. Further, decreased host purine synthesis in the ΔcsrD may decrease the pool of host nucleotides coopted for phage genome synthesis, akin to recently described phage defense systems (Tal, N., Millman, A., Stokar-Avihail, A., Fedorenko, T., Leavitt, A., Melamed, S., Yirmiya, E., Avraham, C., Brandis, A., Mehlman, T., et al. (2022); Hsueh, B. Y., Severin, G. B., Elg, C. A., Waldron, E. J., Kant, A., Wessel, A. J., Dover, J. A., Rhoades, C. R., Ridenhour, B. J., Parent, K. N., et al. (2022)).

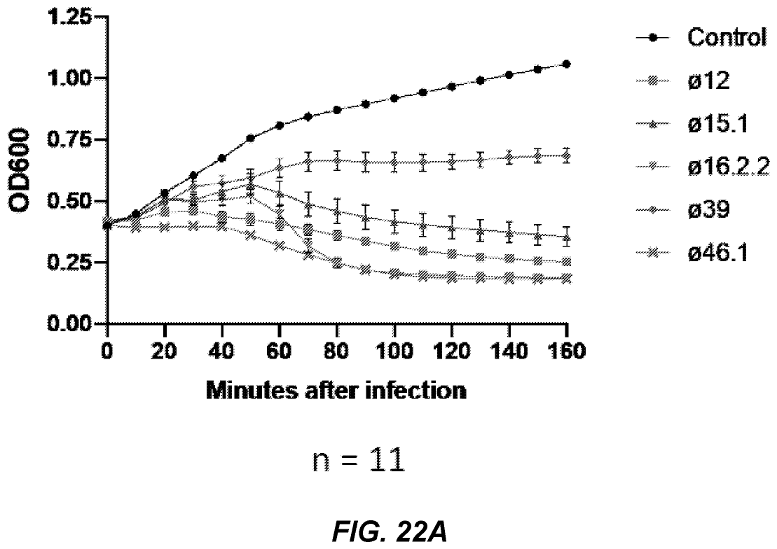

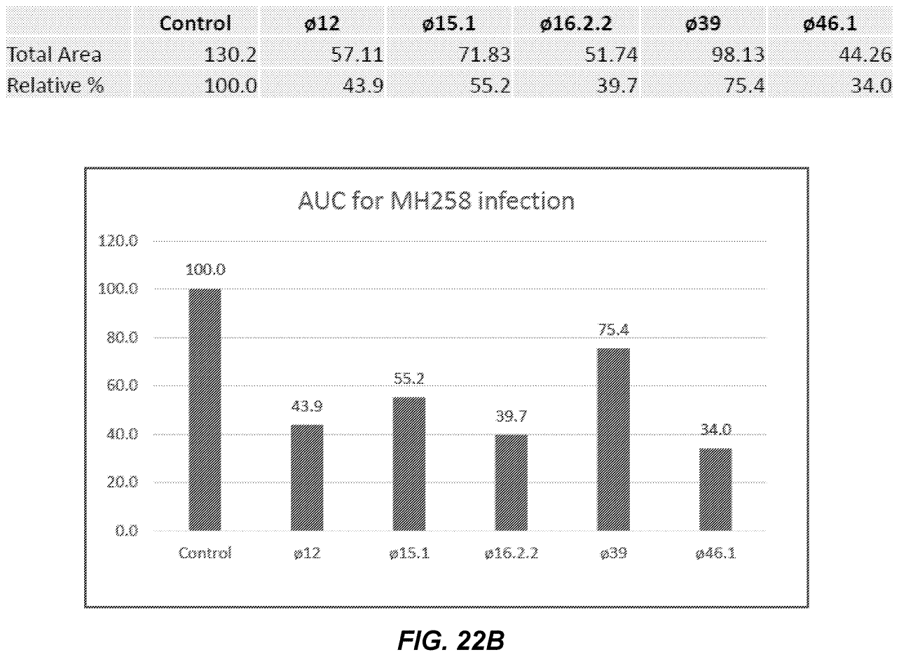

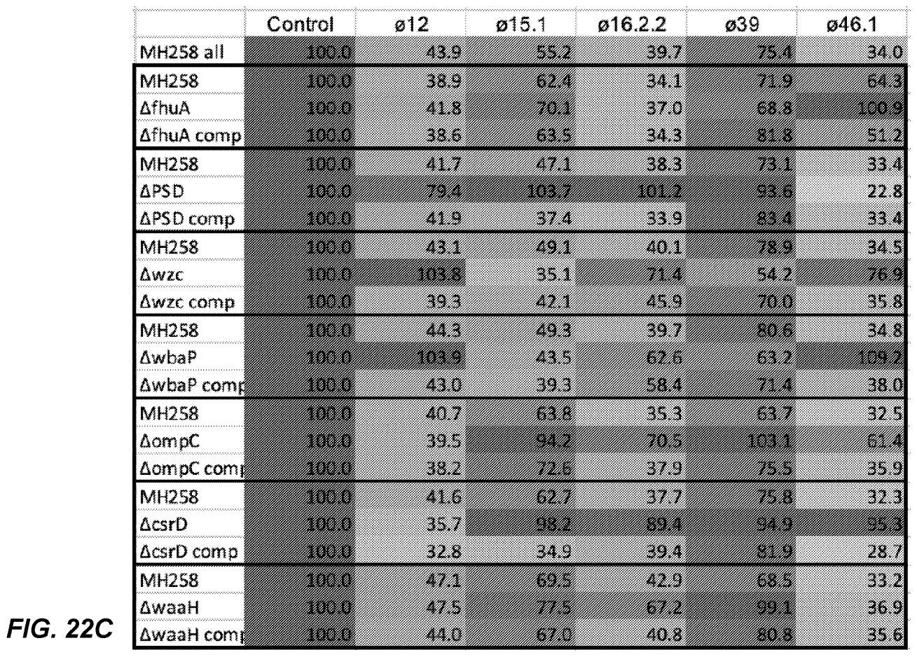

Another way to show lethality of the bacteriophages is to provide an area under curve graph to represent lethality by one number instead of a graph. For example, FIG. 22A shows curves for infection of Klebsiella MH258 by ΦER12, ΦER15, ΦER16, ΦER39, or ΦER46, while FIG. 22B shows the area under each curve of FIG. 22A normalized to untreated control Klebsiella MH258 to provide a numerical representation of lethality of ΦER12, ΦER15, ΦER16, ΦER39, or ΦER46 infection in Klebsiella MH258 (FIG. 22B) or of ΦER12, ΦER15, ΦER16, ΦER39, or ΦER46 infection in Klebsiella MH258 strains in which bacterial genes determined to be required for infection of Klebsiella with ΦER12, ΦER15, ΦER16, ΦER39, or ΦER46 are deleted and then complemented (FIG. 22C).

### Example 5—Phage-Resistant Bacteria are More Susceptible to Macrophage Engulfment & Antibiotic Treatment

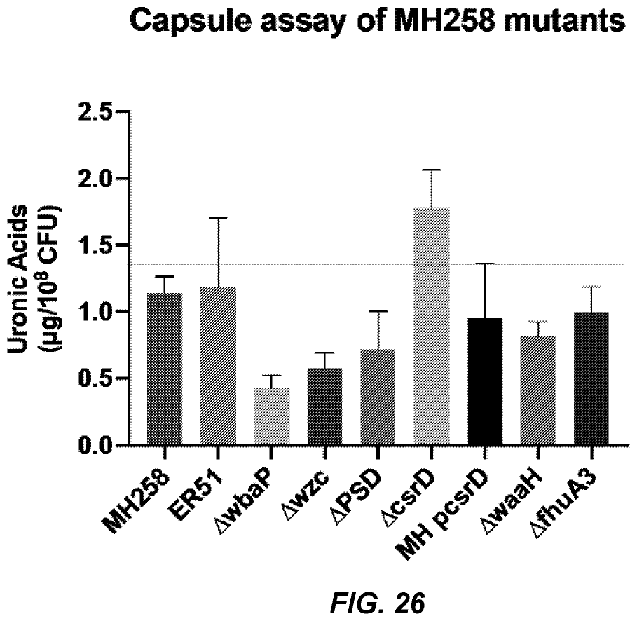

Macrophages in the immune system engulf and digest bacteria, including Klebsiella. One of the hallmarks of Klebsiella species is the copious amount of capsule they produce, which prevents macrophage engulfment. Conversely, non-capsulated Klebsiella are more readily engulfed. FIG. 26 shows capsule amounts of Klebsiella MH258 deletion mutants in which bacterial genes wbaP, wbaN (PSD), wzc, csrD, waaH, or fhuA3 are deleted or a Klebsiella MH258 strain having extra capsule (wzc-G565A; ER51).

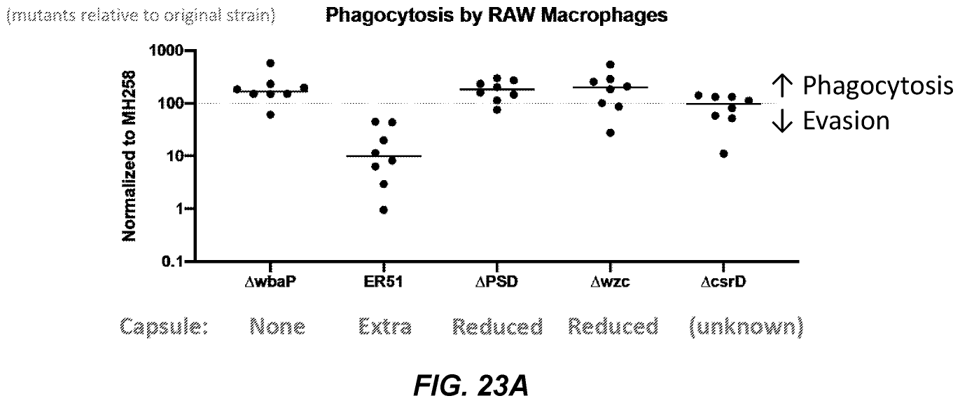

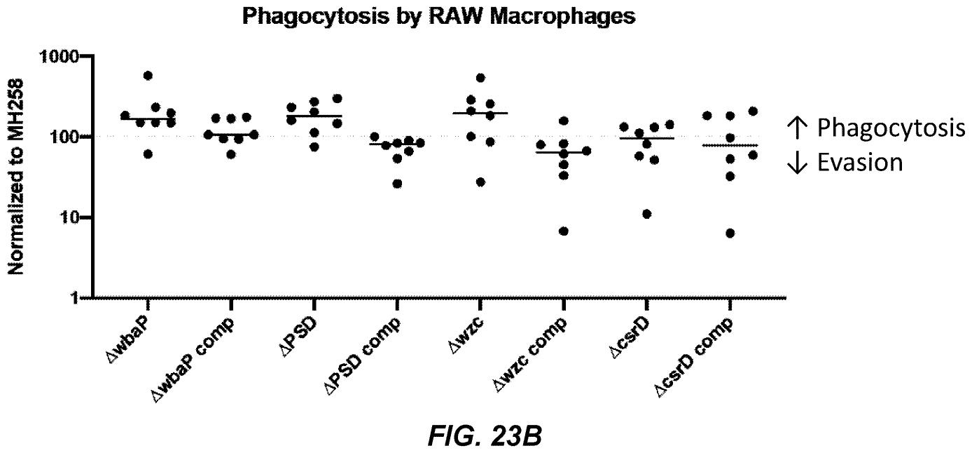

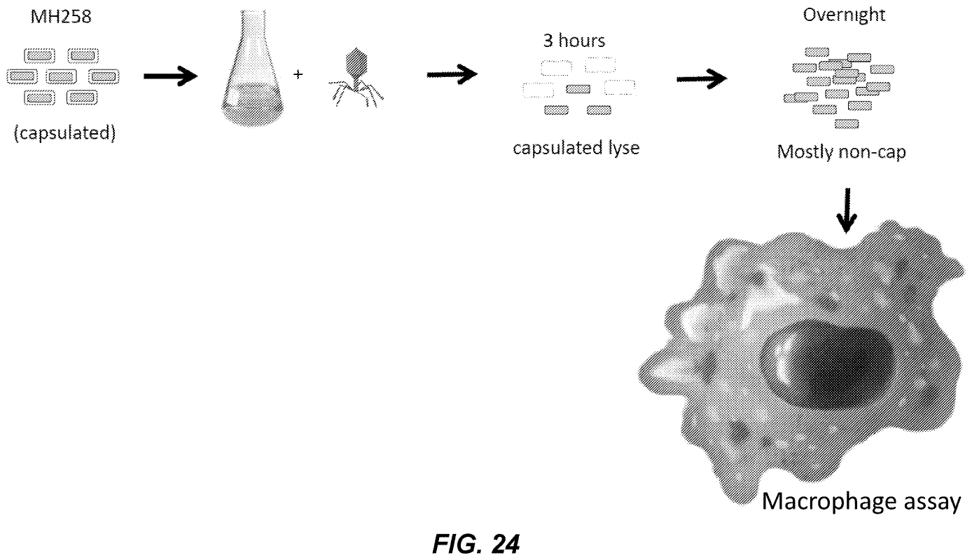

FIG. 24 and FIG. 36A illustrate schematically a method for measuring phagocytosis by macrophages of bacteriophage-treated bacteria, according to some aspects of the disclosure. Mouse RAW 246.7 macrophage cells were provided with wild-type and mutant MH258 strains (either deletion mutants or survivors of MH258 infected with the indicated phage) for 2 hours, treated with a high concentration of gentamicin to eliminate extracellular bacteria, before lysis and bacterial enumeration from the lysate. As shown in FIGS. 23A-23B, FIG. 25, and FIG. 36, in some aspects, bacteriophage treatment or deletion of bacterial genes determined to be required for infection of Klebsiella makes it easier for macrophages to engulf Klebsiella.

A non-capsulated (ΔwbaP) mutant of MH258 and an extra capsulated variant of MH258 (wzc_{G565A}) were tested in a mouse macrophage engulfment assay. MH258 wzc_{G565A} strain was an extra-mucoid survivor of T4-like ΦER8 infection that we identified by whole genome sequencing. Previous reports show this mutation causes an increase in capsule formation that is different from the hyper-mucoid capsule of hyper-virulent strains (Ernst, C. M., Braxton, J. R., Rodriguez-Osorio, C. A., Zagieboylo, A. P., Li, L., Pironti, A., Manson, A. L., Nair, A. V., Benson, M., Cummins, K., et al. (2020)). The wzc_{G565A} allele causes the bacteria to produce extra capsule, making them more difficult to be engulfed, in some aspects.

The non-capsulated ΔwbaP showed a modest 2-5-fold increase in intracellular titer compared to the capsulated parent strain, which was restored upon complementation with wbaP (FIG. 35C). Conversely, in MH258 wzc_{G565A}, a 5-fold decrease in phagocytosis was observed. The capsule-deficient mutants ΔwalW and Δwzc also showed a 2× increase in macrophage engulfment that was partially restored to parental levels by plasmid complementation. ΔcsrD and its complement were also tested in the macrophage assay, however neither was significantly different from MH258 (FIG. 35C), despite the ΔcsrD making more uronic acid (FIG. 35A). In the second set of macrophage experiments, the ΔcsrD was taken up at 1.5× of the parental levels (FIG. 36B).

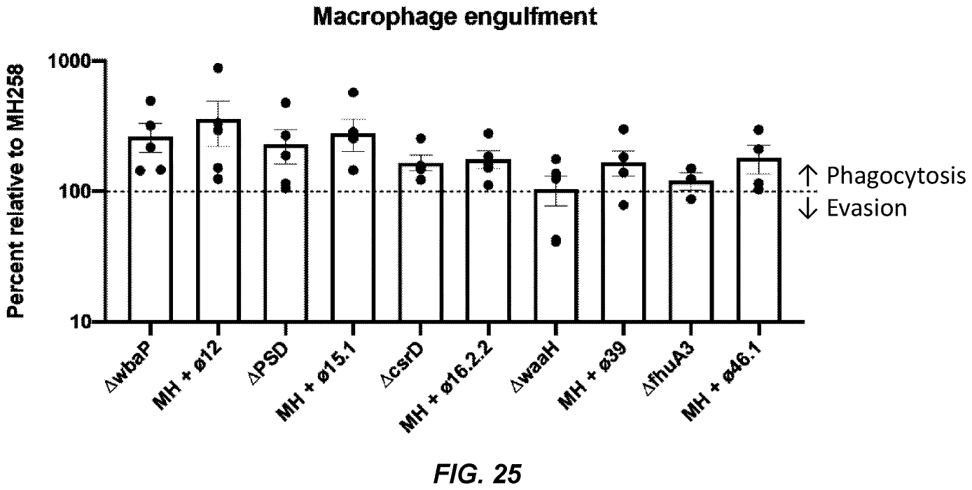

FIGS. 23A-23B show that phagocytosis by mouse macrophages is increased for Klebsiella MH258 strains in which bacterial genes wbaN (PSD) and wzc are deleted and reduced for a Klebsiella MH258 strain having extra capsule (ER51) (FIG. 23A; n=8, bar is median). In some aspects, complementing gene deletions can reduce the ability of macrophages to phagocytose the Klebsiella MH258 deletion mutants (FIG. 23B; n=8, bar is median). FIG. 25 shows that, in some aspects, survivors of phage infection, particularly non-capsulated Klebsiella, are more susceptible to macrophage engulfment.

Tested next was how well macrophages engulfed MH258 that had been treated with different phages (ΦER12, ΦER15, ΦER16e, ΦER39, and ΦER46e) at a multiplicity of infection (MOI) of 1-2. There was an expectation that the phages would kill the majority of wild-type bacteria cells and select for variants with defects in either capsule or LPS production that would render them more susceptible to engulfment. Indeed, ΦER12, ΦER15, ΦER16e and ΦER46e infected cultures, which have capsule and LPS defects as survivors, were readily engulfed by macrophages. Unexpectedly, ΦER39 survivors were also increased over the parental. ΦER39-resistant mutants are likely in waaH or ompC, however a ΔwaaH mutant was taken up at similar levels as the parent strain. Similarly, a ΔfhuA3 strain was also taken up at similar levels to the parent, and a likely mechanism of resistance to ΦER46e is through mutation to fhuA3. Together, these data suggest that the mixed population of survivors of a phage infection have collectively higher susceptibility to phagocytosis than single mutants alone. For example, the presence of LPS-defects in ΦER39-infected and non-capsulated survivors in ΦER46e-infected cultures could have contributed to the increase in macrophage engulfment.

Phage treatment has been previously linked to altered antibiotic susceptibility in surviving bacterial populations (Gurney, J., Brown, S. P., Kaltz, O., and Hochberg, M. E. (2020); Oromi-Bosch, A., Antani, J. D., and Turner, P. E. (2023); Burmeister, A. R., Fortier, A., Roush, C., Lessing, A. J., Bender, R. G., Barahman, R., Grant, R., Chan, B. K., and Turner, P. E. (2020)). Antibiotic resistance was therefore tested via disk diffusion in wild-type and mutant MH258 strains and observed pleiotropic effects (FIG. 36C). At baseline, wild-type MH258 is resistant to all antibiotics tested, including carbapenems, cephalosporins, aminoglycosides, and tetracyclines. Deletion of some essential host factors for phage infection, such as ΔwalW and Δwzc, led to slight sensitization to multiple classes of antibiotics. However, deletion of ompC (and to a lesser extent ΔcsrD and ΔtolB) significantly increased resistance to carbapenem class antibiotics, corroborating previous reports that ompC is necessary for import of carbapenems into the periplasm (Tsai, Y.-K., Fung, C.-P., Lin, J.-C., Chen, J.-H., Chang, F.-Y., Chen, T.-L., and Siu, L. K. (2011); Bulman, Z. P., Krapp, F., Pincus, N. B., Wenzler, E., Murphy, K. R., Qi, C., Ozer, E. A., and Hauser, A. R. (2021)). Genetic complementation of these deletions restored wild-type levels of sensitivity (FIG. 35D). Altogether, phages may select for mutants with increased or decreased susceptibility to antibiotics, suggesting that a careful approach must be taken to design phages that do not interfere with conventional antibiotic therapy. However, in assays with survivors of phage-treated cultures, increased antibiotic resistance was rarely observed, although the number of suppressor colonies in zones of clearing greatly increased. For example, ompC mutations that lead to ΦER39 resistance would lead to increased carbapenem resistance in that population. Overall, phage treatment tended to sensitize strains to various classes of antibiotics, particularly with ΦER39 and ΦER46e which increased sensitivity to imipenem.

### Example 6—Bacteriophage Cocktails Increase Time of Bacterial Resistance

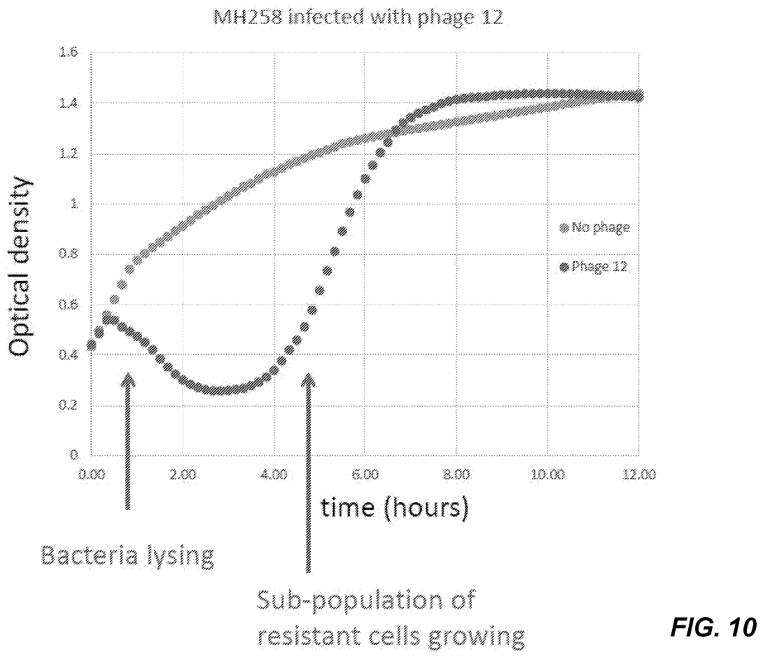

Klebsiella become resistant to bacteriophage quickly in vitro (e.g., ΦER12 resistance can be promoted by loss of the Klebsiella capsule). For infection, since some phages used LPS (Straboviridae), others used capsule (Ackermannviridae and Demerecviridae), and still others relied on outer membrane proteins (Siphoviridae and Demerecviridae), it was reasoned that the different mechanisms of infection would make it difficult to for the cell to overcome by either delaying the time for a population to become resistant or by negatively affecting the fitness of the phage-resistant cells. For example, loss of the Klebsiella capsule, however, may make it easier for a second phage to infect the Klebsiella. This is supported by evidence that many phages are more virulent in ER26 (a capsule mutant of Klebsiella MH258). Time to resistance can be measured by using a plate reader to detect optical density of the Klebsiella MH258 in the presence and absence of bacteriophage, and FIG. 10 shows development of resistance over time by Klebsiella MH258 to ΦER12.

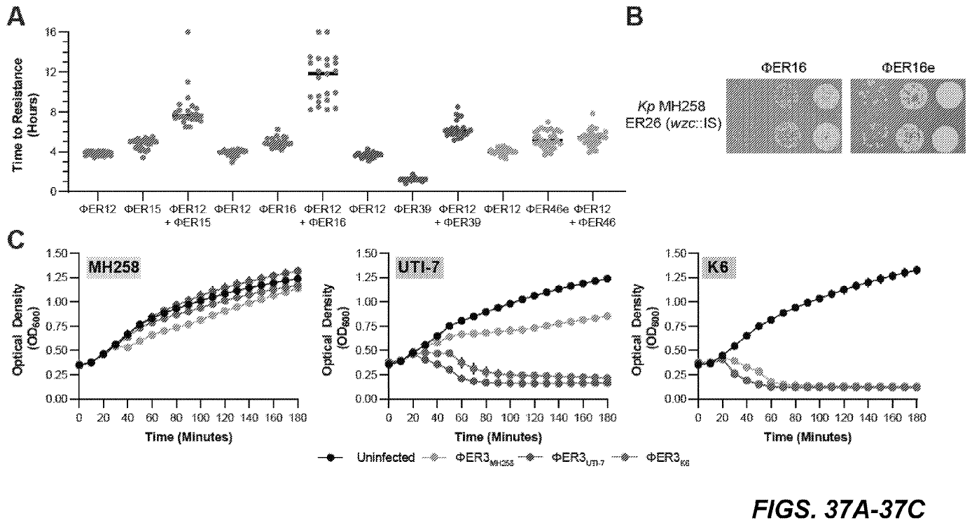

Resistance to ΦER12 occurred exclusively due to loss of the capsule. When grown in liquid culture, it took approximately 3.8 hours for a culture of 2×10⁶ MH258 cells to recover from infection with ΦER12. It was reasoned that if a second capsule-independent phage were added to phage ΦER12, the frequency of double resistant mutants would be lower in the initial population and the time for the culture to become resistant would be delayed. This delay in the outgrowth of resistant mutants would be beneficial for phage therapy allowing more time for the host to clear away a Klebsiella infection. Cultures of MH258 were infected singly with phages with either ΦER15, ΦER16e, ΦER39, or ΦER46e, and doubly with ΦER12 and a second phage (FIG. 37A). The time for resistance with ΦER12 combinations increased to an average of 8.2 hour with the addition of ΦER15; 11.38 hours with ΦER16; 6.28 hours with ΦER39; and to 5.3 hours with ΦER46e (p<0.0001 for all combinations, one-way ANOVA with Tukey's multiple comparison). However, ΦER12 and ΦER46e are both capsule-dependent and their combination (5.3 hours) failed to increase the time for resistance with ER46e incubation alone (5.2 hours), suggesting that ΦER46e resistant variants also confer ΦER12 resistance.

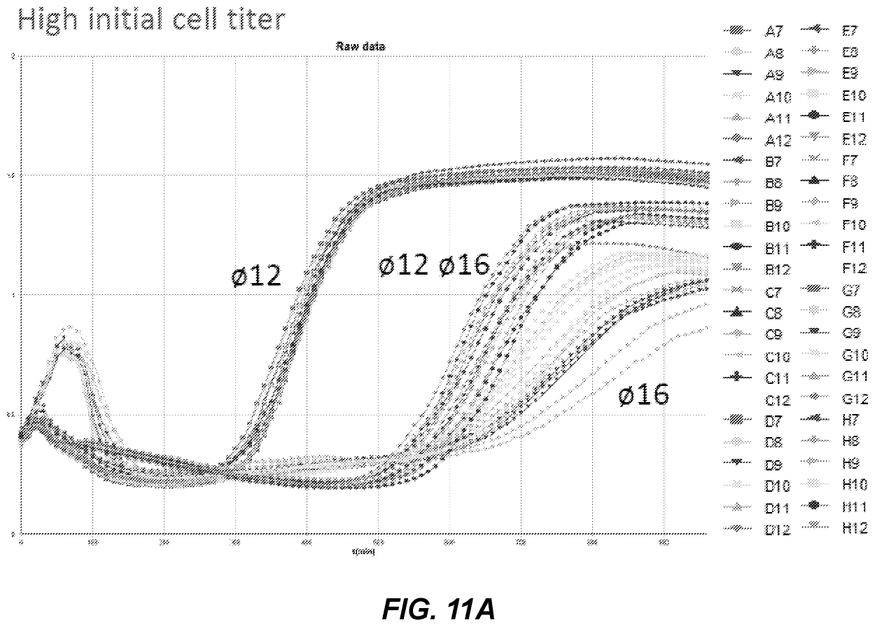

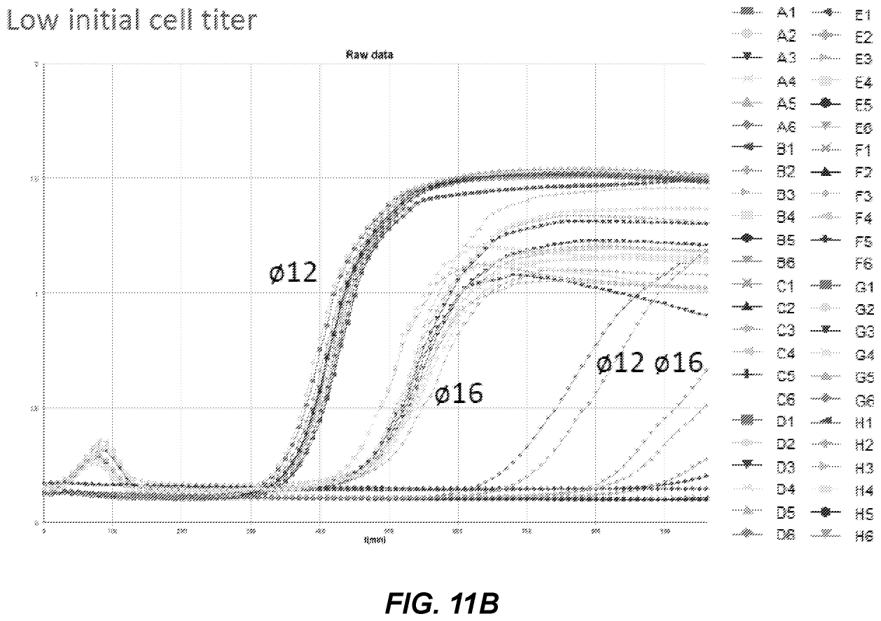

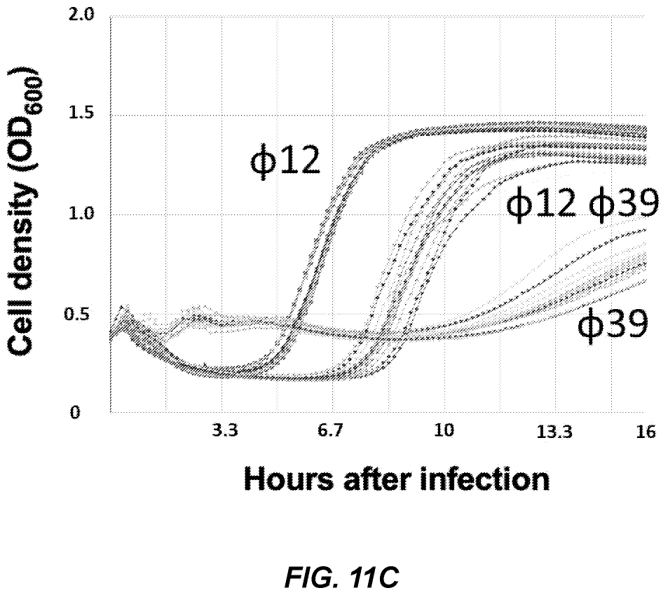

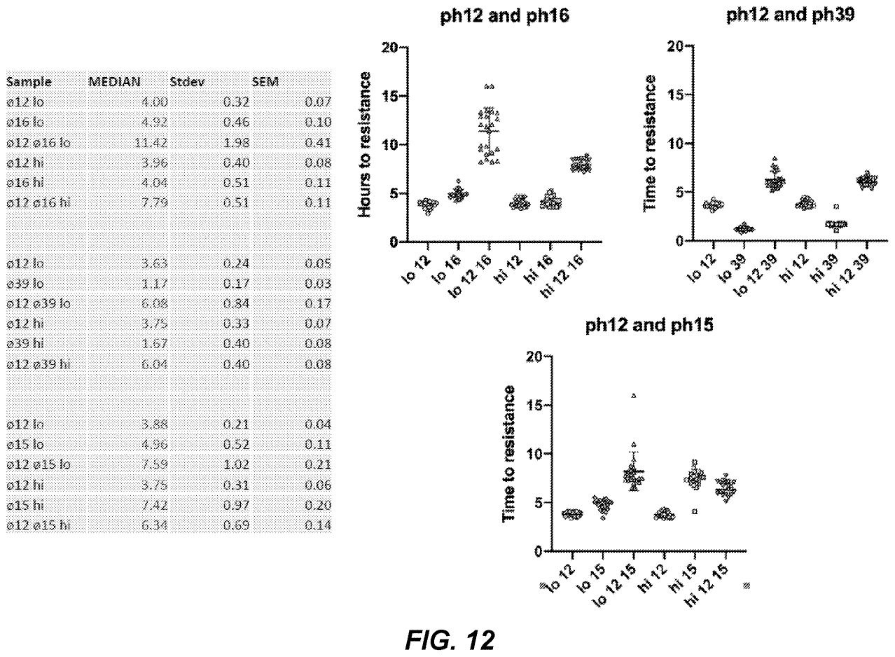

Combining ΦER12 with an additional bacteriophage can therefore delay the time to ΦER12 resistance by the Klebsiella MH258 by over 2 hours. As additional examples, FIG. 11A shows delayed development of resistance by Klebsiella MH258 to ΦER12 when ΦER12 is combined with ΦER16 (high initial cell titer). FIG. 11B shows delayed development of resistance by Klebsiella MH258 to ΦER12 when ΦER12 is combined with ΦER16 (low initial cell titer). FIG. 11C shows delayed development of resistance by Klebsiella MH258 to ΦER12 when ΦER12 is combined with ΦER39. FIG. 12 shows stochastic time to resistance by low (lo; ˜2×10⁷ cfu/ml) or high (hi; ˜2×10⁸ cfu/ml) initial Klebsiella MH258 cell titers to combinations of ΦER12 and ΦER16, ΦER12 and ΦER39, or ΦER12 and ΦER15.

These results demonstrate that, in some aspects, utilizing a combination of bacteriophage can increase time to resistance by Klebsiella MH258 to the bacteriophage.

### Example 7—Optimized Phage Cocktails Kill Multidrug Resistant and Clinical Isolates of Klebsiella pneumoniae In Vitro

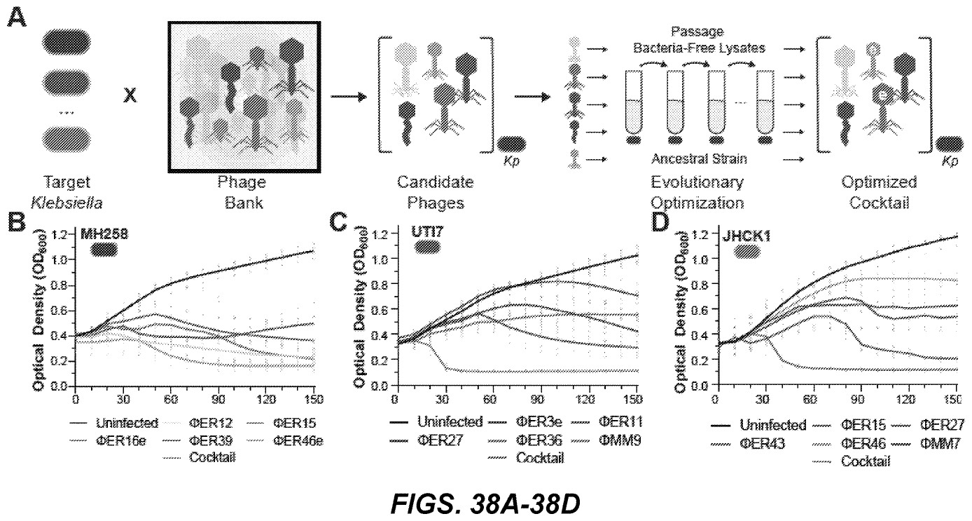

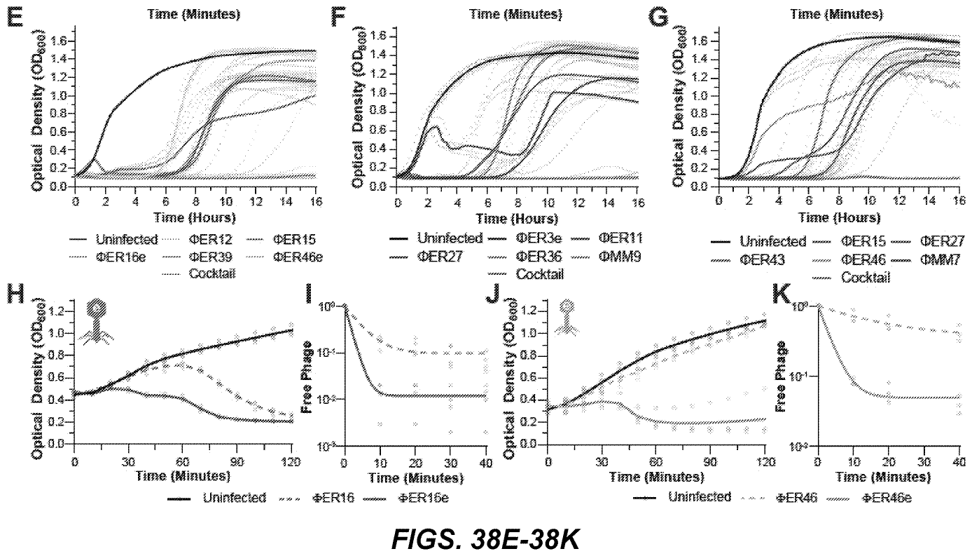

Using the Klebsiella phage isolates, a process to generate an optimized phage cocktail for a given Klebsiella strain was formalized (FIG. 38A). To generate a cocktail that could best kill Klebsiella, five phylogenetically distinct phages predicted to depend on different host factors for infection were selected. As a proof-of-principle, 5-phage cocktails were designed to target carbenem-resistant K. pneumoniae MH258, ESBL-positive JHCK1, and a primary clinical recurrent urinary tract infection isolate K. quasipneumoniae UTI-7. Cocktail selection began by referencing the host range table (FIG. 32) and identifying phages with the highest lytic activity in each taxonomic group. Individual infections were performed with the candidate phages in a plate reader and those that displayed killing within 2.5 hours were selected (FIGS. 38B-38D). For MH258, ΦER12 (Ackermannviridae), ΦER15 (Tevenvirinae), ΦER16e (Slopekvirus), ΦER39 (unclassified Siphoviridae), and ΦER46e (Demerecviridae) were chosen. For UTI-7, ΦMM9 (Autographiviridae), ΦER11 (Tevenvirinae), ΦER27 (Slopekvirus), ER32 (Slopekvirus), and ΦER3 (proposed Purpuraviridae) were chosen. For JHCK1, ΦER15 (Tevenvirinae), ΦER27 (Slopekvirus), ΦER43 (Autographiviridae), ΦER46 (Demerecviridae), and ΦMM7 (proposed Purpuraviridae) were chosen. All these phages were able to infect their respective host individually at MOI=˜1, and the 5-phage cocktails were able to effectively kill the population and prevent significant growth from occurring (FIGS. 38B-38D).

While the individually infected cultures were killed or had reduced titer during a short time frame of several hours, all the cultures eventually became resistant over the course of an overnight infection (FIGS. 38E-38G). Due to the stochastic nature of the spontaneous mutations, there was variability in the time it took for cultures to become resistant. However, the cocktails of all five phages suppressed the incidence of resistance in all (JHCK1) or most cultures (MH258 and UTI-7). The recovered colonies from these 5-phage infections were usually small, non-capsulated, or both. To further investigate the nature of these mutations, whole genome sequencing was performed on an MH258 survivor that exhibited a small and sickly colony phenotype that occasionally reverted back to parental phenotype during streaking. This strain contained an insertion element in the gall/gene responsible for the synthesis of UDP-D-glucose from UTP and α-D-glucose 1-phosphate, an upstream part of both the capsule and LPS biosynthesis pathways. Although this strain was resistant to the five phages in liquid culture, it was able to plaque ΦER46e on a solid lawn.

### Example 8—Evolution of Bacteriophage

Repeatedly passaging a bacteriophage isolate from one bacterial strain into another can improve virulence such that bacteriophage are evolved to better infect host bacteria. Accordingly, to further optimize phage virulence, an adaptive directed evolution strategy (often termed ‘phage training’) was employed, wherein a phage is sequentially passaged on a target pathogen to select for genetic variants that improve phage fitness on that specific bacterium in those specific environmental conditions (Borin, J. M., Avrani, S., Barrick, J. E., Petrie, K. L., and Meyer, J. R. (2021); Burrowes, B. H., Molineux, I. J., and Fralick, J. A. (2019); Friman, V.-P., Soanes-Brown, D., Sierocinski, P., Molin, S., Johansen, H. K., Merabishvili, M., Pirnay, J.-P., De Vos, D., and Buckling, A. (2016)). This strategy relies on natural evolutionary processes and has been used clinically to optimize phages for inclusion in therapeutic cocktails (Eskenazi, A., Lood, C., Wubbolts, J., Hites, M., Balarjishvili, N., Leshkasheli, L., Askilashvili, L., Kvachadze, L., van Noort, V., Wagemans, J., et al. (2022); Castledine, M., Padfield, D., Sierocinski, P., Soria Pascual, J., Hughes, A., Mäkinen, L., Friman, V.-P., Pirnay, J.-P., Merabishvili, M., de Vos, D., et al. (2022); Torres-Barceló, C., Turner, P. E., and Buckling, A. (2022)).

Using the set of MH258 phages as a test-case, ΦER12, ΦER15, ΦER16, and ΦER39 were sequentially passaged on the parental bacterial strain for 7 days. Additionally, ΦER46 was passaged on MH258 overexpressing csrD on a high-copy plasmid, as this strain is sensitized to ΦER46 infection. Virulence of the resultant phage populations were assessed using a virulence index assay (Storms, Z. J., Teel, M. R., Mercurio, K., and Sauvageau, D. (2019)), which captures the magnitude of phage killing at varying MOI.

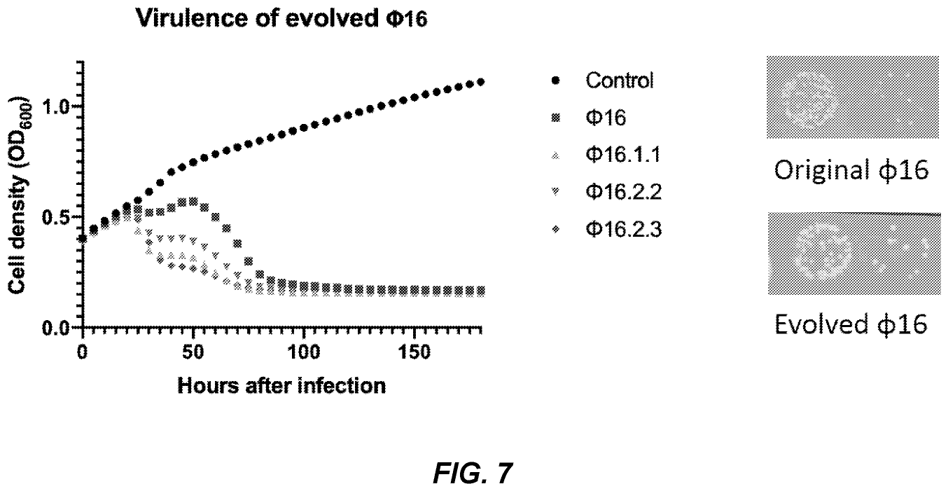

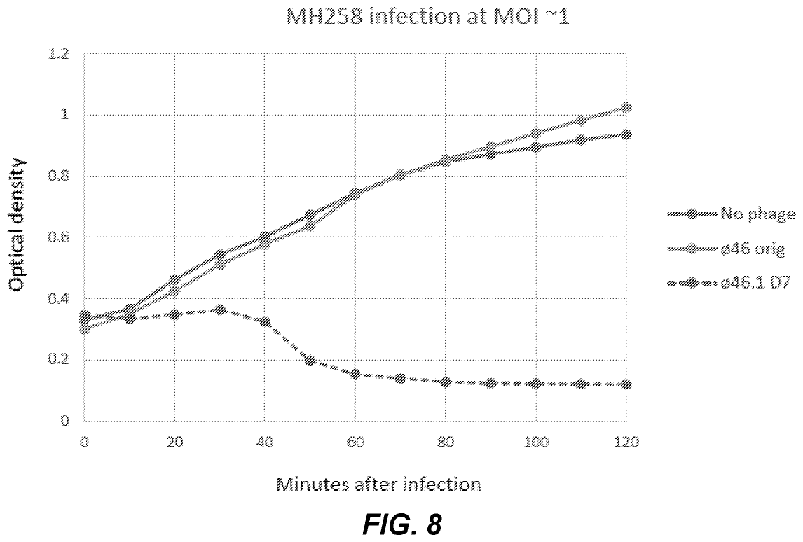

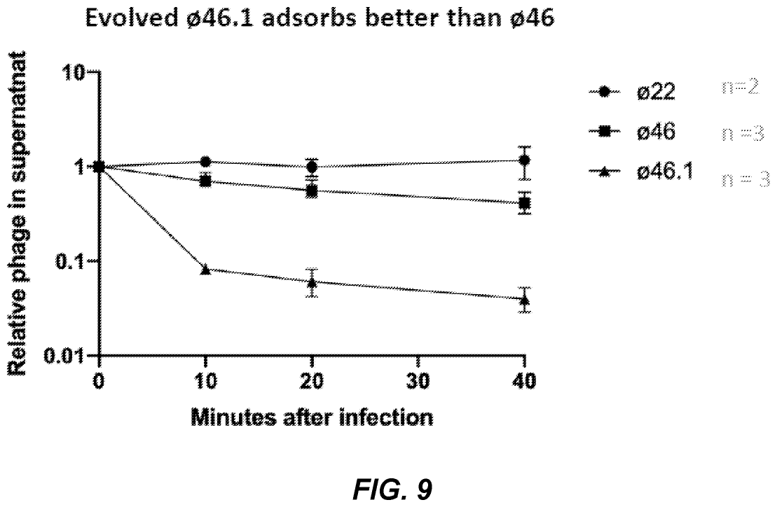

FIGS. 7 and 8 show virulence of a representative bacteriophage (ΦER16, FIG. 7; ΦER46, FIG. 8) evolved over multiple passages (e.g., ΦER16.1.1, ΦER16.2.2, ΦER16.2.3, ΦER46.1). As illustrated by FIGS. 7 and 8, evolved bacteriophage better kill Klebsiella MH258 than original bacteriophage. As shown in FIG. 9, evolved bacteriophage also adsorbs to Klebsiella MH258 better than the original bacteriophage. The negative control 22 is a T1-like Siphoviridae family bacteriophage that does not infect Klebsiella MH258.

For ΦER16 and ΦER46, plaque-purified isolates (ΦER16e and ΦER46e) from the evolved populations displayed a marked increase in virulence compared to ancestral isolates (ΦER16 Vᵢ=0.23; ΦER16e Vᵢ=0.28; ΦER46 Vᵢ=0; ΦER46e Vᵢ=0.69) (FIGS. 38H, 38J). Whole genome sequencing of evolved variants revealed point mutations in tail fiber gene of ΦER16e (ΦER16 CDS_0049 N281T) and a large 404 amino acid deletion in the tail spike of ΦER46e (ΦER46 CDS_0001 Q2652_3055del), suggesting that passaging led to mutations that impact phage binding to bacterial hosts. Adsorption assays of ancestral and evolved phages on MH258 confirmed increased binding affinity (ΦER16=4.8×10⁻¹⁰, ΦER16e=1.2×10⁻⁹, ΦER46=1.2×10⁻¹⁰, ΦER46e=6.6×10⁻¹⁰ mL min⁻¹ cell⁻¹). Visually, it was also apparent that ΦER16e formed larger plaques than ΦER16 (FIG. 37B).

In other cases, such as with ΦER3, the efficiency of infection improved with a single passage on a target strain. ΦER3 that was amplified on UTI-7 infected UTI-7 better than phage that was grown on a K6 or MH258 (FIG. 37C), and vice versa. Whole genome sequencing could not identify any changes to the phage genome, suggesting an epigenetic mechanism.

### Example 9—Effect of Klebsiella Colonization and/or Bacteriophage Treatment on Mouse Microbiome

It was investigated how the mouse gut microbiome is changed upon Klebsiella and/or phage addition. The use of phage cocktails to decolonize carbapenem-resistant K. pneumoniae MH258 from the mouse gut, without affecting the surrounding microbiome, was explored. A compelling use of phage therapy that leverages its specificity is to target patients colonized with multi-drug resistant pathogens that may bloom, dominate the microbiome, and translocate into the bloodstream upon antibiotic use. Human strains of K. pneumoniae do not colonize mice with an intact microbiome (Federici, S., Kredo-Russo, S., Valdés-Mas, R., Kviatcovsky, D., Weinstock, E., Matiuhin, Y., Silberberg, Y., Atarashi, K., Furuichi, M., Oka, A., et al. (2022); Sorbara, M. T., Dubin, K., Littmann, E. R., Moody, T. U., Fontana, E., Seok, R., Leiner, I. M., Taur, Y., Peled, J. U., van den Brink, M. R. M., et al. (2019)) and thus require constant antibiotic perturbation to maintain K. pneumoniae colonization. However, these antibiotic-perturbation models lead to high levels of K. pneumoniae colonization, often comprising the majority of the gut bacterial population as in an antibiotic-mediated bloom.

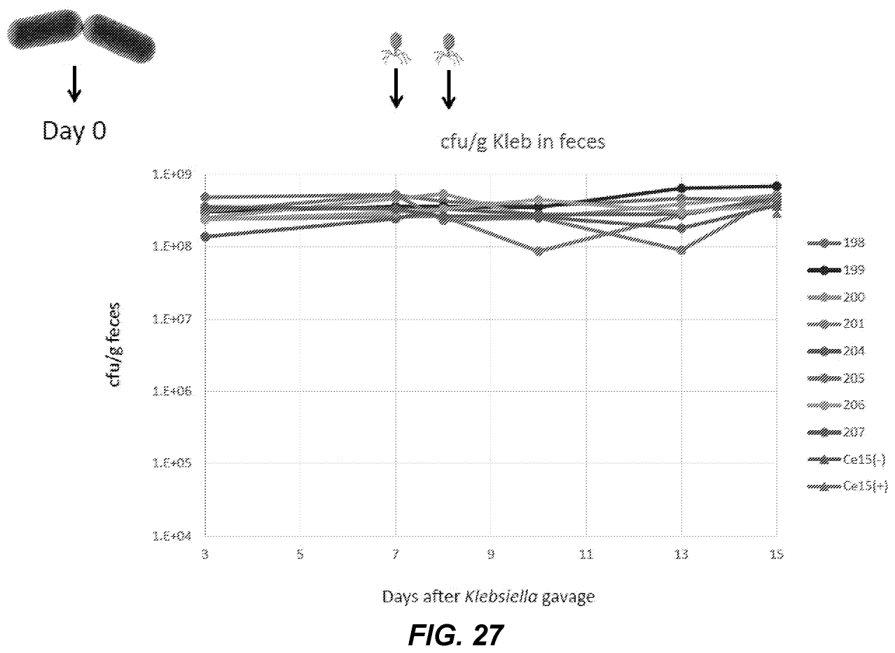

FIG. 27 shows that Klebsiella colonizes the mouse gut microbiome well, the Klebsiella population is stable over time, and bacteriophage treatment does not affect the Klebsiella titer. All mice received Klebsiella MH258 at Day 0. Mice 198, 199, and 200 received no bacteriophage treatment, while mice 201, 204, 205, 206, and 207 received a bacteriophage cocktail at D7 and D8.

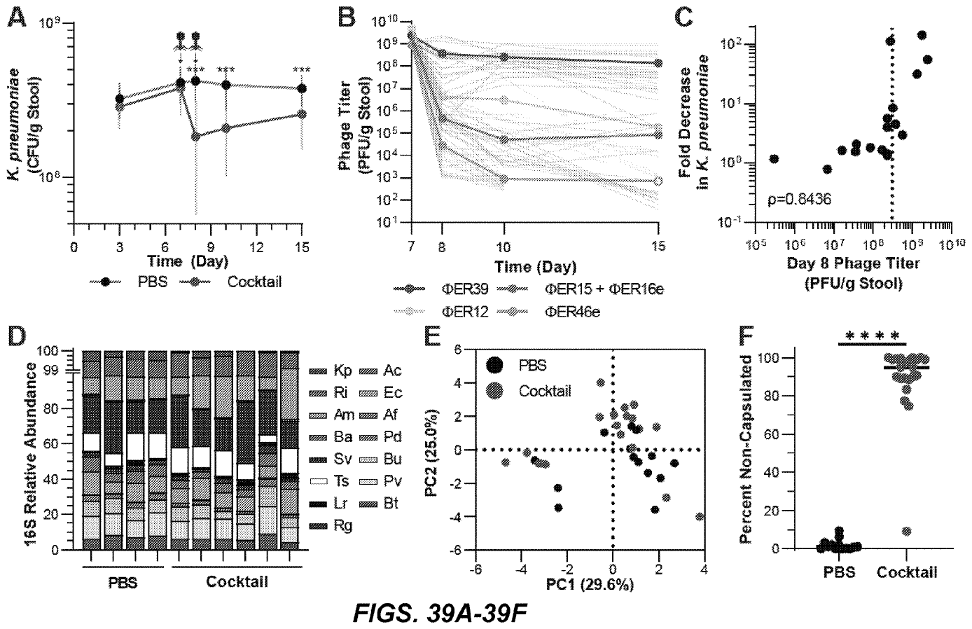

Instead, a gnotobiotic mouse model with a reduced complexity community that permits low level colonization of K. pneumoniae to mimic pathogen carriage was used. To create the community, 13 taxonomically and metabolically diverse human-derived strains were selected and include all 5 major phyla in the human microbiome (see Example 10 for more details). At baseline, 8-12 week old C57BL/6 gnotobiotic mice containing the defined gut microbiome were orally administered ˜5×10⁷ CFU of K. pneumoniae MH258 and given one week for the Klebsiella to engraft and stabilize in the gut community. Bacterial load was enumerated from mouse fecal samples and K. pneumoniae were stably maintained at ˜0.1% relative abundance up to 15 days (FIG. 39A). On Days 7 and 8, the mice were gavaged with either PBS or a cocktail of ΦER12, ΦER15, ΦER16e, ΦER39, and ΦER46e in PBS (each ˜10⁹ PFU/mouse). The K. pneumoniae and phage titers were counted in fecal pellets until the mice were sacrificed on Day 15, where cecal contents were used.

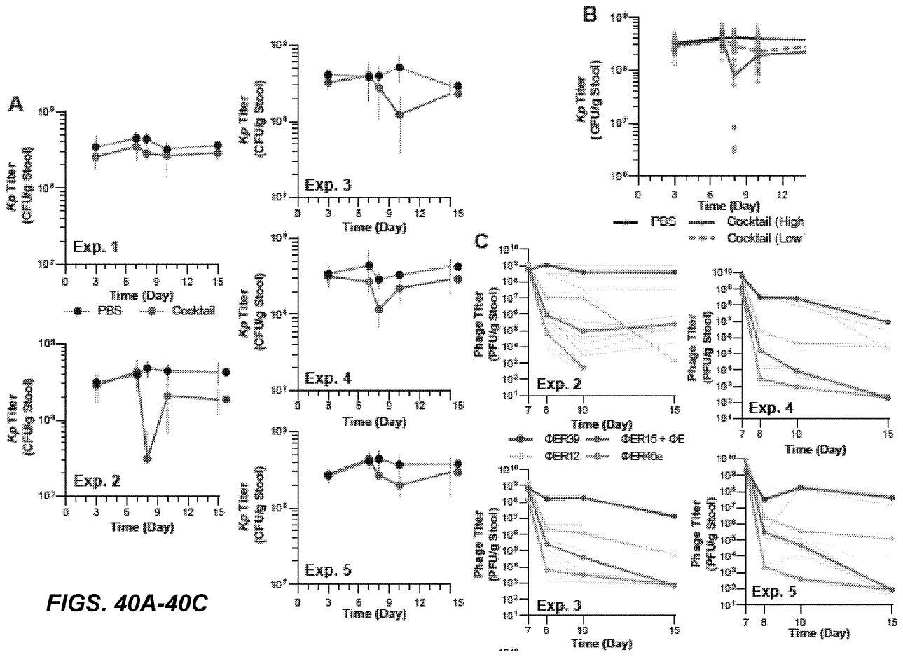

Upon phage treatment, there was a measurable loss in K. pneumoniae titer after administration of the cocktail (FIG. 39A) that peaked on Day 8 and was sustained until the end of the experiment (p<0.001, t test with Welch correction; n=17 PBS, n=23 Φ cocktail). The extent of K. pneumoniae suppression varied by experiment from 2-fold to 50-fold (FIGS. 40A, 40B). Additionally, the phage populations also began to differentially diminish over time as measured by plaquing on indicator strains (FIG. 39B and FIG. 40C). The phage that remained in the highest titer in the mice was ΦER39, at a median value of 4.5×10⁷ PFU/g stool on the last day, followed by ΦER12 at around 1×10⁴ PFU/g stool, then ΦER15, ΦER16e, and ΦER46e at or near the limit of detection of 200 PFU/g stool. The extent of K. pneumoniae inversely correlated with the number of total phages recovered in feces (e.g., a higher phage titer on Day 8 corresponded to a lower K. pneumoniae titer) and a minimum threshold of 2×10⁸ PFU/g stool on Day 8 was necessary for potent suppression of K. pneumoniae burden (FIG. 39C). To assess the effects on the gut microbiome, the composition of the entire gut microbial community was analyzed by 16S rRNA amplicon sequencing at the nadir of K. pneumoniae titer on Day 8. While the relative abundance of K. pneumoniae decreased in phage treated samples, the abundance of no other member of the community was significantly altered following phage treatment (FIGS. 39D, 39E), suggesting that the effects of phage therapy are species-specific and do not compromise community stability.

A striking alteration in the morphology of the surviving K. pneumoniae was observed post-phage treatment. At Day 15, most of the bacteria were non-capsulated (FIG. 39F), ranging from 75% to 100% of the colonies per mouse. All the non-capsulated colonies were resistant to phages ΦER12 and to ΦER46e (in liquid culture; some non-capsulated strains still allowed plaquing of ΦER46e on plates, likely indicating a reduction in capsule like a wzc mutation rather than a capsule null mutation such as wbaP). In one experiment, one of the non-capsulated colonies was resistant to both ΦER15 and ΦER16e, likely indicating a mutation to the LPS. The recovered capsulated colonies were mostly still susceptible to all five phages, perhaps indicating a spatial separation of the bacteria from the phages or that subpopulations of K. pneumoniae were in cellular states that disallow phage infection to exist inside the mice. Colonies were isolated from six mice treated with the cocktail that were partially resistant to ΦER39. Whole genome sequencing was performed on one candidate and revealed it to be a mutation in the O-antigen locus, further suggesting a role of O-antigen in Purpuraviridae infection. In sum, treatment with the phage cocktail caused a modest, sustained loss in titer, but after a week, the resulting K. pneumoniae were less capsulated or had altered LPS. Since both capsule and LPS are key virulence factors in K. pneumoniae pathogenesis (Patro, L. P. P., and Rathinavelan, T. (2019)) and their loss sensitizes strains to phagocytosis (FIG. 36) phage therapy could be used in colonized patients to not only suppress bacterial burden, but also to select for strain variants more readily cleared by the host immune system.

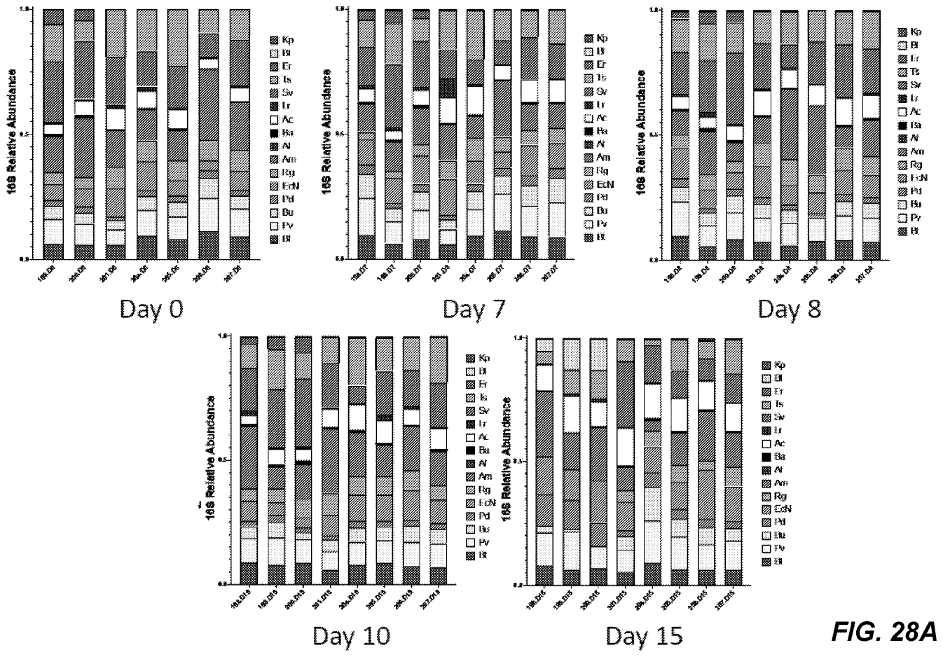

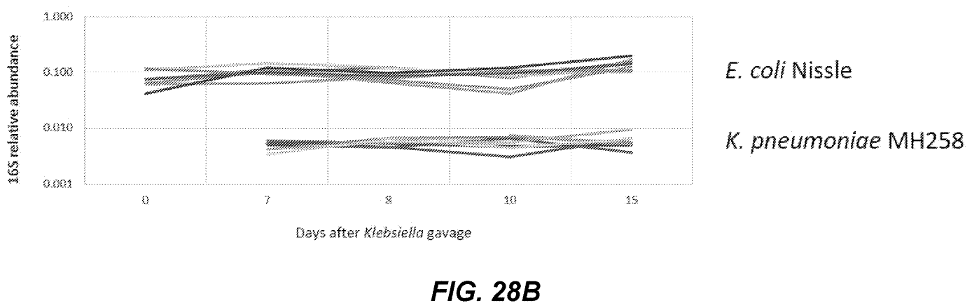

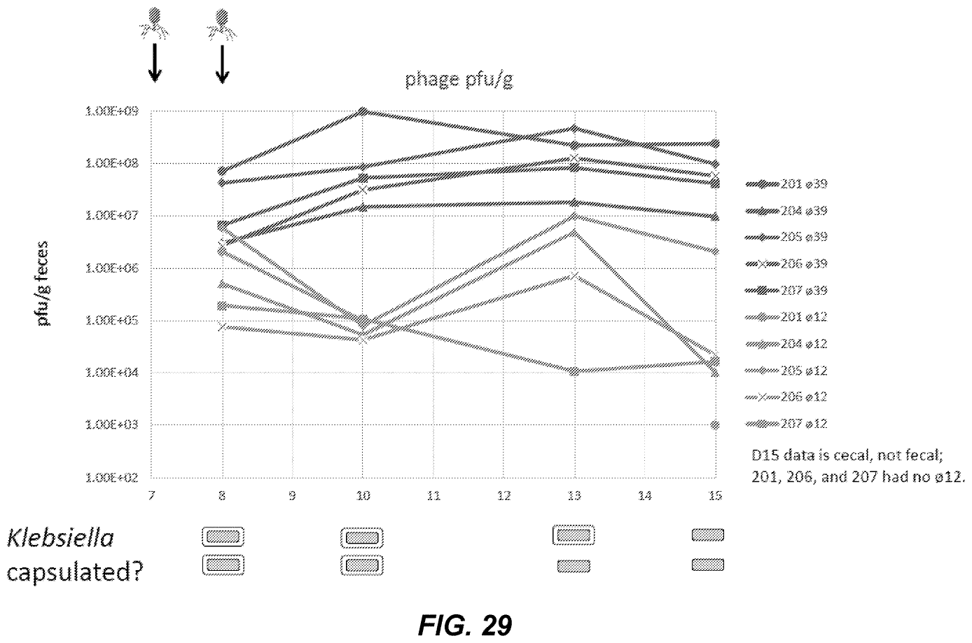

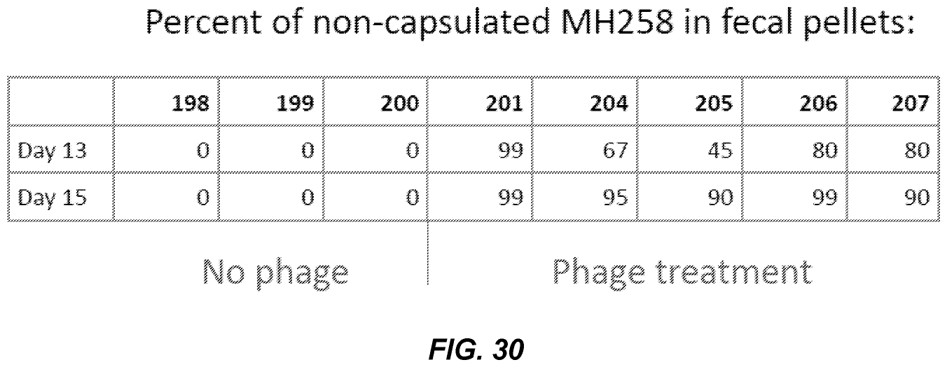

In a second experiment, the distribution of a 14-species gnotobiotic mouse model gavaged with 4×10⁷ cfu Klebsiella MH258 at Day 0 over the course of 15 days was determined and is shown in FIG. 28A, which demonstrates that, in some aspects, introducing Klebsiella does not alter the composition of the mouse microbiome. Klebsiella remained stable over time between 1-5×10⁸ cfu/g feces. FIG. 28B shows that the E. coli composition, specifically, of the mouse microbiome was not changed upon introduction of Klebsiella. FIG. 29 shows that, in mice, Klebsiella and bacteriophage co-exist, and ΦER12 and ΦER39 persist over time. Finally, FIG. 30 shows that mice infected with ΦER12 have Klebsiella populations without a capsule.

Together, the examples described herein demonstrated that, in some aspects, bacteriophage infection of Klebsiella MH258 results in non-capsulated Klebsiella MH258 cells. In some aspects, bacteriophage-treated Klebsiella are more susceptible to phagocytosis by macrophages. In some aspects, treatment with ΦER12 changes the capsule phenotype of Klebsiella in the mouse gut.

### Example 10—Exemplary Materials & Methods

Bacterial Strains and Growth Conditions: All strains used are listed in Table S1. Deidentified primary clinical isolates were streaked from urine samples obtained from recurrent urinary tract infection at the University of Chicago Urogynecology clinic under IRB22-1580.

All strains were grown aerobically in LB media (BD Difco) broth or on plates with 15 grams agar (Fisher Scientific) per Liter. Top agar was 6 grams of agar/Liter. Sucrose plates were 10 g Bacto tryptone (BD), 5 g yeast extract (Gibco) and 5% sucrose (Sigma-Aldrich)/L and low-salt plates contained: 10 g tryptone, 5 g yeast extract and 5 g NaCl (Acros Organics)/L.

Cultures were shaken at 250 rpm at 37° C. Plates were grown at 37° C. for culture streaks and placed at room temperature for colony enumeration. Antibiotics and supplements were provided at the following concentrations: carbenicillin (GoldBio)—100 μg/mL; tetracycline (ThermoFisher Scientific)—25 μg/mL; streptomycin (Fisher Scientific)—100 μg/mL; spectinomycin (Fisher Scientific)—100 μg/mL; ofloxacin (Fisher Scientific)—1.35 μg/mL; hygromycin (GoldBio)—100 μg/mL; L-arabinose (Sigma Aldrich)—0.35%; EDTA (Invitrogen)—2 mM; Sodium Dodecyl Sulfate (ThermoFisher Scientific)—1%. Dilutions of phage and bacteria were done in 1× phosphate buffered saline (PBS; Thermo Scientific).

Bacterial Genome Sequencing: Bacterial DNA was extracted from 750 μl-1 mL of overnight culture using a Qiagen DNeasy Blood & Tissue kit. Whole genome sequencing was performed by SeqCenter/MiGS (Pittsburgh, PA) using 2×150 bp Illumina NovaSeq at approximately 200 Mbp per sample. Genomes were assembled using Velvet (Zerbino, D. R., and Birney, E. (2008)) de novo assembly in the Geneious Prime 2023.1, with 75 kmer and approximately 800,000-1,000,000 reads to generate 200-300 contigs. MLST, capsule and O-antigen type, and predicted antibiotic resistance were determined using the PathogenWatch (Sánchez-Busó, L., Yeats, C. A., Taylor, B., Goater, R. J., Underwood, A., Abudahab, K., Argimón, S., Ma, K. C., Mortimer, T. D., Golparian, D., et al. (2021)) platform, with the Pasteur Database (https://bigsdb.pasteur.fr/), Kaptive (Lam, M. M. C., Wick, R. R., Judd, L. M., Holt, K. E., and Wyres, K. L. (2022)), and Kleborate (Lam, M. M. C., Wick, R. R., Watts, S. C., Cerdeira, L. T., Wyres, K. L., and Holt, K. E. (2021)), respectively.

Phage Methods: Isolation: Raw sewage collected from the Stickney Water Reclamation Plant in Chicago IL was centrifuged for 10,000 g for 10 minutes before filtering through a 0.22 μm filter. 300 μl of filtered sewage was mixed with 300 μl of overnight bacterial culture in 3 mL of 0.6% top agar, which was poured as an overlay on top of an LB plate. The resulting plaques were streak-purified three times on the same host lawn before being amplified into a working stock.

Amplification of Phages: The host bacteria were sub-cultured from an overnight culture and grown to mid-log phase (approximately 1-5×10⁸ CFU/mL or 1.5-2 hours at 37° C.). 10 isolated plaques were picked from the isolation plate if a new phage or 10-500 μl of phage stock (MOI range 0.1-10) were used to infect the growing culture at 37° C. After visible lysis (3-4.5 hours), the cultures were pelleted at 10,000 rpm for 10 minutes, and the resulting supernatant was passed through a 0.22 μm filter. If used for mouse experiments, 1M NaCl and 10% of the weight/volume of PEG-8000 was added to the supernatant and dissolved at 37° C. for 15 minutes before incubation at 4° C. overnight. The phage preparations were then pelleted at 10,000 rpm for 28 minutes, with the resulting pellet dissolved in PBS. Phages stocks were stored in the refrigerator.

For long-term storage of phages, a 1:100 sub-culture of the host bacteria was grown for 2 hours before infection with phage stock (MOI ˜0.01-0.5) for 15 minutes at room temperature. 700 μl of the infected cells was mixed with 300 μl 50% ice cold glycerol and kept on ice until transfer into a freezer box at −80° C.

Phage Electron Microscopy: High titer phage stocks in LB were provide to The University of Chicago Advanced Electron Microscopy Core, where they were strained with 1% uranyl acetate and visualized on either an FEI Spirit or FEI Tecnai F30 scanning transmission electron microscope.

Phage Genomic DNA Extraction: 500 μl of each phage prep greater than 1×10⁹ PFU/mL was transferred to an Eppendorf tube. To each tube, we added 6.25 μl of 1M MgCl2, 0.5 μl RNAse A ((100 mg/mL); Qiagen) and 0.5 μl Turbo DNAse I (Fisher Scientific) and incubated at 37° C. for 30 minutes, followed by 20 μl of 0.5M EDTA (Invitrogen), 2.5 μl Proteinase K (10 mg/mL; Qiagen), and 25 μl 10% SDS (ThermoFisher Scientific) at 55° C. for one hour. DNA was extracted using 550 μl phenol::chloroform::isoamyl alcohol (Thermo Scientific), with the aqueous phase then mixed with 550 μl chloroform and precipitated with twice the volume of cold 100% ethanol (Decon Laboratories) and 1/10 volume of 3M sodium acetate (Fisher Scientific) at pH 5.2, and washed with 70% ethanol. The DNA pellet was re-suspended in EB (10 mM Tris; Qiagen).

Phage Sequencing, Assembly, and Annotation: Phage whole genome sequencing was performed by SeqCenter/MiGS (Pittsburgh, PA) using 2×150 bp Illumina NovaSeq at approximately 200 Mbp per sample. Genomes were assembled using Velvet (Zerbino, D. R., and Birney, E. (2008)) de novo assembly in the Geneious Prime 2023.1, with 75 kmer and approximately 150,000 reads. The phylogenetic tree was generated by submitting whole genome FASTA files to VICTOR (Meier-Kolthoff, J. P., and Göker, M. (2017)). All phage genomes were annotated using Pharokka v0.1.9 (Bouras, G., Nepal, R., Houtak, G., Psaltis, A. J., Wormald, P.-J., and Vreugde, S. (2023)) initialized with default parameters. Pharokka performs coding sequence (CDS), tRNA, tmRNA, and CRISPR prediction using PHANOTATE (McNair, K., Zhou, C., Dinsdale, E. A., Souza, B., and Edwards, R. A. (2019)), tRNAscan-SE 2.0 (Chan, P. P., Lin, B. Y., Mak, A. J., and Lowe, T. M. (2021)), ARAGORN (Laslett, D., and Canback, B. (2004)), and CRT (Bland, C., Ramsey, T. L., Sabree, F., Lowe, M., Brown, K., Kyrpides, N.C., and Hugenholtz, P. (2007)), respectively. Pharokka utilizes MMseqs2 (Steinegger, M., and Söding, J. (2017)) to assign functional annotation by matching CDS to PHROGs (Terzian, P., Olo Ndela, E., Galiez, C., Lossouarn, J., Pérez Bucio, R. E., Mom, R., Toussaint, A., Petit, M.-A., and Enault, F. (2021)), VFDB (Chen, L., Yang, J., Yu, J., Yao, Z., Sun, L., Shen, Y., and Jin, Q. (2005)), and CARD (Alcock, B. P., Raphenya, A. R., Lau, T. T. Y., Tsang, K. K., Bouchard, M., Edalatmand, A., Huynh, W., Nguyen, A.-L. V., Cheng, A. A., Liu, S., et al. (2020)) databases and mash (Ondov, B. D., Treangen, T. J., Melsted, P., Mallonee, A. B., Bergman, N. H., Koren, S., and Phillippy, A. M. (2016)) indexing matched assembled contigs to their closest hit in the INPHARED database (Cook, R., Brown, N., Redgwell, T., Rihtman, B., Barnes, M., Clokie, M., Stekel, D. J., Hobman, J., Jones, M. A., and Millard, A. (2021)). Whole genome alignments were performed using Mauve progressive alignment (Darling, A. E., Mau, B., and Perna, N. T. (2010)).

Host Range Determination: The spot test is a semi-quantitative indicator of susceptibility, due to the high relative multiplicity of infection (MOI) and static nature of cells on the plate. The kill curve in liquid culture is more quantitative, since both the phage and cell titers are known, and allows for movement while the cultures grow. In both cases, phages infect growing cells and taken together, can indicate which phages are already good at killing, which cannot infect even at high titer, and which can infect weakly, but be improved upon.

For spot tests, lawns of bacteria were made by mixing 300 μl overnight culture, 300 μl 1×PBS, and 3 mL of 0.6% top agar as an overlay on LB bottom agar plates. 2.5 μl aliquots of high titer phage stock were dropped on the lawn and dried before overnight incubation at 37° C. Each phage was repeated three times and scored by eye on a scale of 0 (no lysis) to 3 (clear lysis zone). The average of each score was reported by color density with the darker the color representing the highest lysis. In general, phages showing weak lysis fail to robustly clear liquid cultures.

Kinetic Curves of Phage Killing: Bacterial cultures were diluted 1:100 and grown to mid-log phage, to an approximate titer of 1-2×10⁸ CFU/mL (between 1.5 and 2 hours at 37° C.). Concurrently a clear round-bottom 96-well dish (Corning) was filled with 20 μl of LB or phage stock, at an approximate MOI of 1 for 2.5 hour experiments and MOI of 10 for 16 hour experiments. When the cells reached the desired density, 180 μl was added directly to the wells and the plate was incubated at 37° C. in a TECAN Sunrise plate reader with shaking. OD₆₀₀ measurements were taken every 10 or 15 minutes for the duration of the experiment using Magellan software.

Time to Resistance Assays: 8 independent bacterial cultures at a time were diluted 1:100, grown to mid-log phase, and diluted to an approximate titer of 1-2×10⁷ CFU/mL. Phages were added at an approximate MOI of 10 and incubated for 16 hours in a TECAN Sunrise plate reader with heating and shaking. Bacteria growth curve values were analyzed in Microsoft Excel; the nadir of each curve was identified and the first time point where the optical density began to rise was counted as the time to resistance. Duplicate wells were inoculated and averaged for each overnight culture.

To generate a single value for the degree of bacterial suppression during phage infections, we used the Area Under the Curve (AUC) function in GraphPad Prism to compare the uninfected growth curve with phage treated cultures.

Adsorption Assays: Phage stocks were diluted to approximately 2.5×10⁷ PFU/mL in LB and distributed as 30 μl in Eppendorf tubes. Bacteria strains were sub-cultured 1:100 and grown for 2 hours at 37° C. The cultures were given streptomycin to halt growth and distributed by 300 μl to the tubes containing phage. One tube for each set of phages received 300 μl of LB as a control. The phages were allowed to adsorb to the cell surface at room temperature. At the indicated times, the tubes were centrifuged (1.5 minutes at 13.5K rpm), the supernatant was serially diluted, and the PFU was titered on a lawn of a susceptible host. The phage titers were normalized to the titer of the tube without cells. An exponential decay function (Y=Ae^{-Kx}+B)), where Y is free phage, A is free phage at the beginning of the experiment, and K is the decay constant, was used to fit the data using GraphPad Prism 9.5.1. The adsorption rate (mL min⁻¹ cell⁻¹) was calculated by dividing K (mL min⁻¹) with the cell concentration.

Identification of host factors: INSeq: Using an MH258 transposon library (Jung, H.-J., Littmann, E. R., Seok, R., Leiner, I. M., Taur, Y., Peled, J., van den Brink, M., Ling, L., Chen, L., Kreiswirth, B. N., et al. (2019)), bacteria were sub-cultured in LB with carbenicillin in triplicate and grew at 37° C. for 2 hours to ˜1×10⁷ CFU/mL before infecting with ΦER15 or ΦER39 at MOI of 1.5 and 30, respectively. Aliquots were taken from uninfected bacteria, after 3 hours, and after overnight incubation at 37° C. Genomic DNA was extracted using the Qiagen DNeasy Blood and Tissue kit, modified for bacteria, and the INSeq protocol (Goodman, A. L., McNulty, N. P., Zhao, Y., Leip, D., Mitra, R. D., Lozupone, C. a, Knight, R., and Gordon, J. I. (2009)) was followed. The 125 bp amplicons were sent to The University of Chicago Genomics Facility and run on a HiSeq 4000. Read analysis was performed using the pyinseq program, available on the world wide web at github.com/mjmLab/pyinseq.

Arbitrarily Primed POR: The MH258 transposon library was sub-cultured in LB with ofloxacin (to kill any E. coli present) and infected with ΦER12, ΦER15, ΦER16e, ΦER39 or ΦER46e. After overnight growth, the culture was streaked out on LB. Individual colonies were re-streaked and tested to see if they were resistant to the respective infecting phage by both plate reader assay and spot tested for clearing on a lawn. Genomic DNA was extracted from candidates that were resistant to the phage and amplified using degenerate primers with adapters (smd38/ARB1 and erD97/pSam-Kp2-1_out3 for round1; and smD40/ARB2 and erD98/pSam-Kp2-1_out4 for round 2 (github.com/mjmlab/protocols/blob/master/arbitrarily-primed-pcr.md)). PCR products were submitted for Sanger sequencing either at The University of Chicago DNA Sequencing & Genotyping Facility or at Genewiz.

Deletions by Double Cross-Over Homologous Recombination: For waaH and csrD, deletions were made by cloning a gfp cassette and 1 kb of flanking DNA upstream and downstream of the gene of interest, leaving the start codon and last six amino acids (Baba, T., Ara, T., Hasegawa, M., Takai, Y., Okumura, Y., Baba, M., Datsenko, K. A., Tomita, M., Wanner, B. L., and Mori, H. (2006)). We introduced the pieces into plasmid pMM903 before the sacB gene using Gibson Assembly (NEBuilder.com). The plasmids were introduced into conjugative E. coli strain S17.1 and mated into MH258 with approximately a 2:1 donor to recipient ratio of concentrated saturated culture in 25 μl spots on an LB plate at 37° C. for 3.5 hours. The mating spots were swabbed into 1 mL LB and plated onto LB streptomycin-ofloxacin plates. Fluorescent colonies were picked for re-streaking and the integrants were confirmed by PCR. Candidate colonies were then plated on LB low salt sucrose plates and screened for loss of fluorescence. A second PCR confirmed whether the excision of the plasmid resulted in the parental sequence or the deleted gene. Since non-specific integrants were frequent and it was difficult getting the correct integrant to excise, a homologous recombination-mediated genetic engineering approach was utilized.

Deletions by Lambda-Red Recombination: We modified pKD46 (Sharan, S. K., Thomason, L. C., Kuznetsov, S. G., and Court, D. L. (2009)) to express the aadA gene instead of bla and introduced it into MH258 by electroporation, selecting on LB streptomycin. We also modified pKD13 by replacing the kanamycin resistance cassette with tetracycline resistance. MH258 pKD46-aadA was sub-cultured and grown to mid-log phase at 30° C. The Lambda-Red genes on the plasmid were induced with L-arabinose for 10 minutes at 30° C. and 30 minutes at 37° C. before being washed with successively smaller volumes of 10% glycerol to make electrocompetent cells. The cells were electroporated with 0.5-1 μg of PCR product made with 90-mer primers amplifying the tetR gene on pKD13-tet. The primers contained approximately 60-bp of homology to the upstream and downstream regions of the gene of interest. After electroporation, the cells were recovered in 3 mL LB on the benchtop overnight. Transformants were selected on LB tetracycline plates at 37° C. and confirmed for the deletion-insertion by PCR. To eliminate the antibiotic cassette, the cells were made chemically competent with 2×TSS (20% PEG, 40 mM Mg2, 10% DMSO, given 5×KCM salts ((500 mM KCl (Alfa Aesar), 150 mM CaCl₂) (Acros Organics), 250 mM MgCl2 (Acros Organics)), and transformed with pFLP-hyg36 (Addgene). Colonies were selected on LB low salt hyg plates and confirmed for the excision of the antibiotic cassette by PCR. To lose the plasmid, the cells were streaked and inoculated into overnight cultures for several passages.

Complementation of Deletions Strains: To complement the deletions, plasmid pZE4D-GFP (Lutz, R., and Bujard, H. (1997)) had the GFP gene removed and replaced by the gene of choice. The vector was amplified using primers with overlapping regions of the insert using the default settings on the NEBuilder website. The insert DNA was amplified from chromosomal MH258 DNA extracted using the Qiagen DNeasy Blood and Tissue kit. A 3:1 molar ratio of insert to vector was combined with equal volume of 2× Gibson master mix (Gibson, D. G., Young, L., Chuang, R., Venter, J. C., Hutchison, C. A., and Smith, H. O. (2009)) (T5 exonuclease/Phusion polymerase/Taq ligase in ISO buffer) for 1 hour at 50° C. and a subset of the reaction was transformed into NEB10beta E. coli cells for selection and propagation. For two of the candidates (fhuA3 and tolB), vector pZara2 used the backbone of pZE4D-GFP, but replaced the promotor region with the ara C gene and pBAD promoter from plasmid pNR64 (Addgene). These plasmids did not end up needing arabinose for induction and were constitutively expressed.

RNA Sequencing Analysis of Acsrl: MH258 and MH258 ΔcsrD cultures were grown overnight, subcultured 1:100 in LB media, and grown to mid-log phase (approximately 2 hours) as measured by optical density (n=2). RNAProtect (Qiagen) was added to the cells per manufacturers protocols before RNA isolation. Total RNA was isolated from each cell type (in biological duplicate) and purified using RNAeasy RNA extraction kit (Qiagen). RNA was then DNAse treated (TURBO) to remove all DNA and submitted to the UChicago DFI Metagenomics Core for Illumina RNAseq Library Prep. Illumina compatible libraries were generated using NEBNext® Ultra™ II Directional RNA Library Prep Kit. Ribosomal RNA depletion (NEB) was used prior to library preparation. Libraries were sequenced using 2×150 bp reads on NextSeq 1000. After sequencing reads were filtered for quality (FastQC), they were aligned to the MH258 reference genome (Bowtie). Aligned reads counts were binned to reference annotations using featureCounts (Rsubread) and tested for differential expression (DESeq). Differential expression analysis directly compared the wild-type and mutant cell types, generating lists of significantly up- and down-regulated genes (adjusted p-value<0.05 and log 2 (fold change) either >2 or <−2). MH258 genomes were further annotated via PATRIC to add KEGG Pathway and SEED Subsystems. Enrichment analyses was performed using the GSEA( ) function from the clusterProfiler package to search for pathways and subsystems significantly enriched (P-adjusted <0.05) between the two cell types. All analysis was done in Rstudio and plots generated using the tidyverse R package.

Macrophage Phagocytosis Assay: Murine macrophage line RAW264.7 cells were propagated in DMEM without antibiotics and seeded in triplicate in 24-well plates (Thermo) to 5×10⁵ macrophages. Bacterial overnight cultures were pelleted, resuspended in 1×PBS, and diluted to approximately 1×10⁸ CFU/mL. Each macrophage well was given 10 μl of bacterial suspension (MOI=20), spun down at 380 g for 5 minutes, and incubated 2 hours at 37° C. with 5% CO₂. To remove extracellular bacteria, the cells were washed three times with 1×PBS, and treated with 300 μg/mL gentamicin for 30 minutes, followed by three 1×PBS washes and lysis of the macrophages with 0.2% Triton X-100 for 20 minutes at room temperature. A final volume of 1 mL 1×PBS was added to the wells and serial dilutions were performed to enumerate bacterial CFU on LB carbenicillin plates. For phage-treated cultures, the bacteria were sub-cultured 1:100 and grown at 37° C. to approximately 1×10⁸ CFU/mL. Cells were infected with an MOI of 1-2 of phage stocks and allowed to grow overnight to saturation.

Uronic Acid Assay: The uronic acid assay was adapted from previously established methods (Dorman, M. J., Feltwell, T., Goulding, D. A., Parkhill, J., and Short, F. L. (2018)). In brief, cultures were grown to saturation and measured for CFU/mL. 500 mL of culture was mixed with 100 uL capsule extraction buffer (1% Zwittergent 3-10 in 100 mM citric acid, pH 2.0) for 20 minutes at 50° C. The cells were then pelleted at 14,000 g for 5 minutes to remove debris. 250 μl of supernatant was removed twice to a new tube, where 1 mL cold 100% ethanol was added and the mixture allowed to precipitate on ice for 30 minutes before centrifugation (18,000 g at 4° C.). The supernatant was removed, the samples air dried, and resuspended in 200 μl sterile water. Capsule quantification was measured by mixing 100 μl of suspension with 0.6 mL tetraborate solution (12.5 mM disodium tetraborate in sulfuric acid), compared to a standard curve of glucuronic acid. Samples were incubated on ice for 10 minutes, heated at 95° C. for 5 minutes, and cooled on ice for 5 minutes. Sample absorbance was measured at 520 nM with 175 μl of sample before and after addition of 2.5 uL 0.15% 3-hydroxybiphenyl in 0.5% sodium hydroxide.

Directed Evolution of Bacteriophages: Phages were passaged over a period of seven days and ran in triplicate for each phage. On Day 0, a saturated culture of wildtype MH258 (for ΦER16) or MH258 overexpressing csrD (ΦER46) was diluted 1:30 in LB broth. 3 mL of this dilution was inoculated with approximately 10⁵ PFU of phage and incubated at 37° C. overnight. For Days 1-7, 1 mL of the phage-infected overnight was treated with 100 μL of chloroform, incubated at room temperature for 10 minutes, then centrifuged at 400×g for 5 minutes. The supernatant was saved at 4° C. to be used for further passaging and kill curves. The remaining 2 mL of overnight were passed through a 0.22 μm filter and stored at 4° C. for later use in sequencing. Then, a new overnight of MH258 host was diluted 1:30 and inoculated with 3 μL of the day's chloroform-treated phage lysate. These samples were incubated overnight at 37° C., with this process repeating until Day 7. The chloroform-treated isolates were serially diluted 1:10 in PBS and titered on LB-agar plates for every day of the protocol on 0.6% top agar lawns of ΦER26, a capsule mutant of MH258. ΦER16 and ΦER46 were plated on ΦER26 because they had been shown to form clearer plaques on this background. Plates were incubated overnight at 37° C. and titers counted the following day.

Antibiotic sensitivity: Overnight cultures were diluted 1:30 in PBS (approximately 1.5×10⁸ CFU/ml) and swabbed onto an LB plate. Antibiotic disks (Oxoid) containing the following antibiotics were firmly placed on top of the bacteria layer: Aztreonam—30 μg; Colistin—10 μg; Tigecycline—15 μg; Cefepime—30 μg; Tobramycin—10 μg; Amikacin—30 μg; Ciprofloxacin—5 μg; Piperacillin/Tazobactam—110 μg; Meropenem—10 μg; Imipenem—10 μg. After 18-20 hours incubation at 37° C., the diameter of the zone of clearing next to each disk was measured. For strains carrying a complementation plasmid, the cultures were swabbed on LB streptomycin plates to maintain plasmid selection. For phage-treated cultures, the bacteria were sub-cultured 1:100 and grown at 37° C. to approximately 1×10⁸ CFU/mL. The culture was then split, the cells were infected with an MOI of 1-2 of phage stocks, and allowed to grow overnight to saturation.

Mouse Decolonization Experiments: Gnotobiotic mice were bred and housed within the University of Chicago Gnotobiotic Research Animal Facility (GRAF) associated with a stable consortium of 14 microbes (see Table 2). Strains were selected out of an internal library of 65 human-derived bacterial species using a simple, unsupervised feature selection heuristic that maximizes functional metagenomic representation (KEGG, Metacyc, CAZymes) as compared to healthy human microbiome from the Integrated Human Microbiome Project (Lloyd-Price, J., Mahurkar, A., Rahnavard, G., Crabtree, J., Orvis, J., Hall, A. B., Brady, A., Creasy, H. H., McCracken, C., Giglio, M. G., et al. (2017)). This selection scheme resulted in a community that contains all major phyla in the gut microbiota and recapitulates ˜75% of the metabolic pathways found in the healthy human microbiome (Franzosa, E. A., Mclver, L. J., Rahnavard, G., Thompson, L. R., Schirmer, M., Weingart, G., Lipson, K. S., Knight, R., Caporaso, J. G., Segata, N., et al. (2018)). Each fully genome-sequenced, human-derived type strain was introduced into germ-free C57BL6/J (B6) mice to generate a colony with a known, stable microbiota (Akkermansia muciniphila, Alistipes finegoldii, Anaerostipes caccae, Bacteroides thetaiotaomicron, Bacteroides uniformis, Bifidobacterium adolescentis, Escherichia coli, Limosilactobacillus reuteri, Parabacteroides distasonis, Phocaeicola vulgatus, Roseburia inulinivorans, [Ruminoccocus] gnavus, Subdoligranulum variable, Turicibacter sanguinis). Community composition has remained stable for >3 years and is vertically transmitted to pups, as monitored by 16S rRNA sequencing of fecal samples with the aid of the UChicago Duchoissois Family Institute Microbiome Metagenomics Facility (DFI MMF). For 16S rRNA sequencing, DNA was extracted from fecal pellet with QIAamp PowerFecal Pro, V4-V5 16S was PCR amplified using barcoded dual-index primers, libraries were prepped with QIASeq 1-step amplicon library prep, and 2×250 PE MiSeq was used for sequencing at 10⁴ reads/sample. Resulting short reads were analyzed using dada2 and BLAST to local database.

Gnotobiotic C57BL/6 mice were bred and housed in Trexler-style flexible firm isolators (Class Biologically Clean) within Ancare polycarbonate mouse cages (N10HT). Gnotobiotic mice were weaned at 21 days of age and were fed an autoclaved, plant-based mouse chow (LabDiet JL Rat and Mouse/Auto 6F 5K67). All mice were housed in cages containing autoclaved Teklad Pine Shavings (Cat #7088) with a 12-hour light/dark cycle at a standard room temperature of 20-24° C. All mice were euthanized by CO₂ asphyxiation followed by cervical dislocation as a secondary measure. All experiments utilized both male and female mice. All experiments were performed in accordance with the Guide for the Care and Use of Laboratory Animals (8^{th} ed.) and were approved by the Institutional Animal Care and Use Committee of the University of Chicago (Protocol 72610).

8-12 week old C57BL/6 mice colonized with the defined consortia in the UChicago Gnotobiotic Animal Research Facility were given a gavage of 200 μl Klebsiella pneumoniae MH258 at 1-4×10⁸ CFU/mL in PBS on Day 0 and monitored by fecal collections for 15 days. Samples were sent for 16S sequencing at the UChicago DFI MMF and plated for CFU counts on LB carbenicillin plates at room temperature. On Days 7 and 8, mice were gavaged with 200 μl of either PBS or a 5-phage cocktail (ΦER12, ΦER15, ΦER16e, ΦER39, and ΦER46e at ˜10⁹ PFU/mouse). Cell titers were determined by resuspension of the fecal pellet in 1 mL PBS and shaken in a PowerLyzer 24 homogenizer (Qiagen) for 2 minutes at 2,000 rpm, followed by a quick 30 second spin at 300 g. Phage titer was determined by serial dilution of the homogenized fecal slurry supernatant after a 10-minute spin at 10,000 g. Phages were enumerated on differing lawns: both ΦER12 and ΦER39 formed plaques at high titer on MH258 and were further distinguished on a lawn of ΔwaaH, which prevented plaquing of ΦER39. Both ΦER15 and ΦER16e were counted on a non-capsulated ΔwaaH derivative (containing a truncated wzc gene) but were not able to be distinguished morphologically. PCR on plaques confirmed the presence of ΦER15 or ΦER16 and in different experiments, one predominated over the other. ΦER46e was enumerated on galU::IS, which prevented the other four phages from forming plaques. The capsulated status of a strain was visually determined by observation of colony morphology.

Data Analysis and Statistics: Statistical analyses were performed using GraphPad Prism version 10.0.

All of the methods disclosed and claimed herein can be made and executed without undue experimentation in light of the present disclosure. While the compositions and methods of this disclosure have been described in terms of certain aspects, it will be apparent to those of skill in the art that variations may be applied to the methods and in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the disclosure as defined by the appended claims.

### REFERENCES

The following references, to the extent that they provide exemplary procedural or other details supplementary to those set forth herein, are specifically incorporated herein by reference.
1. The Review on Antimicrobial Resistance (2016). Tackling Drug-Resistant Infections Globally: Final Report and Recommendations.
2. Services, U.S.D. of H. and Human (2019). Antibiotic resistance threats in the United States. Centers for Disease Control and Prevention, 1-113.
3. Murray, C. J. L., Ikuta, K. S., Sharara, F., Swetschinski, L., Aguilar, G. R., Gray, A., Han, C., Bisignano, C., Rao, P., Wool, E., et al. (2022). Global burden of bacterial antimicrobial resistance in 2019: a systematic analysis. The Lancet 399, 629-655. 10.1016/S0140-6736 (21) 02724-0.
4. Snitkin, E. S., Zelazny, a. M., Thomas, P. J., Stock, F., Henderson, D. K., Palmore, T. N., and Segre, J. a. (2012). Tracking a Hospital Outbreak of Carbapenem-Resistant Klebsiella pneumoniae with Whole-Genome Sequencing. Science Translational Medicine 4, 148ra116-148ra116. 10.1126/scitranslmed.3004129.
5. Keith, J. W., and Pamer, E. G. (2019). Enlisting commensal microbes to resist antibiotic-resistant pathogens. Journal of Experimental Medicine 216, 10-19. 10.1084/jem.20180399.
6. Federici, S., Kredo-Russo, S., Valdés-Mas, R., Kviatcovsky, D., Weinstock, E., Matiuhin, Y., Silberberg, Y., Atarashi, K., Furuichi, M., Oka, A., et al. (2022). Targeted suppression of human IBD-associated gut microbiota commensals by phage consortia for treatment of intestinal inflammation. Cell 185, 2879-2898.e24. 10.1016/j.cell.2022.07.003.
7. Taur, Y., Xavier, J. B., Lipuma, L., Ubeda, C., Goldberg, J., Gobourne, A., Lee, Y. J., Dubin, K. A., Socci, N. D., Viale, A., et al. (2012). Intestinal domination and the risk of bacteremia in patients undergoing allogeneic hematopoietic stem cell transplantation. Clinical Infectious Diseases 55, 905-914. 10.1093/cid/cis580.
8 Willems, R. P. J., van Dijk, K., Vehreschild, M. J. G. T., Biehl, L. M., Ket, J. C. F., RemmelzwaaH, S., and Vandenbroucke-Grauls, C. M. J. E. (2023). Incidence of infection with multidrug-resistant Gram-negative bacteria and vancomycin-resistant enterococci in carriers: a systematic review and meta-regression analysis. The Lancet Infectious Diseases. 10.1016/S1473-3099 (22) 00811-8.
9. Yuan, J., Chen, C., Cui, J., Lu, J., Yan, C., Wei, X., Zhao, X., Li, N. N., Li, S., Xue, G., et al. (2019). Fatty Liver Disease Caused by High-Alcohol-Producing Klebsiella pneumoniae. Cell Metabolism 30, 675-688.e7. 10.1016/j.cmet.2019.08.018.
10. Atarashi, K., Suda, W., Luo, C., Kawaguchi, T., Motoo, I., Narushima, S., Kiguchi, Y., Yasuma, K., Watanabe, E., Tanoue, T., et al. (2017). Ectopic colonization of oral bacteria in the intestine drives TH1 cell induction and inflammation. Science 358, 359-365. 10.1126/science.aan4526.
11. Hsu, B. B., Gibson, T. E., Yeliseyev, V., Liu, Q., Lyon, L., Bry, L., Silver, P. A., and Gerber, G. K. (2019). Dynamic Modulation of the Gut Microbiota and Metabolome by Bacteriophages in a Mouse Model. Cell Host & Microbe, 1-12. 10.1016/j.chom.2019.05.001.
12. Citorik, R. J., Mimee, M., and Lu, T. K. (2014). Sequence-specific antimicrobials using efficiently delivered RNA-guided nucleases. Nat Biotechnol, 1141-1145. 10.1038/nbt.3011.
13. Mimee, M., Citorik, R. J., and Lu, T. K. (2016). Microbiome therapeutics-Advances and challenges. Advanced Drug Delivery Reviews 105, 44-54. 10.1016/j.addr.2016.04.032.
14. d'Herelle, F. (1917). Sur un microbe invisible antagoniste des bacilles dysentériques. CR Acad. Sci. Paris 165, 373-375.
15. Chanishvili, N. (2012). Phage Therapy-History from Twort and d'Herelle Through Soviet Experience to Current Approaches 1st ed. (Elsevier Inc.) 10.1016/B978-0-12-394438-2.00001-3.
16. Onallah, H., Hazan, R., Nir-Paz, R., and Israeli Phage Therapy Center (IPTC) Study Team (2023). Compassionate Use of Bacteriophages for Failed Persistent Infections During the First 5 Years of the Israeli Phage Therapy Center. Open Forum Infectious Diseases 10, ofad221. 10.1093/ofid/ofad221.
17. Strathdee, S. A., Hatfull, G. F., Mutalik, V. K., and Schooley, R. T. (2023). Phage therapy: From biological mechanisms to future directions. Cell 186, 17-31. 10.1016/j.cell.2022.11.017.
18. Corbellino, M., Kieffer, N., Kutateladze, M., Balarjishvili, N., Leshkasheli, L., Askilashvili, L., Tsertsvadze, G., Rimoldi, S. G., Nizharadze, D., Hoyle, N., et al. (2020). Eradication of a multidrug-resistant, carbapenemase-producing Klebsiella pneumoniae isolate following oral and intra-rectal therapy with a custom made, lytic bacteriophage preparation. Clinical Infectious Diseases 70, 1998-2001. 10.1093/cid/ciz782.
19. Doub, J. B., Johnson, A. J., Nandi, S., Ng, V., Manson, T., Lee, M., and Chan, B. (2022). Experience Using Adjuvant Bacteriophage Therapy for the Treatment of 10 Recalcitrant Periprosthetic Joint Infections: A Case Series. Clinical Infectious Diseases, ciac694. 10.1093/cid/ciac694.
20. Cano, E. J., Caflisch, K. M., Bollyky, P. L., Van Belleghem, J. D., Patel, R., Fackler, J., Brownstein, M. J., Horne, B., Biswas, B., Henry, M., et al. (2021). Phage Therapy for Limb-threatening Prosthetic Knee Klebsiella pneumoniae Infection: Case Report and in Vitro Characterization of Anti-biofilm Activity. Clinical Infectious Diseases 73, E144-E151. 10.1093/cid/ciaa705.
21. Kuipers, S., Ruth, M. M., Mientjes, M., de Sévaux, R. G. L., and van Ingen, J. (2019). A Dutch Case Report of Successful Treatment of Chronic Relapsing Urinary Tract Infection with Bacteriophages in a Renal Transplant Patient. Antimicrobial Agents and Chemotherapy 64. 10.1128/AAC.01281-19.
22. Ichikawa, M., Nakamoto, N., Kredo-Russo, S., Weinstock, E., Weiner, I. N., Khabra, E., Ben-Ishai, N., Inbar, D., Kowalsman, N., Mordoch, R., et al. (2023). Bacteriophage therapy against pathological Klebsiella pneumoniae ameliorates the course of primary sclerosing cholangitis. Nat Commun 14, 3261. 10.1038/s41467-023-39029-9.
23. Nobrega, F. L., Vlot, M., de Jonge, P. A., Dreesens, L. L., Beaumont, H. J. E., Lavigne, R., Dutilh, B. E., and Brouns, S. J. J. (2018). Targeting mechanisms of tailed bacteriophages. Nature Reviews Microbiology 16, 760-773. 10.1038/s41579-018-0070-8.
24. Huiting, E., and Bondy-Denomy, J. (2023). Defining the expanding mechanisms of phage-mediated activation of bacterial immunity. Current Opinion in Microbiology 74, 102325. 10.1016/j.mib.2023.102325.
25. Labrie, S. J., Samson, J. E., and Moineau, S. (2010). Bacteriophage resistance mechanisms. Nature reviews. Microbiology 8, 317-327. 10.1038/nrmicro2315.
26. Jung, H.-J., Littmann, E. R., Seok, R., Leiner, I. M., Taur, Y., Peled, J., van den Brink, M., Ling, L., Chen, L., Kreiswirth, B. N., et al. (2019). Genome-Wide Screening for Enteric Colonization Factors in Carbapenem-Resistant ST258 Klebsiella pneumoniae. mBio 10, 1-15. 10.1128/mBio.02663-18.
27. Gurney, J., Brown, S. P., Kaltz, O., and Hochberg, M. E. (2020). Steering Phages to Combat Bacterial Pathogens. Trends in Microbiology 28, 85-94. 10.1016/j.tim.2019.10.007.
28. Oromi-Bosch, A., Antani, J. D., and Turner, P. E. (2023). Developing Phage Therapy That Overcomes the Evolution of Bacterial Resistance. Annual Review of Virology 10, null. 10.1146/annurev-virology-012423-110530.
29. Wyres, K. L., Lam, M. M. C., and Holt, K. E. (2020). Population genomics of Klebsiella pneumoniae. Nature Reviews Microbiology. 10.1038/s41579-019-0315-1.
30. Li, Y., and Bondy-Denomy, J. (2021). Anti-CRISPRs go viral: The infection biology of CRISPR-Cas inhibitors. Cell Host & Microbe 29, 704-714. 10.1016/j.chom.2020.12.007.
31. Goodman, A. L., McNulty, N. P., Zhao, Y., Leip, D., Mitra, R. D., Lozupone, C. a, Knight, R., and Gordon, J. I. (2009). Identifying genetic determinants needed to establish a human gut symbiont in its habitat. Cell host & microbe 6, 279-289. 10.1016/j.chom.2009.08.003.
32. Mutalik, V. K., Adler, B. A., Rishi, H. S., Piya, D., Zhong, C., Koskella, B., Kutter, E. M., Calendar, R., Novichkov, P. S., Price, M. N., et al. (2020). High-throughput mapping of the phage resistance landscape in E. coli. PLOS Biology 18, e3000877. 10.1371/journal.pbio.3000877.
33. Timmermans, J., and Van Melderen, L. (2009). Conditional Essentiality of the csrA Gene in Escherichia coli. Journal of Bacteriology 191, 1722-1724. 10.1128/jb.01573-08.
34. Klein, G., Müller-Loennies, S., Lindner, B., Kobylak, N., Brade, H., and Raina, S. (2013). Molecular and Structural Basis of Inner Core Lipopolysaccharide Alterations in Escherichia coli. J Biol Chem 288, 8111-8127. 10.1074/jbc.M112.445981.
35. van Aartsen, J. J., and Rajakumar, K. (2011). An optimized method for suicide vector-based allelic exchange in Klebsiella pneumoniae. Journal of Microbiological Methods 86, 313-319. 10.1016/j.mimet.2011.06.006.
36. Huang, T.-W., Lam, I., Chang, H.-Y., Tsai, S.-F., Palsson, B. O., and Charusanti, P. (2014). Capsule deletion via a λ-Red knockout system perturbs biofilm formation and fimbriae expression in Klebsiella pneumoniae MGH 78578. BMC Research Notes 7, 13. 10.1186/1756-0500-7-13.
37. Sharan, S. K., Thomason, L. C., Kuznetsov, S. G., and Court, D. L. (2009). Recombineering: a homologous recombination-based method of genetic engineering. Nat. Protocols 4, 206-223.
38. Baba, T., Ara, T., Hasegawa, M., Takai, Y., Okumura, Y., Baba, M., Datsenko, K. A., Tomita, M., Wanner, B. L., and Mori, H. (2006). Construction of Escherichia coli K-12 in-frame, single-gene knockout mutants: the Keio collection. Mol Syst Biol 2, 2006.0008. 10.1038/msb4100050.
39. Patro, L. P. P., and Rathinavelan, T. (2019). Targeting the Sugary Armor of Klebsiella Species. Frontiers in Cellular and Infection Microbiology 9. 10.3389/fcimb.2019.00367.
40. Tsai, Y.-K., Fung, C.-P., Lin, J.-C., Chen, J.-H., Chang, F.-Y., Chen, T.-L., and Siu, L. K. (2011). Klebsiella pneumoniae Outer Membrane Porins OmpK35 and OmpK36 Play Roles in both Antimicrobial Resistance and Virulence. Antimicrobial Agents and Chemotherapy 55, 1485-1493. 10.1128/aac.01275-10.
41. Yu, F., and Mizushima, S. (1982). Roles of lipopolysaccharide and outer membrane protein OmpC of Escherichia coli K-12 in the receptor function for bacteriophage T4. Journal of Bacteriology 151, 718-722. 10.1128/jb. 151.2.718-722.1982.
42. Clavel, T., Lazzaroni, J. C., Vianney, A., and Portalier, R. (1996). Expression of the tolQRA genes of Escherichia coli K-12 is controlled by the RcsC sensor protein involved in capsule synthesis. Molecular Microbiology 19, 19-25. 10.1046/j. 1365-2958.1996.343880.x.
43. Hirakawa, H., Suzue, K., and Tomita, H. (2022). Roles of the Tol/Pal System in Bacterial Pathogenesis and Its Application to Antibacterial Therapy. Vaccines 10, 422. 10.3390/vaccines 10030422.
44. Esquerré, T., Bouvier, M., Turlan, C., Carpousis, A. J., Girbal, L., and Cocaign-Bousquet, M. (2016). The Csr system regulates genome-wide mRNA stability and transcription and thus gene expression in Escherichia coli. Sci Rep 6, 25057. 10.1038/srep25057.
45. Mike, L. A., Stark, A. J., Forsyth, V. S., Vornhagen, J., Smith, S. N., Bachman, M. A., and Mobley, H. L. T. (2021). A systematic analysis of hypermucoviscosity and capsule reveals distinct and overlapping genes that impact Klebsiella pneumoniae fitness. PLOS Pathogens 17, e1009376. 10.1371/journal.ppat. 1009376.
46. Tal, N., Millman, A., Stokar-Avihail, A., Fedorenko, T., Leavitt, A., Melamed, S., Yirmiya, E., Avraham, C., Brandis, A., Mehlman, T., et al. (2022). Bacteria deplete deoxynucleotides to defend against bacteriophage infection. Nat Microbiol 7, 1200-1209. 10.1038/s41564-022-01158-0.
47. Hsueh, B. Y., Severin, G. B., Elg, C. A., Waldron, E. J., Kant, A., Wessel, A. J., Dover, J. A., Rhoades, C. R., Ridenhour, B. J., Parent, K. N., et al. (2022). Phage defence by deaminase-mediated depletion of deoxynucleotides in bacteria. Nat Microbiol 7, 1210-1220. 10.1038/s41564-022-01162-4.
48. Ernst, C. M., Braxton, J. R., Rodriguez-Osorio, C. A., Zagieboylo, A. P., Li, L., Pironti, A., Manson, A. L., Nair, A. V., Benson, M., Cummins, K., et al. (2020). Adaptive evolution of virulence and persistence in carbapenem-resistant Klebsiella pneumoniae. Nature Medicine 26, 705-711. 10.1038/s41591-020-0825-4.
49. Burmeister, A. R., Fortier, A., Roush, C., Lessing, A. J., Bender, R. G., Barahman, R., Grant, R., Chan, B. K., and Turner, P. E. (2020). Pleiotropy complicates a trade-off between phage resistance and antibiotic resistance. Proceedings of the National Academy of Sciences, 201919888. 10.1073/pnas. 1919888117.
50. Bulman, Z. P., Krapp, F., Pincus, N. B., Wenzler, E., Murphy, K. R., Qi, C., Ozer, E. A., and Hauser, A. R. (2021). Genomic Features Associated with the Degree of Phenotypic Resistance to Carbapenems in Carbapenem-Resistant Klebsiella pneumoniae. mSystems 6, 10.1128/msystems.00194-21. 10.1128/msystems.00194-21.
51. Borin, J. M., Avrani, S., Barrick, J. E., Petrie, K. L., and Meyer, J. R. (2021). Coevolutionary phage training leads to greater bacterial suppression and delays the evolution of phage resistance. Proceedings of the National Academy of Sciences of the United States of America 118, 1-10. 10.1073/pnas.2104592118.
52. Burrowes, B. H., Molineux, I. J., and Fralick, J. A. (2019). Directed in vitro evolution of therapeutic bacteriophages: The appelmans protocol. Viruses/1. 10.3390/v11030241.
53. Friman, V.-P., Soanes-Brown, D., Sierocinski, P., Molin, S., Johansen, H. K., Merabishvili, M., Pirnay, J.-P., De Vos, D., and Buckling, A. (2016). Pre-adapting parasitic phages to a pathogen leads to increased pathogen clearance and lowered resistance evolution with Pseudomonas aeruginosa cystic fibrosis bacterial isolates. Journal of Evolutionary Biology 29, 188-198. 10.1111/jeb. 12774.
54. Eskenazi, A., Lood, C., Wubbolts, J., Hites, M., Balarjishvili, N., Leshkasheli, L., Askilashvili, L., Kvachadze, L., van Noort, V., Wagemans, J., et al. (2022). Combination of pre-adapted bacteriophage therapy and antibiotics for treatment of fracture-related infection due to pandrug-resistant Klebsiella pneumoniae. Nature Communications 13, 302. 10.1038/s41467-021-27656-z.
55. Castledine, M., Padfield, D., Sierocinski, P., Soria Pascual, J., Hughes, A., Mäkinen, L., Friman, V.-P., Pirnay, J.-P., Merabishvili, M., de Vos, D., et al. (2022). Parallel evolution of Pseudomonas aeruginosa phage resistance and virulence loss in response to phage treatment in vivo and in vitro. eLife/1, e73679. 10.7554/eLife.73679.
56. Torres-Barceló, C., Turner, P. E., and Buckling, A. (2022). Mitigation of evolved bacterial resistance to phage therapy. Current Opinion in Virology 53, 101201. 10.1016/j.coviro.2022.101201.

57 Storms, Z. J., Teel, M. R., Mercurio, K., and Sauvageau, D. (2019). The Virulence Index: A Metric for Quantitative Analysis of Phage Virulence. Phage 1, 17-26. 10.1089/phage.2019.0001.
58. Sorbara, M. T., Dubin, K., Littmann, E. R., Moody, T. U., Fontana, E., Seok, R., Leiner, I. M., Taur, Y., Peled, J. U., van den Brink, M. R. M., et al. (2019). Inhibiting antibiotic-resistant Enterobacteriaceae by microbiota-mediated intracellular acidification. Journal of Experimental Medicine 216, 84-98. 10.1084/jem.20181639.
59. Pires, D. P., Cleto, S., Sillankorva, S., Azeredo, J., and Lu, T. K. (2016). Genetically Engineered Phages: a Review of Advances over the Last Decade. Microbiology and Molecular Biology Reviews 80, 523-543. 10.1128/mmbr.00069-15.
60. Ando, H., Lemire, S., Pires, D. P., and Lu, T. K. (2015). Engineering Modular Viral Scaffolds for Targeted Bacterial Population Editing. Cell Systems 1, 187-196. 10.1016/j.cels.2015.08.013.
61. Latka, A., Lemire, S., Grimon, D., Dams, D., Maciejewska, B., Lu, T., Drulis-Kawa, Z., and Briers, Y. (2021). Engineering the Modular Receptor-Binding Proteins of Klebsiella Phages Switches Their Capsule Serotype Specificity. mBio 12, 1-12. 10.1128/mBio.00455-21.
62. Yosef, I., Goren, M. G., Globus, R., Molshanski-Mor, S., and Qimron, U. (2017). Extending the Host Range of Bacteriophage Particles for DNA Transduction. Molecular Cell 66, 721-728.e3. 10.1016/j.molcel.2017.04.025.
63. Yehl, K., Lemire, S., Yang, A. C., Ando, H., Mimee, M., Torres, M. D. T., de la Fuente-Nunez, C., and Lu, T. K. (2019). Engineering Phage Host-Range and Suppressing Bacterial Resistance through Phage Tail Fiber Mutagenesis. Cell 179, 459-469.e9. 10.1016/j.cell.2019.09.015.
64. Huss, P., Meger, A., Leander, M., Nishikawa, K., and Raman, S. (2021). Mapping the functional landscape of the receptor binding domain of T7 bacteriophage by deep mutational scanning. eLife 10, e63775. 10.7554/eLife.63775.
65. Squeglia, F., Maciejewska, B., \\c atka, A., Ruggiero, A., Briers, Y., Drulis-Kawa, Z., and Berisio, R. (2020). Structural and Functional Studies of a Klebsiella Phage Capsule Depolymerase Tailspike: Mechanistic Insights into Capsular Degradation. Structure, 613-624. 10.1016/j.str.2020.04.015.
66. Pan, Y., Lin, T., Chen, C., Tsai, Y., Cheng, Y.-H., Chen, Y.-Y., Hsieh, P.-F., Lin, Y.-T., and Wang, J.-T. (2017). Klebsiella Phage ΦK64-1 Encodes Multiple Depolymerases for Multiple Host Capsular Types. Journal of Virology 91, 1-16. 10.1128/JVI.02457-16.
67. Romero, P. A., and Arnold, F. H. (2009). Exploring protein fitness landscapes by directed evolution. Nat Rev Mol Cell Biol 10, 866-876. 10.1038/nrm2805.
68. Hsu, B. B., Plant, I. N., Lyon, L., Anastassacos, F. M., Way, J. C., and Silver, P. A. (2020). In situ reprogramming of gut bacteria by oral delivery. Nat Commun 11, 5030. 10.1038/s41467-020-18614-2.
69. Lourenço, M., Chaffringeon, L., Lamy-Besnier, Q., Pédron, T., Campagne, P., Eberl, C., Bérard, M., Stecher, B., Debarbieux, L., and De Sordi, L. (2020). The Spatial Heterogeneity of the Gut Limits Predation and Fosters Coexistence of Bacteria and Bacteriophages. Cell Host & Microbe, 1-12. 10.1016/j.chom.2020.06.002.
70. Weiss, M., Denou, E., Bruttin, A., Serra-Moreno, R., Dillmann, M.-L., and Brüssow, H. (2009). In vivo replication of T4 and T7 bacteriophages in germ-free mice colonized with Escherichia coli. Virology 393, 16-23. 10.1016/j.virol.2009.07.020.
71. Baaziz, H., Baker, Z. R., Franklin, H. C., and Hsu, B. B. (2022). Rehabilitation of a misbehaving microbiome: phages for the remodeling of bacterial composition and function. iScience 25, 104146. 10.1016/j.isci.2022.104146.
72. Meier-Kolthoff, J. P., and Göker, M. (2017). VICTOR: genome-based phylogeny and classification of prokaryotic viruses. Bioinformatics 33, 3396-3404. 10.1093/bioinformatics/btx440.
73. Zerbino, D. R., and Birney, E. (2008). Velvet: Algorithms for de novo short read assembly using de Bruijn graphs. Genome Res. 18, 821-829. 10.1101/gr.074492.107.
74. Sánchez-Busó, L., Yeats, C. A., Taylor, B., Goater, R. J., Underwood, A., Abudahab, K., Argimón, S., Ma, K. C., Mortimer, T. D., Golparian, D., et al. (2021). A community-driven resource for genomic epidemiology and antimicrobial resistance prediction of Neisseria gonorrhoeae at Pathogenwatch. Genome Medicine 13, 61. 10.1186/s13073-021-00858-2.
75. Lam, M. M. C., Wick, R. R., Judd, L. M., Holt, K. E., and Wyres, K. L. (2022). Kaptive 2.0: updated capsule and lipopolysaccharide locus typing for the Klebsiella pneumoniae species complex. Microbial Genomics 8, 000800. 10.1099/mgen.0.000800.
76. Lam, M. M. C., Wick, R. R., Watts, S. C., Cerdeira, L. T., Wyres, K. L., and Holt, K. E. (2021). A genomic surveillance framework and genotyping tool for Klebsiella pneumoniae and its related species complex. Nat Commun 12, 4188. 10.1038/s41467-021-24448-3.
77. Bouras, G., Nepal, R., Houtak, G., Psaltis, A. J., Wormald, P.-J., and Vreugde, S. (2023). Pharokka: a fast scalable bacteriophage annotation tool. Bioinformatics 39, btac776. 10.1093/bioinformatics/btac776.
78. McNair, K., Zhou, C., Dinsdale, E. A., Souza, B., and Edwards, R. A. (2019). PHANOTATE: a novel approach to gene identification in phage genomes. Bioinformatics 35, 4537-4542. 10.1093/bioinformatics/btz265.
79. Chan, P. P., Lin, B. Y., Mak, A. J., and Lowe, T. M. (2021). tRNAscan-SE 2.0: improved detection and functional classification of transfer RNA genes. Nucleic Acids Research 49, 9077-9096. 10.1093/nar/gkab688.
80. Laslett, D., and Canback, B. (2004). ARAGORN, a program to detect tRNA genes and tmRNA genes in nucleotide sequences. Nucleic Acids Research 32, 11-16. 10.1093/nar/gkh152.
81. Bland, C., Ramsey, T. L., Sabree, F., Lowe, M., Brown, K., Kyrpides, N. C., and Hugenholtz, P. (2007). CRISPR Recognition Tool (CRT): a tool for automatic detection of clustered regularly interspaced palindromic repeats. BMC Bioinformatics 8, 209. 10.1186/1471-2105-8-209.
82. Steinegger, M., and Söding, J. (2017). MMseqs2 enables sensitive protein sequence searching for the analysis of massive data sets. Nat Biotechnol 35, 1026-1028. 10.1038/nbt.3988.
83. Terzian, P., Olo Ndela, E., Galiez, C., Lossouarn, J., Pérez Bucio, R. E., Mom, R., Toussaint, A., Petit, M.-A., and Enault, F. (2021). PHROG: families of prokaryotic virus proteins clustered using remote homology. NAR Genomics and Bioinformatics 3, lqab067. 10.1093/nargab/lqab067.
84. Chen, L., Yang, J., Yu, J., Yao, Z., Sun, L., Shen, Y., and Jin, Q. (2005). VFDB: a reference database for bacterial virulence factors. Nucleic Acids Research 33, D325-D328. 10.1093/nar/gki008.
85. Alcock, B. P., Raphenya, A. R., Lau, T. T. Y., Tsang, K. K., Bouchard, M., Edalatmand, A., Huynh, W., Nguyen, A.-L. V., Cheng, A. A., Liu, S., et al. (2020). CARD 2020: antibiotic resistome surveillance with the comprehensive antibiotic resistance database. Nucleic Acids Research 48, D517-D525. 10.1093/nar/gkz935.
86 Ondov, B. D., Treangen, T. J., Melsted, P., Mallonee, A. B., Bergman, N. H., Koren, S., and Phillippy, A. M. (2016). Mash: fast genome and metagenome distance estimation using MinHash. Genome Biology 17, 132. 10.1186/s13059-016-0997-x.
87. Cook, R., Brown, N., Redgwell, T., Rihtman, B., Barnes, M., Clokie, M., Stekel, D. J., Hobman, J., Jones, M. A., and Millard, A. (2021). INfrastructure for a PHAge REference Database: Identification of Large-Scale Biases in the Current Collection of Cultured Phage Genomes. PHAGE 2, 214-223. 10.1089/phage.2021.0007.
88. Darling, A. E., Mau, B., and Perna, N. T. (2010). Progressivemauve: Multiple genome alignment with gene gain, loss and rearrangement. PLOS ONE 5. 10.1371/journal.pone.0011147.
89. Lutz, R., and Bujard, H. (1997). Independent and tight regulation of transcriptional units in Escherichia coli via the LacR/O, the TetR/O and AraC/11-12 regulatory elements. Nucleic acids research 25, 1203-1210.
90. Gibson, D. G., Young, L., Chuang, R., Venter, J. C., Hutchison, C. A., and Smith, H. O. (2009). Enzymatic assembly of DNA molecules up to several hundred kilobases. Nature methods 6, 343-345. 10.1038/nmeth.1318.
91. Dorman, M. J., Feltwell, T., Goulding, D. A., Parkhill, J., and Short, F. L. (2018). The capsule regulatory network of Klebsiella pneumoniae defined by density-traDISort. mBio 9. 10.1128/mBio.01863-18.
92. Lloyd-Price, J., Mahurkar, A., Rahnavard, G., Crabtree, J., Orvis, J., Hall, A. B., Brady, A., Creasy, H. H., McCracken, C., Giglio, M. G., et al. (2017). Strains, functions and dynamics in the expanded Human Microbiome Project. Nature. 10.1038/nature23889.
93. Franzosa, E. A., McIver, L. J., Rahnavard, G., Thompson, L. R., Schirmer, M., Weingart, G., Lipson, K. S., Knight, R., Caporaso, J. G., Segata, N., et al. (2018). Species-level functional profiling of metagenomes and metatranscriptomes. Nature Methods 15, 962-968. 10.1038/s41592-018-0176-y.
94. Callahan, B. J., McMurdie, P. J., Rosen, M. J., Han, A. W., Johnson, A. J. A., and Holmes, S. P. (2016). DADA2: High-resolution sample inference from Illumina amplicon data. Nat Methods 13, 581-583. 10.1038/nmeth.3869.

1-153. (canceled)

154. A composition comprising a collection of isolated bacteriophage comprising:
i. an Ackermannviridae bacteriophage, a Tevenvirinae bacteriophage, a Slopekvirus bacteriophage, a Siphoviridae bacteriophage, and/or a Demerecviridae bacteriophage;
ii. an Autographiviridae bacteriophage, a Tevenvirinae bacteriophage, one or more Slopekvirus bacteriophage, and/or a Purpuraviridae bacteriophage; or
iii. an Autographiviridae bacteriophage, a Tevenvirinae bacteriophage, one or more Slopekvirus bacteriophage, a Demerecviridae bacteriophage, and/or a Purpuraviridae bacteriophage.

155. The composition of claim 154, wherein:
for the collection of isolated bacteriophage of (i):
the Ackermannviridae bacteriophage is an isolated bacteriophage comprising a bacteriophage sequence that is at least 99% identical to SEQ ID NO:1 or SEQ ID NO:37;
the Tevenvirinae bacteriophage is an isolated bacteriophage comprising a bacteriophage sequence that is at least 99% identical to SEQ ID NO:2 or SEQ ID NO:40;
the Slopekvirus bacteriophage is an isolated bacteriophage comprising a bacteriophage sequence that is at least 99% identical to SEQ ID NO:3 or SEQ ID NO:41;
the Siphoviridae bacteriophage is an isolated bacteriophage comprising a bacteriophage sequence that is at least 99% identical to SEQ ID NO:4 or SEQ ID NO:52; and
the Demerecviridae bacteriophage is an isolated bacteriophage comprising a bacteriophage sequence that is at least 99% identical to SEQ ID NO:5 or SEQ ID NO:56;
for the collection of isolated bacteriophage of (ii):
the Autographiviridae bacteriophage is an isolated bacteriophage comprising a bacteriophage sequence that is at least 99% identical to SEQ ID NO:74;
the Tevenvirinae bacteriophage is an isolated bacteriophage comprising a bacteriophage sequence that is at least 99% identical to SEQ ID NO:14 or SEQ ID NO:36;
the one or more Slopekvirus bacteriophage comprise two Slopekvirus bacteriophage, and the Slopekvirus are isolated bacteriophage comprising a bacteriophage sequence that is at least 99% identical to SEQ ID NO:20 or SEQ ID NO:47 and SEQ ID NO:22 or SEQ ID NO:49; and
the Purpuraviridae bacteriophage is an isolated bacteriophage comprising a bacteriophage sequence that is at least 99% identical to SEQ ID NO:8 or SEQ ID NO:32; and
for the collection of isolated bacteriophage of (iii):
the Autographiviridae bacteriophage is an isolated bacteriophage comprising a bacteriophage sequence that is at least 99% identical to SEQ ID NO:25 or SEQ ID NO:53;
the Tevenvirinae bacteriophage is an isolated bacteriophage comprising a bacteriophage sequence that is at least 99% identical to SEQ ID NO:2 or SEQ ID NO:40;
the Slopekvirus bacteriophage is an isolated bacteriophage comprising a bacteriophage sequence that is at least 99% identical to SEQ ID NO:20 or SEQ ID NO:47;
the Demerecviridae bacteriophage is an isolated bacteriophage comprising a bacteriophage sequence that is at least 99% identical to SEQ ID NO:5 or SEQ ID NO:46; and
the Purpuraviridae bacteriophage is an isolated bacteriophage comprising a bacteriophage sequence that is at least 99% identical to SEQ ID NO:72.

156. The composition of claim 154, wherein:
for the collection of isolated bacteriophage of (i):
the Ackermannviridae bacteriophage is ΦER12;
the Tevenvirinae bacteriophage is ΦER15;
the Slopekvirus bacteriophage is ΦER16;
the Siphoviridae bacteriophage is ΦER39; and
the Demerecviridae bacteriophage is ΦER46;
for the collection of isolated bacteriophage of (ii):
the Autographiviridae bacteriophage is ΦMM9;
the Tevenvirinae bacteriophage is ΦER11;
the one or more Slopekvirus bacteriophage comprise two Slopekvirus, and the two Slopekvirus are ΦER27 and ΦER36; and
the Purpuraviridae bacteriophage is ΦER3; and
for the collection of isolated bacteriophage of (iii):
the Autographiviridae bacteriophage is ΦER43;
the Tevenvirinae bacteriophage is ΦER15;
the Slopekvirus bacteriophage is ΦER27;
the Demerecviridae bacteriophage is ΦER46; and
the Purpuraviridae bacteriophage is ΦMM7.

157. A composition comprising at least one isolated bacteriophage comprising a bacteriophage sequence that is at least 99% identical to any one of SEQ ID NO:1-74.

158. The composition of claim 157, wherein the composition comprises no more than one, two, three, four, or five isolated bacteriophage.

159. The composition of claim 157, wherein the one, two, three, four, or five different isolated bacteriophage each comprises a bacteriophage sequence that is at least 99.1, 99.2%, 99.3%, 99.4%, 99.5%, 99.6%, 99.7%, 99.8%, or 99.9% identical to any one of SEQ ID NO: 1-74.

160. The composition of claim 157, wherein the one, two, three, four, or five different isolated bacteriophage each comprises a bacteriophage sequence that is at least 99.1, 99.2%, 99.3%, 99.4%, 99.5%, 99.6%, 99.7%, 99.8%, 99.9%, or 99.95% identical to or comprises any one of:
SEQ ID NO:1 or SEQ ID NO:37; SEQ ID NO:2 or SEQ ID NO:40; SEQ ID NO:3 or SEQ ID NO: 41; SEQ ID NO:4 or SEQ ID NO:52; or SEQ ID NO:5 or SEQ ID NO:56;
SEQ ID NO:8 or SEQ ID NO:32, SEQ ID NO: 14 or SEQ ID NO:36, SEQ ID NO:20 or SEQ ID NO:47, SEQ ID NO:22 or SEQ ID NO:49, or SEQ ID NO: 74; or
SEQ ID NO:2 or SEQ ID NO:40; SEQ ID NO:20 or SEQ ID NO:47; SEQ ID NO:25 or SEQ ID NO:53; SEQ ID NO:5 or SEQ ID NO:56; or SEQ ID NO:72.

161. The composition of claim 157, wherein the one, two, three, four, or five different isolated bacteriophage each comprises a bacteriophage sequence with no more than 20 nucleotide changes in any one of SEQ ID NO: 1-74.

162. The composition of claim 157, wherein the isolated bacteriophage further comprises a heterologous sequence comprising a peptide or polypeptide.

163. The composition of claim 157, wherein the composition comprises about 10⁸ to about 10¹² plaque forming units (PFU) per milliliter (PFU/ml) of isolated bacteriophages.

164. The composition of claim 157, further comprising a pharmaceutically acceptable carrier or an antibiotic drug.

165. A method of increasing the susceptibly of a drug-resistant and/or pathogenic Klebsiella strain in a subject to clearance by the subject's immune system or to conventional antibiotic treatment, the method comprising the step of administering to the subject a therapeutically effective amount of the composition of claim 157.

166. A method of reducing the level or virulence of a drug-resistant and/or pathogenic Klebsiella strain or reducing the number of capsulated Klebsiella in a subject in a subject, comprising the step of administering to the subject a therapeutically effective amount of the composition of claim 157.

167. A method for treating and/or preventing a Klebsiella infection in a subject comprising administering to the subject a composition comprising at least one isolated bacteriophage comprising a bacteriophage sequence that is at least 99% identical to any one of SEQ ID NO: 1-74.

168. The method of claim 167, wherein the Klebsiella is drug-resistant, hypervirulent, and/or from Klebsiella pneumoniae, Klebsiella quasipneumoniae, and/or Klebsiella variicola.

169. The method of claim 167, wherein the subject has symptoms of a Klebsiella infection, is at risk for a Klebsiella infection, or has been determined to be infected with Klebsiella.

170. The method of claim 167, wherein the infection is an infection of the urinary tract, blood, gut, abdomen, stomach, lungs, skin, ear, eye, nose, oral, kidney, prostate, bladder, brain, vaginal tract, heart, liver, spleen, tendons, or a wound, or a combination thereof.

171. The method of claim 167, wherein the subject is immunosuppressed or has an immune cell defect, asplenia, impaired splenic function, nephrotic syndrome, or an autoimmune condition.

172. A method of producing the composition of claim 157 comprising infecting bacteria with an isolated bacteriophage comprising a bacteriophage sequence that is at least 99% identical to any one of SEQ ID NO:1-74; culturing the bacteria under conditions to allow bacteriophage to replicate; and isolating bacteriophage from the bacteria culture.

173. A device, comprising, on, in, and/or around the device, the composition of claim 157.
